SEQUENCE LISTING (46 sequences)

SEQ ID NO: 1 - protein, 112 aa, Artificial Sequence
  Description of Artificial Sequence Synthetic polypeptide
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIK

SEQ ID NO: 2 - protein, 17 aa, Artificial Sequence
  Description of Artificial Sequence Synthetic peptide
KSSQSLLYSTNQKNYLA

SEQ ID NO: 3 - protein, 7 aa, Artificial Sequence
  Description of Artificial Sequence Synthetic peptide
WASTRES

SEQ ID NO: 4 - protein, 8 aa, Artificial Sequence
  Description of Artificial Sequence Synthetic peptide
QQYYSYRT

SEQ ID NO: 5 - protein, 122 aa, Artificial Sequence
  Description of Artificial Sequence Synthetic polypeptide
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSS

SEQ ID NO: 6 - protein, 10 aa, Artificial Sequence
  Description of Artificial Sequence Synthetic peptide
GYTFTSYVIH

SEQ ID NO: 7 - protein, 17 aa, Artificial Sequence
  Description of Artificial Sequence Synthetic peptide
YINPYNDGTDYDEKFKG

SEQ ID NO: 8 - protein, 13 aa, Artificial Sequence
  Description of Artificial Sequence Synthetic peptide
EKDNYATGAWFAY

SEQ ID NO: 9 - DNA, 393 bp, Artificial Sequence
  Description of Artificial Sequence Synthetic polynucleotide
atggaatggagcggggtctttatctttctcctgtcagtaactgcaggtgtccactccgacatcgtgatgacccagtcccctgactccctggctgtgtccctgggcgaacgggtcaccatgaactgcaaatcttcccagtccctgctgtactccaccaaccagaagaactacctggcttggtaccagcagaaacctggccagtcccccaaactgctcatctactgggcttccaccagggaaagcggcgtgcctgacagattctccggaagcggcagcggaaccgacttcaccctgaccatctccagcgtgcaggctgaagacgtggctgtctactactgccagcagtactacagctacaggaccttcggcggaggcaccaagctggagatcaag

SEQ ID NO: 10 - DNA, 423 bp, Artificial Sequence
  Description of Artificial Sequence Synthetic polynucleotide
atggaatggagcggggtctttatctttctcctgtcagtaactgcaggtgtccactcccaggtgcaactgcaacagtccggacccgaagtcgtgaaaccaggagcttccgtgaagatgagctgcaaagcatccggatacaccttcaccagctacgtgatccactgggtgaggcagaaacctggccagggcctggactggatcggctacatcaacccttacaacgacggaaccgactacgacgagaaattcaaaggcaaagctaccctgaccagcgacaccagcacctccactgcttacatggagctgtccagcctgaggtccgaagacaccgctgtgtactactgcgctagggagaaggacaactacgctaccggcgcttggttcgcctactggggccagggaaccctggtgaccgtgtccagc

SEQ ID NO: 11 - protein, 567 aa, Unknown
  Description of Unknown Transforming growth factor beta receptor type II sequence
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 12 - protein, 592 aa, Unknown
  Description of Unknown Transforming growth factor beta receptor type IIB sequence
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 13 - DNA, 1701 bp, Unknown
  Description of Unknown Nucleic acid sequence encoding TGF-betaRII
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaa

SEQ ID NO: 14 - DNA, 1776 bp, Unknown
  Description of Unknown Nucleic acid sequence encoding TGF-betaRIIB
atgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaa

SEQ ID NO: 15 - protein, 137 aa, Unknown
  Description of Unknown TGF-betaRII extracellular domain that binds TGF-beta
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 16 - protein, 162 aa, Unknown
  Description of Unknown TGF-betaRIIB extracellular domain that binds TGF-beta
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 17 - protein, 101 aa, Unknown
  Description of Unknown TGF-betaRII or TGF-betaRIIB minimal extracellular domain that binds TGF-beta
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIF

SEQ ID NO: 18 - DNA, 411 bp, Unknown
  Description of Unknown Nucleic acid sequence encoding TGF-betaRII extracellular domain that binds TGF-beta
acgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgac

SEQ ID NO: 19 - DNA, 486 bp, Unknown
  Description of Unknown Nucleic acid sequence encoding TGF-betaRIIB extracellular domain that binds TGF-beta
acgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgac

SEQ ID NO: 20 - DNA, 303 bp, Unknown
  Description of Unknown Nucleic acid sequence encoding TGF-betaRII or TGF-betaRIIB minimal extracellular domain that binds TGF-beta
caactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttc

SEQ ID NO: 21 - DNA, 411 bp, Artificial Sequence
  Description of Artificial Sequence Synthetic polynucleotide
accatcccacctcacgttcagaagtctgtgaataacgacatgatcgtcactgacaacaacggtgcagtcaagtttccacaactgtgtaaattctgtgatgtgagattttccacctgtgacaaccagaaatcctgcatgagcaactgcagcatcacctccatctgtgagaagccacaggaagtctgtgtggctgtgtggagaaagaatgacgagaacatcacactggagacagtgtgccatgaccccaagctcccctaccatgactttattctggaagatgctgcttctccaaagtgcattatgaaggaaaagaagaagcctggcgagactttcttcatgtgttcctgtagctctgatgagtgcaatgacaacatcatcttctccgaagaatataacaccagcaatcctgac

SEQ ID NO: 22 - DNA, 486 bp, Artificial Sequence
  Description of Artificial Sequence Synthetic polynucleotide
accatcccacctcacgttcagaagtctgatgtggaaatggaggcccagaaagatgaaatcatctgccccagctgtaataggactgcccatccactgagacatattaataacgacatgatcgtcactgacaacaacggtgcagtcaagtttccacaactgtgtaaattctgtgatgtgagattttccacctgtgacaaccagaaatcctgcatgagcaactgcagcatcacctccatctgtgagaagccacaggaagtctgtgtggctgtgtggagaaagaatgacgagaacatcacactggagacagtgtgccatgaccccaagctcccctaccatgactttattctggaagatgctgcttctccaaagtgcattatgaaggaaaagaagaagcctggcgagactttcttcatgtgttcctgtagctctgatgagtgcaatgacaacatcatcttctccgaagaatataacaccagcaatcctgac

SEQ ID NO: 23 - DNA, 303 bp, Artificial Sequence
  Description of Artificial Sequence Synthetic polynucleotide
caactgtgtaaattctgtgatgtgagattttccacctgtgacaaccagaaatcctgcatgagcaactgcagcatcacctccatctgtgagaagccacaggaagtctgtgtggctgtgtggagaaagaatgacgagaacatcacactggagacagtgtgccatgaccccaagctcccctaccatgactttattctggaagatgctgcttctccaaagtgcattatgaaggaaaagaagaagcctggcgagactttcttcatgtgttcctgtagctctgatgagtgcaatgacaacatcatcttc

SEQ ID NO: 24 - protein, 448 aa, Artificial Sequence
  Description of Artificial Sequence Synthetic polypeptide
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 25 - protein, 448 aa, Artificial Sequence
  Description of Artificial Sequence Synthetic polypeptide
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 26 - protein, 451 aa, Artificial Sequence
  Description of Artificial Sequence Synthetic polypeptide
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 219 aa, Artificial Sequence
  Description of Artificial Sequence Synthetic polypeptide
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 29 - protein, 330 aa, Homo sapiens [sequence omitted]

SEQ ID NO: 30 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 31 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 32 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 33 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 35 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 36 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 529 aa, Artificial Sequence
  Description of Artificial Sequence Synthetic polypeptide
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARL

SEQ ID NO: 38 - protein, 554 aa, Artificial Sequence
  Description of Artificial Sequence Synthetic polypeptide
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARL

SEQ ID NO: 39 - protein, 199 aa, Artificial Sequence
  Description of Artificial Sequence Synthetic polypeptide
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 40 - protein, 224 aa, Artificial Sequence
  Description of Artificial Sequence Synthetic polypeptide
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 41 - protein, 507 aa, Artificial Sequence
  Description of Artificial Sequence Synthetic polypeptide
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVRIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVRELEHLDRLSGRSCSEEKIPEDGSLNTTK

SEQ ID NO: 42 - protein, 550 aa, Artificial Sequence
  Description of Artificial Sequence Synthetic polypeptide
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDASGIQMVCETLTECWDHDPEARLTAQCVAERFSELEHLDRLSGRSCSEEKIPEDGSLNTTK

SEQ ID NO: 43 - protein, 150 aa, Artificial Sequence
  Description of Artificial Sequence Synthetic polypeptide
  MISC_FEATURE (1)..(150): This sequence may encompass 1-30 “Gly Gly Gly Gly Ser” repeating units
  See specification as filed for detailed description of substitutions and preferred embodiments
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 44 - protein, 5 aa, Artificial Sequence
  Description of Artificial Sequence Synthetic peptide
GGGGS

SEQ ID NO: 45 - protein, 6 aa, Artificial Sequence
  Description of Artificial Sequence Synthetic 6xHis tag
HHHHHH

SEQ ID NO: 46 - protein, 12 aa, Artificial Sequence
  Description of Artificial Sequence Synthetic peptide
GGGSGGGSGGGS

(19) United States
(12) Patent Application Publication
(10) Pub. No.: US 2022/0267442 A1
(43) Pub. Date: Aug. 25, 2022

(54) METHODS AND COMPOSITIONS FOR TARGETING TGF-ß SIGNALING IN CD4+ HELPER T CELLS FOR CANCER IMMUNOTHERAPY

(71) Applicant: Memorial Sloan Kettering Cancer Center, New York, NY (US)

(72) Inventors: Ming Li, New York, NY (US); Shun Li, New York, NY (US); Ming Liu, New York, NY (US)

(21) Appl. No.: 17/626,941

(22) PCT Filed: Jul. 17, 2020
(86) PCT No.: PCT/US2020/042517

§ 371 Date: Jan. 13, 2022

Related U.S. Application Data
(60) Provisional application No. 62/875,778, filed on Jul. 18, 2019.

(51) Int. Cl. C07K 16/28; C07K 14/71; A61P 35/00; A61K 45/06; A61K 38/17; A61K 39/395; C07K 16/24

(52) U.S. Cl. CPC C07K 16/2812; C07K 14/71; A61P 35/00; A61K 45/06; A61K 38/179; A61K 2039/505; C07K 16/247; C07K 16/249; C07K 2319/30; C07K 2317/565; C07K 2317/24; A61K 39/39558

(57) ABSTRACT

The present disclosure provides fusion proteins that specifically inhibit transforming growth factor-β (TGF-β) signaling in CD4+ helper T cells, and engineered CD4+ helper T cells that are deficient in TGF-β signaling, to counteract tumor-induced immune tolerance and promote anti-tumor immunity. The fusion proteins and engineered CD4+ helper T cells of the present technology are useful in methods for treating cancer, and enhancing the efficacy of other therapeutic agents against refractory cancer cells.

### CROSS-REFERENCE OF RELATED APPLICATIONS

This application is a U.S. National Stage Application under 35 U.S.C. § 371 of International Patent Application No. PCT/US2020/042517, filed on Jul. 17, 2020, which claims the benefit of and priority to U.S. Provisional Patent Application No. 62/875,778, filed Jul. 18, 2019, the entire contents of which is incorporated herein by reference.

### SEQUENCE LISTING

The instant application contains a Sequence Listing which has been submitted electronically in ASCII format and is hereby incorporated by reference in its entirety. Said ASCII copy, created on Aug. 7, 2020, is named 115872-0930_SL.txt and is 94,270 bytes in size.

### TECHNICAL FIELD

The present disclosure provides fusion proteins that specifically inhibit transforming growth factor-β (TGF-β) signaling in CD4⁺ helper T cells, and engineered CD4⁺ helper T cells that are deficient in TGF-β signaling, to counteract tumor-induced immune tolerance and promote anti-tumor immunity. The fusion proteins and engineered CD4⁺ helper T cells of the present technology are useful in methods for treating cancer, and enhancing the efficacy of existing therapeutic agents against refractory cancer cells.

### BACKGROUND

The following description of the background of the present technology is provided simply as an aid in understanding the present technology and is not admitted to describe or constitute prior art to the present technology.

Compared to surgery, radiation, and chemotherapy, immunotherapy is based on targeting the immune system rather than a tumor itself for cancer treatment. Indeed, immune checkpoint blockade therapies targeting the inhibitory receptors CTLA-4 and PD-1 have revolutionized cancer patient care, with long-term cancer remission observed in some patients. Sharma, P. & Allison, J. P. Science 348, 56-61 (2015); Callahan, M. K., Postow, M. A. & Wolchok, J. D. Immunity 44, 1069-1078 (2016). CTLA-4 is constitutively expressed on the immunosuppressive regulatory T (Treg) cells, and represses T cell responses by competing with the co-stimulatory receptor CD28 for ligand binding. PD-1 is expressed predominantly on CD8⁺ cytotoxic T lymphocytes following T cell receptor stimulation, and promotes T cell exhaustion in part by inhibiting CD28 signaling. Despite the remarkable clinical success of anti-PD-1 and anti-CTLA-4, many cancer patients fail to respond to these drugs, thereby demonstrating the need for identifying additional therapeutic interventions to counteract tumor-induced T cell tolerance.

### SUMMARY OF THE PRESENT TECHNOLOGY

In one aspect, the present disclosure provides a fusion protein comprising a CD4 targeting moiety fused with an immunomodulatory moiety, wherein: the CD4 targeting moiety comprises a heavy chain immunoglobulin variable domain (V_{H}) and a light chain immunoglobulin variable domain (V_{L}), wherein: (a) the V_{H} comprises a V_{H}-CDR1 sequence of GYTFTSYVIH (SEQ ID NO: 6), a V_{H}-CDR2 sequence of YINPYNDGTDYDEKFKG (SEQ ID NO: 7), and a V_{H}-CDR3 sequence of EKDNYATGAWFAY (SEQ ID NO: 8), and (b) the V_{L} comprises a V_{L}-CDR1 sequence of KSSQSLLYSTNQKNYLA (SEQ ID NO: 2), a V_{L}-CDR2 sequence of WASTRES (SEQ ID NO: 3), and a V_{L}-CDR3 sequence of QQYYSYRT (SEQ ID NO: 4); and the immunomodulatory moiety comprises an amino acid sequence of a TGF-β receptor II (TGF-βRII) selected from the group consisting of SEQ ID NOs: 11-12 and 15-17. In some embodiments, the TGF-β receptor II is encoded by a nucleic acid sequence selected from the group consisting of SEQ ID NOs: 13-14, 18-20, and 21-23. The immunomodulatory moiety may be fused to the C-terminus or the N-terminus of the CD4 targeting moiety. The immunomodulatory moiety may be fused to the CD4 targeting moiety directly, or via a linker. In some embodiments of the fusion protein of the present technology, the V_{H} comprises an amino acid sequence that is at least 80%, at least 85%, at least 95%, or 100% identical to SEQ ID NO: 5; and/or the V_{L} comprises an amino acid sequence that is at least 80%, at least 85%, at least 95%, or 100% identical to SEQ ID NO: 1.

Additionally or alternatively, in some embodiments of the fusion protein of the present technology, the CD4 targeting moiety comprises an antibody or an antigen binding fragment that specifically binds CD4. In some embodiments, the antibody of the CD4 targeting moiety comprises a heavy chain (HC) and a light chain (LC). Additionally or alternatively, in some embodiments, the immunomodulatory moiety is fused to the N-terminus or C-terminus of the antibody HC of the CD4 targeting moiety. Additionally or alternatively, in some embodiments, the immunomodulatory moiety is fused to the N-terminus or C-terminus of the antibody LC of the CD4 targeting moiety. In certain embodiments, the immunomodulatory moiety is fused to the N-terminus of the antibody HC and the N-terminus of the antibody LC of the CD4 targeting moiety. In other embodiments, the immunomodulatory moiety is fused to the C-terminus of the antibody HC and the C-terminus of the antibody LC of the CD4 targeting moiety.

Additionally or alternatively, in some embodiments, the fusion protein may be represented by the formula X-Fc-Y, or X-Z-Y, wherein X is the CD4 targeting moiety, Fc is an immunoglobulin Fc domain, Y is the immunomodulatory moiety, and Z is a linker sequence. In other embodiments, the fusion protein may be represented by the formula Y-Fc-X, Y-X-Fc, or Y-Z-X, wherein X is the CD4 targeting moiety, Fc is an immunoglobulin Fc domain, Y is the immunomodulatory moiety, and Z is a linker sequence. Additionally or alternatively, in some embodiments of the fusion protein disclosed herein, the antibody further comprises a Fc domain of any isotype, e.g., but are not limited to, IgG (including IgG1, IgG2, IgG3, and IgG4), IgA (including IgA₁ and IgA₂), IgD, IgE, or IgM. In certain embodiments, the fusion proteins comprise monoclonal antibodies, chimeric antibodies, or humanized antibodies, wherein the antibodies optionally comprise a human antibody framework region. In other embodiments, the fusion proteins of the present technology include antigen binding fragments selected from the group consisting of Fab, F(ab)′2, Fab′, scFᵥ, and Fᵥ.

Additionally or alternatively, in certain embodiments of the fusion proteins described herein, the antibody or antigen binding fragment comprises an IgG1 constant region comprising one or more amino acid substitutions selected from the group consisting of D265A, N297A, K322A, L234F, L235E and P331S. Additionally or alternatively, in some embodiments, the fusion proteins comprise an IgG4 constant region comprising a S228P mutation.

Additionally or alternatively, in some embodiments, the fusion protein includes an antibody comprising a heavy chain (HC) amino acid sequence of SEQ ID NO: 24, SEQ ID NO: 25, or SEQ ID NO: 26, or a variant thereof having one or more conservative amino acid substitutions. Additionally or alternatively, in some embodiments, the fusion protein includes an antibody comprising a light chain (LC) amino acid sequence of SEQ ID NO: 27, or a variant thereof having one or more conservative amino acid substitutions. In some embodiments, the fusion proteins of the present technology comprise a HC amino acid sequence and a LC amino acid sequence selected from the group consisting of: SEQ ID NO: 24 and SEQ ID NO: 27; SEQ ID NO: 25 and SEQ ID NO: 27; and SEQ ID NO: 26 and SEQ ID NO: 27, respectively.

In some embodiments, the fusion protein includes an antibody comprising (a) a LC sequence that is at least 80%, at least 85%, at least 90%, at least 95%, or at least 99% identical to the LC sequence present in SEQ ID NO: 27; and/or (b) a HC sequence that is at least 80%, at least 85%, at least 90%, at least 95%, or at least 99% identical to the HC sequence present in any one of SEQ ID NO: 24, SEQ ID NO: 25, or SEQ ID NO: 26.

Additionally or alternatively, in some embodiments of the fusion proteins disclosed herein, the CD4 targeting moiety is fused with the immunomodulatory moiety via a linker. In some embodiments, the CD4 targeting moiety is fused with the immunomodulatory moiety via a polypeptide linker. In some embodiments, the polypeptide linker is a Gly-Ser linker. In some embodiments, the polypeptide linker is or comprises a sequence of (GGGGS)ₙ (SEQ ID NO: 43), where n represents the number of repeating GGGGS (“GGGGS” disclosed as SEQ ID NO: 44) units and is 1, 2, 3, 4, 5, 6, 7, 8, 9, 10, 11, 12, 13, 14, 15, 16, 17, 18, 19, 20, 25, 30 or more. In some embodiments, the CD4 targeting moiety is directly fused to the immunomodulatory moiety.

In one aspect, the present disclosure provides a fusion protein comprising (a) an immunomodulatory moiety fused to a first heterodimerization domain, wherein (i) the first heterodimerization domain is incapable of forming a stable homodimer with another first heterodimerization domain, and (ii) the immunomodulatory moiety comprises an amino acid sequence of TGF-β receptor II (TGF-βRII) selected from the group consisting of SEQ ID NOs: 11-12 and 15-17; and (b) a CD4 targeting moiety fused to a second heterodimerization domain, wherein (i) the second heterodimerization domain comprises an amino acid sequence or a nucleic acid sequence that is distinct from the first heterodimerization domain, (ii) the second heterodimerization domain is incapable of forming a stable homodimer with another second heterodimerization domain, (iii) the second heterodimerization domain is configured to form a heterodimer with the first heterodimerization domain, and (iv) the CD4 targeting moiety comprises a heavy chain immunoglobulin variable domain (V_{H}) and a light chain immunoglobulin variable domain (V_{L}), wherein: the V_{H} comprises a V_{H}-CDR1 sequence of GYTFTSYVIH (SEQ ID NO: 6), a V_{H}-CDR2 sequence of YINPYNDGTDYDEKFKG (SEQ ID NO: 7), and a V_{H}-CDR3 sequence of EKDNYATGAWFAY (SEQ ID NO: 8), and the V_{L} comprises a V_{L}-CDR1 sequence of KSSQSLLYSTNQKNYLA (SEQ ID NO: 2), a V_{L}-CDR2 sequence of WASTRES (SEQ ID NO: 3), and a V_{L}-CDR3 sequence of QQYYSYRT (SEQ ID NO: 4). In some embodiments, the TGF-β receptor II is encoded by a nucleic acid sequence selected from the group consisting of SEQ ID NOs: 13-14, 18-20, and 21-23. Additionally or alternatively, in some embodiments, the V_{H} comprises an amino acid sequence that is at least 80%, at least 85%, at least 95%, or 100% identical to SEQ ID NO: 5; and/or the V_{L} comprises an amino acid sequence that is at least 80%, at least 85%, at least 95%, or 100% identical to SEQ ID NO: 1. In certain embodiments, the CD4 targeting moiety of the fusion protein specifically binds a CD4 epitope.

Additionally or alternatively, in some embodiments, the first heterodimerization domain and/or the second heterodimerization domain is a CH2-CH3 domain and has an isotype selected from the group consisting of IgG1, IgG2, IgG3, IgG4, IgA1, IgA2, IgM, IgD, and IgE. In certain embodiments, the first heterodimerization domain is a CH2-CH3 domain comprising T366W/5354 mutations and the second heterodimerization domain is a CH2-CH3 domain comprising T366S/L368A/Y407V/Y349C mutations. In any of the above embodiments of the fusion protein of the present technology, the V_{H} of the CD4 targeting moiety is linked to a CH1 domain and/or the V_{L} of the CD4 targeting moiety is linked to a CL domain. Additionally or alternatively, in certain embodiments, the first heterodimerization domain and/or the second heterodimerization domain comprises one or more amino acid substitutions selected from the group consisting of D265A, N297A, K322A, L234F, L235E and P331S.

In one aspect, the present disclosure provides a fusion protein comprising (a) a TGF-β-specific antigen binding fragment fused to a first heterodimerization domain, wherein (i) the first heterodimerization domain is incapable of forming a stable homodimer with another first heterodimerization domain, and (ii) the TGF-β-specific antigen binding fragment is derived from an anti-TGF-β antibody; and (b) a CD4 targeting moiety fused to a second heterodimerization domain, wherein (i) the second heterodimerization domain comprises an amino acid sequence or a nucleic acid sequence that is distinct from the first heterodimerization domain, (ii) the second heterodimerization domain is incapable of forming a stable homodimer with another second heterodimerization domain, (iii) the second heterodimerization domain is configured to form a heterodimer with the first heterodimerization domain, and (iv) the CD4 targeting moiety comprises a heavy chain immunoglobulin variable domain (V_{H}) and a light chain immunoglobulin variable domain (V_{L}), wherein: the V_{H} comprises a V_{H}-CDR1 sequence of GYTFTSYVIH (SEQ ID NO: 6), a V_{H}-CDR2 sequence of YINPYNDGTDYDEKFKG (SEQ ID NO: 7), and a V_{H}-CDR3 sequence of EKDNYATGAWFAY (SEQ ID NO: 8), and the V_{L} comprises a V_{L}-CDR1 sequence of KSSQSLLYSTNQKNYLA (SEQ ID NO: 2), a V_{L}-CDR2 sequence of WASTRES (SEQ ID NO: 3), and a V_{L}-CDR3 sequence of QQYYSYRT (SEQ ID NO: 4). The TGF-β-specific antigen binding fragment may be derived from any anti-TGF-β antibody known in the art. Examples of useful anti-TGF-β antibodies include fresolimumab (GC1008), lerdelimumab, metelimumab, SAR-439459, XOMA089, as well as those described in U.S. Pat. Nos. 6,492,497, 6,419,928, 10,035,851, 8,012,482, 7,927,593. Additionally or alternatively, in some embodiments, the V_{H} comprises an amino acid sequence that is at least 80%, at least 85%, at least 95%, or 100% identical to SEQ ID NO: 5; and/or the V_{L} comprises an amino acid sequence that is at least 80%, at least 85%, at least 95%, or 100% identical to SEQ ID NO: 1. In certain embodiments, the CD4 targeting moiety of the fusion protein specifically binds a CD4 epitope.

In any of the above embodiments of the fusion protein of the present technology, the CD4 targeting moiety comprises an antibody that includes a heavy chain (HC) amino acid sequence and a light chain (LC) amino acid sequence. In some embodiments of the fusion proteins disclosed herein, the heavy chain (HC) amino acid sequence is at least 80%, at least 85%, at least 90%, at least 95%, or at least 99% identical to the HC sequence present in any one of SEQ ID NOs: 24-26; and/or the LC sequence is at least 80%, at least 85%, at least 90%, at least 95%, or at least 99% to the LC sequence present in SEQ ID NO: 27. Additionally or alternatively, in some embodiments of the fusion protein, the heavy chain (HC) amino acid sequence is SEQ ID NO: 24, SEQ ID NO: 25, or SEQ ID NO: 26, or a variant thereof having one or more conservative amino acid substitutions, and/or the light chain (LC) amino acid sequence is SEQ ID NO: 27, or a variant thereof having one or more conservative amino acid substitutions. In certain embodiments, the HC amino acid sequence and the LC amino acid sequence is selected from the group consisting of: SEQ ID NO: 24 and SEQ ID NO: 27; SEQ ID NO: 25 and SEQ ID NO: 27; and SEQ ID NO: 26 and SEQ ID NO: 27, respectively.

In another aspect, the present disclosure provides a CD4 fusion protein that binds to the same CD4 epitope as any fusion protein of the present technology, wherein the CD4 fusion protein comprises a CD4 binding domain fused with an immunomodulatory moiety.

In one aspect, the present technology provides a recombinant nucleic acid sequence encoding any of the fusion proteins described herein. In another aspect, the present technology provides a host cell or vector expressing any nucleic acid sequence encoding any of the fusion proteins described herein.

In one aspect, the present disclosure provides compositions comprising fusion proteins of the present technology and a pharmaceutically-acceptable carrier, wherein the fusion proteins may be optionally conjugated to an agent selected from the group consisting of isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof.

In one aspect, the present disclosure provides a method for treating a cancer in a subject in need thereof, comprising administering to the subject an effective amount of a CD4 targeting fusion protein of the present technology. In some embodiments, the cancer is refractory or recurrent. In another aspect, the present disclosure provides a method for increasing tumor sensitivity to a therapy in a subject suffering from cancer comprising (a) administering an effective amount of a CD4 targeting fusion protein of the present technology to the subject; and (b) administering an effective amount of an anti-cancer therapeutic agent to the subject. In some embodiments, the cancer is refractory or recurrent.

Examples of cancers that can be treated by the fusion proteins of the present technology include, but are not limited to: prostate cancer, pancreatic cancer, biliary cancer, colon cancer, rectal cancer, liver cancer, kidney cancer, lung cancer, testicular cancer, breast cancer, ovarian cancer, brain cancer, bladder cancer, head and neck cancers, melanoma, sarcoma, multiple myeloma, leukemia, lymphoma, and the like. In some embodiments of the methods disclosed herein, the subject is human.

The fusion proteins of the present technology may be employed in conjunction with other therapeutic agents useful in the treatment of cancer. For example, the CD4 targeting fusion proteins of the present technology may be separately, sequentially or simultaneously administered with at least one additional therapeutic agent. Examples of such additional therapeutic agents include, but are not limited to, targeted therapies, immunotherapies (e.g., checkpoint inhibitors), antiangiogenic agents or chemotherapies. Targeted therapy agents include, but are not limited to, apoptosis-inducing proteasome inhibitor (e.g., Bortezomib), Selective estrogen-receptor modulator (e.g., Tamoxifen), BCR-ABL inhibitors (e.g., Imatinib, Dasatinib and Nilotinib), BTK inhibitor (e.g., Ibrutinib), EGFR inhibitors (e.g., Gefitinib, Erlotinib, Lapatinib, Neratinib, Osimertinib, Vandetanib, Dacomitinib), Janus kinase inhibitors (e.g., Ruxolitinib, Tofacitinib, Oclacitinib, baricitinib and Peficitinib), ALK inhibitors (e.g., Crizotinib, Ceritinib, Alectinib, Brigatinib and Lorlatinib), Bcl-2 inhibitors (e.g., Obatoclax, Navitoclax and Gossypol), PARP inhibitors (e.g., Iniparib, Olaparib and Talazoparib), PI3K inhibitors (e.g., Idelalisib, Copanlisib, Duvelisib and Alpelisib), MEK inhibitors (e.g., Trametinib, Binimetinib), CDK inhibitors (e.g., Palbociclib, Ribociclib and Abemaciclib), Hsp90 inhibitors (e.g., Gamitrinib and Luminespib), DNA-targeting agent (e.g., dianhydrogalactitol), NTRK inhibitors (e.g., Entrectinib and Larotrectinib), mTOR inhibitors (e.g., Temsirolimus and Everolimus), BRAF inhibitors (e.g., Vemurafenib, Dabrafenib, Encorafenib and Sorafenib), aromatase inhibitors, cytostatic alkaloids, cytotoxic antibiotics, antimetabolites, endocrine/hormonal agents, topoisomerase inhibitors, bisphosphonate therapy agents and targeted biological therapy agents (e.g., therapeutic peptides described in U.S. Pat. No. 6,306,832, WO 2012007137, WO 2005000889, WO 2010096603 etc.). Targeted therapy monoclonal antibodies include, but are not limited to, EGFR antibodies (e.g., Cetuximab, Panitumumab, Necitumumab), Her2/neu antibodies (e.g., Trastuzumab, Pertuzumab and Margetuximab), CD52 antibodies (e.g., Alemtuzumab), CD20 antibodies (e.g., Rituximab, Ofatumumab), GD2 antibodies (e.g., Dinutuximab), RANKL antibodies (e.g., Denosumab). Cancer immunotherapies include, but are not limited to, anti-PD-1 (e.g., Pembrolizumab, Nivolumab, Cemiplimab), anti-PD-L1 (e.g., atezolizumab, Avelumab, Durvalumab), anti-CTLA-4 (e.g., Ipilimumab, Tremelimumab), CD3/CD19 (e.g., Blinatumomab). Antiangiogenic agents include, but are not limited to, Axitinib, Bevacizumab, Cabozantinib, Everolimus, Lenalidomide, Lenvatinib mesylate, Pazopanib, Ramucirumab, Regorafenib, Sorafenib, Sunitinib, Thalidomide, Vandetanib, Ziv-aflibercept. In some embodiments, the at least one additional therapeutic agent is a chemotherapeutic agent. Specific chemotherapeutic agents include, but are not limited to, cyclophosphamide, fluorouracil (or 5-fluorouracil or 5-FU), Leucovorin, methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, tykerb, anthracyclines (e.g., daunorubicin and doxorubicin), oxaliplatin, melphalan, etoposide, mechlorethamine, bleomycin, microtubule poisons, annonaceous acetogenins, or combinations thereof.

In another aspect, the present disclosure provides a method for monitoring cancer progression in a patient in need thereof comprising (a) administering to the patient an effective amount of a fusion protein of the present technology; and (b) detecting tumor growth in the patient, wherein a reduction in tumor size relative to that observed in the patient prior to administration of the fusion protein is indicative of cancer arrest or cancer regression. Methods for detecting tumor growth are known in the art and include positron emission tomography, magnetic resonance imaging (MRI), ultrasound, computer tomography, or single photon emission computed tomography.

In one aspect, the present disclosure provides an engineered helper T cell, wherein the cell lacks detectable expression or activity of a TGF-β receptor II that comprises an amino acid sequence of any one of SEQ ID NOs: 11-12. In another aspect, the present disclosure provides an engineered helper T cell, wherein the cell expresses an inhibitory nucleic acid that specifically targets and inhibits the expression of a TGF-β receptor II nucleic acid sequence selected from among SEQ ID NOs: 13-14, 18-20, and 21-23. The inhibitory nucleic acid may be an antisense oligonucleotide, a siRNA, a sgRNA or a shRNA. Additionally or alternatively, in some embodiments of the engineered helper T cells of the present technology, the cells comprise a transgene that encodes a dominant negative TGF-β receptor II or the inhibitory nucleic acid. The transgene may be operably linked to an ubiquitous promoter, a constitutive promoter, a T cell-specific promoter, or an inducible promoter. In one aspect, the present disclosure provides an engineered helper T cell comprising a deletion, insertion, inversion, or frameshift mutation in a TGF-β receptor II gene encoded by the nucleic acid sequence of SEQ ID NO: 13 or SEQ ID NO: 14. Additionally or alternatively, in some embodiments, the engineered helper T cell is derived from an autologous donor or an allogeneic donor.

In one aspect, the present disclosure provides a method for inhibiting tumor growth or metastasis in a subject with cancer comprising administering to the subject an effective amount of any of the engineered helper T cells described herein. The engineered helper T cells may be administered intravenously, intraperitoneally, subcutaneously, intramuscularly, or intratumorally. The cancer may be prostate cancer, pancreatic cancer, biliary cancer, colon cancer, rectal cancer, liver cancer, kidney cancer, lung cancer, testicular cancer, breast cancer, ovarian cancer, brain cancer, bladder cancer, head and neck cancers, melanoma, sarcoma, multiple myeloma, leukemia, or lymphoma.

Additionally or alternatively, in some embodiments, the method further comprises administering an additional cancer therapy. Examples of additional cancer therapies include, but are not limited to, chemotherapy, radiation therapy, immunotherapy, monoclonal antibodies, anti-cancer nucleic acids or proteins, anti-cancer viruses or microorganisms, and any combinations thereof. In some embodiments, the additional therapeutic agent is one or more of targeted therapies (e.g. apoptosis-inducing proteasome inhibitor, selective estrogen-receptor modulator, BCR-ABL inhibitors, BTK inhibitor, EGFR inhibitors, Janus kinase inhibitors, ALK inhibitors, Bcl-2 inhibitors, PARP inhibitors, PI3K inhibitors, MEK inhibitors, CDK inhibitors, Hsp90 inhibitors, DNA-targeting agent, NTRK inhibitors, mTOR inhibitors, BRAF inhibitors, aromatase inhibitors, cytostatic alkaloids, cytotoxic antibiotics, antimetabolites, endocrine/hormonal agents, topoisomerase inhibitors, bisphosphonate therapy agents), antiangiogenic agents (e.g., VEGF/VEGFR inhibitors), cancer immunotherapies (e.g. anti-PD-1, anti-PD-L1, anti-CTLA-4) or chemotherapeutic agents.

Additionally or alternatively, in certain embodiments, the method further comprises administering a cytokine agonist or antagonist to the subject. In some embodiments, the cytokine agonist or antagonist is administered prior to, during, or subsequent to administration of the one or more engineered helper T cells. In some embodiments, the cytokine agonist or antagonist is selected from a group consisting of interferon α, interferon β, interferon γ, complement C5a, IL-2, TNFalpha, CD40L, Ox40, IL-7, IL-18, IL-12, IL-23, IL-15, IL-17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

Additionally or alternatively, in some embodiments, the method further comprises sequentially, separately, or simultaneously administering to the subject at least one chemotherapeutic agent. Examples of chemotherapeutic agents include, but are not limited to, cyclophosphamide, fluorouracil (or 5-fluorouracil or 5-FU), Leucovorin, methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, tykerb, anthracyclines (e.g., daunorubicin and doxorubicin), oxaliplatin, melphalan, etoposide, mechlorethamine, bleomycin, microtubule poisons, annonaceous acetogenins, or combinations thereof.

In another aspect, the present disclosure provides methods for preparing immune cells for cancer therapy comprising isolating helper T cells from a donor subject; transducing the helper T cells with (a) an inhibitory nucleic acid that specifically targets and inhibits the expression of a TGF-β receptor II nucleic acid sequence selected from among SEQ ID NOs: 13-14, 18-20, and 21-23, and/or (b) an expression vector that encodes a dominant negative TGF-β receptor II having an amino acid sequence of any one of SEQ ID NOs: 15-17 or 37-42. The inhibitory nucleic acid is an antisense oligonucleotide, a siRNA, a sgRNA or a shRNA.

In one aspect, the present disclosure provides a method of treatment comprising isolating helper T cells from a donor subject; transducing the helper T cells with (a) an inhibitory nucleic acid that specifically targets and inhibits the expression of a TGF-β receptor II nucleic acid sequence selected from among SEQ ID NOs: 13-14, 18-20, and 21-23, and/or (b) an expression vector that encodes a dominant negative TGF-β receptor II having an amino acid sequence of any one of SEQ ID NOs: 15-17 or 37-42; and administering the transduced helper T cells to a recipient subject. In some embodiments, the donor subject and the recipient subject are the same. In other embodiments, the donor subject and the recipient subject are different. In some embodiments, the method further comprises administering an additional cancer therapy.

Also disclosed herein are kits for the treatment of cancers (e.g., refractory cancers), comprising at least one fusion protein of the present technology, or a functional variant (e.g., substitutional variant) thereof and instructions for use. Also provided herein are kits for the treatment of cancers (e.g., refractory cancers), comprising any of the engineered helper T cells described herein, and instructions for use.

### BRIEF DESCRIPTION OF THE DRAWINGS

FIG. 1 shows transforming growth factor-β receptor II (TGF-βRII) expression on CD4⁺ T cells and CD8⁺ T cells from the tumor-draining lymph nodes of Tgfbr2^{fl/fl}PyMT and CD8^{Cre}Tgfbr2^{fl/fl}PyMT mice.

FIG. 2 shows representative flow cytometry plots of CD62L and CD44 expression and statistical analyses of the gated populations in conventional CD4⁺Foxp3⁻ T cells (top panel), CD4⁺Foxp3⁺ regulatory T cells (middle panel) and CD8⁺ T cells (bottom panel) from the tumor-draining lymph nodes of Tgfbr2^{fl/fl}PyMT and CD8^{Cre}Tgfbr2^{fl/fl}PyMT mice. All statistical data are shown as mean±SEM. **: P<0.01; ****: P<0.0001; and ns: not significant.

FIG. 3 shows representative flow cytometry plots and statistical analyses of programmed cell death protein 1 (PD-1) and Granzyme B (GzmB) expression in tumor-infiltrating CD8⁺ T cells from Tgfbr2^{fl/fl}PyMT and CD8^{Cre}Tgfbr2^{fl/fl}PyMT mice. All statistical data are shown as mean±SEM. *: P<0.05; ***: P<0.001; and ns: not significant.

FIG. 4 shows tumor measurements from Tgfbr2^{fl/fl}PyMT (n=8) and CD8^{Cre}Tgfbr2^{fl/fl}PyMT (n=7) mice.

FIG. 5 shows representative flow cytometry plots of CD49a and CD103 expression and statistical analyses of the gated populations in tumor-infiltrating CD8⁺ T cells from Tgfbr2^{fl/fl}PyMT and CD8^{Cre}Tgfbr2^{fl/fl}PyMT mice. All statistical data are shown as mean±SEM. ****: P<0.0001.

FIG. 6 shows TGF-βRII expression on CD4⁺ T cells and CD8⁺ T cells from the tumor-draining lymph nodes of Tgfbr2^{fl/fl}PyMT and ThPOK^{Cre}Tgfbr2^{fl/fl}PyMT mice.

FIG. 7 shows representative flow cytometry plots of CD62L and CD44 expression and statistical analyses of the gated populations in conventional CD4⁺Foxp3⁻ T cells (top panel), CD4⁺Foxp3⁺ regulatory T cells (middle panel) and CD8⁺ T cells (bottom panel) from the tumor-draining lymph nodes of Tgfbr2^{fl/fl}PyMT (wild-type, WT) and ThPOK^{Cre}Tgfbr2^{fl/fl}PyMT (knockout, KO) mice. All statistical data are shown as mean±SEM. ***: P<0.001; ****: P<0.0001; and ns: not significant.

FIG. 8 shows representative flow cytometry plots of CD49a and CD103 expression and statistical analyses of the gated populations in tumor-infiltrating CD8⁺ T cells from Tgfbr2^{fl/fl}PyMT (wild-type, WT) and ThPOK^{Cre}Tgfbr2^{fl/fl}PyMT (knockout, KO) mice. All statistical data are shown as mean±SEM. ***: P<0.001; and ns: not significant.

FIG. 9 shows representative flow cytometry plots and statistical analyses of PD-1 and GzmB expression in tumor-infiltrating CD8⁺ T cells from Tgfbr2^{fl/fl}PyMT and ThPOK^{Cre}Tgfbr2^{fl/fl}PyMT mice. All statistical data are shown as mean±SEM. *: P<0.05; **: P<0.01; ****: P<0.0001; and ns: not significant.

FIG. 10 shows tumor measurements from Tgfbr2^{fl/fl}PyMT (n=7), ThPOK^{Cre}Tgfbr2^{fl/fl}PyMT (n=5), CD8^{-/-}Tgfbr2^{fl/fl}PyMT (n=3) and CD8^{-/-} ThPOK^{Cre}Tgfbr2^{fl/fl}PyMT (n=5) mice. All statistical data are shown as mean±SEM. **: P<0.01; and ns: not significant.

FIG. 11 shows representative flow cytometry plots of CD4 and CD8 expression on TCRβ⁺NK 1.1⁻ cells in the tumor-draining lymph nodes of CD8^{-/-} Tgfbr2^{fl/fl}PyMT (CD8^{-/-}) and CD8^{-/-}ThPOK^{Cre}Tgfbr2^{fl/fl}PyMT (CD8^{-/-} knockout, CD8^{-/-}KO) mice.

FIG. 12 shows representative flow cytometry plots of CD62L and CD44 expression and statistical analyses of the gated populations in conventional CD4⁺Foxp3⁻ T cells (top panel) and CD4⁺Foxp3⁺ regulatory T cells (bottom panel) from CD8^{-/-} and CD8^{-/-}KO mice. All statistical data are shown as mean SEM. *: P<0.05; **: P<0.01; ***: P<0.001; and ns: not significant.

FIG. 13 shows representative immunofluorescence images and statistical analyses of E-Cadherin (green), Ki67 (red) and cleaved Caspase 3 (CC3, cyan) expression in mammary tumor tissues from 8- and 23-week-old Tgfbr2^{fl/fl}PyMT (wild-type, WT) and ThPOK^{Cre}Tgfbr2^{fl/fl}PyMT (knockout, KO) mice. The percentage of Ki67⁺E-Cadherin⁺ cells over total E-Cadherin⁺ epithelial cells was calculated from multiple 0.02 mm² regions (n=10 and 7 for WT and KO tumor tissues, respectively). The percentage of CC3⁺ areas over total E-Cadherin⁺ areas was calculated from multiple 0.02 mm² regions (n=10 for WT and KO tumor tissues). All statistical data are shown as mean±SEM. ***: P<0.001; and ns: not significant.

FIG. 14 shows representative immunofluorescence images and statistical analyses of E-Cadherin (green), Ki67 (red) and cleaved Caspase 3 (CC3, cyan) expression in mammary tumor tissues from 23-week-old Tgfbr2^{fl/fl}PyMT and CD8^{Cre}Tgfbr2^{fl/fl}PyMT mice. The percentage of Ki67⁺E-Cadherin⁺ cells over total E-Cadherin⁺ epithelial cells was calculated from multiple 0.02 mm² regions (n=9). The percentage of CC3⁺ areas over total E-Cadherin⁺ areas was calculated from multiple 0.02 mm² regions (n=9). All statistical data are shown as mean±SEM. ns: not significant.

FIG. 15 shows representative immunofluorescence images and statistical analyses of E-Cadherin (green), Ki67 (red) and cleaved Caspase 3 (CC3, cyan) expression in mammary tumor tissues from 23-week-old CD8^{-/-} Tgfbr2^{fl/fl}PyMT (CD8^{-/-}) and CD8^{-/-}ThPOK^{Cre}Tgfbr2^{fl/fl}PyMT (CD8^{-/-} knockout, CD8^{-/-}KO) mice. The percentage of Ki67⁺E-Cadherin⁺ cells over total E-Cadherin⁺ epithelial cells was calculated from multiple 0.02 mm² regions (n=9). The percentage of CC3⁺ areas over total E-Cadherin⁺ areas was calculated from multiple 0.02 mm² regions (n=9). All statistical data are shown as mean±SEM. ****: P<0.0001, and ns: not significant.

FIG. 16 shows representative immunofluorescence images and statistical analyses of E-Cadherin (green), CD4 (red) and CC3 (cyan) expression in mammary tumor tissues from 8- and 23-week-old Tgfbr2^{fl/fl}PyMT (wild-type, WT) and ThPOK^{Cre}Tgfbr2^{fl/fl}PyMT (knockout, KO) mice. Intratumoral (white arrows) and stromal (yellow arrows) CD4⁺ T cells were counted from multiple 0.1 mm² regions (n=8 for WT and KO tumor tissues). All statistical data are shown as mean±SEM. **: P<0.01; ***: P<0.001; and ns: not significant.

FIG. 17 shows representative immunofluorescence images and statistical analyses of E-Cadherin (green), CD45 (red) and cleaved Caspase 3 (CC3, cyan) in mammary tumor tissues from 23-week-old Tgfbr2^{fl/fl}PyMT (wild-type, WT) and ThPOK^{Cre}Tgfbr2^{fl/fl}PyMT (knockout, KO) mice. Intratumoral (white arrows) and stromal (yellow arrows) CD45⁺ T cells were counted from multiple 0.1 mm² regions (n=8 for WT and KO tumor tissues). All statistical data are shown as mean±SEM. ***: P<0.001.

FIG. 18 shows representative immunofluorescence images of fibrinogen (Fg, white), CD31 (red), cleaved Caspase 3 (CC3, cyan) and E-Cadherin (green) in mammary tumor tissues from 23-week-old Tgfbr2^{fl/fl}PyMT (wild-type, WT) and ThPOK^{Cre}Tgfbr2^{fl/fl}PyMT (knockout, KO) mice. Extravascular (EV) Fg deposition events (magenta arrows) were calculated from multiple 1 mm² regions (n=9 for WT and KO tumor tissues). Isolated CD31⁺ endothelial cells (yellow arrows) were counted from multiple 1 mm² regions (n=6 for WT and KO tumor tissues). All statistical data are shown as mean±SEM. **: P<0.01; ****: P<0.0001.

FIG. 19 shows quantification of CD31⁺ endothelial cells in mammary tumor tissues from 23-week-old Tgfbr2^{fl/fl}PyMT (wild-type, WT) and ThPOK^{Cre}Tgfbr2^{fl/fl}PyMT (knockout, KO) mice. The percentage of CD31⁺ areas was calculated from multiple 1 mm² regions (n=6 for WT and KO tumor tissues). All statistical data are shown as mean±SEM. ns: not significant.

FIG. 20 shows representative immunofluorescence images of NG2⁺ pericytes (white), CD31⁺ endothelial cells (red), GP38⁺ fibroblasts (cyan) and E-Cadherin (green) in mammary tumor tissues from 23-week-old Tgfbr2^{fl/fl}PyMT (wild-type, WT) and ThPOK^{Cre}Tgfbr2^{fl/fl}PyMT (knockout, KO) mice. NG2-unbound (magenta arrows) or GP38-unbound (yellow arrows) isolated CD31⁺ endothelial cells were counted from multiple 1 mm² regions (n=9 for WT and KO tumor tissues). All statistical data are shown as mean±SEM. ****: P<0.0001.

FIG. 21 shows representative immunofluorescence images of collagen IV (Col IV, white), CD31 (red), fibronectin (FN, cyan) and E-Cadherin (green) in mammary tumor tissues from Tgfbr2^{fl/fl}PyMT (wild-type, WT) and ThPOK^{Cre}Tgfbr2^{fl/fl}PyMT (knockout, KO) mice. The average continuous lengths of Col IV and FN were measured in multiple 1 mm² regions (n=9 for WT and KO tumor tissues). All statistical data are shown as mean±SEM. ****: P<0.0001.

FIG. 22 shows representative immunofluorescence images of Hypoxic probe (HPP, white), CD31 (red), CC3 (cyan) and E-Cadherin (green) in mammary tumor tissues from Tgfbr2^{fl/fl}PyMT (wild-type, WT) and ThPOK^{Cre}Tgfbr2^{fl/fl}PyMT (knockout, KO) mice. The percentage of HPP⁺E-Cadherin⁺ regions over E-Cadherin⁺ epithelial regions was calculated from multiple 1 mm² regions (n=9 for WT and KO tumor tissues). The shortest distance of HPP⁺ regions (magenta dashed lines) or CC3⁺ regions (yellow dashed lines) to CD31⁺ endothelial cells was measured in tumor tissues from KO mice (n=9). The dashed boxes coupled with dashed lines show high magnification of selected tissue regions. All statistical data are shown as mean±SEM ****: P<0.0001.

FIG. 23 shows representative flow cytometry plots of TCRβ, NK1.1, CD4, CD8 and Foxp3 expression in tumor-infiltrating leukocytes from 23-week-old Tgfbr2^{fl/fl}PyMT (wild-type, WT) and ThPOK^{Cre}Tgfbr2^{fl/fl}PyMT (knockout, KO) mice as well as statistical analyses of the gated populations. All statistical data are shown as mean±SEM. **: P<0.01; ***: P<0.001; and ns: not significant.

FIG. 24 shows average Z-score values of genes significantly upregulated in TGF-βRII-deficient T cells. Tumor-infiltrating CD4⁺CD25⁻ T cells from 23-week-old Tgfbr2^{fl/fl}PyMT (wild-type, WT) and ThPOK^{Cre}Tgfbr2^{fl/fl}PyMT (knockout, KO) mice were purified, and their transcriptome probed by RNA sequencing. Genes are grouped based on the localization and function of their encoded proteins.

FIG. 25 shows average Z-score values of genes significantly downregulated in TGF-βRII-deficient T cells. Tumor-infiltrating CD4⁺CD25″ T cells from 23-week-old Tgfbr2^{fl/fl}PyMT (wild-type, WT) and ThPOK^{Cre}Tgfbr2^{fl/fl}PyMT (knockout, KO) mice were purified, and their transcriptome probed by RNA sequencing. Genes are grouped based on the localization and function of their encoded proteins.

FIG. 26 shows representative flow cytometry plots and statistical analyses of IL-4 and IFN-γ expression in CD4⁺Foxp3⁻ T cells from the tumor-draining lymph nodes of 23-week-old (wild-type, WT) and ThPOK^{Cre}Tgfbr2^{fl/fl}PyMT (knockout, KO) mice. All statistical data are shown as mean±SEM. ***: P<0.001.

FIG. 27 shows tumor measurements from Ifng^{-/-} Tgfbr2^{fl/fl}PyMT (Ifng^{-/-}, n=3), Ifng^{-/-}ThPOK^{Cre}Tgfbr2^{fl/fl}PyMT (Ifng^{-/-}KO, n=6), Il-4^{-/-} Tgfbr2^{fl/fl}PyMT (Il-4^{-/-}, n=4) and Il-4^{-/-}ThPOK^{Cre}Tgfbr2^{fl/fl}PyMT (Il-4^{-/-}KO, n=4) mice. All statistical data are shown as mean±SEM. *: P<0.05; **: P<0.01; and ns: not significant.

FIG. 28 shows representative flow cytometry plots of CD62L and CD44 expression and statistical analyses of the gated populations in conventional CD4⁺Foxp3⁻ T cells (top panel) and CD4⁺Foxp3⁺ regulatory T cells (bottom panel) from Ifng^{-/-} Tgfbr2^{fl/fl}PyMT (Ifng^{-/-}) and Ifng^{-/-}ThPOK^{Cre}Tgfbr2^{fl/fl}PyMT (Ifng^{-/-}KO) mice. All statistical data are shown as mean±SEM. *: P<0.05; **: P<0.01; and ns: not significant.

FIG. 29 shows representative flow cytometry plots of TCRβ, NK11.1, CD4, CD8 and Foxp3 expression in tumor-infiltrating leukocytes from 23-week-old Ifng^{-/-} Tgfbr2^{fl/fl}PyMT (Ifng^{-/-}) and Ifng^{-/-}ThPOK^{Cre}Tgfbr2^{fl/fl}PyMT (Ifng^{-/-}KO) mice as well as statistical analyses of the gated populations. All statistical data are shown as mean±SEM. *: P<0.05; **: P<0.01; and ns: not significant.

FIG. 30 shows representative immunofluorescence images of fibrinogen (Fg, white), CD31 (red), cleaved Caspase 3 (CC3, cyan) and E-Cadherin (green) in mammary tumor tissues from 23-week-old Ifng^{-/-} Tgfbr2^{fl/fl}PyMT (Ifng^{-/-}), Ifng^{-/-}ThPOK^{Cre}Tgfbr2^{fl/fl}PyMT (Ifng^{-/-}KO), Il-4^{-/-} Tgfbr2^{fl/fl}PyMT (Il-4^{-/-}) and Il-4^{-/-}ThPOK^{Cre}Tgfbr2^{fl/fl}PyMT (Il-4^{-/-}KO) mice. Extravascular (EV) Fg deposition events (magenta arrows) were calculated from multiple 1 mm² regions (n=9 for Ifng^{-/-}, Ifng^{-/-}KO, Il-4^{-/-} and Il-4^{-/-}KO tumor tissues). Isolated CD31⁺ endothelial cells (yellow arrows) were counted from multiple 1 mm² regions (n=9 for Ifng^{-/-}, Ifng^{-/-}KO, Il-4^{-/-} and Il-4^{-/-}KO tumor tissues). All statistical data are shown as mean±SEM. ****: P<0.0001; and ns: not significant.

FIG. 31 shows representative immunofluorescence images of Hypoxic probe (HPP, white), CD31 (red), cleaved Caspase 3 (CC3, cyan) and E-Cadherin (green) in mammary tumor tissues from Ifng^{-/-} Tgfbr2^{fl/fl}PyMT (Ifng^{-/-}), Ifng^{-/-}ThPOK^{Cre}Tgfbr2^{fl/fl}PyMT (Ifng^{-/-}KO), Il-4^{-/-} Tgfbr2^{fl/fl}PyMT (Il-4^{-/-}) and Il-4^{-/-}ThPOK^{Cre}Tgfbr2^{fl/fl}PyMT (Il-4^{-/-}KO) mice. The percentage of HPP⁺E-Cadherin⁺ regions over E-Cadherin⁺ epithelial regions was calculated from multiple 1 mm² regions (n=9 for Ifng^{-/-}, Ifng^{-/-}KO, Il-4^{-/-} and Il-4^{-/-}KO tumor tissues). The shortest distance of HPP⁺ regions (magenta dashed lines) or CC3⁺ regions (yellow dashed lines) to CD31⁺ endothelial cells was measured in tumor tissues from Ifng^{-/-}KO mice (n=9). All statistical data are shown as mean±SEM. ****: P<0.0001; and ns: not significant.

FIG. 32 shows representative flow cytometry plots of CD62L and CD44 expression and statistical analyses of the gated populations in conventional CD4⁺Foxp3⁻ T cells (top panel) and CD4⁺Foxp3⁺ regulatory T cells (bottom panel) from Il-4^{-/-} Tgfbr2^{fl/fl}PyMT (Il-4^{-/-}) and Il-4^{-/-}ThPOK^{Cre}Tgfbr2^{fl/fl}PyMT (Il-4^{-/-}KO) mice. All statistical data are shown as mean SEM. *: P<0.05; **: P<0.01, ***: P<0.001; ****: P<0.0001, and ns: not significant.

FIG. 33 shows representative flow cytometry plots of TCRβ, NK1.1, CD4, CD8 and Foxp3 expression in tumor-infiltrating leukocytes from 23-week-old Il-4^{-/-} Tgfbr2^{fl/fl}PyMT (Il-4^{-/-}) and Il-4^{-/-}ThPOK^{Cre}Tgfbr2^{fl/fl}PyMT (Il-4^{-/-}KO) mice as well as statistical analyses of the gated populations. All statistical data are shown as mean±SEM, ns: not significant.

FIG. 34 shows representative immunofluorescence images and statistical analyses of CD31 (white), Ki67 (red) and E-Cadherin (green) expression in mammary tumor tissues from PyMT mice harboring unpalpable, 5×5 mm, or 9×9 mm tumors. The percentage of Ki67⁺E-Cadherin⁺ cells over total E-Cadherin⁺ epithelial cells was calculated from multiple 0.02 mm² regions (n=9 for each group). Isolated CD31⁺ endothelial cells in the tumor parenchyma (yellow arrows) were counted from multiple 1 mm² regions (n=9 for each group). All statistical data are shown as mean±SEM. *: P<0.05; and ****: P<0.0001.

FIG. 35 shows transforming growth factor-β receptor II (TGF-βRII) expression on CD4⁺ T cells and CD8⁺ T cells from the tumor-draining lymph nodes of Tgfbr2^{fl/fl}PyMT and CD4^{CreERT2}Tgfbr2^{fl/fl}PyMT mice treated with tamoxifen.

FIG. 36 shows representative flow cytometry plots and statistical analyses of IL-4 and IFN-γ expression in CD4⁺Foxp3⁻ T cells from the tumor-draining lymph nodes of Tgfbr2^{fl/fl}PyMT and CD4^{CreERT2}Tgfbr2^{fl/fl}PyMT mice treated with tamoxifen. All statistical data are shown as mean±SEM. *: P<0.05; and **: P<0.01.

FIG. 37 shows representative flow cytometry plots of TCRβ, NK1.1, CD4, CD8 and Foxp3 expression in tumor-infiltrating leukocytes from Tgfbr2^{fl/fl}PyMT and CD4^{CreERT2}Tgfbr2^{fl/fl}PyMT mice treated with tamoxifen as well as statistical analyses of the gated populations. All statistical data are shown as mean±SEM, *: P<0.05; and ns: not significant.

FIG. 38 shows tumor growth measurements for Tgfbr2^{fl/fl}PyMT and CD4^{CreERT2}Tgfbr2^{fl/fl}PyMT mice bearing 5×5 mm tumors which were left untreated or treated with Tamoxifen (Tam) (n=4, 3, 4 and 5) twice a week for 6 weeks. All statistical data are shown as mean±SEM. **: P<0.01; and ***: P<0.001.

FIG. 39 shows representative immunofluorescence images and statistical analyses of E-Cadherin (green), Ki67 (red), and cleaved Caspase 3 (CC3, blue) expression in mammary tumor tissues from Tgfbr2^{fl/fl}PyMT and CD4^{CreERT2}Tgfbr2^{fl/fl}PyMT mice treated with tamoxifen. The percentage of Ki67⁺E-Cadherin⁺ cells over total E-Cadherin⁺ epithelial cells was calculated from multiple 0.02 mm² regions (n=9). The percentage of CC3⁺ areas over total E-Cadherin⁺ areas was calculated from multiple 0.02 mm² regions (n=10). All statistical data are shown as mean±SEM. **: P<0.01; and ns: not significant.

FIG. 40 shows representative immunofluorescence images of fibrinogen (Fg, white), CD31 (red), cleaved Caspase 3 (CC3, cyan) and E-Cadherin (green) in mammary tumor tissues from Tgfbr2^{fl/fl}PyMT and CD4^{CreERT2}Tgfbr2^{fl/fl}PyMT mice treated with tamoxifen. Extravascular (EV) Fg deposition events (magenta arrows) were calculated from multiple 1 mm² regions (n=9 for each group). Isolated CD31⁺ endothelial cells (yellow arrows) were counted from multiple 1 mm² regions (n=9 for each group). All statistical data are shown as mean±SEM. ****: P<0.0001.

FIG. 41 shows representative immunofluorescence images of NG2⁺ pericytes (white), CD31⁺ endothelial cells (red), GP38⁺ fibroblasts (blue) and E-Cadherin (green) in mammary tumor tissues from Tgfbr2^{fl/fl}PyMT and CD4^{CreERT2}Tgfbr2^{fl/fl}PyMT mice treated with tamoxifen. NG2-unbound (magenta arrows) or GP38-unbound (yellow arrows) isolated CD31⁺ endothelial cells were counted from multiple 1 mm² regions (n=9 for each group). All statistical data are shown as mean±SEM. ***: P<0.001; ****: P<0.0001.

FIG. 42 shows representative immunofluorescence images of collagen IV (Col IV, white), CD31 (red), fibronectin (FN, cyan) and E-Cadherin (green) in mammary tumor tissues from Tgfbr2^{fl/fl}PyMT and CD4^{CreERT2}Tgfbr2^{fl/fl}PyMT mice treated with tamoxifen. The average continuous lengths of Col IV and FN were measured in multiple 1 mm² regions (n=9 for each group). All statistical data are shown as mean±SEM. ***: P<0.001; ****: P<0.0001.

FIG. 43 shows representative immunofluorescence images of hypoxic probe (HPP, white), CD31 (red), CC3 (cyan) and E-Cadherin (green) in mammary tumor tissues from Tgfbr2^{fl/fl}PyMT and CD4^{CreERT2}Tgfbr2^{fl/fl}PyMT mice treated with tamoxifen. The percentage of HPP⁺E-Cadherin⁺ areas over E-Cadherin⁺ epithelial areas was calculated from multiple 1 mm² regions (n=9 for each group). The shortest distance of HP1³⁺ regions (magenta dashed lines) or CC3⁺ regions (yellow dashed lines) to CD31⁺ endothelial cells was measured in tumor tissues from CD4^{CreERT2}Tgfbr201PyMT mice treated with tamoxifen (n=9). All statistical data are shown as mean±SEM. ****: P<0.0001.

FIG. 44 shows representative flow cytometric plots of CD4 and CD25 expression in CD4⁺ lymphocytes isolated from the lymph nodes and spleen of Tgfbr2^{fl/fl} (wild-type, WT) and ThPOK^{Cre}Tgfbr2^{fl/fl} (knockout, KO) mice.

FIG. 45 shows tumor measurements of tumor-bearing PyMT mice adoptively transferred with CD4⁺CD25⁻ T cells from Tgfbr2^{fl/fl} (wild-type, WT) and ThPOK^{Cre}Tgfbr2^{fl/fl} (knockout, KO) mice, respectively. All statistical data are shown as mean±SEM. *: P<0.05.

FIG. 46 shows a schematic of interactions between the ibalizumab antigen-binding (Fab) fragment in cyan and human CD4 in green revealed by structural analysis (pdb3O2D. The MHC-II binding site is localized on the CD4 D1 domain highlighted in red. CD4 D1 and D2 domains are boxed in dashed squares.

FIG. 47 shows a ribbon and surface structural display of TGF-β1-TGF-βRII-TGF-βRI hexameric complex (pdb3kfd) with homodimeric TGF-β1 colored in magenta and red, TGF-βRII extracellular domain (ECD) in green, and TGF-βRI ECD in yellow.

FIG. 48 shows a schematic representation of ibalizumab Fab and TGF-βRII ECD fusion proteins in a murine IgG1 framework. The star indicates a D265A substitution in the CH2 domain, and the semi-circle and moon shapes indicate knob-into-hole (KIH) modifications in the CH3 domain to enable heavy chain heterodimerization. The gray or colored parts indicate mouse or human sequences, respectively.

FIG. 49 shows a plot of yield of ibalizumab Fab and TGF-βRII ECD fusion proteins produced in a FreeStyle HEK293-F cell transient expression system. FreeStyle HEK293-F cells transfected with plasmids encoding the indicated fusion antibodies were cultured for 4 days, and the supernatant was collected. Protein G affinity purification and size exclusion chromatography were used to purify these antibodies.

FIG. 50 shows a plot of aggregation percentage of ibalizumab Fab and TGF-βRII ECD fusion proteins produced in a FreeStyle HEK293-F cell transient expression system. FreeStyle HEK293-F cells transfected with plasmids encoding the indicated fusion antibodies were cultured for 4 days, and the supernatant was collected. Protein G affinity purification and size exclusion chromatography were used to purify these antibodies.

FIG. 51 shows a schematic representation of 4T-Trap. The gray or colored parts indicate mouse or human sequences, respectively. The star indicates a D265A substitution in the CH2 domain.

FIG. 52 shows schematic representations of antibody structures for 4T-Trap, TGF-β-Trap, αCD4 and mGO53.

FIG. 53 shows size exclusion chromatography analyses of mGO53, TGF-β-Trap, αCD4 and 4T-Trap antibodies.

FIG. 54 shows molecular weights of αCD4, mGO53, 4T-Trap and TGF-β-Trap antibodies detected by Coomassie Bright Blue staining of samples run in a SDS-PAGE gel under non-reduced or reduced conditions. Molecular size markers (kDa) are shown on the left. (HC=heavy chain; LC=light chain.)

FIG. 55 shows a schematic representation of human CD4 structure and purity examination of recombinant soluble CD4 (sCD4) by SDS-PAGE followed by Coomassie Bright Blue staining.

FIG. 56 shows SPR sensorgrams of 4T-Trap and αCD4 binding to immobilized CD4 (left panel) as well as 4T-Trap and αTGF-β (1D11 clone) binding to immobilized TGF-β1 (right panel). RU, response unit.

FIG. 57 shows the binding affinities of 4T-Trap and αCD4 to human CD4 as well as 4T-Trap and αTGF-β (1D11 clone) to human TGF-β1, as determined by surface plasmon resonance.

FIG. 58 shows binding of 4T-Trap to human CD4 ectopically expressed on HEK293 cells. Cells were incubated with serial dilutions of 4T-Trap and αCD4 antibodies followed by a fluorophore-conjugated anti-mouse IgG secondary antibody. Samples were analyzed by flow cytometry. The measured mean fluorescence intensity (MFI) was quantified (n=3 technical replicates).

FIG. 59 shows TGF-β signaling inhibitory functions of 4T-Trap and αTGF-β. HEK293 cells transfected with a TGF-β/SMAD firefly luciferase reporter plasmid and a pRL-TK Renilla luciferase reporter plasmid were incubated with the indicated antibodies for 30 min and treated with 10 ng/mL recombinant human TGF-β1 for 12 hours before subjected to the luciferase assay (n=3 technical replicate). (RU=relative unit of normalized Firefly luciferase activity to Renilla luciferase activity.) All statistical data are shown as mean±SEM.

FIG. 60 shows results from enzyme-linked immunosorbent assay (ELISA) experiments to assess 4T-Trap, TGF-β-Trap, αCD4 and mGO53 binding to CD4, TGF-β1, or both molecules. Serial dilutions of 4T-Trap or control antibodies were incubated with plate-bound CD4 (left panel), TGF-β1 (middle panel), or CD4 followed by TGF-β1 (right panel). The binding activities were determined via an anti-mouse IgG (left and middle panels) or a biotinylated anti-human TGF-β1 IgG (right panel) ELISA (n=3 technical replicates). Optical densities (OD) were detected at 450 nm with background correction at 570 nm. All statistical data are shown as mean±SEM

FIG. 61 shows TGF-β signaling inhibitory functions of 4T-Trap and control antibodies in HEK293-hCD4 cells. HEK293-hCD4 cells transfected with a TGF-β/SMAD Firefly luciferase reporter plasmid and a pRL-TK Renilla luciferase reporter plasmid were incubated with varying doses of antibodies for 30 min, washed and treated with 10 ng/mL TGF-β1 for 12 h before subject to the luciferase assay (n=3 technical replicates). RU, relative unit of normalized Firefly luciferase activity to Renilla luciferase activity. All statistical data are shown as mean±SEM.

FIG. 62 shows a schematic representation of recombineering a bacterial artificial chromosome (BAC) DNA containing the human CD4 locus with the proximal enhancer (PE) element replaced by its murine equivalent. The shuttle plasmid contains the mouse Cd4 PE flanked by two homologous arms of the human CD4 gene (250 bps), the E. coli. RecA gene to mediate homologous recombination, the SacB gene to mediate negative selection on sucrose, an Ampicillin resistance locus to mediate positive selection and a conditional R6Kγ replication origin.

FIG. 63 shows flow cytometry analyses of human CD4 expression on leukocyte populations from wild-type or human CD4 transgenic mice. CD4⁺ T cells (CD45⁺TCRβ⁺CD4⁺), CD8⁺ T cells (CD45⁺TCRβ⁺CD8⁺), and NK cells (CD45⁺TCRγ⁻TCRβ⁻NKp46⁺NK1.1⁺) were isolated from lymph nodes. B cells (CD45⁺MHCII⁺Ly6C⁻B220⁺), XCR1⁺ DCs (CD45⁺Lin⁻F4/80⁻Ly6C⁻CD11c⁺MHCII⁺XCR1⁺), CD11b⁺ DCs (CD45⁺Lin⁻F4/80⁻Ly6C⁻CD11c⁺MHCII⁺CD11b⁺), Monocytes (CD45⁺Lin⁻F4/80⁺Ly6C⁺CD11b⁺) and Macrophages (CD45⁺Lin⁻F4/80⁺CD11b⁻Ly6C⁻) were isolated from spleens.

FIG. 64 shows a schematic representation of biotinylated 4T-Trap and control antibodies.

FIG. 65 shows antibody serum concentrations, measured by ELISA, at different time points for mice that were administered with a single dose of 150 μg 4T-Trap, αCD4, TGF-β-Trap or mGO53 by intravenous injection.

FIG. 66 shows antibody serum concentrations post-injection, measured by ELISA, for mice that were administered with a single dose of 50 μg, 100 μg, 150 μg or 450 μg 4T-Trap by intravenous injection.

FIG. 67 shows percentage of human CD4 molecule occupancy, as measured by flow cytometry, for mice that were administered with a single dose of 50 μg, 100 μg, 150 μg or 450 μg 4T-Trap by intravenous injection.

FIG. 68 shows immunoblotting analyses of TGF-β-induced SMAD2/3 phosphorylation in mouse CD4⁺ T cells isolated from human CD4 transgenic mice with different levels of 4T-Trap human CD4 (hCD4) target occupancy (TO). Numbers under lanes indicate SMAD2/3 or pSMAD2/3 band intensity.

FIG. 69 shows a schematic representation of a treatment scheme with 4T-Trap and control antibodies. hCD4PyMT mice bearing 5×5 mm tumors were administered with 100 antibodies by intravenous injection twice a week for 5 weeks.

FIG. 70 shows tumor measurements from hCD4PyMT mice treated with 4T-Trap, αCD4, TGF-β-Trap or mGO53 (n=5 for each group). All statistical data are shown as mean±SEM. **: P<0.01; ***: P<0.001; and ns: not significant.

FIG. 71 shows representative immunofluorescence images of fibrinogen (Fg, white), CD31 (red), cleaved Caspase 3 (CC3, blue) and E-Cadherin (green) in mammary tumor tissues from hCD4PyMT mice treated with 4T-Trap, αCD4, TGF-β-Trap or mGO53 antibodies. Extravascular (EV) Fg deposition events (magenta arrows) were calculated from multiple 1 mm² regions (n=13 for each group). Isolated CD31⁺ endothelial cells (yellow arrows) were counted from multiple 1 mm² regions (n=13 for each group). All statistical data are shown as mean±SEM. ****: P<0.0001.

FIG. 72 shows representative immunofluorescence images of NG2⁺ pericytes (white), CD31⁺ endothelial cells (red), GP38⁺ fibroblasts (cyan) and E-Cadherin (green) in mammary tumor tissues from hCD4PyMT mice treated with 4T-Trap, αCD4, TGF-β-Trap or mGO53 antibodies. NG2-unbound (magenta arrows) or GP38-unbound (yellow arrows) isolated CD31⁺ endothelial cells were counted from multiple 1 mm² regions (n=13 for each group). All statistical data are shown as mean±SEM. ****: P<0.0001

FIG. 73 shows representative immunofluorescence images of collagen IV (Col IV, white), CD31 (red), fibronectin (FN, cyan) and E-Cadherin (green) in mammary tumor tissues from hCD4PyMT mice treated with 4T-Trap, αCD4, TGF-β-Trap or mGO53 antibodies. The average continuous lengths of Col IV and FN were measured in multiple 1 mm² regions (n=13 for each group). All statistical data are shown as mean±SEM. ****: P<0.0001.

FIG. 74 shows representative immunofluorescence images of hypoxic probe (HPP, white), CD31 (red), cleaved Caspase 3 (CC3, blue) and E-Cadherin (green) in mammary tumor tissues from mice treated with the indicated antibodies and time points. The percentage of CD31⁺ areas, HPP⁺ without (W/O) CC3⁺ areas or HPP⁺ with (W/) CC3⁺ areas over E-Cadherin⁺ epithelial regions was calculated from multiple 1 mm² regions (n=5 for each group). Isolated CD31⁺ endothelial cells (yellow arrows) were counted from multiple 1 mm² regions (n=5 for each group). All statistical data are shown as mean±SEM. **: P<0.01; ***: P<0.001; ****: P<0.0001; and ns: not significant.

FIG. 75A shows a schematic representation of treatment with 4T-Trap and control antibodies. hCD4PyMT mice bearing 9×9 mm tumors were administered with 100 μg antibodies by intravenous injection twice a week for 4 weeks. FIG. 75B shows singular tumor measurements from hCD4PyMT mice treated with 4T-Trap, αCD4, TGF-β-Trap or mGO53 (n=7, 6, 7 and 5). FIG. 75C shows representative immunofluorescence images of hypoxia probe (HPP, white), CD31 (red), cleaved Caspase 3 (CC3, blue) and E-Cadherin (green) in mammary tumor tissues from mice treated with 4T-Trap at the indicated time points. All statistical data are shown as mean±SEM. ***: P<0.001.

FIG. 76 shows representative flow cytometry plots of IFN-γ and IL-4 expression in conventional CD4⁺Foxp3⁻ T cells from the tumor-draining lymph nodes of hCD4PyMT mice treated with 4T-Trap, αCD4, TGF-β-Trap or mGO53 antibodies as well as statistical analyses of the gated populations (n=3 for each group). All statistical data are shown as mean±SEM. **: P<0.01; and ns: not significant.

FIG. 77A shows representative immunofluorescence images of CD4 (white) and Biotin (red) staining in the tumor-draining lymph nodes of mice treated with the indicated biotinylated antibodies. FIG. 77B shows flow cytometry analyses of pSmad2 expression on resting or activated CD4⁺ T cells from the tumor-draining lymph nodes of mice treated with the indicated antibodies. CD4⁺ T cells were left untreated (resting) or treated with PMA/ionomycin for 4 hr (activated) before pSmad2 staining. FIG. 77C shows representative flow cytometry plots and statistical analyses of CD62L and CD44 expression in conventional CD4⁺Foxp3⁻ T cells from the tumor-draining lymph nodes of hCD4PyMT mice treated with 4T-Trap, αCD4, TGF-β-Trap or mGO53 antibodies (n=3 for each group). All statistical data are shown as mean±SEM. **: P<0.01; ***: P<0.001 and ns: not significant.

FIG. 78 shows representative flow cytometry plots of TCRβ, NK1.1, CD4, CD8, and Foxp3 expression in tumor-infiltrating leukocytes from hCD4PyMT mice treated with 4T-Trap, αCD4, TGF-β-Trap or mGO53 antibodies as well as statistical analyses of the gated populations. All statistical data are shown as mean±SEM. **: P<0.01; and ns: not significant.

FIG. 79 shows tumor measurements from hCD4PyMT mice treated with mGO53 or 4T-Trap in the absence or presence of an IL-4 neutralizing antibody (αIL-4) (n=5 for each group). All statistical data are shown as mean±SEM. ***: P<0.001; ****: P<0.0001.

FIG. 80 shows tumor measurements from hCD4PyMT mice treated with mGO53 or 4T-Trap in the absence or presence of an IFN-γ neutralizing antibody (αIFN-γ) (n=5 for each group). All statistical data are shown as mean±SEM. ***: P<0.001.

FIG. 81 shows representative immunofluorescence images of hypoxic probe (HPP, white), CD31 (red), cleaved Caspase 3 (CC3, blue) and E-Cadherin (green) in mammary tumor tissues from hCD4PyMT mice treated with mGO53 or 4T-Trap in the absence or presence of αIL-4 or αIFN-γ.

FIG. 82 shows representative immunofluorescence images of CD31 (white), hypoxic probe (HPP, red) and VEGFA (green) in mammary tumor tissues from hCD4PyMT mice treated with mGO53 or 4T-Trap. The percentage of HPP⁺VEGFA^{hi} areas was calculated from multiple 1 mm² regions (n=6 for each group). All statistical data are shown as mean±SEM. ****: P<0.0001.

FIG. 83 shows a schematic representation of human VEGFR1, VEGFR2 and VEGF-Trap as well as purity examination of recombinant VEGF-Trap by SDS-PAGE followed by Coomassie Bright Blue staining.

FIG. 84 shows VEGF signaling inhibitory function of VEGF-Trap. HEK293 cells transfected with a VEGF/NFAT firefly luciferase reporter plasmid, together with a VEGFR2 expression plasmid and a pRL-TK Renilla luciferase reporter plasmid, were incubated with different concentrations of VEGF-Trap for 30 min followed by 10 ng/mL recombinant human VEGF 165 for 12 h before subject to the luciferase assay (n=3 technical replicates). (RU=relative unit of normalized Firefly luciferase activity to Renilla luciferase activity.) All statistical data are shown as mean±SEM.

FIG. 85 shows representative immunofluorescence images of hypoxic probe (HPP, white), CD31 (red), cleaved Caspase 3 (CC3, blue) and E-Cadherin (green) in mammary tumor tissues from mice treated with mGO53, 4T-Trap, VEGF-Trap or 4T-Trap and VEGF-Trap. The percentage of CD31⁺ areas, HPP⁺ areas or CC3⁺ areas over E-Cadherin⁺ epithelial regions was calculated from multiple 1 mm² regions (n=10 for each group). Isolated CD31⁺ endothelial cells were counted from multiple 1 mm² regions (n=10 for each group). All statistical data are shown as mean±SEM. ****: P<0.0001.

FIG. 86 shows representative high magnification immunofluorescence images of hypoxic probe (HPP, white), CD31 (red), CC3 (blue) and E-Cadherin (green) in mammary tumor tissues from mice treated with 4T-Trap or 4T-Trap and VEGF-Trap. The shortest distance of HPP³⁺ regions (magenta dashed lines) or CC3⁺ regions (yellow dashed lines) to CD31⁺ endothelial cells was measured and plotted. All statistical data are shown as mean±SEM. ****: P<0.0001; and ns: not significant.

FIG. 87 shows tumor measurements from hCD4PyMT mice treated with mGO53, 4T-Trap, VEGF-Trap or 4T-Trap and VEGF-Trap (n=5 for each group). All statistical data are shown as mean±SEM. **: P<0.01; ***: P<0.001; and ns: not significant.

FIG. 88 shows a Kaplan-Meier survival curve for hCD4PyMT mice treated with mGO53, 4T-Trap, VEGF-Trap or 4T-Trap and VEGF-Trap. mGO53, n=10; 4T-Trap, n=9; VEGF-Trap, n=10; 4T-Trap and VEGF-Trap, n=4.

FIG. 89 shows a schematic representation of single-chain variable fragment (ScFv)-Fc fusion. Anti-CD4 ScFv is adapted from ibalizumab.

FIG. 90 shows an αCD4 single-chain variable fragment (ScFv) labeling test in CD4⁺ T cells and CD8⁺ T cells from human blood.

FIG. 91 shows a schematic representation of a bi-specific modality combined an anti-CD4 single-chain variable fragment (ScFv) with an anti-TGF-β ScFv. Anti-TGF-β ScFv is adapted from fresolimumab.

FIG. 92 shows SDS-PAGE analysis of purified recombinant bi-specific antibody (αCD4/αTGF-β). Left: reduced condition; right: non-reduced condition.

FIG. 93 shows functional validation of αCD4/αTGF-β in vitro. 293-hCD4 cells were left untreated or incubated with αCD4 or αCD4/αTGF-β for 20 min, washed 3 times to remove unbound antibodies, left untreated or treated with hTGF-β1 (5 ng/ml) for 1 h before SDS-PAGE and Western blot experiments with the indicated antibodies.

FIG. 94 shows tumor measurements from hCD4PyMT mice treated with αCD4 or αCD4/αTGF-β (n=3 for each group). All statistical data are shown as mean±SEM. ***: P<0.001; and ****: P<0.0001.

FIG. 95 shows representative immunofluorescence images of CD31 (red), cleaved Caspase 3 (CC3, blue) and E-Cadherin (green) in mammary tumor tissues from hCD4PyMT mice treated with αCD4 or αCD4/αTGF-β.

FIG. 96 shows exemplary V_{L} and V_{H} amino acid sequences of the CD4 targeting moiety present in the CD4 targeting fusion proteins described herein (SEQ ID NO: 1 and SEQ ID NO: 5). The CDR1, CDR2 and CDR3 regions of the V_{L} and V_{H} domains are underlined and are represented by SEQ ID NOs: 2-4 and 6-8.

FIG. 97 shows exemplary nucleic acid sequences encoding the V_{L} and V_{H} amino acid sequences of the CD4 targeting moiety present in the CD4 targeting fusion proteins described herein (SEQ ID NO: 9 and SEQ ID NO: 10).

FIG. 98 shows exemplary amino acid sequences of (i) Transforming growth factor beta receptor type II (TGF-βRII) (SEQ ID NO: 11), and (ii) Transforming growth factor beta receptor type IIB (TGF-βRIIB) (SEQ ID NO: 12).

FIG. 99 shows exemplary nucleic acid sequences encoding (i) TGF-βRII (SEQ ID NO: 13), and (ii) TGF-βRIIB (SEQ ID NO: 14).

FIG. 100 shows exemplary amino acid sequences of (i) TGF-βRII extracellular domain that binds to TGF-β (SEQ ID NO: 15); (ii) TGF-βRIIB extracellular domain that binds to TGF-β (SEQ ID NO: 16); and (iii) TGF-βRII or TGF-βRIIIB minimal extracellular domain that binds to TGF-β (SEQ ID NO: 17).

FIG. 101 shows exemplary nucleic acid sequences encoding (i) TGF-βRII extracellular domain that binds to TGF-β (SEQ ID NO: 18); (ii) TGF-βRIIIB extracellular domain that binds to TGF-β (SEQ ID NO: 19); and (iii) TGF-βRII or TGF-βRIIIB minimal extracellular domain that binds to TGF-β (SEQ ID NO: 20).

FIG. 102 shows codon-optimized nucleic acid sequences encoding (i) TGF-βRII extracellular domain that binds to TGF-β (SEQ ID NO: 21); (ii) TGF-βRIIIB extracellular domain that binds to TGF-β (SEQ ID NO: 22); and (iii) TGF-βRII or TGF-βRIIIB minimal extracellular domain that binds to TGF-β (SEQ ID NO: 23).

FIG. 103 shows p-values of differentially expressed genes in tumor-infiltrating CD4⁺CD25⁻ T cells from Tgfbr2^{fl/fl}PyMT compared to those from ThPOK^{Cre}Tgfbr2^{fl/fl}PyMT mice.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al., eds. (2007) Current Protocols in Molecular Biology; the series Methods in Enzymology (Academic Press, Inc., N.Y.); MacPherson et al., (1991) PCR 1: A Practical Approach (IRL Press at Oxford University Press); MacPherson et al., (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al., eds (1996) Weir's Handbook of Experimental Immunology.

While passive immunotherapy of cancer with tumor-targeted monoclonal antibodies has demonstrated clinical efficacy, the goal of active therapeutic vaccination to induce T cell-mediated immunity and establish immunological memory against tumor cells has remained challenging. Cancer cells are able to escape elimination by chemotherapeutic agents or tumor-targeted antibodies via specific immunosuppressive mechanisms (i.e., immune tolerance). The present disclosure is based on the seminal discovery that blockade of TGF-β signaling in CD4⁺ helper T cells, but not CD8⁺ T cells, results in profound inhibition of tumor growth. Accordingly, the present disclosure provides compositions and methods that counteract tumor-associated immune tolerance and promote T cell-mediated adaptive antitumor immunity for maintenance of durable long-term protection against recurrent or refractory cancers.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

More particularly, antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds CD4 protein will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, the term “antibody-related polypeptide” means antigen-binding antibody fragments, including single-chain antibodies, that can comprise the variable region(s) alone, or in combination, with all or part of the following polypeptide elements: hinge region, CH₁, CH₂, and CH₃ domains of an antibody molecule. Also included in the technology are any combinations of variable region(s) and hinge region, CH₁, CH₂, and CH₃ domains. Antibody-related molecules useful in the present methods, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). As such “antibody fragments” or “antigen binding fragments” can comprise a portion of a full length antibody, generally the antigen binding or variable region thereof. Examples of antibody fragments or antigen binding fragments include Fab, Fab′, F(ab′)₂, and Fv fragments; diabodies; linear antibodies; single-chain antibody molecules; and multispecific antibodies formed from antibody fragments.

As used herein, the terms “single-chain antibodies” or “single-chain Fv (scFv)” refer to an antibody fusion molecule of the two domains of the Fv fragment, V_{L} and V_{H}. Single-chain antibody molecules may comprise a polymer with a number of individual molecules, for example, dimer, trimer or other polymers. Furthermore, although the two domains of the Fᵥ fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single-chain Fᵥ (scFᵥ)). Bird et al. (1988) Science 242:423-426 and Huston et al. (1988) Proc. Natl. Acad Sci. USA 85:5879-5883. Such single-chain antibodies can be prepared by recombinant techniques or enzymatic or chemical cleavage of intact antibodies.

Any of the above-noted antibody fragments are obtained using conventional techniques known to those of skill in the art, and the fragments are screened for binding specificity and neutralization activity in the same manner as are intact antibodies.

As used herein, an “antigen” refers to a molecule to which an antibody (or antigen binding fragment thereof) can selectively bind. The target antigen may be a protein, carbohydrate, nucleic acid, lipid, hapten, or other naturally occurring or synthetic compound. In some embodiments, the target antigen may be a polypeptide (e.g., a CD4 polypeptide). An antigen may also be administered to an animal to generate an immune response in the animal.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, scFvFc, Fab, Fab′ and F(ab′)₂, but are not limited thereto.

By “binding affinity” is meant the strength of the total noncovalent interactions between a single binding site of a molecule (e.g., an antigen binding fragment or a receptor) and its binding partner (e.g., an antigen/antigenic peptide or a ligand). The affinity of a molecule X for its partner Y can generally be represented by the dissociation constant (K_{D}). Affinity can be measured by standard methods known in the art, including those described herein. A low-affinity complex contains a molecule that generally tends to dissociate readily from its partner, whereas a high-affinity complex contains a molecule that generally tends to remain bound to its partner for a longer duration.

The term “binding molecule” refers to a polypeptide (e.g., an antibody, an antigen binding fragment, a fusion protein including a targeting moiety) that binds to an epitope or region within a target polypeptide.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, the term “CDR-grafted antibody” means an antibody in which at least one CDR of an “acceptor” antibody is replaced by a CDR “graft” from a “donor” antibody possessing a desirable antigen specificity.

As used herein, the term “chimeric antibody” means an antibody in which the Fc constant region of a monoclonal antibody from one species (e.g., a mouse Fc constant region) is replaced, using recombinant DNA techniques, with an Fc constant region from an antibody of another species (e.g., a human Fc constant region). See generally, Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 0125,023; Better et al., Science 240: 1041-1043, 1988; Liu et al., Proc. Natl. Acad. Sci. USA 84: 3439-3443, 1987; Liu et al., J. Immunol 139: 3521-3526, 1987; Sun et al., Proc. Natl. Acad. Sci. USA 84: 214-218, 1987; Nishimura et al., Cancer Res 47: 999-1005, 1987; Wood et al., Nature 314: 446-449, 1885; and Shaw et al., J. Natl. Cancer Inst. 80: 1553-1559, 1988.

As used herein, the term “consensus FR” means a framework (FR) antibody region in a consensus immunoglobulin sequence. The FR regions of an antibody do not contact the antigen.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “effector cell” means an immune cell which is involved in the effector phase of an immune response, as opposed to the cognitive and activation phases of an immune response. Exemplary immune cells include a cell of a myeloid or lymphoid origin, e.g., lymphocytes (e.g., B cells, T cells including helper T (Th) cells and cytolytic T cells (CTLs), and natural killer cells), myeloid cells (e.g., dendritic cells, macrophages, monocytes, eosinophils, neutrophils, basophils and mast cells). Effector cells express specific Fc receptors and carry out specific immune functions. An effector cell can induce antibody-dependent cell-mediated cytotoxicity (ADCC) or antibody-dependent cell-mediated phagocytosis (ADCP). For example, natural killer cells, macrophages, dendritic cells, neutrophils, and eosinophils which express FcαR are involved in specific killing of target cells and presenting antigens to other components of the immune system.

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. In some embodiments, an “epitope” of the CD4 protein is a region of the protein to which the CD4 targeting moiety of the fusion proteins of the present technology specifically bind. In some embodiments, the epitope is a conformational epitope or a non-conformational epitope.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, the term “guide RNA (gRNA)” refers to an RNA which can be specific for a target DNA and can form a complex with a Cas protein. A guide RNA can comprise a guide sequence, or spacer sequence, that specifies a target site and guides an RNA/Cas complex to a specified target DNA for cleavage. Site-specific cleavage of a target DNA occurs at locations determined by both 1) base-pairing complementarity between a guide RNA and a target DNA (also called a protospacer) and 2) a short motif in a target DNA referred to as a protospacer adjacent motif (PAM).

As used herein, the term “CD4⁺ helper T cells” refer to CD4 expressing T cells that recognize an MHC class II-antigenic peptide complex that is expressed on antigen presenting cells (APCs) such as dendritic cells, B-cells, macrophages etc., and release effector T cell cytokines. Examples of CD4⁺ helper T cells include T_{H}1 and T_{H}2 cells, but exclude Tregs.

As used herein, a “heterodimerization domain that is incapable of forming a stable homodimer” refers to a member of a pair of distinct but complementary chemical motifs (e.g., amino acids, nucleotides, sugars, lipids, synthetic chemical structures, or any combination thereof) which either exclusively self-assembles as a heterodimer with the second complementary member of the pair, or shows at least a 10⁴ fold preference for assembling into a heterodimer with the second complementary member of the pair, or forms a homodimer with an identical member that is not stable under reducing conditions such as >2 mM 2-MEA at room temperature for 90 minutes (see e.g., Labrijn, A. F. et al., Proc. Natl. Acad. Sci. 110, 5145-50 (2013). Examples of such heterodimerization domains include, but are not limited to CH2-CH3 that include any of the Fc variants/mutations described herein, WinZip-A1B1, a pair of complementary oligonucleotides, and a CH-1 and CL pair.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, “humanized” forms of non-human (e.g., murine) antibodies are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some embodiments, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains (e.g., Fab, Fab′, F(ab′)₂, or Fv), in which all or substantially all of the hypervariable loops correspond to those of a non-human immunoglobulin and all or substantially all of the FR regions are those of a human immunoglobulin consensus FR sequence although the FR regions may include one or more amino acid substitutions that improve binding affinity. The number of these amino acid substitutions in the FR are typically no more than 6 in the H chain, and in the L chain, no more than 3. The humanized antibody optionally may also comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992). See e.g., Ahmed & Cheung, FEBS Letters 588(2):288-297 (2014).

As used herein, the term “hypervariable region” refers to the amino acid residues of an antibody which are responsible for antigen-binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the term “immunomodulatory moiety” refers to a polypeptide that binds a specific component of a Treg cell, or myeloid cell and modulates the number or function of Treg cells or myeloid cells. In an additional aspect, the “immunomodulatory moiety” specifically binds a cytokine, cytokine receptor, co-stimulatory molecule, or co-inhibitory molecule that modulates the immune system. In some embodiments, the immunomodulatory moiety is an antagonist that inhibits the function of the targeted molecule. Additionally or alternatively, in some embodiments, the immunomodulatory moiety specifically binds TGF-β or transforming growth factor-β receptor (TGF-βR). The immunomodulatory moiety may comprise an extracellular domain or ligand-binding sequence of one of the following receptors: transforming growth factor-β receptor II (TGF-βRII, or TGF-βRIIB) The extracellular domain of the specific receptor may bind the cognate ligand and inhibit the interaction of the ligand with its native receptor. The immunomodulatory moiety may be fused to the C-terminus or the N-terminus of the targeting moiety. In certain embodiments, the fusion protein is represented by X-Fc-Y, Y-Fc-X, X-Z-Y, Y-X-Fc, or Y-Z-X, wherein X is the targeting moiety, Fc is an immunoglobulin Fc region, Y is the immunomodulatory moiety, and Z is a linker sequence.

As used herein, the terms “individual”, “patient”, or “subject” can be an individual organism, a vertebrate, a mammal, or a human. In some embodiments, the individual, patient or subject is a human.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present methods may be made by the hybridoma method first described by Kohler et al., Nature 256:495 (1975), or may be made by recombinant DNA methods (See, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, “specifically binds” refers to a molecule (e.g., an antibody or antigen binding fragment thereof) which recognizes and binds another molecule (e.g., an antigen), but that does not substantially recognize and bind other molecules. The terms “specific binding,” “specifically binds to,” or is “specific for” a particular molecule (e.g., a polypeptide, or an epitope on a polypeptide), as used herein, can be exhibited, for example, by a molecule having a K_{D} for the molecule to which it binds to of about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. The term “specifically binds” may also refer to binding where a molecule (e.g., an antibody or antigen binding fragment thereof) binds to a particular polypeptide (e.g., a CD4 polypeptide), or an epitope on a particular polypeptide, without substantially binding to any other polypeptide, or polypeptide epitope.

As used herein, the term “simultaneous” therapeutic use refers to the administration of at least two active ingredients by the same route and at the same time or at substantially the same time.

As used herein, “targeting moiety” refers to a molecule that has the ability to localize and bind to a specific molecule or cellular component. The targeting moiety can be an antibody, antibody fragment, polypeptide, or any combination thereof and/or can bind to a molecule present in a cell or tissue, for example an immune cell. In some embodiments, the targeting moiety can bind a target molecule that modulates the immune response.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Amino acid sequence modification(s) of the fusion proteins described herein are contemplated. For example, it may be desirable to improve the binding affinity and/or other biological properties of the fusion protein. Amino acid sequence variants of a fusion protein are prepared by introducing appropriate nucleotide changes into its corresponding nucleic acid, or by peptide synthesis. Such modifications include, for example, deletions from, and/or insertions into and/or substitutions of, residues within the amino acid sequences of the fusion protein. Any combination of deletion, insertion, and substitution is made to obtain the fusion protein of interest, as long as the obtained fusion protein possesses the desired properties. The modification also includes the change of the pattern of glycosylation of the protein. The sites of greatest interest for substitutional mutagenesis include the hypervariable regions of the antigen binding fragments of the fusion proteins of the present technology, but FR alterations are also contemplated. “Conservative substitutions” are shown in the Table below.

TABLE 1
Amino Acid Substitutions

| | Exemplary | Conservative |
|---|---|---|
| Original Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

### CD4 Targeting Fusion Proteins of the Present Technology

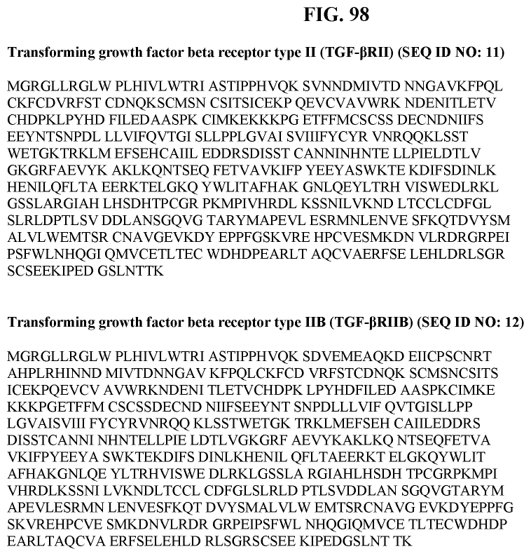

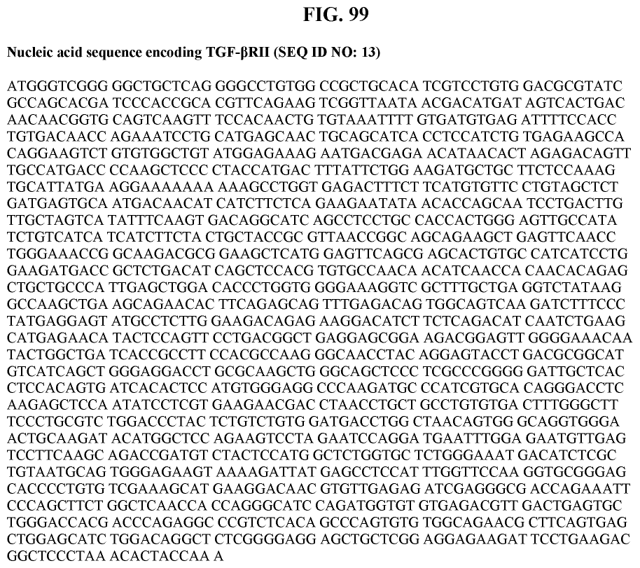

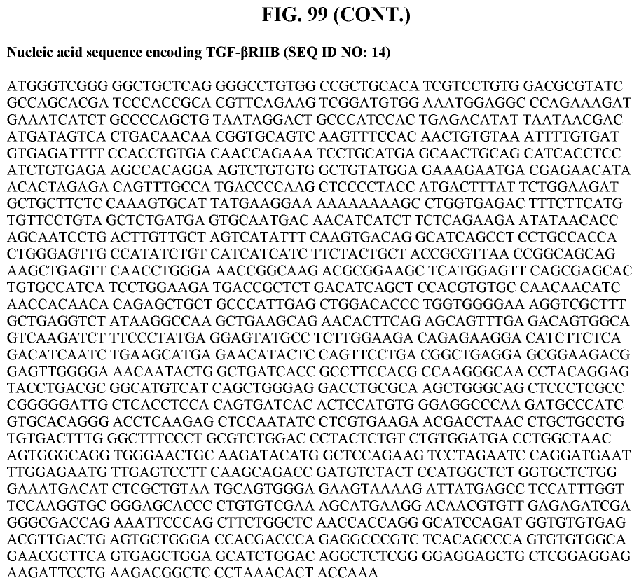

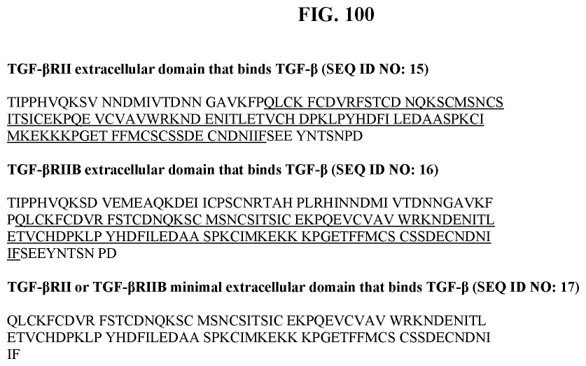

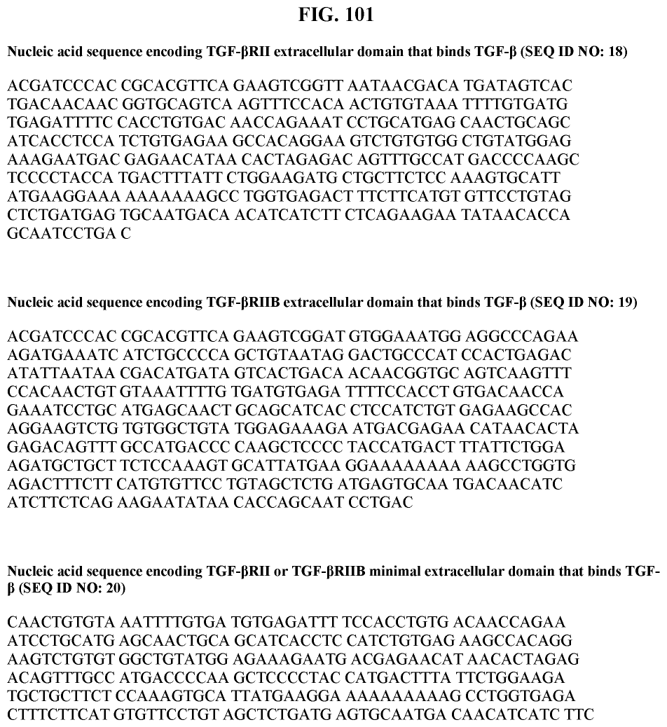

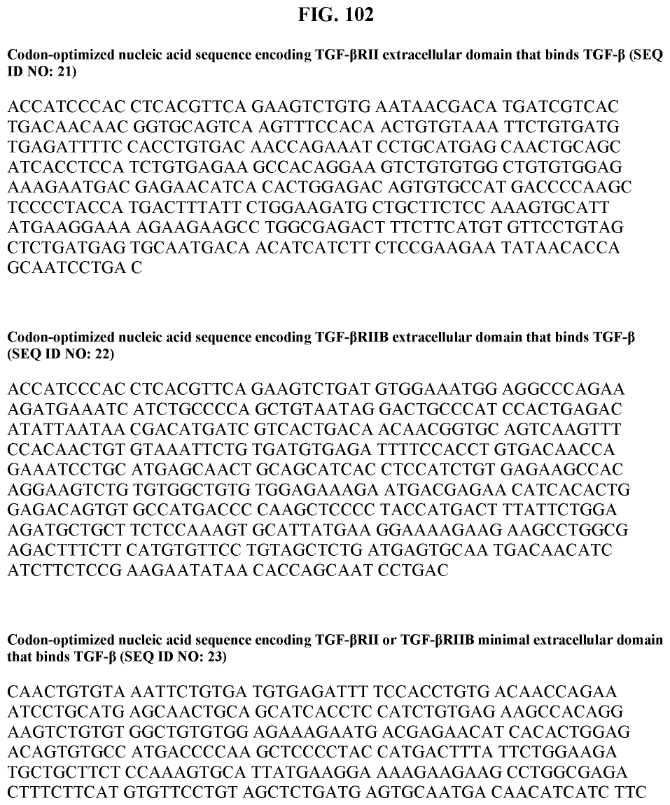

The cytokine TGF-β regulates a plethora of biological processes including development, fibrosis, carcinogenesis and immune responses. Although TGF-β overexpression is often associated with tumor progression and poor cancer patient prognosis (Fabregat et al., Current pharmaceutical design 20, 2934-2947 (2014)), systemic neutralizing of TGF-β has not been effective in clinical studies (Neuzillet, C. et al. Pharmacol Ther 147, 22-31 (2015), reflecting the pleiotropic functions of TGF-β in cancer (Massague, J. Cell 134, 215-230 (2008)). FIG. 98 shows exemplary amino acid sequences of transforming growth factor-β receptor II (TGF-βRII) (SEQ ID NO: 11), and (ii) Transforming growth factor-β receptor IIB (TGF-βRIIIB) (SEQ ID NO: 12).

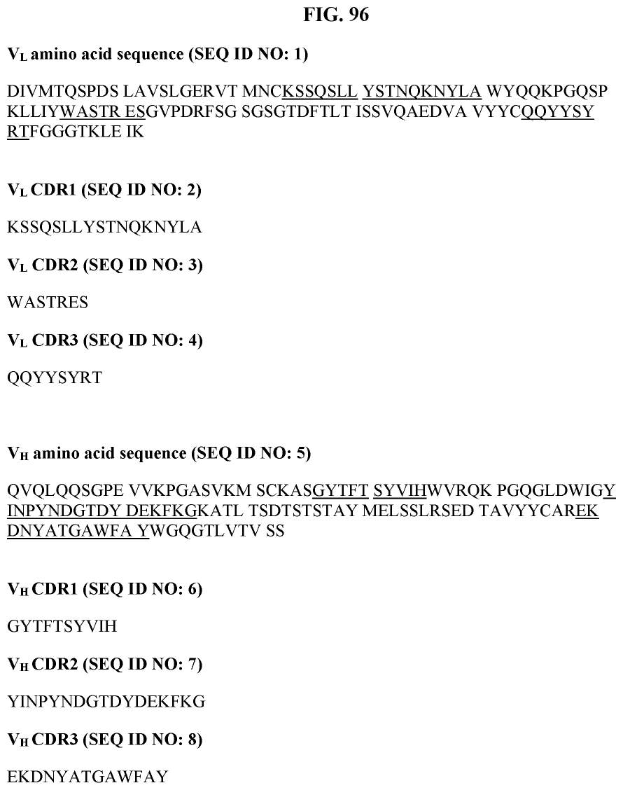

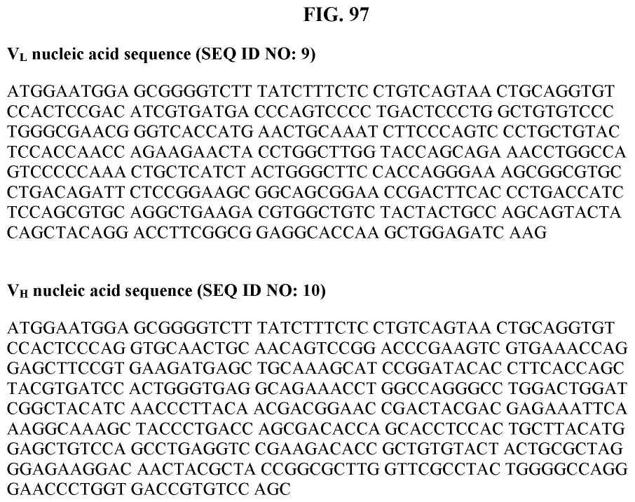

The present disclosure demonstrates that blockade of TGF-β signaling in CD4⁺ helper T cells, but not CD8⁺ T cells, results in profound inhibition of tumor growth. Accordingly, the present disclosure provides compositions that selectively inhibit TGF-β signaling in CD4⁺ helper T cells, thereby inhibiting tumor growth. Accordingly, the CD4 targeting fusion proteins of the present disclosure may be useful in the treatment of cancer. CD4 targeting fusion proteins within the scope of the present technology may comprise for example monoclonal, chimeric, or humanized antibodies that specifically bind CD4 polypeptide, a homolog, derivative or a fragment thereof. The present disclosure also provides CD4 targeting fusion proteins that include antigen binding fragments that specifically bind to CD4, wherein the antigen binding fragment is selected from the group consisting of Fab, F(ab)′2, Fab′, scFᵥ, and Fᵥ. FIGS. 96-97 show exemplary V_{L} and Vu amino acid sequences and nucleic acid sequences of a CD4 targeting moiety that are useful for generating the CD4 targeting fusion proteins of the present technology. Exemplary heavy chain (HC) and light chain (LC) amino acid sequences include:

HC1
(SEQ ID NO: 24)
QVQLQQSGPE VVKPGASVKM SCKASGYTFT SYVIHWVRQK
PGQGLDWIGY INPYNDGTDY DEKFKGKATL TSDTSTSTAY
MELSSLRSED TAVYYCAREK DNYATGAWFA YWGQGTLVTV
SSASTKGPSV FPLAPCSRST SESTAALGCL VKDYFPEPVT
VSWNSGALTS GVHTFPAVLQ SSGLYSLSSV VTVPSSSLGT
KTYTCNVDHK PSNTKVDKRV ESKYGPPCPP CPAPEFLGGP
SVFLFPPKPK DTLMISRTPE VTCVVVDVSQ EDPEVQFNWY
VDGVEVHNAK TKPREEQFNS TYRVVSVLTV LHQDWLNGKE
YKCKVSNKGL PSSIEKTISK AKGQPREPQV YTLPPSQEEM
TKNQVSLTCL VKGFYPSDIA VEWESNGQPE NNYKTTPPVL
DSDGSFFLYS RLTVDKSRWQ EGNVFSCSVM HEALHNHYTQ
KSLSLSLG
HC2
(SEQ ID NO: 25)
QVQLQQSGPE VVKPGASVKM SCKASGYTFT SYVIHWVRQK
PGQGLDWIGY INPYNDGTDY DEKFKGKATL TSDTSTSTAY
MELSSLRSED TAVYYCAREK DNYATGAWFA YWGQGTLVTV
SSASTKGPSV FPLAPCSRST SESTAALGCL VKDYFPEPVT
VSWNSGALTS GVHTFPAVLQ SSGLYSLSSV VTVPSSSLGT
KTYTCNVDHK PSNTKVDKRV ESKYGPPCPP CPAPEFEGGP
SVFLFPPKPK DTLMISRTPE VTCVVVDVSQ EDPEVQFNWY
VDGVEVHNAK TKPREEQFNS TYRVVSVLTV LHQDWLNGKE
YKCKVSNKGL PSSIEKTISK AKGQPREPQV YTLPPSQEEM
TKNQVSLTCL VKGFYPSDIA VEWESNGQPE NNYKTTPPVL
DSDGSFFLYS RLTVDKSRWQ EGNVFSCSVM HEALHNHYTQ
KSLSLSLG
HC3
(SEQ ID NO: 26)
QVQLQQSGPE VVKPGASVK SCKASGYTFT SYVIHWVRQK
PGQGLDWIGY INPYNDGTDY DEKFKGKATL TSDTSTSTAY
MELSSLRSED TAVYYCAREK DNYATGAWFA YWGQGTLVTV
SSASTKGPSV FPLAPSSKST SGGTAALGCL VKDYFPEPVT
VSWNSGALTS GVHTFPAVLQ SSGLYSLSSV VTVPSSSLGT
QTYICNVNHK PSNTKVDKKV EPKSCDKTHT CPPCPAPEFE
GGPSVFLFPP KPKDTLMISR TPEVTCVVVD VSHEDPEVKF
NWYVDGVEVH NAKTKPREEQ YNSTYRVVSV LTVLHQDWLN
GKEYKCKVSN KALPASIEKT ISKAKGQPRE PQVYTLPPSR
DELTKNQVSL TCLVKGFYPS DIAVEWESNG QPENNYKTTP
PVLDSDGSFF LYSKLTVDKS RWQQGNVFSC SVMHEALHNH
YTQKSLSLSP G
LC1
(SEQ ID NO: 27)
DIVMTQSPDS LAVSLGERVT MNCKSSQSLL YSTNQKNYLA
WYQQKPGQSP KLLIYWASTR ESGVPDRFSG SGSGTDFTLT
ISSVQAEDVA VYYCQQYYSY RTFGGGTKLE IKRTVAAPSV
FIFPPSDEQL KSGTASVVCL LNNFYPREAK VQWKVDNALQ
SGNSQESVTE QDSKDSTYSL SSTLTLSKAD YEKHKVYACE
VTHQGLSSPV TKSFNRGEC

In one aspect, the present disclosure provides a fusion protein comprising a CD4 targeting moiety fused with an immunomodulatory moiety, wherein: the CD4 targeting moiety comprises a heavy chain immunoglobulin variable domain (V_{H}) and a light chain immunoglobulin variable domain (V_{L}), wherein: (a) the V_{H} comprises a V_{H}-CDR1 sequence of GYTFTSYVIH (SEQ ID NO: 6), a V_{H}-CDR2 sequence of YINPYNDGTDYDEKFKG (SEQ ID NO: 7), and a V_{H}-CDR3 sequence of EKDNYATGAWFAY (SEQ ID NO: 8), and (b) the V_{L} comprises a V_{L}-CDR1 sequence of KSSQSLLYSTNQKNYLA (SEQ ID NO: 2), a V_{L}-CDR2 sequence of WASTRES (SEQ ID NO: 3), and a V_{L}-CDR3 sequence of QQYYSYRT (SEQ ID NO: 4); and the immunomodulatory moiety comprises an amino acid sequence of a TGF-β receptor II (TGF-βRII) selected from the group consisting of SEQ ID NOs: 11-12 and 15-17. In some embodiments, the TGF-β receptor II is encoded by a nucleic acid sequence selected from the group consisting of SEQ ID NOs: 13-14, 18-20, and 21-23. The immunomodulatory moiety may be fused to the C-terminus or the N-terminus of the CD4 targeting moiety. In some embodiments of the fusion protein of the present technology, the V_{H} comprises an amino acid sequence that is at least 80%, at least 85%, at least 95%, or 100% identical to SEQ ID NO: 5; and/or the V_{L} comprises an amino acid sequence that is at least 80%, at least 85%, at least 95%, or 100% identical to SEQ ID NO: 1. In another aspect, one or more amino acid residues in the fusion proteins provided herein are substituted with another amino acid. The substitution may be a “conservative substitution” as defined herein.

Additionally or alternatively, in some embodiments of the fusion protein of the present technology, the CD4 targeting moiety comprises an antibody or an antigen binding fragment that specifically binds CD4. In some embodiments, the antibody of the CD4 targeting moiety comprises a heavy chain (HC) and a light chain (LC). Additionally or alternatively, in some embodiments, the immunomodulatory moiety is fused to the N-terminus or C-terminus of the antibody HC of the CD4 targeting moiety. Additionally or alternatively, in some embodiments, the immunomodulatory moiety is fused to the N-terminus or C-terminus of the antibody LC of the CD4 targeting moiety. In certain embodiments, the immunomodulatory moiety is fused to the N-terminus of the antibody HC and the N-terminus of the antibody LC of the CD4 targeting moiety. In other embodiments, the immunomodulatory moiety is fused to the C-terminus of the antibody HC and the C-terminus of the antibody LC of the CD4 targeting moiety.

Additionally or alternatively, in some embodiments, the fusion protein may be represented by the formula X-Fc-Y or X-Z-Y, wherein X is the CD4 targeting moiety, Fc is an immunoglobulin Fc domain, Y is the immunomodulatory moiety and Z is a linker sequence. In other embodiments, the fusion protein may be represented by the formula Y-Fc-X, Y-X-Fc, or Y-Z-X, wherein X is the CD4 targeting moiety, Fc is an immunoglobulin Fc domain, Y is the immunomodulatory moiety and Z is a linker sequence. Additionally or alternatively, in some embodiments of the fusion protein disclosed herein, the antibody further comprises a Fc domain of any isotype, e.g., but are not limited to, IgG (including IgG1, IgG2, IgG3, and IgG4), IgA (including IgA₁ and IgA₂), IgD, IgE, or IgM, and IgY. Non-limiting examples of constant region sequences include:

Human IgD constant region, Uniprot: P01880
(SEQ ID NO: 28)
APTKAPDVFPIISGCRHPKDNSPVVLACLITGYHPTSVTVTWYMGTQS
QPQRTFPEIQRRDSYYMTSSQLSTPLQQWRQGEYKCVVQHTASKSKKEI
FRWPESPKAQASSVPTAQPQAEGSLAKATTAPATTRNTGRGGEEKKKEK
EKEEQEERETKTPECPSHTQPLGVYLLTPAVQDLWLRDKATFTCFVVGS
DLKDAHLTWEVAGKVPTGGVEEGLLERHSNGSQSQHSRLTLPRSLWNAG
TSVTCTLNHPSLPPQRLMALREPAAQAPVKLSLNLLASSDPPEAASWLL
CEVSGFSPPNILLMWLEDQREVNTSGFAPARPPPQPGSTTFWAWSVLRV
PAPPSPQPATYTCVVSHEDSRTLLNASRSLEVSYVTDHGPMK
Human IgG1 constant region, Uniprot: P01857
(SEQ ID NO: 29)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKK
VEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKL
TVDKSRWQQGNVFSCSVMHEALHNHYTQKSLSLSPGK
Human IgG2 constant region, Uniprot: P01859
(SEQ ID NO: 30)
ASTKGPSVFPLAPCSRSTSESTAALGCLVKDYFPEPVTVSWNSGALTSG
VHTFPAVLQSSGLYSLSSVVTVPSSNFGTQTYTCNVDHKPSNTKVDKTV
ERKCCVECPPCPAPPVAGPSVFLFPPKPKDTLMISRTPEVTCVVVDVSH
EDPEVQFNWYVDGVEVHNAKTKPREEQFNSTFRVVSVLTVVHQDWLNGK
EYKCKVSNKGLPAPIEKTISKTKGQPREPQVYTLPPSREEMTKNQVSLT
CLVKGFYPSDISVEWESNGQPENNYKTTPPMLDSDGSFFLYSKLTVDKS
RWQQGNVFSCSVMHEALHNHYTQKSLSLSPGK
Human IgG3 constant region, Uniprot: P01860
(SEQ ID NO: 31)
ASTKGPSVFPLAPCSRSTSGGTAALGCLVKDYFPEPVTVSWNSGALTS
GVHTFPAVLQSSGLYSLSSVVTVPSSSLGTQTYTCNVNHKPSNTKVDKR
VELKTPLGDTTHTCPRCPEPKSCDTPPPCPRCPEPKSCDTPPPCPRCPE
PKSCDTPPPCPRCPAPELLGGPSVFLFPPKPKDTLMISRTPEVTCVVVD
VSHEDPEVQFKWYVDGVEVHNAKTKPREEQYNSTFRVVSVLTVLHQDWL
NGKEYKCKVSNKALPAPIEKTISKTKGQPREPQVYTLPPSREEMTKNQV
SLTCLVKGFYPSDIAVEWESSGQPENNYNTTPPMLDSDGSFFLYSKLTV
DKSRWQQGNIFSCSVMHEALHNRFTQKSLSLSPGK
Human IgM constant region, Uniprot: P01871
(SEQ ID NO: 32)
GSASAPTLFPLVSCENSPSDTSSVAVGCLAQDFLPDSITLSWKYKNNSD
ISSTRGFPSVLRGGKYAATSQVLLPSKDVMQGTDEHVVCKVQHPNGNKE
KNVPLPVIAELPPKVSVFVPPRDGFFGNPRKSKLICQATGFSPRQIQVS
WLREGKQVGSGVTTDQVQAEAKESGPTTYKVTSTLTIKESDWLGQSMFT
CRVDHRGLTFQQNASSMCVPDQDTAIRVFAIPPSFASIFLTKSTKLTCL
VTDLTTYDSVTISWTRQNGEAVKTHTNISESHPNATFSAVGEASICEDD
WNSGERFTCTVTHTDLPSPLKQTISRPKGVALHRPDVYLLPPAREQLNL
RESATITCLVTGFSPADVFVQWMQRGQPLSPEKYVTSAPMPEPQAPGRY
FAHSILTVSEEEWNTGETYTCVAHEALPNRVTERTVDKSTGKPTLYNVS
LVMSDTAGTCY
Human IgG4 constant region, Uniprot: P01861
(SEQ ID NO: 33)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK
Human IgA1 constant region, Uniprot: P01876
(SEQ ID NO: 34)
ASPTSPKVFPLSLCSTQPDGNVVIACLVQGFFPQEPLSVTWSESGQGVT
ARNFPPSQDASGDLYTTSSQLTLPATQCLAGKSVTCHVKHYTNPSQDVT
VPCPVPSTPPTPSPSTPPTPSPSCCHPRLSLHRPALEDLLLGSEANLTC
TLTGLRDASGVTFTWTPSSGKSAVQGPPERDLCGCYSVSSVLPGCAEPW
NHGKTFTCTAAYPESKTPLTATLSKSGNTFRPEVHLLPPPSEELALNEL
VTLTCLARGFSPKDVLVRWLQGSQELPREKYLTWASRQEPSQGTTTFAV
TSILRVAAEDWKKGDTFSCMVGHEALPLAFTQKTIDRLAGKPTHVNVSV
VMAEVDGTCY
Human IgA2 constant region, Uniprot: P01877
(SEQ ID NO: 35)
ASPTSPKVFPLSLDSTPQDGNVVVACLVQGFFPQEPLSVTWSESGQNV
TARNFPPSQDASGDLYTTSSQLTLPATQCPDGKSVTCHVKHYTNPSQDV
TVPCPVPPPPPCCHPRLSLHRPALEDLLLGSEANLTCTLTGLRDASGAT
FTWTPSSGKSAVQGPPERDLCGCYSVSSVLPGCAQPWNHGETFTCTAAH
PELKTPLTANITKSGNTFRPEVHLLPPPSEELALNELVTLTCLARGFSP
KDVLVRWLQGSQELPREKYLTWASRQEPSQGTTTFAVTSILRVAAEDWK
KGDTFSCMVGHEALPLAFTQKTIDRMAGKPTHVNVSVVMAEVDGTCY
Human Ig kappa constant region, Uniprot: P01834
(SEQ ID NO: 36)
TVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVT
KSFNRGEC

In some embodiments, the fusion proteins of the present technology comprise a heavy chain constant region that is at least 80%, at least 85%, at least 90%, at least 95%, at least 99%, or is 100% identical to SEQ ID NOs: 28-35. Additionally or alternatively, in some embodiments, the fusion proteins of the present technology comprise a light chain constant region that is at least 80%, at least 85%, at least 90%, at least 95%, at least 99%, or is 100% identical to SEQ ID NO: 36.

In any of the embodiments disclosed herein, the fusion proteins of the present technology bind specifically to at least one CD4 polypeptide. In some embodiments, the fusion proteins of the present technology bind at least one CD4 polypeptide with a dissociation constant (K_{D}) of about 10⁻³ M, 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In certain embodiments, the fusion proteins comprise monoclonal antibodies, chimeric antibodies, or humanized antibodies, wherein the antibodies optionally comprise a human antibody framework region.

Additionally or alternatively, in certain embodiments of the fusion proteins described herein, the antibody or antigen binding fragment comprises an IgG1 constant region comprising one or more amino acid substitutions selected from the group consisting of D265A, N297A, K322A, L234F, L235E and P331S. Additionally or alternatively, in some embodiments, the fusion proteins comprise an IgG4 constant region comprising a S228P mutation.

Additionally or alternatively, in some embodiments, the fusion protein includes an antibody comprising a heavy chain (HC) amino acid sequence of SEQ ID NO: 24, SEQ ID NO: 25, or SEQ ID NO: 26, or a variant thereof having one or more conservative amino acid substitutions. Additionally or alternatively, in some embodiments, the fusion protein includes an antibody comprising a light chain (LC) amino acid sequence of SEQ ID NO: 27, or a variant thereof having one or more conservative amino acid substitutions. In some embodiments, the fusion proteins of the present technology comprise a HC amino acid sequence and a LC amino acid sequence selected from the group consisting of: SEQ ID NO: 24 and SEQ ID NO: 27; SEQ ID NO: 25 and SEQ ID NO: 27; and SEQ ID NO: 26 and SEQ ID NO: 27, respectively.

In some embodiments, the fusion protein includes an antibody comprising (a) a LC sequence that is at least 80%, at least 85%, at least 90%, at least 95%, or at least 99% identical to the LC sequence present in SEQ ID NO: 27; and/or (b) a HC sequence that is at least 80%, at least 85%, at least 90%, at least 95%, or at least 99% identical to the HC sequence present in any one of SEQ ID NO: 24, SEQ ID NO: 25, or SEQ ID NO: 26.

In any of the above embodiments of the fusion proteins, the V_{H} and V_{L} form an antigen binding site that binds to the extracellular domain of CD4. In some embodiments of the fusion proteins disclosed herein, the V_{H} and V_{L} are components of the same polypeptide chain. In other embodiments, the V_{H} and V_{L} are components of different polypeptide chains. In certain embodiments, the fusion protein of the present technology comprises a full-length antibody.

Additionally or alternatively, in some embodiments of the fusion proteins disclosed herein, the CD4 targeting moiety is fused with the immunomodulatory moiety via a linker. Any suitable linker known in the art can be used. In some embodiments, the CD4 targeting moiety is fused with the immunomodulatory moiety via a polypeptide linker. Any polypeptide linker known in the art may be used in the fusion proteins of the present technology. In some embodiments, the polypeptide linker is a Gly-Ser linker. In some embodiments, the polypeptide linker is or comprises a sequence of (GGGGS)ₙ (SEQ ID NO: 43), where n represents the number of repeating GGGGS (“GGGGS” disclosed as SEQ ID NO: 44) units and is 1, 2, 3, 4, 5, 6, 7, 8, 9, 10, 11, 12, 13, 14, 15, 16, 17, 18, 19, 20, 25, 30 or more. In some embodiments, the CD4 targeting moiety is directly fused to the immunomodulatory moiety.

In some aspects, the fusion proteins described herein contain structural modifications to facilitate rapid binding and cell uptake and/or slow release. In some aspects, the fusion protein of the present technology (e.g., CD4 targeting fusion protein) may contain a deletion in a CH2 constant heavy chain region to facilitate rapid binding and cell uptake and/or slow release. In some aspects, a Fab fragment is used to facilitate rapid binding and cell uptake and/or slow release. In some aspects, a F(ab)′2 fragment is used to facilitate rapid binding and cell uptake and/or slow release.

In another aspect, the present disclosure provides CD4 fusion proteins that bind to the same CD4 epitope as any fusion protein disclosed herein, wherein the CD4 fusion protein comprises a CD4 binding domain fused with an immunomodulatory moiety (e.g., comprising an extracellular domain of a TGF-β receptor II (TGF-βRII)).

In one aspect, the present technology provides a recombinant nucleic acid sequence encoding any of the fusion proteins described herein.

In another aspect, the present technology provides a host cell or vector expressing any nucleic acid sequence encoding any of the fusion proteins described herein.

The fusion proteins of the present technology can further be recombinantly fused to a heterologous polypeptide at the N- or C-terminus or chemically conjugated (including covalently and non-covalently conjugations) to polypeptides or other compositions. For example, the fusion proteins of the present technology can be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

In one aspect, the present disclosure provides compositions comprising fusion proteins of the present technology and a pharmaceutically-acceptable carrier, wherein the fusion proteins may be optionally conjugated to an agent selected from the group consisting of isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof. For a chemical bond or physical bond, a functional group on the fusion protein typically associates with a functional group on the agent. Alternatively, a functional group on the agent associates with a functional group on the fusion protein.

The functional groups on the agent and fusion protein can associate directly. For example, a functional group (e.g., a sulfhydryl group) on an agent can associate with a functional group (e.g., sulfhydryl group) on a fusion protein to form a disulfide. Alternatively, the functional groups can associate through a cross-linking agent (i.e., linker). Some examples of cross-linking agents are described below. The cross-linker can be attached to either the agent or the fusion protein. The number of agents or fusion proteins in a conjugate is also limited by the number of functional groups present on the other. For example, the maximum number of agents associated with a conjugate depends on the number of functional groups present on the fusion protein. Alternatively, the maximum number of fusion proteins associated with an agent depends on the number of functional groups present on the agent.

In yet another embodiment, the conjugate comprises one fusion protein associated to one agent. In one embodiment, a conjugate comprises at least one agent chemically bonded (e.g., conjugated) to at least one fusion protein. The agent can be chemically bonded to a fusion protein by any method known to those in the art. For example, a functional group on the agent may be directly attached to a functional group on the fusion protein. Some examples of suitable functional groups include, for example, amino, carboxyl, sulfhydryl, maleimide, isocyanate, isothiocyanate and hydroxyl.

The agent may also be chemically bonded to the fusion protein by means of cross-linking agents, such as dialdehydes, carbodiimides, dimaleimides, and the like. Cross-linking agents can, for example, be obtained from Pierce Biotechnology, Inc., Rockford, Ill. The Pierce Biotechnology, Inc. web-site can provide assistance. Additional cross-linking agents include the platinum cross-linking agents described in U.S. Pat. Nos. 5,580,990; 5,985,566; and 6,133,038 of Kreatech Biotechnology, B.V., Amsterdam, The Netherlands.

Alternatively, the functional group on the agent and fusion protein can be the same. Homobifunctional cross-linkers are typically used to cross-link identical functional groups. Examples of homobifunctional cross-linkers include EGS (i.e., ethylene glycol bis[succinimidylsuccinate]), DSS (i.e., disuccinimidyl suberate), DMA (i.e., dimethyl adipimidate.2HCl), DTSSP (i.e., 3,3′-dithiobis[sulfosuccinimidylpropionate])), DPDPB (i.e., 1,4-di-[3′-(2′-pyridyldithio)-propionamido]butane), and BMH (i.e., bis-maleimidohexane). Such homobifunctional cross-linkers are also available from Pierce Biotechnology, Inc.

In other instances, it may be beneficial to cleave the agent from the fusion protein. The web-site of Pierce Biotechnology, Inc. described above can also provide assistance to one skilled in the art in choosing suitable cross-linkers which can be cleaved by, for example, enzymes in the cell. Thus the agent can be separated from the fusion protein. Examples of cleavable linkers include SMPT (i.e., 4-succinimidyloxycarbonyl-methyl-a-[2-pyridyldithio]toluene), Sulfo-LC-SPDP (i.e., sulfosuccinimidyl 6-(3-[2-pyridyldithio]-propionamido)hexanoate), LC-SPDP (i.e., succinimidyl 6-(3-[2-pyridyldithio]-propionamido)hexanoate), Sulfo-LC-SPDP (i.e., sulfosuccinimidyl 6-(3-[2-pyridyldithio]-propionamido)hexanoate), SPDP (i.e., N-succinimidyl 3-[2-pyridyldithio]-propionamidohexanoate), and AEDP (i.e., 3-[(2-aminoethyl)dithio]propionic acid HCl).

In another embodiment, a conjugate comprises at least one agent physically bonded with at least one fusion protein. Any method known to those in the art can be employed to physically bond the agents with the fusion proteins. For example, the fusion proteins and agents can be mixed together by any method known to those in the art. The order of mixing is not important. For instance, agents can be physically mixed with fusion proteins by any method known to those in the art. For example, the fusion proteins and agents can be placed in a container and agitated, by for example, shaking the container, to mix the fusion proteins and agents. The fusion proteins can be modified by any method known to those in the art. For instance, the fusion protein may be modified by means of cross-linking agents or functional groups, as described above.

### Methods of Preparing the Fusion Proteins of the Present Technology

General Overview. CD4 targeting fusion proteins of the present technology that can be subjected to the techniques set forth herein may comprise monoclonal antibodies, and antibody fragments such as Fab, Fab′, F(ab′)₂, Fd, scFv, diabodies, antibody light chains, antibody heavy chains and/or antibody fragments. An antibody may be raised against the full-length CD4 protein, or to a portion of the extracellular domain of the CD4 protein. Techniques for generating antibodies directed to such target polypeptides are well known to those skilled in the art. Examples of such techniques include, for example, but are not limited to, those involving display libraries, xeno or human mice, hybridomas, and the like. Methods useful for the high yield production of antibody Fv-containing polypeptides, e.g., Fab′ and F(ab′)₂ antibody fragments have been described. See U.S. Pat. No. 5,648,237. Generally, an antibody is obtained from an originating species. More particularly, the nucleic acid or amino acid sequence of the variable portion of the light chain, heavy chain or both, of an originating species antibody having specificity for a target polypeptide antigen is obtained. An originating species is any species which was useful to generate the antibody of the present technology or library of antibodies, e.g., rat, mouse, rabbit, chicken, monkey, human, and the like. It should be understood that recombinantly engineered antibodies and antibody fragments, e.g., antibody-related polypeptides, which are directed to CD4 protein and fragments thereof are suitable for use in accordance with the present disclosure.

Phage or phagemid display technologies are useful techniques to derive antibody components of the fusion proteins of the present technology. Techniques for generating and cloning monoclonal antibodies are well known to those skilled in the art. Expression of sequences encoding antibody components of the fusion proteins of the present technology, can be carried out in E. coli.

Due to the degeneracy of nucleic acid coding sequences, other sequences which encode substantially the same amino acid sequences as those of the naturally occurring proteins may be used in the practice of the present technology These include, but are not limited to, nucleic acid sequences including all or portions of the nucleic acid sequences encoding the above polypeptides, which are altered by the substitution of different codons that encode a functionally equivalent amino acid residue within the sequence, thus producing a silent change. It is appreciated that the nucleotide sequence of a CD4 targeting fusion protein according to the present technology tolerates sequence homology variations of up to 25% as calculated by standard methods (“Current Methods in Sequence Comparison and Analysis,” Macromolecule Sequencing and Synthesis, Selected Methods and Applications, pp. 127-149, 1998, Alan R. Liss, Inc.) so long as such a variant forms an operative structure which recognizes CD4 proteins. For example, one or more amino acid residues within a polypeptide sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Also included within the scope of the present technology are proteins or fragments or derivatives thereof which are differentially modified during or after translation, e.g., by glycosylation, proteolytic cleavage, linkage to an antibody molecule or other cellular ligands, etc. Additionally, a fusion protein encoding nucleic acid sequence can be mutated in vitro or in vivo to create and/or destroy translation, initiation, and/or termination sequences or to create variations in coding regions and/or form new restriction endonuclease sites or destroy pre-existing ones, to facilitate further in vitro modification. Any technique for mutagenesis known in the art can be used, including but not limited to in vitro site directed mutagenesis, J. Biol. Chem. 253:6551, use of Tab linkers (Pharmacia), and the like.

Fusion Proteins. The CD4 targeting fusion proteins of the present technology may include not only heterologous signal sequences, but also other heterologous functional regions. The fusion does not necessarily need to be direct, but can occur through linker sequences. Moreover, fusion proteins of the present technology can also be engineered to improve physical characteristics. For instance, a region of additional amino acids, particularly charged amino acids, can be added to the N-terminus of the CD4 targeting fusion protein to improve stability and persistence during purification from the host cell or subsequent handling and storage. Also, peptide moieties can be added to a CD4 targeting fusion protein to facilitate purification. Such regions can be removed prior to final preparation of the CD4 targeting fusion protein. The addition of peptide moieties to facilitate handling of polypeptides are familiar and routine techniques in the art. The CD4 targeting fusion protein of the present technology can be fused to marker sequences, such as a peptide which facilitates purification of the fused polypeptide. In select embodiments, the marker amino acid sequence is a hexa-histidine peptide (SEQ ID NO: 45), such as the tag provided in a pQE vector (QIAGEN, Inc., Chatsworth, Calif.), among others, many of which are commercially available. As described in Gentz et al., Proc. Natl. Acad. Sci. USA 86: 821-824, 1989, for instance, hexa-histidine (SEQ ID NO: 45) provides for convenient purification of the fusion protein. Another peptide tag useful for purification, the “HA” tag, corresponds to an epitope derived from the influenza hemagglutinin protein. Wilson et al., Cell 37: 767, 1984.

Thus, any of these above fusion proteins can be engineered using the polynucleotides or the polypeptides of the present technology. Also, in some embodiments, the fusion proteins described herein show an increased half-life in vivo.

Fusion proteins having disulfide-linked dimeric structures (due to the IgG) can be more efficient in binding and neutralizing other molecules compared to the monomeric secreted protein or protein fragment alone. Fountoulakis et al., J. Biochem. 270: 3958-3964, 1995.

Similarly, EP-A-0 464 533 (Canadian counterpart 2045869) discloses fusion proteins comprising various portions of constant region of immunoglobulin molecules together with another human protein or a fragment thereof. In many cases, the Fc part in a fusion protein is beneficial in therapy and diagnosis, and thus can result in, e.g., improved pharmacokinetic properties. See EP-A 0232 262. Alternatively, deleting or modifying the Fc part after the fusion protein has been expressed, detected, and purified, may be desired. For example, the Fc portion can hinder therapy and diagnosis if the fusion protein is used as an antigen for immunizations. In drug discovery, e.g., human proteins, such as hIL-5, have been fused with Fc portions for the purpose of high-throughput screening assays to identify antagonists of hIL-5. Bennett et al., J. Molecular Recognition 8: 52-58, 1995; Johanson et al., J. Biol. Chem., 270: 9459-9471, 1995.

Single-Chain Antibodies. In one embodiment, the CD4 targeting fusion protein of the present technology comprises a single-chain anti-CD4 antibody. According to the present technology, techniques can be adapted for the production of single-chain antibodies specific to a CD4 protein (See, e.g., U.S. Pat. No. 4,946,778). Examples of techniques which can be used to produce single-chain Fvs and fusion proteins of the present technology include those described in U.S. Pat. Nos. 4,946,778 and 5,258,498; Huston et al., Methods in Enzymology, 203: 46-88, 1991; Shu, L. et al., Proc. Natl. Acad. Sci. USA, 90: 7995-7999, 1993; and Skerra et al., Science 240: 1038-1040, 1988.

Chimeric and Humanized Antibodies. In one embodiment, the CD4 targeting fusion protein of the present technology comprises a chimeric anti-CD4 antibody. In one embodiment, the CD4 targeting fusion protein of the present technology comprises a humanized anti-CD4 antibody. In one embodiment of the present technology, the donor and acceptor antibodies are monoclonal antibodies from different species. For example, the acceptor antibody is a human antibody (to minimize its antigenicity in a human), in which case the resulting CDR-grafted antibody is termed a “humanized” antibody.

Recombinant CD4 targeting fusion proteins including chimeric or humanized monoclonal antibodies that comprise both human and non-human portions, can be made using standard recombinant DNA techniques, and are within the scope of the present technology. For some uses, including in vivo use of the CD4 targeting fusion protein of the present technology in humans as well as use of these agents in in vitro detection assays, it is possible to use CD4 targeting fusion proteins comprising chimeric or humanized monoclonal antibodies. Such chimeric and humanized monoclonal antibodies can be produced by recombinant DNA techniques known in the art. Such useful methods include, e.g., but are not limited to, methods described in International Application No. PCT/US86/02269; U.S. Pat. No. 5,225,539; European Patent No. 184187; European Patent No. 171496; European Patent No. 173494; PCT International Publication No. WO 86/01533; U.S. Pat. Nos. 4,816,567; 5,225,539; European Patent No. 125023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314: 446-449; Shaw, et al., 1988. J. Natl. Cancer Inst. 80: 1553-1559; Morrison (1985) Science 229: 1202-1207; Oi, et al. (1986) BioTechniques 4: 214; Jones, et al., 1986. Nature 321: 552-525; Verhoeyan, et al., 1988. Science 239: 1534; Morrison, Science 229: 1202, 1985; Oi et al., BioTechniques 4: 214, 1986; Gillies et al., J. Immunol. Methods, 125: 191-202, 1989; U.S. Pat. No. 5,807,715; and Beidler, et al., 1988. J. Immunol. 141: 4053-4060. For example, antibodies can be humanized using a variety of techniques including CDR-grafting (EP 0 239 400; WO 91/09967; U.S. Pat. Nos. 5,530,101; 5,585,089; 5,859,205; 6,248,516; EP460167), veneering or resurfacing (EP 0 592 106; EP 0 519 596; Padlan E. A., Molecular Immunology, 28: 489-498, 1991; Studnicka et al., Protein Engineering 7: 805-814, 1994; Roguska et al., PNAS 91: 969-973, 1994), and chain shuffling (U.S. Pat. No. 5,565,332). In one embodiment, a cDNA encoding a murine anti-CD4 monoclonal antibody is digested with a restriction enzyme selected specifically to remove the sequence encoding the Fc constant region, and the equivalent portion of a cDNA encoding a human Fc constant region is substituted (See Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 125,023; Better et al. (1988) Science 240: 1041-1043; Liu et al. (1987) Proc. Natl. Acad. Sci. USA 84: 3439-3443; Liu et al. (1987) J Immunol 139: 3521-3526; Sun et al. (1987) Proc. Natl. Acad. Sci. USA 84: 214-218; Nishimura et al. (1987) Cancer Res 47: 999-1005; Wood et al. (1985) Nature 314: 446-449; and Shaw et al. (1988) J. Natl. Cancer Inst. 80: 1553-1559; U.S. Pat. Nos. 6,180,370; 6,300,064; 6,696,248; 6,706,484; 6,828,422.

In one embodiment, the present technology provides the construction of CD4 targeting fusion proteins comprising humanized antibodies that are unlikely to induce a human anti-mouse antibody (hereinafter referred to as “HAMA”) response, while still having an effective antibody effector function. As used herein, the terms “human” and “humanized”, in relation to antibodies, relate to any antibody which is expected to elicit a therapeutically tolerable weak immunogenic response in a human subject.

CDR Antibodies. In some embodiments, the CD4 targeting fusion protein of the present technology comprises an anti-CD4 CDR antibody. Generally the donor and acceptor antibodies used to generate the anti-CD4 CDR antibody are monoclonal antibodies from different species; typically the acceptor antibody is a human antibody (to minimize its antigenicity in a human), in which case the resulting CDR-grafted antibody is termed a “humanized” antibody. The graft may be of a single CDR (or even a portion of a single CDR) within a single V_{H} or V_{L} of the acceptor antibody, or can be of multiple CDRs (or portions thereof) within one or both of the V_{H} and V_{L}. Frequently, all three CDRs in all variable domains of the acceptor antibody will be replaced with the corresponding donor CDRs, though one needs to replace only as many as necessary to permit adequate binding of the resulting CDR-grafted antibody to CD4 protein. Methods for generating CDR-grafted and humanized antibodies are taught by Queen et al. U.S. Pat. Nos. 5,585,089; 5,693,761; 5,693,762; and Winter U.S. Pat. No. 5,225,539; and EP 0682040. Methods useful to prepare V_{H} and V_{L} polypeptides are taught by Winter et al., U.S. Pat. Nos. 4,816,397; 6,291,158; 6,291,159; 6,291,161; 6,545,142; EP 0368684; EP0451216; and EP0120694.

After selecting suitable framework region candidates from the same family and/or the same family member, either or both the heavy and light chain variable regions are produced by grafting the CDRs from the originating species into the hybrid framework regions. Assembly of hybrid antibodies or hybrid antibody fragments having hybrid variable chain regions with regard to either of the above aspects can be accomplished using conventional methods known to those skilled in the art. For example, DNA sequences encoding the hybrid variable domains described herein (i.e., frameworks based on the target species and CDRs from the originating species) can be produced by oligonucleotide synthesis and/or PCR. The nucleic acid encoding CDR regions can also be isolated from the originating species antibodies using suitable restriction enzymes and ligated into the target species framework by ligating with suitable ligation enzymes. Alternatively, the framework regions of the variable chains of the originating species antibody can be changed by site-directed mutagenesis.

Since the hybrids are constructed from choices among multiple candidates corresponding to each framework region, there exist many combinations of sequences which are amenable to construction in accordance with the principles described herein. Accordingly, libraries of hybrids can be assembled having members with different combinations of individual framework regions. Such libraries can be electronic database collections of sequences or physical collections of hybrids.

This process typically does not alter the acceptor antibody's FRs flanking the grafted CDRs. However, one skilled in the art can sometimes improve antigen binding affinity of the resulting anti-CD4 CDR-grafted antibody by replacing certain residues of a given FR to make the FR more similar to the corresponding FR of the donor antibody. Suitable locations of the substitutions include amino acid residues adjacent to the CDR, or which are capable of interacting with a CDR (See, e.g., U.S. Pat. No. 5,585,089, especially columns 12-16). Or one skilled in the art can start with the donor FR and modify it to be more similar to the acceptor FR or a human consensus FR. Techniques for making these modifications are known in the art. Particularly if the resulting FR fits a human consensus FR for that position, or is at least 90% or more identical to such a consensus FR, doing so may not increase the antigenicity of the resulting modified anti-CD4 CDR-grafted antibody significantly compared to the same antibody with a fully human FR.

Monoclonal Antibody. In one embodiment of the present technology, the CD4 targeting fusion protein of the present technology comprises an anti-CD4 monoclonal antibody. For example, in some embodiments, the anti-CD4 monoclonal antibody may be a human or a mouse anti-CD4 monoclonal antibody. For preparation of monoclonal antibodies directed towards the CD4 protein, or derivatives, fragments, analogs or homologs thereof, any technique that provides for the production of antibody molecules by continuous cell line culture can be utilized. Such techniques include, but are not limited to, the hybridoma technique (See, e.g., Kohler & Milstein, 1975. Nature 256: 495-497); the trioma technique; the human B-cell hybridoma technique (See, e.g., Kozbor, et al., 1983. Immunol. Today 4: 72) and the EBV hybridoma technique to produce human monoclonal antibodies (See, e.g., Cole, et al., 1985. In: MONOCLONAL ANTIBODIES AND CANCER THERAPY, Alan R. Liss, Inc., pp. 77-96). Human monoclonal antibodies can be utilized in the practice of the present technology and can be produced by using human hybridomas (See, e.g., Cote, et al., 1983. Proc. Natl. Acad. Sci. USA 80: 2026-2030) or by transforming human B-cells with Epstein Barr Virus in vitro (See, e.g., Cole, et al., 1985. In: MONOCLONAL ANTIBODIES AND CANCER THERAPY, Alan R. Liss, Inc., pp. 77-96). For example, a population of nucleic acids that encode regions of antibodies can be isolated. PCR utilizing primers derived from sequences encoding conserved regions of antibodies is used to amplify sequences encoding portions of antibodies from the population and then DNAs encoding antibodies or fragments thereof, such as variable domains, are reconstructed from the amplified sequences. Such amplified sequences also can be fused to DNAs encoding other proteins e.g., a bacteriophage coat, or a bacterial cell surface protein for expression and display of the fusion polypeptides on phage or bacteria. Amplified sequences can then be expressed and further selected or isolated based, e.g., on the affinity of the expressed antibody or fragment thereof for an antigen or epitope present on the CD4 protein. Alternatively, hybridomas expressing anti-CD4 monoclonal antibodies can be prepared by immunizing a subject and then isolating hybridomas from the subject's spleen using routine methods. See, e.g., Milstein et al., (Galfre and Milstein, Methods Enzymol (1981) 73: 3-46). Screening the hybridomas using standard methods will produce monoclonal antibodies of varying specificity (i.e., for different epitopes) and affinity. A selected monoclonal antibody with the desired properties, e.g., CD4 binding, can be used as expressed by the hybridoma, it can be bound to a molecule such as polyethylene glycol (PEG) to alter its properties, or a cDNA encoding it can be isolated, sequenced and manipulated in various ways. Synthetic dendromeric trees can be added to reactive amino acid side chains, e.g., lysine, to enhance the immunogenic properties of CD4 protein. Also, CPG-dinucleotide techniques can be used to enhance the immunogenic properties of the CD4 protein. Other manipulations include substituting or deleting particular amino acyl residues that contribute to instability of the antibody during storage or after administration to a subject, and affinity maturation techniques to improve affinity of the antibody of the CD4 protein.

Hybridoma Technique. In some embodiments, the CD4 targeting fusion protein of the present technology comprises an anti-CD4 monoclonal antibody produced by a hybridoma which includes a B cell obtained from a transgenic non-human animal, e.g., a transgenic mouse, having a genome comprising a human heavy chain transgene and a light chain transgene fused to an immortalized cell. Hybridoma techniques include those known in the art and taught in Harlow et al., Antibodies: A Laboratory Manual Cold Spring Harbor Laboratory, Cold Spring Harbor, N.Y., 349 (1988); Hammerling et al., Monoclonal Antibodies And T-Cell Hybridomas, 563-681 (1981). Other methods for producing hybridomas and monoclonal antibodies are well known to those of skill in the art.

Phage Display Technique. As noted above, the CD4 targeting fusion proteins of the present technology can be produced through the application of recombinant DNA and phage display technology. For example, a CD4 targeting fusion protein including an anti-CD4 antibody can be prepared using various phage display methods known in the art. In phage display methods, functional antibody domains are displayed on the surface of a phage particle which carries polynucleotide sequences encoding them. Phages with a desired binding property are selected from a repertoire or combinatorial antibody library (e.g., human or murine) by selecting directly with an antigen, typically an antigen bound or captured to a solid surface or bead. Phages used in these methods are typically filamentous phage including fd and M13 with Fab, Fv or disulfide stabilized Fv antibody domains that are recombinantly fused to either the phage gene III or gene VIII protein. In addition, methods can be adapted for the construction of Fab expression libraries (See, e.g., Huse, et al., Science 246: 1275-1281, 1989) to allow rapid and effective identification of monoclonal Fab fragments with the desired specificity for a CD4 polypeptide, e.g., a polypeptide or derivatives, fragments, analogs or homologs thereof. Other examples of phage display methods that can be used to make CD4 targeting fusion proteins of the present technology comprising an anti-CD4 antibody include those disclosed in Huston et al., Proc. Natl. Acad. Sci U.S.A., 85: 5879-5883, 1988; Chaudhary et al., Proc. Natl. Acad. Sci U.S.A., 87: 1066-1070, 1990; Brinkman et al., J Immunol. Methods 182: 41-50, 1995; Ames et al., Immunol. Methods 184: 177-186, 1995; Kettleborough et al., Eur. J. Immunol. 24: 952-958, 1994; Persic et al., Gene 187: 9-18, 1997; Burton et al., Advances in Immunology 57: 191-280, 1994; PCT/GB91/01134; WO 90/02809; WO 91/10737; WO 92/01047; WO 92/18619; WO 93/11236; WO 95/15982; WO 95/20401; WO 96/06213; WO 92/01047 (Medical Research Council et al.); WO 97/08320 (Morphosys); WO 92/01047 (CAT/MRC); WO 91/17271 (Affymax); and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743. Methods useful for displaying polypeptides on the surface of bacteriophage particles by attaching the polypeptides via disulfide bonds have been described by Lohning, U.S. Pat. No. 6,753,136. As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax et al., BioTechniques 12: 864-869, 1992; and Sawai et al., AJRI 34: 26-34, 1995; and Better et al., Science 240: 1041-1043, 1988.

Generally, hybrid antibodies or hybrid antibody fragments that are cloned into a display vector can be selected against the appropriate antigen in order to identify variants that maintain good binding activity, because the antibody or antibody fragment will be present on the surface of the phage or phagemid particle. See, e.g., Barbas III et al., Phage Display, A Laboratory Manual (Cold Spring Harbor Laboratory Press, Cold Spring Harbor, N.Y., 2001). However, other vector formats could be used for this process, such as cloning the antibody fragment library into a lytic phage vector (modified T7 or Lambda Zap systems) for selection and/or screening.

Labeled CD4 targeting fusion proteins. In one embodiment, the CD4 targeting fusion protein of the present technology is coupled with a label moiety, i.e., detectable group. The particular label or detectable group conjugated to the CD4 targeting fusion protein is not a critical aspect of the technology, so long as it does not significantly interfere with the specific binding of the CD4 targeting fusion protein of the present technology to the CD4 protein. The detectable group can be any material having a detectable physical or chemical property. Such detectable labels have been well-developed in the field of immunoassays and imaging. In general, almost any label useful in such methods can be applied to the present technology. Thus, a label is any composition detectable by spectroscopic, photochemical, biochemical, immunochemical, electrical, optical or chemical means. Labels useful in the practice of the present technology include magnetic beads (e.g., Dynabeads™), fluorescent dyes (e.g., fluorescein isothiocyanate, Texas red, rhodamine, and the like), radiolabels (e.g., ³H, ¹⁴C, ³⁵S, ¹²⁵I, ¹²¹I, ¹³¹I, ¹¹²In, ⁹⁹mTc), other imaging agents such as microbubbles (for ultrasound imaging), ¹⁸F, ¹¹C, ¹⁵O, (for Positron emission tomography), ⁹⁹mTC, ¹¹¹In (for Single photon emission tomography), enzymes (e.g., horse radish peroxidase, alkaline phosphatase and others commonly used in an ELISA), and calorimetric labels such as colloidal gold or colored glass or plastic (e.g., polystyrene, polypropylene, latex, and the like) beads. Patents that describe the use of such labels include U.S. Pat. Nos. 3,817,837; 3,850,752; 3,939,350; 3,996,345; 4,277,437; 4,275,149; and 4,366,241, each incorporated herein by reference in their entirety and for all purposes. See also Handbook of Fluorescent Probes and Research Chemicals (6^{th} Ed., Molecular Probes, Inc., Eugene Oreg.).

The label can be coupled directly or indirectly to the desired component of an assay according to methods well known in the art. As indicated above, a wide variety of labels can be used, with the choice of label depending on factors such as required sensitivity, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, e.g., biotin, thyroxine, and cortisol, it can be used in conjunction with the labeled, naturally-occurring anti-ligands.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds useful as labeling moieties, include, but are not limited to, e.g., fluorescein and its derivatives, rhodamine and its derivatives, dansyl, umbelliferone, and the like. Chemiluminescent compounds useful as labeling moieties, include, but are not limited to, e.g., luciferin, and 2,3-dihydrophthalazinediones, e.g., luminol. For a review of various labeling or signal-producing systems which can be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels can be detected by providing the appropriate substrates for the enzyme and detecting the resulting reaction product. Finally, simple colorimetric labels can be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the CD4 targeting fusion proteins. In this case, antigen-coated particles are agglutinated by samples comprising the CD4 targeting fusion protein. In this format, none of the components need be labeled and the presence of the CD4 targeting fusion protein is detected by simple visual inspection.

Expression of Recombinant CD4 targeting fusion proteins. The fusion proteins of the present technology can be produced through the application of recombinant DNA technology. Recombinant polynucleotide constructs encoding a CD4 targeting fusion protein of the present technology typically include an expression control sequence operably-linked to the coding sequences of the CD4 targeting fusion protein, including naturally-associated or heterologous promoter regions. As such, another aspect of the technology includes vectors containing one or more nucleic acid sequences encoding a CD4 targeting fusion protein of the present technology. For recombinant expression of one or more of the polypeptides of the present technology, the nucleic acid containing all or a portion of the nucleotide sequence encoding the CD4 targeting fusion protein is inserted into an appropriate cloning vector, or an expression vector (i.e., a vector that contains the necessary elements for the transcription and translation of the inserted polypeptide coding sequence) by recombinant DNA techniques well known in the art and as detailed below. Methods for producing diverse populations of vectors have been described by Lerner et al., U.S. Pat. Nos. 6,291,160 and 6,680,192.

In general, expression vectors useful in recombinant DNA techniques are often in the form of plasmids. In the present disclosure, “plasmid” and “vector” can be used interchangeably as the plasmid is the most commonly used form of vector. However, the present technology is intended to include such other forms of expression vectors that are not technically plasmids, such as viral vectors (e.g., replication defective retroviruses, adenoviruses and adeno-associated viruses), which serve equivalent functions. Such viral vectors permit infection of a subject and expression of a construct in that subject. In some embodiments, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences encoding the CD4 targeting fusion protein, and the collection and purification of the CD4 targeting fusion protein, e.g., cross-reacting CD4 targeting fusion proteins. See generally, U.S. 2002/0199213. These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences. Vectors can also encode signal peptide, e.g., pectate lyase, useful to direct the secretion of extracellular antibody fragments. See U.S. Pat. No. 5,576,195.

The recombinant expression vectors of the present technology comprise a nucleic acid encoding a protein with CD4 binding properties in a form suitable for expression of the nucleic acid in a host cell, which means that the recombinant expression vectors include one or more regulatory sequences, selected on the basis of the host cells to be used for expression that is operably-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, e.g., in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, etc. Typical regulatory sequences useful as promoters of recombinant polypeptide expression (e.g., CD4 targeting fusion protein), include, e.g., but are not limited to, promoters of 3-phosphoglycerate kinase and other glycolytic enzymes. Inducible yeast promoters include, among others, promoters from alcohol dehydrogenase, isocytochrome C, and enzymes responsible for maltose and galactose utilization. In one embodiment, a polynucleotide encoding a CD4 targeting fusion protein of the present technology is operably-linked to an ara B promoter and expressible in a host cell. See U.S. Pat. No. 5,028,530. The expression vectors of the present technology can be introduced into host cells to thereby produce polypeptides or peptides, including fusion polypeptides, encoded by nucleic acids as described herein (e.g., CD4 targeting fusion protein, etc.).

Another aspect of the present technology pertains to CD4 targeting fusion protein-expressing host cells, which contain a nucleic acid encoding one or more CD4 targeting fusion proteins. The recombinant expression vectors of the present technology can be designed for expression of a CD4 targeting fusion protein in prokaryotic or eukaryotic cells. For example, a CD4 targeting fusion protein can be expressed in bacterial cells such as Escherichia coli, insect cells (using baculovirus expression vectors), fungal cells, e.g., yeast, yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, e.g., using T7 promoter regulatory sequences and T7 polymerase. Methods useful for the preparation and screening of polypeptides having a predetermined property, e.g., CD4 targeting fusion protein, via expression of stochastically generated polynucleotide sequences has been previously described. See U.S. Pat. Nos. 5,763,192; 5,723,323; 5,814,476; 5,817,483; 5,824,514; 5,976,862; 6,492,107; 6,569,641.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (i) to increase expression of recombinant polypeptide; (ii) to increase the solubility of the recombinant polypeptide; and (iii) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding polypeptide, or polypeptide A, respectively, to the target recombinant polypeptide.

Examples of suitable inducible non-fusion E. coli expression vectors include pTrc (Amrann et al., (1988) Gene 69: 301-315) and pET 11d (Studier et al., GENE EXPRESSION TECHNOLOGY: METHODS IN ENZYMOLOGY 185, Academic Press, San Diego, Calif. (1990) 60-89). Methods for targeted assembly of distinct active peptide or protein domains to yield multifunctional polypeptides via polypeptide fusion has been described by Pack et al., U.S. Pat. Nos. 6,294,353; 6,692,935. One strategy to maximize recombinant polypeptide expression, e.g., a CD4 targeting fusion protein, in E. coli is to express the polypeptide in host bacteria with an impaired capacity to proteolytically cleave the recombinant polypeptide. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in the expression host, e.g., E. coli (See, e.g., Wada, et al., 1992. Nucl. Acids Res. 20: 2111-2118). Such alteration of nucleic acid sequences of the present technology can be carried out by standard DNA synthesis techniques.

In another embodiment, the CD4 targeting fusion protein expression vector is a yeast expression vector. Examples of vectors for expression in yeast Saccharomyces cerevisiae include pYepSec1 (Baldari, et al., 1987. EMBO J. 6: 229-234), pMFa (Kurjan and Herskowitz, Cell 30: 933-943, 1982), pJRY88 (Schultz et al., Gene 54: 113-123, 1987), pYES2 (Invitrogen Corporation, San Diego, Calif.), and picZ (Invitrogen Corp, San Diego, Calif.). Alternatively, a CD4 targeting fusion protein can be expressed in insect cells using baculovirus expression vectors. Baculovirus vectors available for expression of polypeptides, e.g., CD4 targeting fusion protein, in cultured insect cells (e.g., SF9 cells) include the pAc series (Smith, et al., Mol. Cell. Biol. 3: 2156-2165, 1983) and the pVL series (Lucklow and Summers, 1989. Virology 170: 31-39).

In yet another embodiment, a nucleic acid encoding a CD4 targeting fusion protein of the present technology is expressed in mammalian cells using a mammalian expression vector. Examples of mammalian expression vectors include, e.g., but are not limited to, pCDM8 (Seed, Nature 329: 840, 1987) and pMT2PC (Kaufman, et al., EMBO 6: 187-195, 1987). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells that are useful for expression of the CD4 targeting fusion protein of the present technology, see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid in a particular cell type (e.g., tissue-specific regulatory elements). Tissue-specific regulatory elements are known in the art. Non-limiting examples of suitable tissue-specific promoters include the albumin promoter (liver-specific; Pinkert, et al., Genes Dev. 1: 268-277, 1987), lymphoid-specific promoters (Calame and Eaton, Adv. Immunol. 43: 235-275, 1988), promoters of T cell receptors (Winoto and Baltimore, EMBO J. 8: 729-733, 1989) and immunoglobulins (Banerji, et al., 1983. Cell 33: 729-740; Queen and Baltimore, Cell 33: 741-748, 1983.), neuron-specific promoters (e.g., the neurofilament promoter; Byrne and Ruddle, Proc. Natl. Acad. Sci. USA 86: 5473-5477, 1989), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, Science 249: 374-379, 1990) and the α-fetoprotein promoter (Campes and Tilghman, Genes Dev. 3: 537-546, 1989).

Another aspect of the present methods pertains to host cells into which a recombinant expression vector of the present technology has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a CD4 targeting fusion protein can be expressed in bacterial cells such as E. coli, insect cells, yeast or mammalian cells. Mammalian cells are a suitable host for expressing nucleotide segments encoding immunoglobulins or fragments thereof. See Winnacker, From Genes To Clones, (VCH Publishers, NY, 1987). A number of suitable host cell lines capable of secreting intact heterologous proteins have been developed in the art, and include Chinese hamster ovary (CHO) cell lines, various COS cell lines, HeLa cells, L cells and myeloma cell lines. In some embodiments, the cells are non-human. Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Queen et al., Immunol. Rev. 89: 49, 1986. Illustrative expression control sequences are promoters derived from endogenous genes, cytomegalovirus, SV40, adenovirus, bovine papillomavirus, and the like. Co et al., J Immunol. 148: 1149, 1992. Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation, biolistics or viral-based transfection. Other methods used to transform mammalian cells include the use of polybrene, protoplast fusion, liposomes, electroporation, and microinjection (See generally, Sambrook et al., Molecular Cloning). Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals. The vectors containing the DNA segments of interest can be transferred into the host cell by well-known methods, depending on the type of cellular host.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the CD4 targeting fusion protein or can be introduced on a separate vector. Cells stably transfected with the introduced nucleic acid can be identified by drug selection (e.g., cells that have incorporated the selectable marker gene will survive, while the other cells die).

A host cell that includes a CD4 targeting fusion protein of the present technology, such as a prokaryotic or eukaryotic host cell in culture, can be used to produce (i.e., express) recombinant CD4 targeting fusion protein. In one embodiment, the method comprises culturing the host cell (into which a recombinant expression vector encoding the CD4 targeting fusion protein has been introduced) in a suitable medium such that the CD4 targeting fusion protein is produced. In another embodiment, the method further comprises the step of isolating the CD4 targeting fusion protein from the medium or the host cell. Once expressed, collections of the CD4 targeting fusion protein, e.g., the CD4 targeting fusion proteins or the CD4 targeting fusion protein-related polypeptides are purified from culture media and host cells. The CD4 targeting fusion protein can be purified according to standard procedures of the art, including HPLC purification, column chromatography, gel electrophoresis and the like. In one embodiment, the CD4 targeting fusion protein is produced in a host organism by the method of Boss et al., U.S. Pat. No. 4,816,397. Usually, CD4 targeting fusion protein chains are expressed with signal sequences and are thus released to the culture media. However, if the CD4 targeting fusion protein chains are not naturally secreted by host cells, the CD4 targeting fusion protein chains can be released by treatment with mild detergent. Purification of recombinant polypeptides is well known in the art and includes ammonium sulfate precipitation, affinity chromatography purification technique, column chromatography, ion exchange purification technique, gel electrophoresis and the like (See generally Scopes, Protein Purification (Springer-Verlag, N.Y., 1982).

Polynucleotides encoding CD4 targeting fusion proteins, e.g., the CD4 targeting fusion protein coding sequences, can be incorporated in transgenes for introduction into the genome of a transgenic animal and subsequent expression in the milk of the transgenic animal. See, e.g., U.S. Pat. Nos. 5,741,957, 5,304,489, and 5,849,992. Suitable transgenes include coding sequences for light and/or heavy chains in operable linkage with a promoter and enhancer from a mammary gland specific gene, such as casein or β-lactoglobulin. For production of transgenic animals, transgenes can be microinjected into fertilized oocytes, or can be incorporated into the genome of embryonic stem cells, and the nuclei of such cells transferred into enucleated oocytes.

Fc Modifications. In some embodiments, the CD4 targeting fusion proteins of the present technology comprise a variant Fc region, wherein said variant Fc region comprises at least one amino acid modification relative to a wild-type Fc region (or the parental Fc region), such that said molecule has an altered affinity for an Fc receptor (e.g., an FcγR), provided that said variant Fc region does not have a substitution at positions that make a direct contact with Fc receptor based on crystallographic and structural analysis of Fc-Fc receptor interactions such as those disclosed by Sondermann et al., Nature, 406:267-273 (2000). Examples of positions within the Fc region that make a direct contact with an Fc receptor such as an FcγR, include amino acids 234-239 (hinge region), amino acids 265-269 (B/C loop), amino acids 297-299 (C7E loop), and amino acids 327-332 (F/G) loop.

In some embodiments, a CD4 targeting fusion protein of the present technology has an altered affinity for activating and/or inhibitory receptors, having a variant Fc region with one or more amino acid modifications, wherein said one or more amino acid modification is a N297 substitution with alanine, or a K322 substitution with alanine.

Heterodimerization Domains. With respect to Fc-Fc-interactions, an amino acid substitution (preferably a substitution with an amino acid comprising a bulky side group forming a ‘knob’, e.g., tryptophan) can be introduced into the CH2 or CH3 domain of an Fc region such that steric interference will prevent interaction with a similarly mutated domain and will obligate the mutated domain to pair with a domain into which a complementary, or accommodating mutation has been engineered, i.e., ‘the hole’ (e.g., a substitution with glycine). Such sets of mutations can be engineered into a pair of polypeptides that are included within the fusion proteins disclosed herein, and further, engineered into any portion of the polypeptides chains of said pair. Methods of protein engineering to favor heterodimerization over homodimerization are well known in the art, in particular with respect to the engineering of immunoglobulin-like molecules, and are encompassed herein (see e.g., Ridgway et al., 1996, Protein Engr. 9:617-621, Atwell et al., 1997, J. Mol. Biol. 270: 26-35, and Xie et al., 2005, J. Immunol. Methods 296:95-101; each of which is hereby incorporated herein by reference in its entirety).

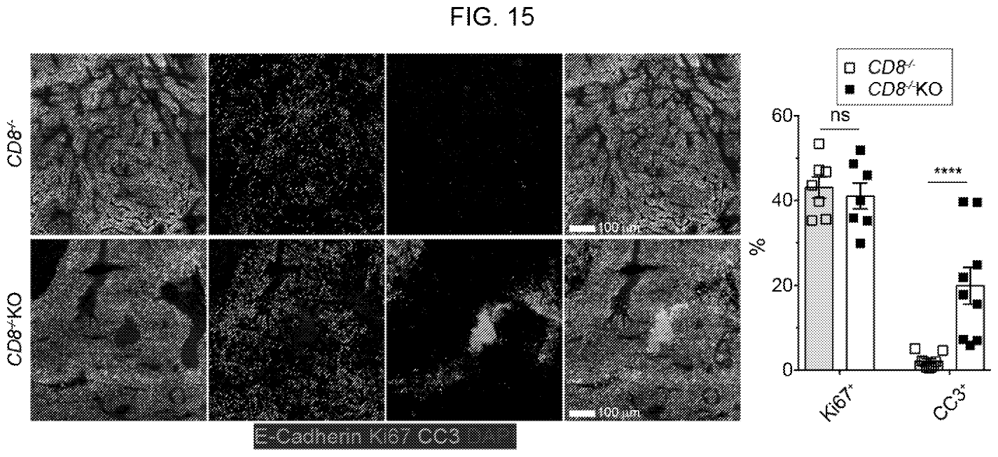

The design of variant Fc heterodimers from wild-type homodimers is illustrated by the concept of positive and negative design in the context of protein engineering by balancing stability vs. specificity, where mutations are introduced with the goal of driving heterodimer formation over homodimer formation when the polypeptides are expressed in cell culture conditions. Negative design strategies maximize unfavorable interactions for the formation of homodimers, by either introducing bulky sidechains on one chain and small sidechains on the opposite, for example the knobs-into-holes strategy developed by Genentech (Ridgway J B, Presta L G, Carter P. Protein Eng. 1996 July; 9(7):617-21; Atwell S, Ridgway J B, Wells J A, Carter P. J Mol. Biol. 270(1):26-35 (1997))), or by electrostatic engineering that leads to repulsion of homodimer formation, for example the electrostatic steering strategy developed by Amgen (Gunaskekaran K, et al. JBC 285 (25): 19637-19646 (2010)). In these two examples, negative design asymmetric point mutations are introduced into the wild-type CH3 domain to drive heterodimer formation. Other heterodimerization approaches are described in US 20120149876 (e.g., at Tables 1, 6 and 7), and US 20140294836 (e.g., at FIGS. 15A-B, 16A-B, and 17). Methods for engineering Fc heterodimers using electrostatic steering are described in detail in U.S. Pat. No. 8,592,562.

In some embodiments, the fusion proteins disclosed herein comprise a first CH2-CH3 domain and a second CH2-CH3 domain respectively (e.g., FIG. 48), wherein the first CH2-CH3 domain and the second CH2-CH3 domain comprise amino acid modifications selected from the group consisting of: T366Y and Y407T respectively; F405A and T394W respectively; Y349C/T366S/L368A/Y407V and S354C/T366W respectively; K409D/K392D and D399K respectively; T366S/L368A/Y407V and T366W respectively; K409D/K392D and D399K/E356K respectively; L351Y/Y407A and T366A/K409F respectively; L351Y/Y407A and T366V/K409F respectively; Y407A and T366A/K409F respectively; D399R/S400R/Y407A and T366A/K409F/K392E/T411E respectively; L351Y/F405A/Y407V and T394W respectively; L351Y/F405A/Y407V and T366L respectively; F405A/Y407V and T366I/K392M/T394W respectively; F405A/Y407V and T366L/K392M/T394W respectively; F405A/Y407V and T366L/T394W respectively; F405A/Y407V and T366I/T394W respectively; T366W/S354C and T366S/L368A/Y407V/Y349C, respectively; and K409R and F405L respectively.

In some embodiments, the fusion proteins disclosed herein comprise a first CH2-CH3 domain and a second CH2-CH3 domain respectively (e.g., FIG. 48), wherein the first CH2-CH3 domain comprises an amino acid modification at position F405 and amino acid modifications L351Y and Y407V, and the second CH2-CH3 domain comprises amino acid modification T394W. In some embodiments, the amino acid modification at position F405 is F405A, F405I, F405M, F405T, F4055, F405V or F405W.

In some embodiments, the fusion proteins disclosed herein comprise a first CH2-CH3 domain and a second CH2-CH3 domain respectively (e.g., FIG. 48), wherein the first CH2-CH3 domain comprises amino acid modifications at positions L351 and Y407, and the second CH2-CH3 domain comprises an amino acid modification at position T366 and amino acid modification K409F. In some embodiments, the amino acid modification at position L351 is L351Y, L351I, L351D, L351R or L351F. In some embodiments, the amino acid modification at position Y407 is Y407A, Y407V or Y4075. In certain embodiments, the amino acid modification at position T366 is T366A, T366I, T366L, T366M, T366Y, T366S, T366C, T366V or T366W.

In some embodiments, the fusion proteins disclosed herein comprise a first CH2-CH3 domain and a second CH2-CH3 domain respectively (e.g., FIG. 48), wherein the first CH2-CH3 domain or the second CH2-CH3 domain comprises an amino acid modification at positions K392, T411, T366, L368 or 5400. The amino acid modification at position K392 may be K392V, K392M, K392R, K392L, K392F or K392E. The amino acid modification at position T411 may be T411N, T411R, T411Q, T411K, T411D, T411E or T411W. The amino acid modification at position S400 may be S400E, S400D, S400R or S400K. The amino acid modification at position T366 may be T366A, T366I, T366L, T366M, T366Y, T366S, T366C, T366V or T366W. The amino acid modification at position L368 may be L368D, L368R, L368T, L368M, L368V, L368F, L368S and L368A.

In some embodiments, the fusion proteins disclosed herein comprise a first CH2-CH3 domain and a second CH2-CH3 domain respectively (e.g., FIG. 48), wherein the first CH2-CH3 domain comprises amino acid modifications L351Y and Y407A and the second CH2-CH3 domain comprises amino acid modifications T366A and K409F, and optionally wherein the first CH2-CH3 domain or the second CH2-CH3 domain comprises one or more amino acid modifications at position T411, D399, S400, F405, N390, or K392. The amino acid modification at position T411 may be T411N, T411R, T411Q, T411K, T411D, T411E or T411W. The amino acid modification at position D399 may be D399R, D399W, D399Y or D399K. The amino acid modification at position 5400 may be S400E, S400D, S400R, or S400K. The amino acid modification at position F405 may be F405I, F405M, F405T, F4055, F405V or F405W. The amino acid modification at position N390 may be N390R, N390K or N390D. The amino acid modification at position K392 may be K392V, K392M, K392R, K392L, K392F or K392E.

Glycosylation Modifications. In some embodiments, CD4 targeting fusion proteins of the present technology have an Fc region with variant glycosylation as compared to a parent Fc region. In some embodiments, variant glycosylation includes the absence of fucose; in some embodiments, variant glycosylation results from expression in GnT1-deficient CHO cells.

In some embodiments, the CD4 targeting fusion proteins of the present technology, may have a modified glycosylation site relative to an appropriate reference fusion protein that binds to an antigen of interest (e.g., CD4), without altering the functionality of the fusion protein, e.g., binding activity to the antigen. As used herein, “glycosylation sites” include any specific amino acid sequence in a binding molecule to which an oligosaccharide (i.e., carbohydrates containing two or more simple sugars linked together) will specifically and covalently attach.

Oligosaccharide side chains are typically linked to the backbone of a binding molecule via either N- or O-linkages. N-linked glycosylation refers to the attachment of an oligosaccharide moiety to the side chain of an asparagine residue. O-linked glycosylation refers to the attachment of an oligosaccharide moiety to a hydroxyamino acid, e.g., serine, threonine. For example, an Fc-glycoform (hCD4-IgGln) that lacks certain oligosaccharides including fucose and terminal N-acetylglucosamine may be produced in special CHO cells and exhibit enhanced ADCC effector function.

In some embodiments, the carbohydrate content of a fusion protein composition disclosed herein is modified by adding or deleting a glycosylation site. Methods for modifying the carbohydrate content of binding molecules are well known in the art and are included within the present technology, see, e.g., U.S. Pat. No. 6,218,149; EP 0359096B1; U.S. Patent Publication No. US 2002/0028486; International Patent Application Publication WO 03/035835; U.S. Patent Publication No. 2003/0115614; U.S. Pat. Nos. 6,218,149; 6,472,511; all of which are incorporated herein by reference in their entirety. In some embodiments, the carbohydrate content of a binding molecule is modified by deleting one or more endogenous carbohydrate moieties of the binding molecule. In some certain embodiments, the present technology includes deleting the glycosylation site of the Fc region of a CD4 targeting fusion protein of the present technology, by modifying position 297 from asparagine to alanine.

Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example N-acetylglucosaminyltransferase III (GnTIII), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al., 1999, Nat. Biotechnol. 17: 176-180; Davies et al., 2001, Biotechnol. Bioeng. 74:288-294; Shields et al., 2002, 1 Biol. Chem. 277:26733-26740; Shinkawa et al., 2003, J Biol. Chem. 278:3466-3473; U.S. Pat. No. 6,602,684; U.S. patent application Ser. No. 10/277,370; U.S. patent application Ser. No. 10/113,929; International Patent Application Publications WO 00/61739A1; WO 01/292246A1; WO 02/311140A1; WO 02/30954A1; POTILLEGENT™ technology (Biowa, Inc. Princeton, N.J.); GLYCOMAB™ glycosylation engineering technology (GLYCART biotechnology AG, Zurich, Switzerland); each of which is incorporated herein by reference in its entirety. See, e.g., International Patent Application Publication WO 00/061739; U.S. Patent Application Publication No. 2003/0115614; Okazaki et al., 2004, JMB, 336: 1239-49.

### Uses of the Fusion Proteins of the Present Technology

General. The CD4 targeting fusion proteins of the present technology are useful in methods known in the art relating to the localization and/or quantitation of CD4 protein (e.g., for use in measuring levels of the CD4 protein within appropriate physiological samples, for use in diagnostic methods, for use in imaging the polypeptide, and the like). Fusion proteins of the present technology may be useful to isolate a CD4 protein by standard techniques. Moreover, CD4 targeting fusion proteins can be used to detect an immunoreactive CD4 protein (e.g., in plasma, a cellular lysate or cell supernatant) in order to evaluate the abundance and pattern of expression of the immunoreactive polypeptide. The CD4 targeting fusion proteins of the present technology can be used diagnostically to monitor immunoreactive CD4 protein levels in tissue as part of a clinical testing procedure. The detection can be facilitated by coupling (i.e., physically linking) the CD4 targeting fusion proteins of the present technology to a detectable substance.

Detection of CD4 protein. An exemplary method for detecting the presence or absence of an immunoreactive CD4 protein in a biological sample involves obtaining a biological sample from a test subject and contacting the biological sample with a CD4 targeting fusion protein of the present technology capable of detecting an immunoreactive CD4 protein such that the presence of an immunoreactive CD4 protein is detected in the biological sample. Detection may be accomplished by means of a detectable label attached to the fusion protein.

The term “labeled” with regard to the CD4 targeting fusion protein is intended to encompass direct labeling of the fusion protein by coupling (i.e., physically linking) a detectable substance to the antibody, as well as indirect labeling of the fusion protein by reactivity with another compound that is directly labeled, such as a secondary antibody. Examples of indirect labeling include detection of a primary antibody using a fluorescently-labeled secondary antibody and end-labeling of a DNA probe with biotin such that it can be detected with fluorescently-labeled streptavidin.

In some embodiments, the CD4 targeting fusion proteins disclosed herein are conjugated to one or more detectable labels. For such uses, CD4 targeting fusion proteins may be detectably labeled by covalent or non-covalent attachment of a chromogenic, enzymatic, radioisotopic, isotopic, fluorescent, toxic, chemiluminescent, nuclear magnetic resonance contrast agent or other label.

Examples of suitable chromogenic labels include diaminobenzidine and 4-hydroxyazo-benzene-2-carboxylic acid. Examples of suitable enzyme labels include malate dehydrogenase, staphylococcal nuclease, Δ-5-steroid isomerase, yeast-alcohol dehydrogenase, α-glycerol phosphate dehydrogenase, triose phosphate isomerase, peroxidase, alkaline phosphatase, asparaginase, glucose oxidase, β-galactosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd, etc. ¹¹¹In is an exemplary isotope where in vivo imaging is used since its avoids the problem of dehalogenation of the ¹²⁵I or ¹³¹I-labeled antibodies by the liver. In addition, this isotope has a more favorable gamma emission energy for imaging (Perkins et al, Eur. J. Nucl. Med. 70:296-301 (1985); Carasquillo et al., J. Nucl. Med. 25:281-287 (1987)). For example, ¹¹¹In coupled to monoclonal antibodies with 1-(P-isothiocyanatobenzyl)-DPTA exhibits little uptake in non-tumorous tissues, particularly the liver, and enhances specificity of tumor localization (Esteban et al., J. Nucl. Med. 28:861-870 (1987)). Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include an ¹⁵²Eu label, a fluorescein label, an isothiocyanate label, a rhodamine label, a phycoerythrin label, a phycocyanin label, an allophycocyanin label, a Green Fluorescent Protein (GFP) label, an o-phthaldehyde label, and a fluorescamine label. Examples of suitable toxin labels include diphtheria toxin, ricin, and cholera toxin.

Examples of chemiluminescent labels include a luminol label, an isoluminol label, an aromatic acridinium ester label, an imidazole label, an acridinium salt label, an oxalate ester label, a luciferin label, a luciferase label, and an aequorin label. Examples of nuclear magnetic resonance contrasting agents include heavy metal nuclei such as Gd, Mn, and iron.

The detection method of the present technology can be used to detect an immunoreactive CD4 protein in a biological sample in vitro as well as in vivo. In vitro techniques for detection of an immunoreactive CD4 protein include enzyme linked immunosorbent assays (ELISAs), Western blots, immunoprecipitations, radioimmunoassay, and immunofluorescence. Furthermore, in vivo techniques for detection of an immunoreactive CD4 protein include introducing into a subject a labeled CD4 targeting fusion protein. For example, the CD4 targeting fusion protein can be labeled with a radioactive marker whose presence and location in a subject can be detected by standard imaging techniques. In one embodiment, the biological sample contains CD4 protein molecules from the test subject.

Immunoassay and Imaging. A CD4 targeting fusion protein of the present technology can be used to assay immunoreactive CD4 protein levels in a biological sample (e.g., human plasma) using antibody-based techniques. For example, protein expression in tissues can be studied with classical immunohistological methods. Jalkanen, M. et al., J. Cell. Biol. 101: 976-985, 1985; Jalkanen, M. et al., J. Cell. Biol. 105: 3087-3096, 1987. Other antibody-based methods useful for detecting protein gene expression include immunoassays, such as the enzyme linked immunosorbent assay (ELISA) and the radioimmunoassay (RIA). Suitable antibody-based assay labels are known in the art and include enzyme labels, such as, glucose oxidase, and radioisotopes or other radioactive agent, such as iodine (¹²⁵I, ¹²¹I, ¹³¹I), carbon (¹⁴C), sulfur (³⁵S), tritium (³H), indium (¹¹²In), and technetium (⁹⁹mTc), and fluorescent labels, such as fluorescein, rhodamine, and green fluorescent protein (GFP), as well as biotin.

In addition to assaying immunoreactive CD4 protein levels in a biological sample, CD4 targeting fusion proteins of the present technology may be used for in vivo imaging of CD4. Fusion proteins useful for this method include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the CD4 targeting fusion proteins by labeling of nutrients for the relevant scFv clone.

A CD4 targeting fusion protein which has been labeled with an appropriate detectable imaging moiety, such as a radioisotope (e.g., ¹³¹I, ¹¹²In, ⁹⁹mTc), a radio-opaque substance, or a material detectable by nuclear magnetic resonance, is introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled CD4 targeting fusion protein will then accumulate at the location of cells which contain the specific target polypeptide. For example, labeled CD4 targeting fusion proteins of the present technology will accumulate within the subject in cells and tissues in which the CD4 protein has localized.

Thus, the present technology provides a diagnostic method of a medical condition, which involves: (a) assaying the expression of immunoreactive CD4 protein by measuring binding of a CD4 targeting fusion protein of the present technology in cells or body fluid of an individual; (b) comparing the amount of immunoreactive CD4 protein present in the sample with a standard reference, wherein an increase or decrease in immunoreactive CD4 protein levels compared to the standard is indicative of a medical condition.

Affinity Purification. The CD4 targeting fusion proteins of the present technology may be used to purify immunoreactive CD4 protein from a sample. In some embodiments, the fusion proteins are immobilized on a solid support. Examples of such solid supports include plastics such as polycarbonate, complex carbohydrates such as agarose and sepharose, acrylic resins and such as polyacrylamide and latex beads. Techniques for coupling polypeptides to such solid supports are well known in the art (Weir et al., “Handbook of Experimental Immunology” 4th Ed., Blackwell Scientific Publications, Oxford, England, Chapter 10 (1986); Jacoby et al., Meth. Enzym. 34 Academic Press, N.Y. (1974)).

The simplest method to bind the target polypeptide (e.g., CD4) to the fusion protein-support matrix is to collect the beads in a column and pass the solution containing the target polypeptide down the column. The efficiency of this method depends on the contact time between the immobilized fusion protein and the target polypeptide, which can be extended by using low flow rates. The immobilized fusion protein captures the target polypeptide as it flows past. Alternatively, a target polypeptide can be contacted with the fusion protein-support matrix by mixing the solution containing the target polypeptide with the support (e.g., beads) and rotating or rocking the slurry, allowing maximum contact between the target polypeptide and the immobilized fusion protein. After the binding reaction has been completed, the slurry is passed into a column for collection of the beads. The beads are washed using a suitable washing buffer and then the pure or substantially pure target polypeptide is eluted.

A fusion protein or target polypeptide can be conjugated to a solid support, such as a bead. In addition, a first solid support such as a bead can also be conjugated, if desired, to a second solid support, which can be a second bead or other support, by any suitable means, including those disclosed herein for conjugation of a polypeptide to a support. Accordingly, any of the conjugation methods and means disclosed herein with reference to conjugation of a polypeptide to a solid support can also be applied for conjugation of a first support to a second support, where the first and second solid support can be the same or different.

Appropriate linkers, which can be cross-linking agents, for use for conjugating a polypeptide to a solid support include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl)propionic acid can be employed as a means for cleaving a polypeptide from a solid support. (Brown et al., Mol. Divers, pp, 4-12 (1995); Rothschild et al., Nucl. Acids Res., 24:351-66 (1996); and U.S. Pat. No. 5,643,722). Other cross-linking reagents are well-known in the art. (See, e.g., Wong (1991), supra; and Hermanson (1996), supra).

A fusion protein or target polypeptide can be immobilized on a solid support, such as a bead, through a covalent amide bond formed between a carboxyl group functionalized bead and the amino terminus of the fusion protein or target polypeptide or, conversely, through a covalent amide bond formed between an amino group functionalized bead and the carboxyl terminus of the fusion protein or target polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoroacetic acid to ensure that the fusion protein or target polypeptide is cleaved and can be removed. In such a case, the fusion protein or target polypeptide can be deposited as a beadless patch at the bottom of a well of a solid support or on the flat surface of a solid support. After addition of a matrix solution, the fusion protein or target polypeptide can be desorbed into a MS.

Hydrophobic trityl linkers can also be exploited as acid-labile linkers by using a volatile acid or an appropriate matrix solution, e.g., a matrix solution containing 3-HPA, to cleave an amino linked trityl group from the fusion protein or target polypeptide. Acid lability can also be changed. For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the fusion protein or target polypeptide. Accordingly, a fusion protein or target polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

Orthogonally cleavable linkers can also be useful for binding a first solid support, e.g., a bead to a second solid support, or for binding a polypeptide of interest to a solid support. Using such linkers, a first solid support, e.g., a bead, can be selectively cleaved from a second solid support, without cleaving the polypeptide from the support; the polypeptide then can be cleaved from the bead at a later time. For example, a disulfide linker, which can be cleaved using a reducing agent, such as DTT, can be employed to bind a bead to a second solid support, and an acid cleavable bi-functional trityl group could be used to immobilize a polypeptide to the support. As desired, the linkage of the polypeptide to the solid support can be cleaved first, e.g., leaving the linkage between the first and second support intact. Trityl linkers can provide a covalent or hydrophobic conjugation and, regardless of the nature of the conjugation, the trityl group is readily cleaved in acidic conditions.

For example, a bead can be bound to a second support through a linking group which can be selected to have a length and a chemical nature such that high density binding of the beads to the solid support, or high density binding of the polypeptides to the beads, is promoted. Such a linking group can have, e.g., “tree-like” structure, thereby providing a multiplicity of functional groups per attachment site on a solid support. Examples of such linking group; include polylysine, polyglutamic acid, penta-erythrole and tris-hydroxy-aminomethane.

Noncovalent Binding Association. A fusion protein or target polypeptide (e.g., CD4) can be conjugated to a solid support, or a first solid support can also be conjugated to a second solid support, through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide or a second solid support containing a complementary binding moiety. For example, a bead coated with avidin or with streptavidin can be bound to a polypeptide having a biotin moiety incorporated therein, or to a second solid support coated with biotin or derivative of biotin, such as iminobiotin.

It should be recognized that any of the binding members disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

### A. Diagnostic Uses of CD4 Targeting Fusion Proteins of the Present Technology

General. The CD4 targeting fusion proteins of the present technology are useful in diagnostic methods. As such, the present technology provides methods using the fusion proteins in the diagnosis of CD4 activity in a subject. CD4 targeting fusion proteins of the present technology may be selected such that they have any level of epitope binding specificity and very high binding affinity to a CD4 protein. In general, the higher the binding affinity of a fusion protein the more stringent wash conditions can be performed in an immunoassay to remove nonspecifically bound material without removing target polypeptide. Accordingly, CD4 targeting fusion proteins of the present technology useful in diagnostic assays usually have binding affinities of about 10⁸M⁻¹, 10⁹ M⁻¹, 10¹⁰ M⁻¹, 10¹¹ M⁻¹ or 10¹² M⁻¹. Further, it is desirable that CD4 targeting fusion proteins used as diagnostic reagents have a sufficient kinetic on-rate to reach equilibrium under standard conditions in at least 12 h, at least five (5) h, or at least one (1) hour.

CD4 targeting fusion proteins can be used to detect an immunoreactive CD4 protein in a variety of standard assay formats. Such formats include immunoprecipitation, Western blotting, ELISA, radioimmunoassay, and immunometric assays. See Harlow & Lane, Antibodies, A Laboratory Manual (Cold Spring Harbor Publications, New York, 1988); U.S. Pat. Nos. 3,791,932; 3,839,153; 3,850,752; 3,879,262; 4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Biological samples can be obtained from any tissue or body fluid of a subject. In certain embodiments, the subject is at an early stage of cancer. In certain embodiments, the sample is selected from the group consisting of urine, blood, serum, plasma, saliva, amniotic fluid, cerebrospinal fluid (CSF), and biopsied body tissue.

Immunometric or sandwich assays are one format for the diagnostic methods of the present technology. See U.S. Pat. Nos. 4,376,110, 4,486,530, 5,914,241, and 5,965,375. Such assays use one binding molecule, e.g., a CD4 targeting fusion protein or an anti-CD4 antibody, immobilized to a solid phase, and another binding molecule, e.g., CD4 targeting fusion protein or an anti-CD4 antibody, in solution. Typically, the solution binding molecule is labeled.

If a population of binding molecules is used, the population can contain binding molecules that bind to different epitopes within the target polypeptide. Accordingly, the same population can be used for both the solid phase and solution binding molecule. If CD4 targeting fusion proteins are used, first and second CD4 targeting fusion proteins having different binding specificities are used for the solid and solution phase. Solid phase (also referred to as “capture”) and solution (also referred to as “detection”) fusion proteins can be contacted with target polypeptide in either order or simultaneously. If the solid phase fusion protein is contacted first, the assay is referred to as being a forward assay. Conversely, if the solution fusion protein is contacted first, the assay is referred to as being a reverse assay. If the target is contacted with both fusion proteins simultaneously, the assay is referred to as a simultaneous assay. After contacting the CD4 protein with the CD4 targeting fusion protein, a sample is incubated for a period that usually varies from about 10 min to about 24 hr and is usually about 1 hr. A wash step is then performed to remove components of the sample not specifically bound to the CD4 targeting fusion protein being used as a diagnostic reagent. When solid phase and solution fusion proteins are bound in separate steps, a wash can be performed after either or both binding steps. After washing, binding is quantified, typically by detecting a label linked to the solid phase through binding of labeled solution fusion protein. Usually for a given pair of fusion proteins or populations of fusion proteins and given reaction conditions, a calibration curve is prepared from samples containing known concentrations of target polypeptide. Concentrations of the immunoreactive CD4 protein in samples being tested are then read by interpolation from the calibration curve (i.e., standard curve). Analyte can be measured either from the amount of labeled solution fusion protein bound at equilibrium or by kinetic measurements of bound labeled solution fusion protein at a series of time points before equilibrium is reached. The slope of such a curve is a measure of the concentration of the CD4 protein in a sample.

Suitable supports for use in the above methods include, e.g., nitrocellulose membranes, nylon membranes, and derivatized nylon membranes, and also particles, such as agarose, a dextran-based gel, dipsticks, particulates, microspheres, magnetic particles, test tubes, microtiter wells, SEPHADEX™ (Amersham Pharmacia Biotech, Piscataway N.J.), and the like. Immobilization can be by absorption or by covalent attachment. Optionally, CD4 targeting fusion proteins can be joined to a linker molecule, such as biotin for attachment to a surface bound linker, such as avidin.

In some embodiments, the present disclosure provides a CD4 targeting fusion protein of the present technology conjugated to a diagnostic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to the targeting moiety of a fusion protein described herein, e.g., antibody or antibody fragment, or subfragment, and is useful in diagnosing or monitoring a disease by locating the cells containing the target antigen.

Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). U.S. Pat. No. 6,331,175 describes Mill technique and the preparation of binding molecules conjugated to a MM enhancing agent and is incorporated in its entirety by reference. In some embodiments, the diagnostic agents are selected from the group consisting of radioisotopes, enhancing agents for use in magnetic resonance imaging, and fluorescent compounds. In order to load a binding molecule (e.g., fusion protein of the present technology) component with radioactive metals or paramagnetic ions, it may be necessary to react it with a reagent having a long tail to which are attached a multiplicity of chelating groups for binding the ions. Such a tail can be a polymer such as a polylysine, polysaccharide, or other derivatized or derivatizable chain having pendant groups to which can be bound chelating groups such as, e.g., ethylenediaminetetraacetic acid (EDTA), diethylenetriaminepentaacetic acid (DTPA), porphyrins, polyamines, crown ethers, bis-thiosemicarbazones, polyoximes, and like groups known to be useful for this purpose. Chelates may be coupled to the fusion proteins of the present technology using standard chemistries. The chelate is normally linked to the fusion protein by a group which enables formation of a bond to the molecule with minimal loss of immunoreactivity and minimal aggregation and/or internal cross-linking. Other methods and reagents for conjugating chelates to binding molecules are disclosed in U.S. Pat. No. 4,824,659. Particularly useful metal-chelate combinations include 2-benzyl-DTPA and its monomethyl and cyclohexyl analogs, used with diagnostic isotopes for radio-imaging. The same chelates, when complexed with non-radioactive metals, such as manganese, iron and gadolinium are useful for MRI, when used along with the CD4 fusion proteins of the present technology. Macrocyclic chelates such as NOTA (1,4,7-triaza-cyclononane-N,N′,N″-triacetic acid), DOTA, and TETA (p-bromoacetamido-benzyl-tetraethylaminetetraacetic acid) are of use with a variety of metals and radiometals, such as radionuclides of gallium, yttrium and copper, respectively. Such metal-chelate complexes can be stabilized by tailoring the ring size to the metal of interest. Other ring-type chelates such as macrocyclic polyethers, which are of interest for stably binding nuclides, such as ²²³Ra for RAIT are also contemplated.

### B. Therapeutic Use of CD4 Targeting Fusion Proteins of the Present Technology

The CD4 targeting fusion proteins of the present technology are useful for the treatment of cancers. Such treatment can be used in patients identified as having refractory cancers, or tumor-induced immunotolerance.

In one aspect, the present disclosure provides a method for treating a cancer in a subject in need thereof, comprising administering to the subject an effective amount of a CD4 targeting fusion protein of the present technology. In some embodiments, the cancer is refractory or recurrent. In another aspect, the present disclosure provides a method for increasing tumor sensitivity to a therapy in a subject suffering from cancer comprising (a) administering an effective amount of a CD4 targeting fusion protein of the present technology to the subject; and (b) administering an effective amount of an anti-cancer therapeutic agent to the subject. In some embodiments, the cancer is refractory or recurrent.

Examples of cancers that can be treated by the fusion proteins of the present technology include, but are not limited to: prostate cancer, pancreatic cancer, biliary cancer, colon cancer, rectal cancer, liver cancer, kidney cancer, lung cancer, testicular cancer, breast cancer, ovarian cancer, brain cancer, bladder cancer, head and neck cancers, melanoma, sarcoma, multiple myeloma, leukemia, lymphoma, and the like. In some embodiments of the methods disclosed herein, the subject is human.

The compositions of the present technology may be employed in conjunction with other therapeutic agents useful in the treatment of cancer. For example, the CD4 targeting fusion proteins of the present technology may be separately, sequentially or simultaneously administered with at least one additional therapeutic agent. Examples of such additional therapeutic agents include, but are not limited to, targeted therapies, immunotherapies (e.g., checkpoint inhibitors), antiangiogenic agents or chemotherapies. Targeted therapy agents include, but are not limited to, apoptosis-inducing proteasome inhibitor (e.g., Bortezomib), Selective estrogen-receptor modulator (e.g., Tamoxifen), BCR-ABL inhibitors (e.g., Imatinib, Dasatinib and Nilotinib), BTK inhibitor (e.g., Ibrutinib), EGFR inhibitors (e.g., Gefitinib, Erlotinib, Lapatinib, Neratinib, Osimertinib, Vandetanib, Dacomitinib), Janus kinase inhibitors (e.g., Ruxolitinib, Tofacitinib, Oclacitinib, baricitinib and Peficitinib), ALK inhibitors (e.g., Crizotinib, Ceritinib, Alectinib, Brigatinib and Lorlatinib), Bcl-2 inhibitors (e.g., Obatoclax, Navitoclax and Gossypol), PARP inhibitors (e.g., Iniparib, Olaparib and Talazoparib), PI3K inhibitors (e.g., Idelalisib, Copanlisib, Duvelisib and Alpelisib), MEK inhibitors (e.g., Trametinib, Binimetinib), CDK inhibitors (e.g., Palbociclib, Ribociclib and Abemaciclib), Hsp90 inhibitors (e.g., Gamitrinib and Luminespib), DNA-targeting agent (e.g., dianhydrogalactitol), NTRK inhibitors (e.g., Entrectinib and Larotrectinib), mTOR inhibitors (e.g., Temsirolimus and Everolimus), BRAF inhibitors (e.g., Vemurafenib, Dabrafenib, Encorafenib and Sorafenib), aromatase inhibitors, cytostatic alkaloids, cytotoxic antibiotics, antimetabolites, endocrine/hormonal agents, topoisomerase inhibitors, bisphosphonate therapy agents and targeted biological therapy agents (e.g., therapeutic peptides described in U.S. Pat. No. 6,306,832, WO 2012007137, WO 2005000889, WO 2010096603 etc.). Targeted therapy monoclonal antibodies include, but are not limited to, EGFR antibodies (e.g., Cetuximab, Panitumumab, Necitumumab), Her2/neu antibodies (e.g., Trastuzumab, Pertuzumab and Margetuximab), CD52 antibodies (e.g., Alemtuzumab), CD20 antibodies (e.g., Rituximab, Ofatumumab), GD2 antibodies (e.g., Dinutuximab), RANKL antibodies (e.g., Denosumab). Cancer immunotherapies include, but are not limited to, anti-PD-1 (e.g., Pembrolizumab, Nivolumab, Cemiplimab), anti-PD-L1 (e.g., atezolizumab, Avelumab, Durvalumab), anti-CTLA-4 (e.g., Ipilimumab, Tremelimumab), CD3/CD19 (e.g., Blinatumomab). Antiangiogenic agents include, but are not limited to, Axitinib, Bevacizumab, Cabozantinib, Everolimus, Lenalidomide, Lenvatinib mesylate, Pazopanib, Ramucirumab, Regorafenib, Sorafenib, Sunitinib, Thalidomide, Vandetanib, Ziv-aflibercept. In some embodiments, the at least one additional therapeutic agent is a chemotherapeutic agent. Specific chemotherapeutic agents include, but are not limited to, cyclophosphamide, fluorouracil (or 5-fluorouracil or 5-FU), Leucovorin, methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, tykerb, anthracyclines (e.g., daunorubicin and doxorubicin), oxaliplatin, melphalan, etoposide, mechlorethamine, bleomycin, microtubule poisons, annonaceous acetogenins, or combinations thereof.

In another aspect, the present disclosure provides a method for monitoring cancer progression in a patient in need thereof comprising (a) administering to the patient an effective amount of a fusion protein of the present technology; and (b) detecting tumor growth in the patient, wherein a reduction in tumor size relative to that observed in the patient prior to administration of the fusion protein is indicative of cancer arrest or cancer regression. Methods for detecting tumor growth are known in the art and include positron emission tomography, magnetic resonance imaging (MRI), ultrasound, computer tomography, or single photon emission computed tomography.

The CD4 targeting fusion proteins of the present technology may optionally be administered as a single bolus to a subject in need thereof. Alternatively, the dosing regimen may comprise multiple administrations performed at various times after the appearance of tumors.

Administration can be carried out by any suitable route, including orally, intranasally, parenterally (intravenously, intramuscularly, intraperitoneally, or subcutaneously), rectally, intracranially, intratumorally, intrathecally, or topically. Administration includes self-administration and the administration by another. It is also to be appreciated that the various modes of treatment of medical conditions as described are intended to mean “substantial”, which includes total but also less than total treatment, and wherein some biologically or medically relevant result is achieved.

In some embodiments, the CD4 targeting fusion proteins of the present technology comprise pharmaceutical formulations which may be administered to subjects in need thereof in one or more doses. Dosage regimens can be adjusted to provide the desired response (e.g., a therapeutic response).

Typically, an effective amount of the fusion protein compositions of the present technology, sufficient for achieving a therapeutic effect, range from about 0.000001 mg per kilogram body weight per day to about 10,000 mg per kilogram body weight per day. Typically, the dosage ranges are from about 0.0001 mg per kilogram body weight per day to about 100 mg per kilogram body weight per day. For administration of CD4 targeting fusion proteins, the dosage ranges from about 0.0001 to 100 mg/kg, and more usually 0.01 to 5 mg/kg every week, every two weeks or every three weeks, of the subject body weight. For example, dosages can be 1 mg/kg body weight or 10 mg/kg body weight every week, every two weeks or every three weeks or within the range of 1-10 mg/kg every week, every two weeks or every three weeks. In one embodiment, a single dosage of fusion protein ranges from 0.1-10,000 micrograms per kg body weight. In one embodiment, fusion protein concentrations in a carrier range from 0.2 to 2000 micrograms per delivered milliliter. An exemplary treatment regime entails administration once per every two weeks or once a month or once every 3 to 6 months. CD4 targeting fusion proteins may be administered on multiple occasions. Intervals between single dosages can be hourly, daily, weekly, monthly or yearly. Intervals can also be irregular as indicated by measuring blood levels of the fusion protein in the subject. In some methods, dosage is adjusted to achieve a serum fusion protein concentration in the subject of from about 75 μg/mL to about 125 μg/mL, 100 μg/mL to about 150 μg/mL, from about 125 μg/mL to about 175 μg/mL, or from about 150 μg/mL to about 200 μg/mL. Alternatively, CD4 targeting fusion proteins can be administered as a sustained release formulation, in which case less frequent administration is required. Dosage and frequency vary depending on the half-life of the fusion protein in the subject. The dosage and frequency of administration can vary depending on whether the treatment is prophylactic or therapeutic. In prophylactic applications, a relatively low dosage is administered at relatively infrequent intervals over a long period of time. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

Toxicity. Optimally, an effective amount (e.g., dose) of a CD4 targeting fusion protein described herein will provide therapeutic benefit without causing substantial toxicity to the subject. Toxicity of the CD4 targeting fusion protein described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the CD4 targeting fusion protein described herein lies within a range of circulating concentrations that include the effective dose with little or no toxicity. The dosage can vary within this range depending upon the dosage form employed and the route of administration utilized. The exact formulation, route of administration and dosage can be chosen by the individual physician in view of the subject's condition. See, e.g., Fingl et al., In: The Pharmacological Basis of Therapeutics, Ch. 1 (1975).

Formulations of Pharmaceutical Compositions. According to the methods of the present technology, the CD4 targeting fusion protein can be incorporated into pharmaceutical compositions suitable for administration. The pharmaceutical compositions generally comprise recombinant or substantially purified antibody and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the fusion protein compositions (See, e.g., Remington's Pharmaceutical Sciences, Mack Publishing Co., Easton, Pa. 18^{th} ed., 1990). The pharmaceutical compositions are generally formulated as sterile, substantially isotonic and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically-acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. “Pharmaceutically-acceptable salts and esters” means salts and esters that are pharmaceutically-acceptable and have the desired pharmacological properties. Such salts include salts that can be formed where acidic protons present in the composition are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically-acceptable esters include esters formed from carboxy, sulfonyloxy, and phosphonoxy groups present in the CD4 targeting fusion protein, e.g., C₁₋₆ alkyl esters. When there are two acidic groups present, a pharmaceutically-acceptable salt or ester can be a mono-acid-mono-salt or ester or a di-salt or ester; and similarly where there are more than two acidic groups present, some or all of such groups can be salified or esterified. A CD4 targeting fusion protein named in this technology can be present in unsalified or unesterified form, or in salified and/or esterified form, and the naming of such CD4 targeting fusion protein is intended to include both the original (unsalified and unesterified) compound and its pharmaceutically-acceptable salts and esters. Also, certain embodiments of the present technology can be present in more than one stereoisomeric form, and the naming of such CD4 targeting fusion protein is intended to include all single stereoisomers and all mixtures (whether racemic or otherwise) of such stereoisomers. A person of ordinary skill in the art, would have no difficulty determining the appropriate timing, sequence and dosages of administration for particular drugs and compositions of the present technology.

Examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the CD4 targeting fusion protein, use thereof in the compositions is contemplated. Supplementary active compounds can also be incorporated into the compositions.

A pharmaceutical composition of the present technology is formulated to be compatible with its intended route of administration. The CD4 targeting fusion protein compositions of the present technology can be administered by parenteral, topical, intravenous, oral, subcutaneous, intraarterial, intradermal, transdermal, rectal, intracranial, intrathecal, intraperitoneal, intranasal; or intramuscular routes, or as inhalants. The CD4 targeting fusion protein can optionally be administered in combination with other agents that are at least partly effective in treating various cancers.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfate; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be desirable to include isotonic compounds, e.g., sugars, polyalcohols such as manitol, sorbitol, sodium chloride in the composition. Prolonged absorption of the injectable compositions can be brought about by including in the composition a compound which delays absorption, e.g., aluminum monostearate and gelatin.

Sterile injectable solutions can be prepared by incorporating a CD4 targeting fusion protein of the present technology in the required amount in an appropriate solvent with one or a combination of ingredients enumerated above, as required, followed by filtered sterilization. Generally, dispersions are prepared by incorporating the CD4 targeting fusion protein into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. The antibodies of the present technology can be administered in the form of a depot injection or implant preparation which can be formulated in such a manner as to permit a sustained or pulsatile release of the active ingredient.

Oral compositions generally include an inert diluent or an edible carrier. They can be enclosed in gelatin capsules or compressed into tablets. For the purpose of oral therapeutic administration, the CD4 targeting fusion protein can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

For administration by inhalation, the CD4 targeting fusion protein is delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the CD4 targeting fusion protein is formulated into ointments, salves, gels, or creams as generally known in the art.

The CD4 targeting fusion protein can also be prepared as pharmaceutical compositions in the form of suppositories (e.g., with conventional suppository bases such as cocoa butter and other glycerides) or retention enemas for rectal delivery.

In one embodiment, the CD4 targeting fusion protein is prepared with carriers that will protect the CD4 targeting fusion protein against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically-acceptable carriers. These can be prepared according to methods known to those skilled in the art, e.g., as described in U.S. Pat. No. 4,522,811.

### Compositions and Methods for Cellular Therapy

In one aspect, the present disclosure provides compositions and methods for adoptive cell therapy comprising engineered helper T cells that either express a dominant negative TGF-β receptor II and/or that lack detectable expression or activity of a wild-type TGF-β receptor II. An engineered helper T cell may comprise one or more disruptions in endogenous genes encoding a TGF-β receptor II (e.g., CRISPR knockouts) and/or one or more transgenes that inhibit expression or activity of a TGF-β receptor II (e.g., a dominant negative TGF-β receptor II, or an inhibitory RNA (e.g., shRNA, siRNA) targeting a TGF-β receptor II).

Gene suppression can be performed in a number of ways. For example, gene expression can be suppressed by knock out, altering a promoter of a gene, and/or by inhibiting transcriptional or translational activity. This can be done at an organism level or at a tissue, organ, and/or cellular level. Gene suppression methods may comprise overexpressing a dominant negative protein. This method can result in overall decreased function of a functional wild-type gene. Additionally, expressing a dominant negative gene can result in a phenotype that is similar to that of a knockout and/or knockdown. Sometimes a stop codon can be inserted or created (e.g., by nucleotide replacement), in one or more genes, which can result in a nonfunctional transcript or protein (sometimes referred to as knockout). For example, if a stop codon is created within the middle of one or more genes, the resulting transcription and/or protein can be truncated, and can be nonfunctional. However, in some cases, truncation can lead to an active (a partially or overly active) protein. If a protein is overly active, this can result in a dominant negative protein.

Alternatively, one or more genes can be suppressed by administering inhibitory nucleic acids, e.g., siRNA, shRNA, antisense or microRNA. For example, an inhibitory nucleic acid (e.g., siRNA, shRNA, antisense or microRNA) or a nucleic acid expressing a dominant negative protein can be stably transfected into a cell to knockdown expression. Alternatively, an inhibitory nucleic acid (e.g., siRNA, shRNA, antisense or microRNA) or a nucleic acid expressing a dominant negative protein can be integrated into the genome of a helper T cell, thus knocking down a gene within the T cell.

In some embodiments, an engineered helper T cell comprises a nucleic acid encoding an exogenous dominant negative TGF-β receptor II. Expression of dominant negative transgenes can suppress expression and/or function of a wild-type counterpart of the dominant negative transgene. Thus, for example, a helper T cell comprising a dominant negative TGF-β receptor II transgene can have similar phenotypes compared to a different helper T cell in which the expression of the TGF-β receptor II is suppressed. Examples of dominant negative TGF-β receptor II include truncated mutants of Transforming growth factor beta Receptor II (TGF-βRII) or TGF-βRIIB that only contain the Extracellular domain (ECD) region that binds TGF-β (e.g., SEQ ID NOs: 15-17). Other examples of dominant negative TGF-β receptor II include (i) TGF-βRII (ΔC terminus): TGF-βRII lacking the last 38 amino acids from the C-terminus (SEQ ID NO: 37) and TGF-βRIIB (ΔC terminus): TGF-βRIIB lacking the last 38 as from the C-terminus (SEQ ID NO: 38); (ii) (Δcyt): TGF-βRII lacking the kinase domain & juxtamembrane region (SEQ ID NO: 39) and TGF-βRIIB (Δcyt): TGF-βRIIB lacking the kinase domain & juxtamembrane region (SEQ ID NO: 40); and (iii) inactive kinase mutants of TGF-βRII (SEQ ID NO: 41) and TGF-βRIIB (SEQ ID NO: 42). Expression of such partial sequences can lead to the production of a nonfunctional protein that competes with a functional (endogenous or exogenous) protein (a dominant negative protein).

TGF-βRII (ΔC terminus): TGF-βRII lacking the last 38 amino acids from the
C-terminus
(SEQ ID NO: 37)
MGRGLLRGLW PLHIVLWTRI ASTIPPHVQK SVNNDMIVTD NNGAVKFPQL
CKFCDVRFST CDNQKSCMSN CSITSICEKP QEVCVAVWRK NDENITLETV
CHDPKLPYHD FILEDAASPK CIMKEKKKPG ETFFMCSCSS DECNDNIIFS
EEYNTSNPDL LLVIFQVTGI SLLPPLGVAI SVIIIFYCYR VNRQQKLSST
WETGKTRKLM EFSEHCAIIL EDDRSDISST CANNINHNTE LLPIELDTLV
GKGRFAEVYK AKLKQNTSEQ FETVAVKIFP YEEYASWKTE KDIFSDINLK
HENILQFLTA EERKTELGKQ YWLITAFHAK GNLQEYLTRH VISWEDLRKL
GSSLARGIAH LHSDHTPCGR PKMPIVHRDL KSSNILVKND LTCCLCDFGL
SLRLDPTLSV DDLANSGQVG TARYMAPEVL ESRMNLENVE SFKQTDVYSM
ALVLWEMTSR CNAVGEVKDY EPPFGSKVRE HPCVESMKDN VLRDRGRPEI
PSFWLNHQGI QMVCETLTEC WDHDPEARL
TGF-βRIIB (ΔC terminus): TGF-βRIIB lacking the last 38 as from the
C-terminus
(SEQ ID NO: 38)
MGRGLLRGLW PLHIVLWTRI ASTIPPHVQK SDVEMEAQKD EIICPSCNRT
AHPLRHINND MIVTDNNGAV KFPQLCKFCD VRFSTCDNQK SCMSNCSITS
ICEKPQEVCV AVWRKNDENI TLETVCHDPK LPYHDFILED AASPKCIMKE
KKKPGETFFM CSCSSDECND NIIFSEEYNT SNPDLLLVIF QVTGISLLPP
LGVAISVIII FYCYRVNRQQ KLSSTWETGK TRKLMEFSEH CAIILEDDRS
DISSTCANNI NHNTELLPIE LDTLVGKGRF AEVYKAKLKQ NTSEQFETVA
VKIFPYEEYA SWKTEKDIFS DINLKHENIL QFLTAEERKT ELGKQYWLIT
AFHAKGNLQE YLTRHVISWE DLRKLGSSLA RGIAHLHSDH TPCGRPKMPI
VHRDLKSSNI LVKNDLTCCL CDFGLSLRLD PTLSVDDLAN SGQVGTARYM
APEVLESRMN LENVESFKQT DVYSMALVLW EMTSRCNAVG EVKDYEPPFG
SKVREHPCVE SMKDNVLRDR GRPEIPSFWL NHQGIQMVCE TLTECWDHDP
EARL
(Δcyt): TGF-βRII lacking the kinase domain & juxtamembrane region
(SEQ ID NO: 39)
MGRGLLRGLW PLHIVLWTRI ASTIPPHVQK SVNNDMIVTD NNGAVKFPQL
CKFCDVRFST CDNQKSCMSN CSITSICEKP QEVCVAVWRK NDENITLETV
CHDPKLPYHD FILEDAASPK CIMKEKKKPG ETFFMCSCSS DECNDNIIFS
EEYNTSNPDL LLVIFQVTGI SLLPPLGVAI SVIIIFYCYR VNRQQKLSS
TGF-βRIIB (Δcyt): TGF-βRIIB lacking the kinase domain & juxtamembrane region
(SEQ ID NO: 40)
MGRGLLRGLW PLHIVLWTRI ASTIPPHVQK SDVEMEAQKD EIICPSCNRT
AHPLRHINND MIVTDNNGAV KFPQLCKFCD VRFSTCDNQK SCMSNCSITS
ICEKPQEVCV AVWRKNDENI TLETVCHDPK LPYHDFILED AASPKCIMKE
KKKPGETFFM CSCSSDECND NIIFSEEYNT SNPDLLLVIF QVTGISLLPP
LGVAISVIII FYCYRVNRQQ KLSS
TGF-βRII (K277R) contains a point mutation in its ATP-binding site and is
inactive as a kinase
(SEQ ID NO: 41)
MGRGLLRGLW PLHIVLWTRI ASTIPPHVQK SVNNDMIVTD NNGAVKFPQL
CKFCDVRFST CDNQKSCMSN CSITSICEKP QEVCVAVWRK NDENITLETV
CHDPKLPYHD FILEDAASPK CIMKEKKKPG ETFFMCSCSS DECNDNIIFS
EEYNTSNPDL LLVIFQVTGI SLLPPLGVAI SVIIIFYCYR VNRQQKLSST
WETGKTRKLM EFSEHCAIIL EDDRSDISST CANNINHNTE LLPIELDTLV
GKGRFAEVYK AKLKQNTSEQ FETVAVRIFP YEEYASWKTE KDIFSDINLK
HENILQFLTA EERKTELGKQ YWLITAFHAK GNLQEYLTRH VISWEDLRKL
GSSLARGIAH LHSDHTPCGR PKMPIVHRDL KSSNILVKND LTCCLCDFGL
SLRLDPTLSV DDLANSGQVG TARYMAPEVL ESRMNLENVE SFKQTDVYSM
ALVLWEMTSR CNAVGEVKDY EPPFGSKVRE LEHLDRLSGR SCSEEKIPED
GSLNTTK
Transforming growth factor beta Receptor II (Δi)-TGF-βRII (Δi2) contains a
deletion of amino acids 498 to 508 and is inactive as a kinase
(SEQ ID NO: 42)
MGRGLLRGLW PLHIVLWTRI ASTIPPHVQK SVNNDMIVTD NNGAVKFPQL
CKFCDVRFST CDNQKSCMSN CSITSICEKP QEVCVAVWRK NDENITLETV
CHDPKLPYHD FILEDAASPK CIMKEKKKPG ETFFMCSCSS DECNDNIIFS
EEYNTSNPDL LLVIFQVTGI SLLPPLGVAI SVIIIFYCYR VNRQQKLSST
WETGKTRKLM EFSEHCAIIL EDDRSDISST CANNINHNTE LLPIELDTLV
GKGRFAEVYK AKLKQNTSEQ FETVAVKIFP YEEYASWKTE KDIFSDINLK
HENILQFLTA EERKTELGKQ YWLITAFHAK GNLQEYLTRH VISWEDLRKL
GSSLARGIAH LHSDHTPCGR PKMPIVHRDL KSSNILVKND LTCCLCDFGL
SLRLDPTLSV DDLANSGQVG TARYMAPEVL ESRMNLENVE SFKQTDVYSM
ALVLWEMTSR CNAVGEVKDY EPPFGSKVRE HPCVESMKDA SGIQMVCETL
TECWDHDPEA RLTAQCVAER FSELEHLDRL SGRSCSEEKI PEDGSLNTTK

Transgenes can be useful for expressing, e.g., overexpressing, exogenous dominant negative genes at a level greater than background, i.e., a cell that has not been transfected with a transgene. Nucleic acids comprising transgenes that encode transgene products can be placed into an organism, cell, tissue, or organ. Accordingly, in some embodiments, the engineered helper T cells comprises a transgene that encodes a dominant negative TGF-β receptor II (e.g., a transgene encoding SEQ ID NOs: 37-42). In other embodiments, the engineered helper T cells comprises a transgene that is at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 37-42. A transgene of a dominant negative TGF-β receptor II can refer to a transgene comprising a nucleotide sequence encoding the dominant negative TGF-β receptor II. An engineered helper T cell may comprise about 1, 2, 3, 4, 5, or more dominant negative TGF-β receptor II transgenes.

Also provided herein are engineered helper T cells comprising one or more transgenes that encode one or more inhibitory nucleic acids that can suppress genetic expression, e.g., can knockdown a gene. RNAs that suppress genetic expression can comprise, but are not limited to, antisense, shRNA, siRNA, RNAi, and microRNA. For example, transgenes encoding siRNA, RNAi, and/or microRNA can be delivered to a helper T cell to suppress genetic expression. For example, an engineered helper T cell may comprise a transgene encoding an inhibitory nucleic acid (e.g., siRNA, RNAi, antisense etc.) that specifically targets and inhibits the expression of one or more nucleic acid sequences selected from among SEQ ID NOs: 13-14, 18-20, and 21-23. An engineered helper T cell may comprise about 1, 2, 3, 4, 5, or more transgenes encoding one or more inhibitory nucleic acids that suppress the activity and/or expression of a wild-type TGF-β receptor II.

Transgenes of the present technology can be incorporated into a cell. When inserted into a cell, a transgene can be either a complementary DNA (cDNA) segment, which is a copy of messenger RNA (mRNA), or a genomic DNA segment (with or without introns). A transgene may be inserted within a coding genomic region or a noncoding genomic region. A transgene may be inserted into a genome with or without homologous recombination.

One or more of the transgenes disclosed herein can be derived from different species. For example, one or more transgenes can comprise a human gene, a mouse gene, a rat gene, a pig gene, a bovine gene, a dog gene, a cat gene, a monkey gene, a chimpanzee gene, or any combination thereof. For example, a transgene can be from a human, having a human genetic sequence. One or more transgenes can comprise human genes.

A transgene of the present technology can be inserted into a genome of a helper T cell in a random or site-specific manner. For example, a transgene can be inserted to a random locus in a genome of a T cell. A transgene can include its own promoter or can be inserted into a position where it is under the control of an endogenous promoter. Alternatively, a transgene can be inserted into a gene, such as an intron of a gene or an exon of a gene, a promoter, or a non-coding region. A transgene can be inserted such that the insertion disrupts a gene, e.g., an endogenous gene. A transgene insertion can be guided by recombination arms that can flank a transgene. Sometimes, more than one copy of a transgene can be inserted into a random locus in a genome. For example, multiple copies can be inserted into a random locus in a genome. This can lead to increased overall expression than if a transgene was randomly inserted once. Alternatively, a copy of a transgene can be inserted into a gene, and another copy of a transgene can be inserted into a different gene. A transgene can be targeted so that it could be inserted to a specific locus in a genome of a helper T cell.

Expression of any transgene disclosed herein can be controlled by one or more promoters. A promoter can be an ubiquitous promoter, a constitutive promoter, a tissue-specific promoter or an inducible promoter. Expression of a transgene that is inserted adjacent to or near a promoter can be regulated. For example, a transgene can be inserted near or next to a ubiquitous promoter. Examples of ubiquitous promoters include, but are not limited to, a CAGGS promoter, an hCMV promoter, a PGK promoter, an SV40 promoter, or a ROSA26 promoter. A promoter may be endogenous or exogenous. For example, one or more transgenes can be inserted adjacent or near to an endogenous or exogenous ROSA26 promoter. Tissue specific promoter or cell-specific promoters can be used to control the location of expression. Inducible promoters can be used as well. These inducible promoters can be turned on and off when desired, by adding or removing an inducing agent. Examples of inducible promoters include, but are not limited to, Lac, tac, trc, trp, araBAD, phoA, recA, proU, cst-1, tetA, cadA, nar, PL, cspA, T7, VHB, Mx, and/or Trex.

In another aspect, a helper T cell can be engineered to knock out endogenous genes. For example, knocking out one or more genes may comprise deleting one or more genes from a genome of a helper T cell (e.g., TGF-β receptor II or TGF-β receptor IIB). Knocking out can also comprise removing all or a part of a gene sequence (e.g., deletion) from a helper T cell (e.g., TGF-β receptor II or TGF-β receptor IIB). It is also contemplated that knocking out can comprise replacing all or a part of a gene in a genome of a helper T cell with one or more nucleotides. Knocking out one or more genes can also comprise inserting a sequence in one or more genes (e.g., insertion), thereby disrupting expression of the one or more genes. For example, inserting a sequence can generate a stop codon in the middle of one or more genes (e.g., nonsense mutation). Inserting a sequence can also shift the open reading frame of one or more genes (e.g., frameshift mutation).

By way of example only, one or more endogenous genes may be knocked out using an endonuclease selected from the group consisting of a CRISPR system (e.g., a Cas endonuclease), TALEN, Zinc Finger, transposon-based, ZEN, meganuclease, Mega-TAL, and any combination thereof.

CRISPR System. Methods described herein can take advantage of a CRISPR system. There are at least five types of CRISPR systems which all incorporate RNAs and Cas proteins. Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guiding RNA.

The general mechanism and recent advances of CRISPR system is discussed in Cong, L. et al., Science, 339 (6121): 819-823 (2013); Fu, Y. et al., Nature Biotechnology, 31, 822-826 (2013); Chu, V T et al., Nature Biotechnology 33, 543-548 (2015); Shmakov, S. et al., Molecular Cell, 60, 1-13 (2015); Makarova, K S et al., Nature Reviews Microbiology, 13, 1-15 (2015). Site-specific cleavage of a target DNA occurs at locations determined by both 1) base-pairing complementarity between the guide RNA and the target DNA (also called a protospacer) and 2) a short motif in the target DNA referred to as the protospacer adjacent motif (PAM). For example, an engineered cell can be generated using a CRISPR system, e.g., a type II CRISPR system. A Cas enzyme used in the methods disclosed herein can be Cas9, which catalyzes DNA cleavage. Enzymatic action by Cas9 derived from Streptococcus pyogenes or any closely related Cas9 can generate double stranded breaks at target site sequences which hybridize to about 20 nucleotides of a guide sequence and that have a protospacer-adjacent motif (PAM) following the about 20 nucleotides of the target sequence.

a. Cas Protein. A vector can be operably linked to an enzyme-coding sequence encoding a CRISPR enzyme, such as a Cas protein (CRISPR-associated protein). Non-limiting examples of Cas proteins can include Cas1, Cas1B, Cas2, Cas3, Cas4, Cas5, Cash, Cas7, Cas8, Cas9 (also known as Csn1 or Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, or modified versions thereof. In some embodiments, the Cas protein is Cas9. A Cas9 endonuclease may create a double strand break in at least one gene (e.g., a TGF-β receptor II gene). In some cases, a double strand break can be repaired using homology directed repair (HDR), non-homologous end joining (NHEJ), microhomology-mediated end joining (MMEJ), or any combination or derivative thereof.

An unmodified CRISPR enzyme can have DNA cleavage activity, such as Cas9. A CRISPR enzyme can direct cleavage of one or both strands at a target sequence, such as within a target sequence and/or within a complement of a target sequence. For example, a CRISPR enzyme can direct cleavage of one or both strands within or within about 1, 2, 3, 4, 5, 6, 7, 8, 9, 10, 15, 20, 25, 50, 100, 200, 500, or more base pairs from the first or last nucleotide of a target sequence. A vector that encodes a CRISPR enzyme that is mutated with respect to a corresponding wild-type enzyme such that the mutated CRISPR enzyme lacks the ability to cleave one or both strands of a target polynucleotide containing a target sequence can be used. A Cas protein can be a high fidelity cas protein such as Cas9HiFi.

A vector that encodes a CRISPR enzyme comprising one or more nuclear localization sequences (NLSs), such as 1, 2, 3, 4, 5, 6, 7, 8, 9, 10, or more NLSs can be used. For example, a CRISPR enzyme can comprise 1, 2, 3, 4, 5, 6, 7, 8, 9, 10, or more NLSs at or near the amino-terminus, or at or near the carboxyl-terminus, or any combination of these (e.g., one or more NLS at the amino-terminus and one or more NLS at the carboxyl terminus). When more than one NLS is present, each can be selected independently of others, such that a single NLS can be present in more than one copy and/or in combination with one or more other NLSs present in one or more copies.

Cas9 can refer to a polypeptide with at least about 50%, 60%, 70%, 80%, 90%, 100% sequence identity and/or sequence similarity to a wild-type exemplary Cas9 polypeptide (e.g., Cas9 from S. pyogenes). Cas9 can refer to a polypeptide with at most about 50%, 60%, 70%, 80%, 90%, 100% sequence identity and/or sequence similarity to a wild-type exemplary Cas9 polypeptide (e.g., from S. pyogenes). Cas9 can refer to the wild-type or a modified form of the Cas9 protein that can comprise an amino acid change such as a deletion, insertion, substitution, variant, mutation, fusion, chimera, or any combination thereof.

A polynucleotide encoding an endonuclease (e.g., a Cas protein such as Cas9) can be codon optimized for expression in particular cells, such as eukaryotic cells. This type of optimization can entail the mutation of foreign-derived (e.g., recombinant) DNA to mimic the codon preferences of the intended host organism or cell while encoding the same protein.

CRISPR enzymes used in the methods can comprise NLSs. The NLS can be located anywhere within the polypeptide chain, e.g., near the N- or C-terminus. For example, the NLS can be within or within about 1, 2, 3, 4, 5, 10, 15, 20, 25, 30, 40, 50 amino acids along a polypeptide chain from the N- or C-terminus. Sometimes the NLS can be within or within about 50 amino acids or more, e.g., 100, 200, 300, 400, 500, 600, 700, 800, 900, or 1000 amino acids from the N- or C-terminus.

An endonuclease can comprise an amino acid sequence having at least about 50%, 60%, 70%, 75%, 80%, 85%, 90%, 95%, 99%, or 100%, amino acid sequence identity to the nuclease domain of a wild-type exemplary site-directed polypeptide (e.g., Cas9 from S. pyogenes). In some cases, a different non-Cas9 endonuclease may be used to target certain genomic targets. In some cases, synthetic SpCas9-derived variants with non-NGG PAM sequences may be used.

Additionally, other Cas9 orthologues from various species have been identified and these “non-SpCas9s” bind a variety of PAM sequences that could also be useful for the present technology (e.g., Staphylococcus aureus Cas9 (SaCas9)).

Alternatives to S. pyogenes Cas9 may include RNA-guided endonucleases from the Cpf1 family that display cleavage activity in mammalian cells. Unlike Cas9 nucleases, the result of Cpf1-mediated DNA cleavage is a double-strand break with a short 3′ overhang. Cpf1's staggered cleavage pattern may open up the possibility of directional gene transfer, analogous to traditional restriction enzyme cloning, which may increase the efficiency of gene editing. Like the Cas9 variants and orthologues described above, Cpf1 may also expand the number of sites that can be targeted by CRISPR to AT-rich regions or AT-rich genomes that lack the NGG PAM sites favored by SpCas9.

Any functional concentration of Cas protein can be introduced to a cell. For example, 15 micrograms of Cas mRNA can be introduced to a cell. In other cases, a Cas mRNA can be introduced from 0.5 micrograms to 100 micrograms. A Cas mRNA can be introduced from 0.5, 5, 10, 15, 20, 25, 30, 35, 40, 45, 50, 55, 60, 65, 70, 75, 80, 85, 90, 95, or 100 micrograms.

b. Guide RNA. A method disclosed herein also can comprise introducing into a cell (e.g., a helper T cell) at least one guide RNA or nucleic acid, e.g., DNA encoding at least one guide RNA. A guide RNA can interact with a RNA-guided endonuclease to direct the endonuclease to a specific target site, at which site the 5′ end of the guide RNA base pairs with a specific protospacer sequence in a chromosomal sequence.

A guide RNA may comprise a CRISPR RNA (crRNA) and a transactivating crRNA (tracrRNA). A guide RNA can sometimes comprise a single-guide RNA (sgRNA) formed by fusion of a portion (e.g., a functional portion) of crRNA and tracrRNA. A guide RNA can also be a dual RNA comprising a crRNA and a tracrRNA. A guide RNA can comprise a crRNA and lack a tracrRNA. In some embodiments, a crRNA can hybridize with a target DNA or protospacer sequence.

A guide RNA can be an expression product. For example, a DNA that encodes a guide RNA can be a vector comprising a sequence coding for the guide RNA. A guide RNA can be transferred into a cell or organism by transfecting the cell or organism with an isolated guide RNA or plasmid DNA comprising a sequence coding for the guide RNA and a promoter. A guide RNA can also be transferred into a cell or organism in other way, such as using virus-mediated gene delivery. In other embodiments, a guide RNA can be isolated. For example, a guide RNA can be transfected in the form of an isolated RNA into a cell or organism. A guide RNA can be prepared by in vitro transcription using any in vitro transcription system. A guide RNA can be transferred to a cell in the form of isolated RNA rather than in the form of plasmid comprising encoding sequence for a guide RNA.

A guide RNA can comprise a DNA-targeting segment and a protein binding segment. A DNA-targeting segment (or DNA-targeting sequence, or spacer sequence) comprises a nucleotide sequence that can be complementary to a specific sequence within a target DNA (e.g., a protospacer). A protein-binding segment (or protein-binding sequence) can interact with a site-directed modifying polypeptide, e.g. an RNA-guided endonuclease such as a Cas protein. By “segment” it is meant a segment/section/region of a molecule, e.g., a contiguous stretch of nucleotides in an RNA. A segment can also mean a region/section of a complex such that a segment may comprise regions of more than one molecule. For example, in some cases a protein-binding segment of a DNA-targeting RNA is one RNA molecule and the protein-binding segment therefore comprises a region of that RNA molecule. In other cases, the protein-binding segment of a DNA-targeting RNA comprises two separate molecules that are hybridized along a region of complementarity.

A guide RNA can comprise two separate RNA molecules or a single RNA molecule. An exemplary single molecule guide RNA comprises both a DNA-targeting segment and a protein-binding segment. An exemplary two-molecule DNA-targeting RNA can comprise a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A first RNA molecule can be a crRNA-like molecule (targeter-RNA), that can comprise a DNA-targeting segment (e.g., spacer) and a stretch of nucleotides that can form one half of a double-stranded RNA (dsRNA) duplex comprising the protein-binding segment of a guide RNA.

A second RNA molecule can be a corresponding tracrRNA-like molecule (activator-RNA) that can comprise a stretch of nucleotides that can form the other half of a dsRNA duplex of a protein-binding segment of a guide RNA. In other words, a stretch of nucleotides of a crRNA-like molecule can be complementary to and can hybridize with a stretch of nucleotides of a tracrRNA-like molecule to form a dsRNA duplex of a protein-binding domain of a guide RNA. As such, each crRNA-like molecule can be said to have a corresponding tracrRNA-like molecule. A crRNA-like molecule additionally can provide a single stranded DNA-targeting segment, or spacer sequence. Thus, a crRNA-like and a tracrRNA-like molecule (as a corresponding pair) can hybridize to form a guide RNA. A subject two-molecule guide RNA can comprise any corresponding crRNA and tracrRNA pair.

A DNA-targeting segment or spacer sequence of a guide RNA can be complementary to sequence at a target site in a chromosomal sequence, e.g., protospacer sequence) such that the DNA-targeting segment of the guide RNA can base pair with the target site or protospacer. In some cases, a DNA-targeting segment of a guide RNA can comprise from about 10 nucleotides to from about 25 nucleotides or more. For example, a region of base pairing between a first region of a guide RNA and a target site in a chromosomal sequence can be about 10, 11, 12, 13, 14, 15, 16, 17, 18, 19, 20, 22, 23, 24, 25, or more than 25 nucleotides in length. Sometimes, a first region of a guide RNA can be about 19, 20, or 21 nucleotides in length.

A guide RNA can target a nucleic acid sequence of about 20 nucleotides. A target nucleic acid can be less than about 20 nucleotides. A target nucleic acid can be at least about 5, 10, 15, 16, 17, 18, 19, 20, 21, 22, 23, 24, 25, 30 or more nucleotides. A target nucleic acid can be at most about 5, 10, 15, 16, 17, 18, 19, 20, 21, 22, 23, 24, 25, 30 or more nucleotides. A target nucleic acid sequence can be about 20 bases immediately 5′ of the first nucleotide of the PAM.

A guide nucleic acid, for example, a guide RNA, can refer to a nucleic acid that can hybridize to another nucleic acid, for example, the target nucleic acid or protospacer in a genome of a cell. A guide nucleic acid can be RNA. A guide nucleic acid can be DNA. The guide nucleic acid can be programmed or designed to specifically bind to a sequence at a nucleic acid site. A guide nucleic acid can comprise a polynucleotide chain and can be called a single guide nucleic acid. A guide nucleic acid can comprise two polynucleotide chains and can be called a double guide nucleic acid.

A guide nucleic acid can comprise one or more chemical or physical modifications. A guide nucleic acid can comprise a nucleic acid affinity tag. A guide nucleic acid may comprise one or more synthetic nucleotides, synthetic nucleotide analogs, nucleotide derivatives, and/or modified nucleotides. A guide nucleic acid can comprise a nucleotide sequence (e.g., a spacer), for example, at or near the 5′ end or 3′ end, that can hybridize to a sequence in a target nucleic acid (e.g., a protospacer). A spacer of a guide nucleic acid can interact with a target nucleic acid in a sequence-specific manner via hybridization (i.e., base pairing). A spacer sequence can hybridize to a target nucleic acid that is located 5′ or 3′ to a protospacer adjacent motif (PAM). The length of a spacer sequence can be at least about 5, 10, 15, 16, 17, 18, 19, 20, 21, 22, 23, 24, 25, 30 or more nucleotides. The length of a spacer sequence can be at most about 5, 10, 15, 16, 17, 18, 19, 20, 21, 22, 23, 24, 25, 30 or more nucleotides.

A guide RNA may also comprise a dsRNA duplex region that forms a secondary structure. For example, a secondary structure formed by a guide RNA can comprise a stem (or hairpin) and a loop. A length of a loop and a stem can vary. For example, a loop can range from about 3 to about 10 nucleotides in length, and a stem can range from about 6 to about 20 base pairs in length. A stem can comprise one or more bulges of 1 to about 10 nucleotides. The overall length of a second region can range from about 16 to about 60 nucleotides in length. For example, a loop can be about 4 nucleotides in length and a stem can be about 12 base pairs. A dsRNA duplex region can comprise a protein-binding segment that can form a complex with an RNA-binding protein, such as a RNA-guided endonuclease, e.g., Cas protein.

A guide RNA can also comprise a tail region at the 5′ or 3′ end that can be single-stranded. For example, a tail region is sometimes not complementarity to any chromosomal sequence in a cell of interest and is sometimes not complementarity to the rest of a guide RNA. Further, the length of a tail region can vary. A tail region can be more than about 4 nucleotides in length. For example, the length of a tail region can range from about 5 to about 60 nucleotides in length.

A guide RNA can be introduced into a cell or embryo as an RNA molecule. For example, a RNA molecule can be transcribed in vitro and/or can be chemically synthesized. A guide RNA can then be introduced into a cell or embryo as an RNA molecule. A guide RNA can also be introduced into a cell or embryo in the form of a non-RNA nucleic acid molecule, e.g., DNA molecule. For example, a DNA encoding a guide RNA can be operably linked to promoter control sequence for expression of the guide RNA in a cell of interest. A DNA molecule encoding a guide RNA may be linear or circular.

When both a RNA-guided endonuclease and a guide RNA are introduced into a cell as DNA molecules, each can be part of a separate molecule (e.g., one vector containing the RNA-guided endonuclease coding sequence and a second vector containing the guide RNA coding sequence) or both can be part of a same molecule (e.g., one vector containing coding (and regulatory) sequence for both a RNA-guided endonuclease and a guide RNA).

A Cas protein, such as a Cas9 protein or any derivative thereof, can be pre-complexed with a guide RNA to form a ribonucleoprotein (RNP) complex. The RNP complex can facilitate homology directed repair (HDR). The RNP complex can be introduced into primary helper T cells. Introduction of the RNP complex can be timed. The cell can be synchronized with other cells at Gl, S, and/or M phases of the cell cycle. The RNP complex can be delivered at a cell phase such that HDR is enhanced.

A guide RNA can also be modified. The modifications can comprise chemical alterations, synthetic modifications, nucleotide additions, and/or nucleotide subtractions. The modifications can also enhance CRISPR genome engineering. A modification can alter chirality of a gRNA. In some cases, chirality may be uniform or stereopure after a modification. A guide RNA can be synthesized. The synthesized guide RNA can enhance CRISPR genome engineering. A guide RNA can also be truncated. Truncation can be used to reduce undesired off-target mutagenesis. The truncation can comprise any number of nucleotide deletions. For example, the truncation can comprise 1, 2, 3, 4, 5, 10, 15, 20, 25, 30, 40, 50 or more nucleotides. A guide RNA can comprise a region of target complementarity of any length. For example, a region of target complementarity can be less than 20 nucleotides in length. A region of target complementarity can be more than 20 nucleotides in length.

In some cases, a modification is on a 5′ end, a 3′ end, from a 5′ end to a 3′ end, a single base modification, a 2′-ribose modification, or any combination thereof. A modification can be selected from a group consisting of base substitutions, insertions, deletions, chemical modifications, physical modifications, stabilization, purification, and any combination thereof. In some embodiments, a modification is a chemical modification. A modification can be selected from 5′ adenylate, 5′ guanosine-triphosphate cap, 5′N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencer 2, DABCYL SE, dT-DABCYL, IRDye QC-1, QSY-21, QSY-35, QSY-7, QSY-9, carboxyl linker, thiol linkers, 2′ deoxyribonucleoside analog purine, 2′ deoxyribonucleoside analog pyrimidine, ribonucleoside analog, 2′-O-methyl ribonucleoside analog, sugar modified analogs, wobble/universal bases, fluorescent dye label, 2′ fluoro RNA, 2′ O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl 3phosphorothioate or any combinations thereof. A modification may be a pseudouride modification. In some cases, a modification is a 2-O-methyl 3 phosphorothioate addition. A 2-O-methyl 3 phosphorothioate addition can be performed from 1 base to 150 bases. A 2-O-methyl 3 phosphorothioate addition can be performed from 1 base to 4 bases. A 2-O-methyl 3 phosphorothioate addition can be performed on 2 bases. A 2-O-methyl 3 phosphorothioate addition can be performed on 4 bases. A modification can also be a truncation. A truncation can be a 5 base truncation. In some cases, a 5 base truncation can prevent a Cas protein from performing a cut.

In some cases, a dual nickase approach may be used to introduce a double stranded break. Cas proteins can be mutated at known amino acids within either nuclease domains, thereby deleting activity of one nuclease domain and generating a nickase Cas protein capable of generating a single strand break. A nickase along with two distinct guide RNAs targeting opposite strands may be utilized to generate a DSB within a target site (often referred to as a “double nick” or “dual nickase” CRISPR system). This approach may dramatically increase target specificity, since it is unlikely that two off-target nicks will be generated within close enough proximity to cause a DSB.

A gRNA can be introduced at any functional concentration. For example, a gRNA can be introduced to a cell at 10 micrograms. In other cases, a gRNA can be introduced from 0.5 micrograms to 100 micrograms. A gRNA can be introduced from 0.5, 5, 10, 15, 20, 25, 30, 35, 40, 45, 50, 55, 60, 65, 70, 75, 80, 85, 90, 95, or 100 micrograms.

A DNA sequence encoding a guide RNA or transgene disclosed herein can also be part of a vector. Some examples of vectors include plasmid vectors, phagemids, cosmids, artificial/mini-chromosomes, transposons, and viral vectors. Further, a vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like.

In one aspect, the present disclosure provides an engineered helper T cell, wherein the cell lacks detectable expression or activity of a TGF-β receptor II that comprises an amino acid sequence of any one of SEQ ID NOs: 11-12. In another aspect, the present disclosure provides an engineered helper T cell, wherein the cell expresses an inhibitory nucleic acid that specifically targets and inhibits the expression of a TGF-β receptor II nucleic acid sequence selected from among SEQ ID NOs: 13-14, 18-20, and 21-23. The inhibitory nucleic acid may be an antisense oligonucleotide, a siRNA, a sgRNA or a shRNA. Additionally or alternatively, in some embodiments of the engineered helper T cells of the present technology, the cells comprise a transgene that encodes a dominant negative TGF-β receptor II or the inhibitory nucleic acid. The transgene may be operably linked to an ubiquitous promoter, a constitutive promoter, a T cell-specific promoter, or an inducible promoter. In one aspect, the present disclosure provides an engineered helper T cell comprising a deletion, insertion, inversion, or frameshift mutation in a TGF-β receptor II gene encoded by the nucleic acid sequence of SEQ ID NO: 13 or SEQ ID NO: 14. In certain embodiments, the deletion, insertion, inversion, or frameshift mutation in a TGF-β receptor II gene is generated using at least one sgRNA and at least one endonuclease (e.g., Cas9 endonuclease). Additionally or alternatively, in some embodiments, the engineered helper T cell is derived from an autologous donor or an allogeneic donor.

In one aspect, the present disclosure provides a method for inhibiting tumor growth or metastasis in a subject with cancer comprising administering to the subject an effective amount of any of the engineered helper T cells described herein. The engineered helper T cells may be administered intravenously, intraperitoneally, subcutaneously, intramuscularly, or intratumorally. The cancer may be prostate cancer, pancreatic cancer, biliary cancer, colon cancer, rectal cancer, liver cancer, kidney cancer, lung cancer, testicular cancer, breast cancer, ovarian cancer, brain cancer, bladder cancer, head and neck cancers, melanoma, sarcoma, multiple myeloma, leukemia, or lymphoma.

Additionally or alternatively, in some embodiments, the method further comprises administering an additional cancer therapy. Examples of additional cancer therapies include, but are not limited to, chemotherapy, radiation therapy, immunotherapy, monoclonal antibodies, anti-cancer nucleic acids or proteins, anti-cancer viruses or microorganisms, and any combinations thereof. In some embodiments, the additional therapeutic agent is one or more of targeted therapies (e.g. apoptosis-inducing proteasome inhibitor, selective estrogen-receptor modulator, BCR-ABL inhibitors, BTK inhibitor, EGFR inhibitors, Janus kinase inhibitors, ALK inhibitors, Bcl-2 inhibitors, PARP inhibitors, PI3K inhibitors, MEK inhibitors, CDK inhibitors, Hsp90 inhibitors, DNA-targeting agent, NTRK inhibitors, mTOR inhibitors, BRAF inhibitors, aromatase inhibitors, cytostatic alkaloids, cytotoxic antibiotics, antimetabolites, endocrine/hormonal agents, topoisomerase inhibitors, bisphosphonate therapy agents), antiangiogenic agents (e.g., VEGF/VEGFR inhibitors), cancer immunotherapies (e.g. anti-PD-1, anti-PD-L1, anti-CTLA-4) or chemotherapeutic agents.

Additionally or alternatively, in certain embodiments, the method further comprises administering a cytokine agonist or antagonist to the subject. In some embodiments, the cytokine agonist or antagonist is administered prior to, during, or subsequent to administration of the one or more engineered helper T cells. In some embodiments, the cytokine agonist or antagonist is selected from a group consisting of interferon α, interferon β, interferon γ, complement C5a, IL-2, TNFalpha, CD40L, Ox40, IL-7, IL-18, IL-12, IL-23, IL-15, IL-17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

Additionally or alternatively, in some embodiments, the method further comprises sequentially, separately, or simultaneously administering to the subject at least one chemotherapeutic agent. Examples of chemotherapeutic agents include, but are not limited to, cyclophosphamide, fluorouracil (or 5-fluorouracil or 5-FU), Leucovorin, methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, tykerb, anthracyclines (e.g., daunorubicin and doxorubicin), oxaliplatin, melphalan, etoposide, mechlorethamine, bleomycin, microtubule poisons, annonaceous acetogenins, or combinations thereof.

In another aspect, the present disclosure provides methods for preparing immune cells for cancer therapy comprising isolating helper T cells from a donor subject; transducing the helper T cells with (a) an inhibitory nucleic acid that specifically targets and inhibits the expression of a TGF-β receptor II nucleic acid sequence selected from among SEQ ID NOs: 13-14, 18-20, and 21-23, and/or (b) an expression vector that encodes a dominant negative TGF-β receptor II having an amino acid sequence of any one of SEQ ID NOs: 15-17 or 37-42. The inhibitory nucleic acid is an antisense oligonucleotide, a siRNA, a sgRNA or a shRNA.

Disclosed herein is a method for making engineered helper T cells comprising: introducing at least one single guide RNA (sgRNA) and at least one endonuclease into a helper T cell under conditions to produce a deletion, an insertion, an inversion, or a frameshift mutation in a TGF-β receptor II gene, wherein the helper T cell comprises an endogenous genome and wherein the sgRNA comprises at least one sequence that is complementary to a TGF-β receptor II nucleic acid sequence in the endogenous genome of the helper T cell. The at least one endonuclease may be Cas1, Cas1B, Cas2, Cas3, Cas4, Cas5, Cash, Cas7, Cas8, Cas9 (also known as Csn1 or Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, or modified versions thereof. In some embodiments, the engineered helper T cells lack detectable expression or activity of a wild-type TGF-β receptor II.

In one aspect, the present disclosure provides a method of treatment comprising isolating helper T cells from a donor subject; transducing the helper T cells with (a) an inhibitory nucleic acid that specifically targets and inhibits the expression of a TGF-β receptor II nucleic acid sequence selected from among SEQ ID NOs: 13-14, 18-20, and 21-23, and/or (b) an expression vector that encodes a dominant negative TGF-β receptor II having an amino acid sequence of any one of SEQ ID NOs: 15-17 or 37-42; and administering the transduced helper T cells to a recipient subject. In some embodiments, the donor subject and the recipient subject are the same. In other embodiments, the donor subject and the recipient subject are different. In some embodiments, the method further comprises administering an additional cancer therapy.

### Kits

The present technology provides kits for the treatment of cancers (e.g., refractory cancers), comprising at least one fusion protein of the present technology, or a functional variant (e.g., substitutional variant) thereof. Also provided herein are kits for the treatment of cancers (e.g., refractory cancers), comprising any of the engineered helper T cells described herein, and instructions for use. Optionally, the above described components of the kits of the present technology are packed in suitable containers and labeled for treatment of cancers. The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kits are useful for detecting the presence of an immunoreactive CD4 protein in a biological sample, e.g., any body fluid including, but not limited to, e.g., serum, plasma, lymph, cystic fluid, urine, stool, cerebrospinal fluid, ascitic fluid or blood and including biopsy samples of body tissue. For example, the kit can comprise: one or more CD4 targeting fusion proteins of the present technology capable of binding a CD4 protein in a biological sample; means for determining the amount of the CD4 protein in the sample; and means for comparing the amount of the immunoreactive CD4 protein in the sample with a standard. One or more of the CD4 targeting fusion proteins may be labeled. The kit components, (e.g., reagents) can be packaged in a suitable container. The kit can further comprise instructions for using the kit to detect the immunoreactive CD4 protein.

For antibody-based kits, the kit can comprise, e.g., 1) a first CD4 targeting fusion protein, attached to a solid support, which binds to a CD4 protein; and, optionally; 2) a second, different antibody which binds to either the CD4 protein or to the first CD4 targeting fusion protein, and is conjugated to a detectable label.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit, e.g., for detection of a CD4 protein in vitro or in vivo, or for treatment of cancer in a subject in need thereof. In certain embodiments, the use of the reagents can be according to the methods of the present technology.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Experimental Materials and Methods (for Examples 2-6)

Mice. CD8^{-/-}, Ifng^{-/-} and mice were purchased from the Jackson Laboratory (Bar Harbor, Me.). ThPOK^{Cre} mice were provided by Dr. Ichiro Taniuchi. (See Mucida, D. et al., Transcriptional reprogramming of mature CD4(+) helper T cells generates distinct MHC class II-restricted cytotoxic T lymphocytes, Nat. Immunol. 14, 281-289, doi:10.1038/ni.2523 (2013).) CD8^{Cre}, Tgfrbr2^{fl/fl} and MMTV-PyMT (PyMT) mice were maintained in the laboratory as previously described. (See Donkor, M. K., Sarkar, A. & Li, M. O., Oncoimmunology 1, 162-171, doi:10.4161/onci.1.2.18481 (2012); Sarkar, A., Donkor, M. K. & Li, M. O., Oncotarget 2, 1339-1351 (2011); Ouyang, W., Beckett, O., Ma, Q. & Li, M. O., Transforming growth factor-beta signaling curbs thymic negative selection promoting regulatory T cell development, Immunity 32, 642-653, doi:10.1016/j.immuni.2010.04.012 (2010).) All mice were backcrossed to the C57BL/6 background and maintained under specific pathogen-free conditions. Animal experimentation was conducted in accordance with procedures approved by the Institutional Animal Care and Use Committee of Memorial Sloan Kettering Cancer Center.

Tumor measurement. Starting from 13 weeks of age, mammary tumors in female PyMT mice were measured weekly with a caliper. Tumor burden was calculated using the equation [(L×W²)×(π/6)], in which L and W denote length and width. Total tumor burden was calculated by summing up individual tumor volumes of each mouse with an end-point defined when total burden reached 3,000 mm³ or one tumor reached 2,000 mm³, typically around 23 weeks of age. Researchers were blinded to genotypes of mice during measurements.

Immune cell isolation from tissues. Single-cell suspensions were prepared from lymph nodes by tissue disruption with glass slides. The dissociated cells were passed through 70 μm filters and pelleted. Tumor-infiltrating immune cells were isolated from mammary tumors as previously described. (See Franklin, R. A. et al., The cellular and molecular origin of tumor-associated macrophages, Science 344, 921-925, doi:10.1126/science.1252510 (2014).) Briefly, tumor tissues were minced with a razor blade then digested in 280 U/mL Collagenase Type 3 (Worthington Biochemical) and 4 μg/mL DNase I (Sigma) in HBSS at 37° C. for 1 h and 15 min with periodic vortex every 20 min. Digested tissues were passed through 70 μm filters and pelleted. Cells were resuspended in 40% Percoll (Sigma) and were layered above 60% Percoll. The sample was centrifuged at 1,900 g at 4° C. for 30 min without brake. Cells at the interface were collected, stained, and analyzed by flow cytometry or were used for sorting.

Flow cytometry. Fluorochrome-conjugated, biotinylated antibodies against CD4 (RM4-5), CD8 (53-6.7), CD44 (IM7), CD62L (MEL-14), Foxp3 (FJK-165), IFN-γ (XMG1.2), IL-4 (BVD6-24G2), NK1.1 (PK136), PD-1 (RMP1-130) and TCRβ (H57-595) were purchased from eBiocience. Antibodies against CD45 (clone 30-F11), CD49a (Ha31/8), CD103 (M290) were obtained from BD Biosciences. Antibody against GzmB (GB11) was obtained from Invitrogen. All antibodies were tested with their respective isotype controls. Cell-surface staining was conducted by incubating cells with antibodies for 30 min on ice in the presence of 2.4G2 mAb to block FcγR binding. A transcription factor-staining kit (Tonbo Biosciences) was used for Foxp3 and granzyme B staining. To assess cytokine production, T cells were stimulated with 50 ng/mL phorbol 12-myristate 13-acetate (Sigma), 1 mM ionomycin (Sigma) in the presence of Golgi-Stop (BD Biosciences) for 4 h at 37° C. as previously described. (See Oh, S. A. et al., Proc. Nat'l Acad. Sci. U.S.A., doi:10.1073/pnas.1706356114 (2017)) T cells were subsequently stained for cell surface markers before intracellular cytokine staining. All data were acquired using an LSRII flow cytometer (Becton Dickinson) and analyzed with FlowJo software (Tree Star, Inc.).

Cell sorting, RNA extraction, and sequencing, T cells were FACS sorted to Trizol LS (Invitrogen) and snap frozen in liquid nitrogen, RNA was prepared with a miRNeasy Mini Kit according to the manufacturer's instructions (Qiagen), and subject to quality control by Agilent BioAnalyzer. 0.7-1 ng total RNA with an integrity index from 8.3 to 9.9 was amplified using the SMART-Seq v4 Ultra Low Input RNA Kit (Clontech), with 12 cycles of amplification. 2.7 ng of amplified cDNA was used to prepare libraries with the KAPA Hyper Prep Kit (Kapa Biosystems KK8504). Samples were barcoded and used for 50 bp/50 bp paired end runs with the TruSeq SBS Kit v4 (Illumina) on a HiSeq 2500 sequencer. An average of 45 million paired reads were generated per sample. The percentage of mRNA bases per sample ranged from 75% to 81%.

Transcriptome analysis of differentially expressed genes. The raw sequencing FASTQ files were aligned against the mm10 assembly by STAR. Gene level count values were computed by the summarizeOverlaps function from the R package “GenomicAlignments” with mm10 KnownGene as the base gene model for mouse samples. The Union counting mode was used, and only mapped paired-reads were considered. FPKM (Fragments Per Kilobase Million) values were then computed from gene level counts by using fpkm function from the R package “DESeq2.” Differentially expressed gene analysis was performed through the R DESeq2 package. Given the raw count data and gene model used, DESeq2 normalized the expression raw count data by sample specific size factor and took specified covariates into account while testing for genes found with significantly different expression between the experimental group and the control group samples.

Immunofluorescence staining. Antibodies against CD31 (MEC13.3) and GP38 (8.1.1) were purchased from Biolegend. Antibody against CD45 (30-F11) was from BD Pharmingen. Antibodies against Col IV (Cat. #2150-1470), Fibrinogen (Cat. #4440-8004) and NG2 (Cat. #AB5320) were from Bio-rad. Antibody against Fibronectin (Cat. #AB2033) was obtained from EMD. Antibody against Cleaved caspase 3 (Cat. #9661S) was purchased from CST. Antibodies against E-Cadherin (DECMA-1) and Ki67 (SolA15) were obtained from eBioscience. Hypoxia detection kit was purchased from Hypoxyprobe. Tumor tissues were frozen in O.C.T. medium (Sakura Finetek USA) and sectioned at the thickness of 10 prn. Tumor sections were fixed and stained with antibodies. Subsequently, they were mounted with VECTORSHIELD anti-fade mounting media (Vector Laboratories) and scanned by Pannoramic Digital Slide Scanners (3DHISTECH LTD). Immunofluorescence images were analyzed with CaseViewer and Fiji software, and further processed in Adobe Photoshop and Illustrator software.

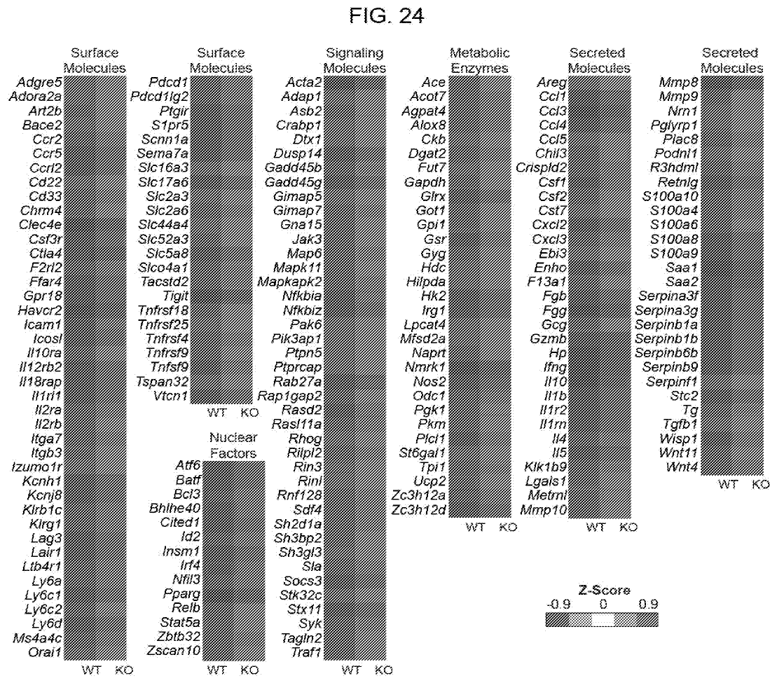

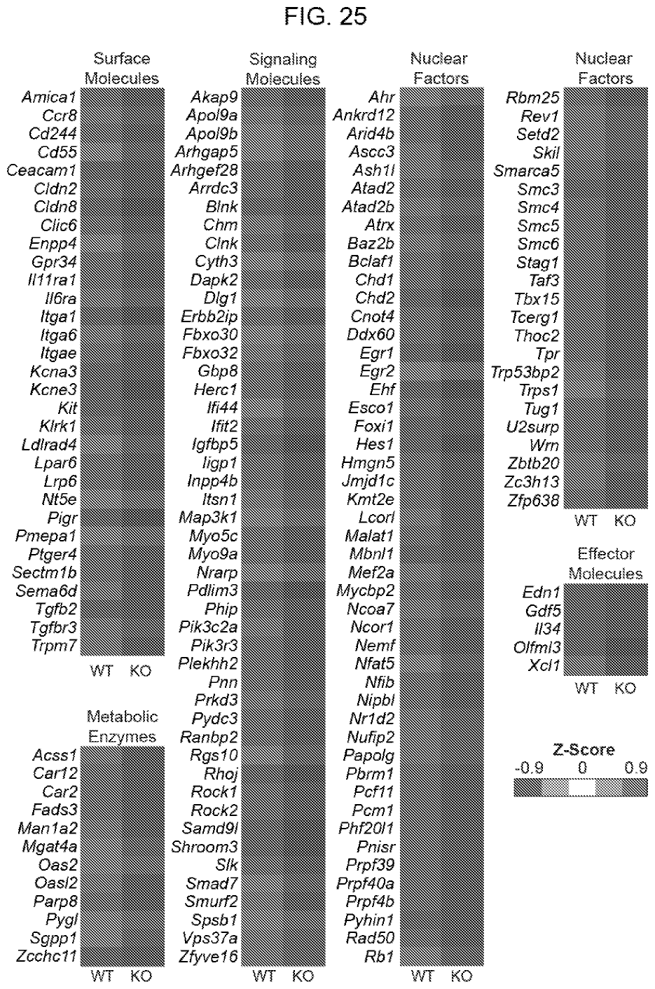

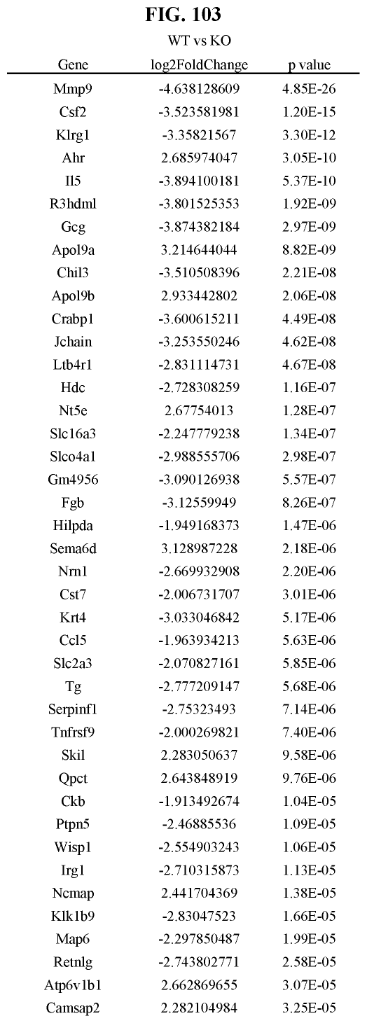

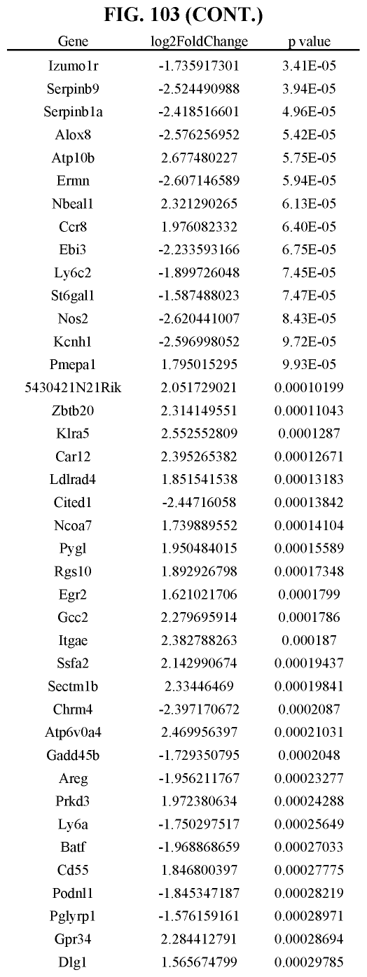

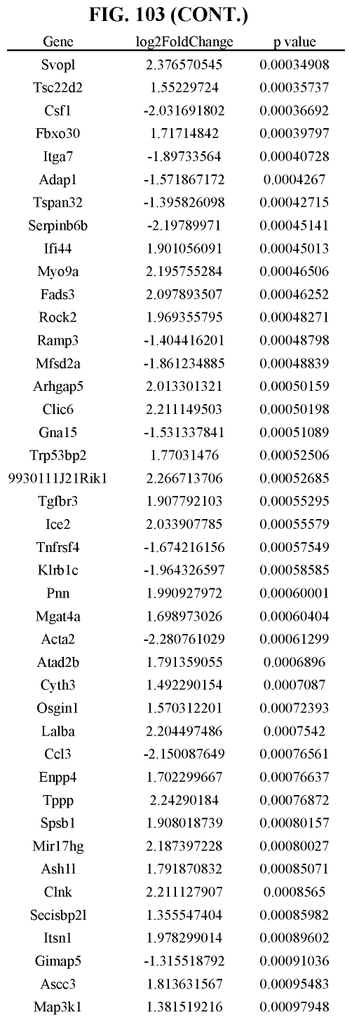

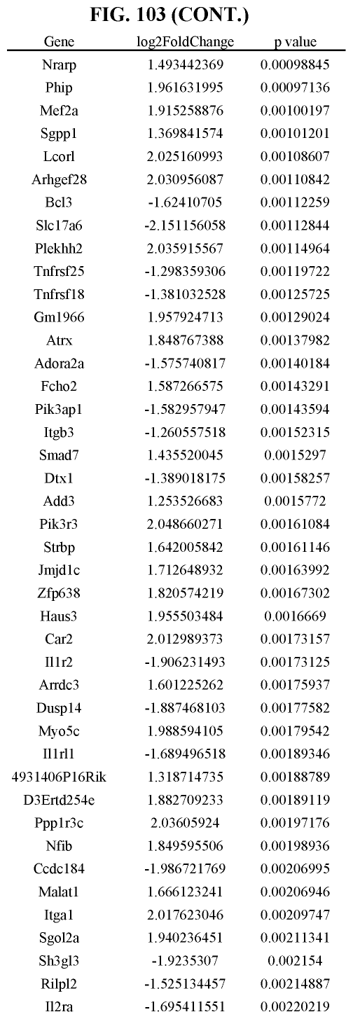

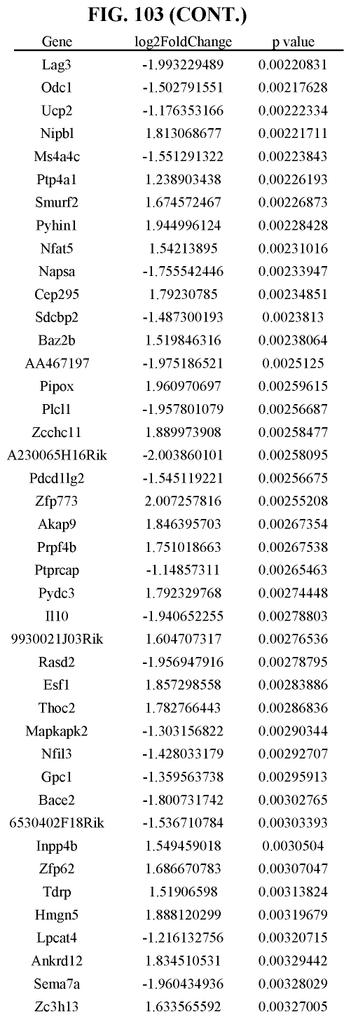

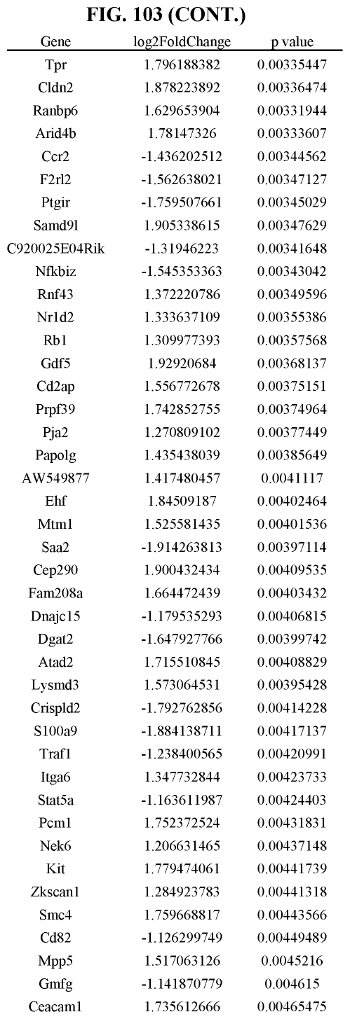

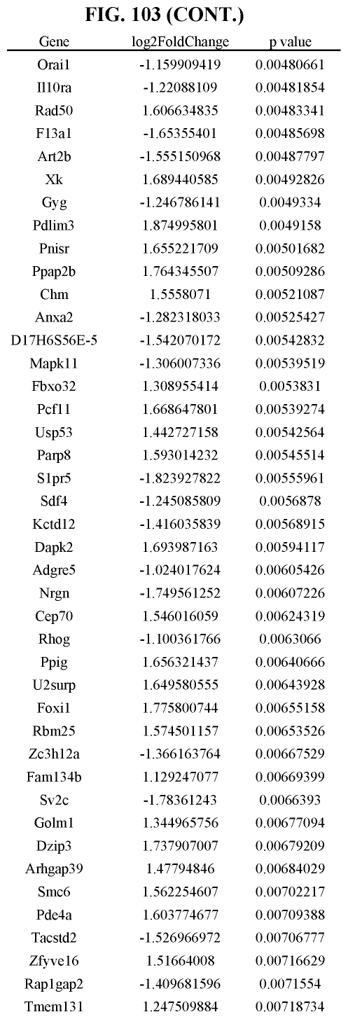

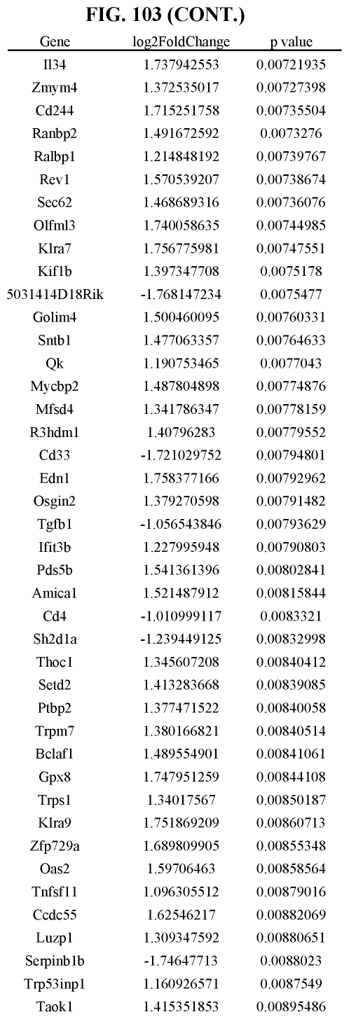

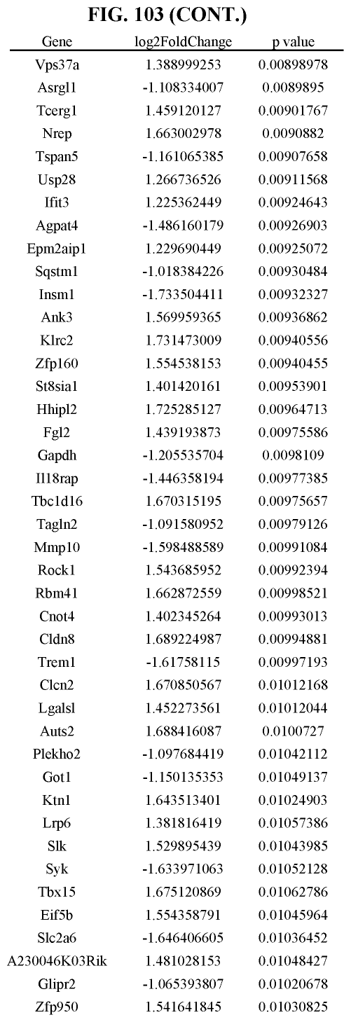

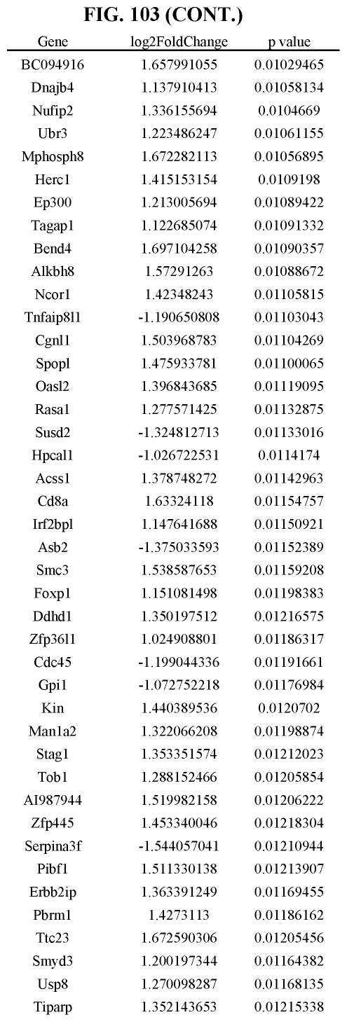

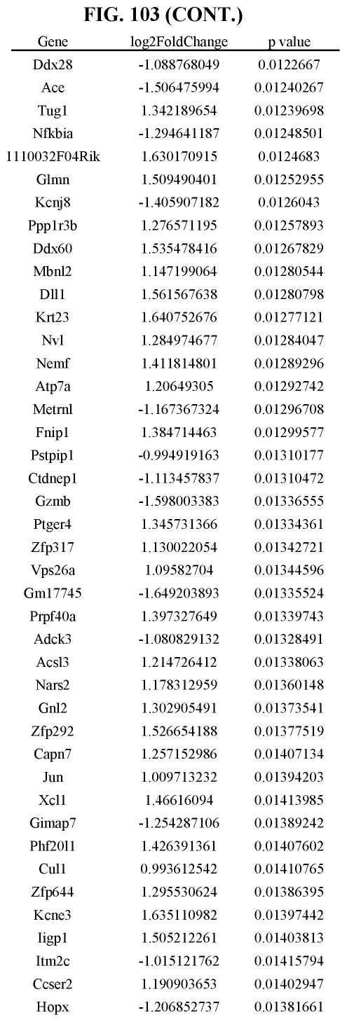

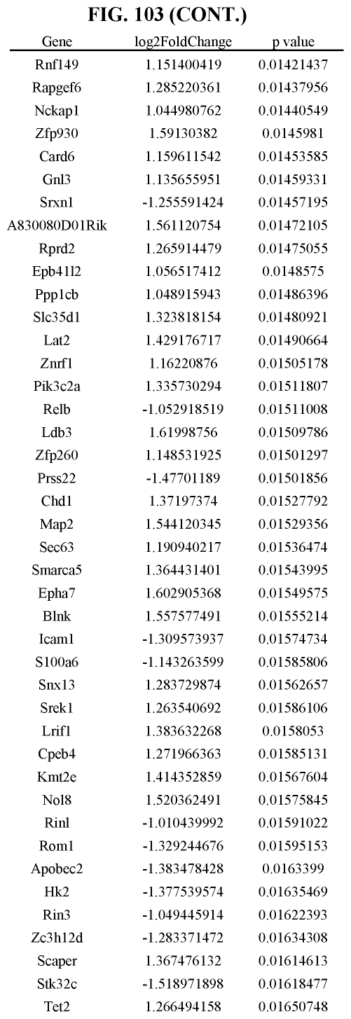

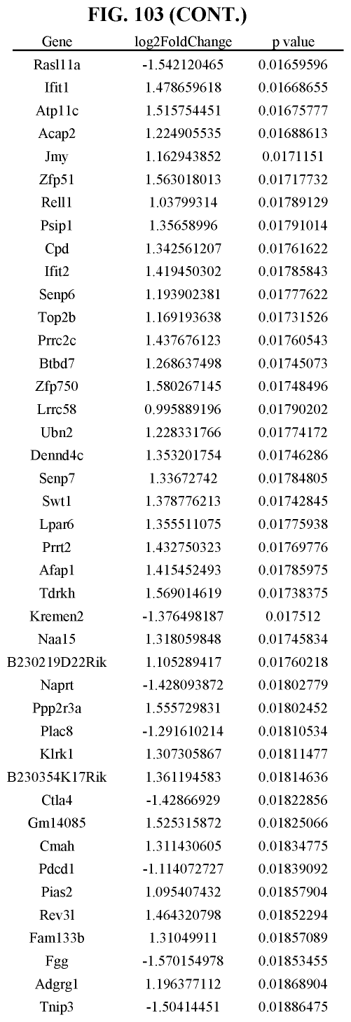

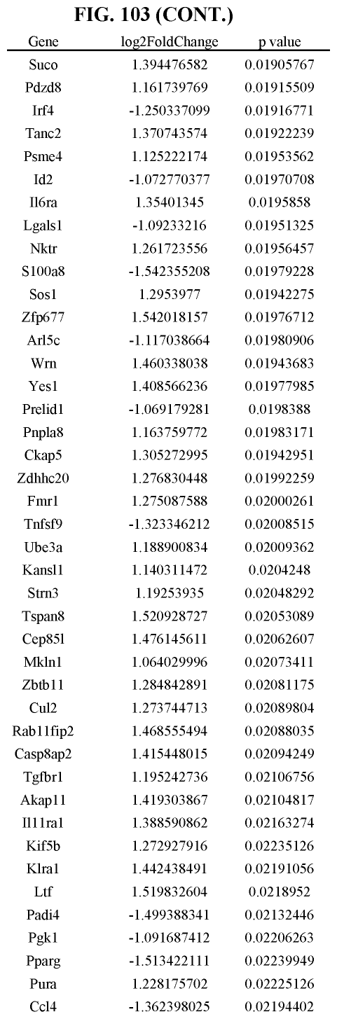

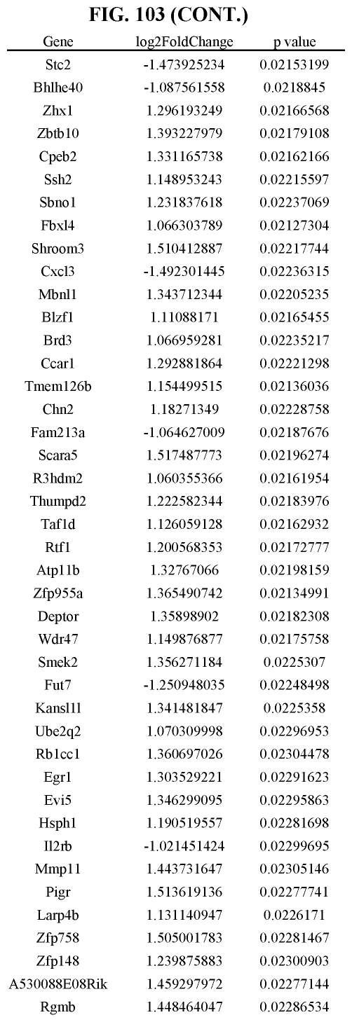

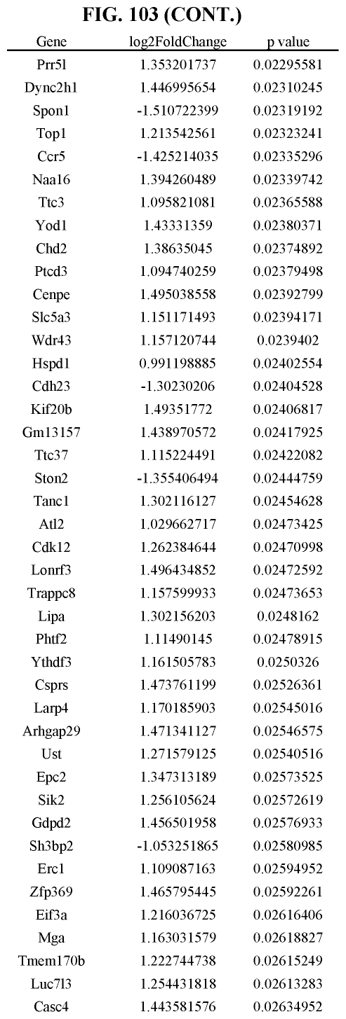

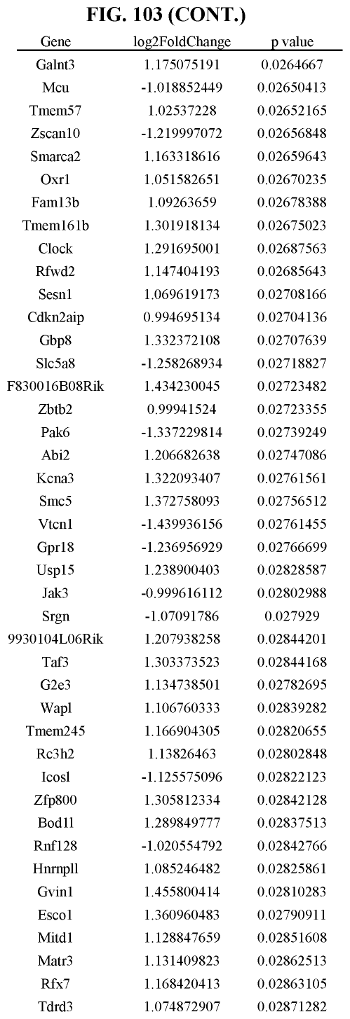

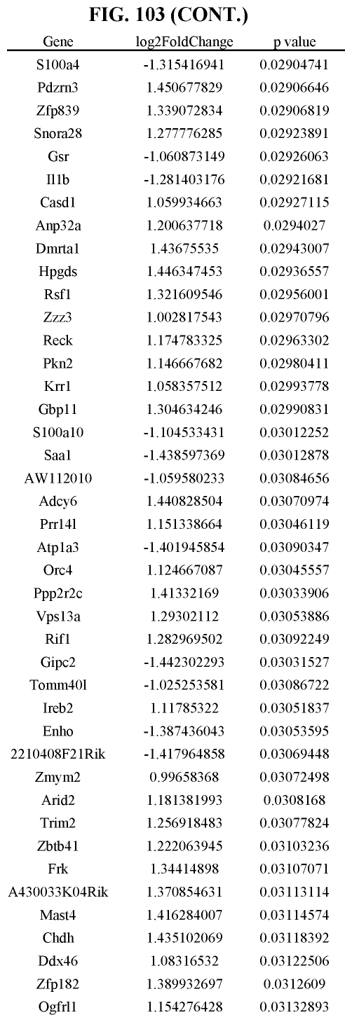

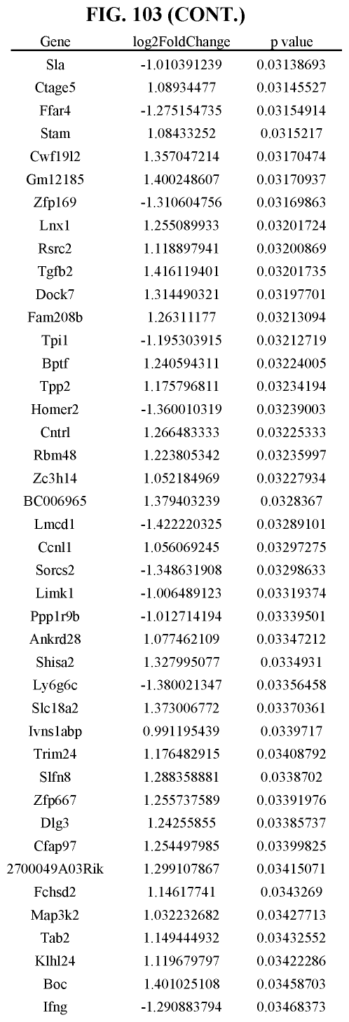

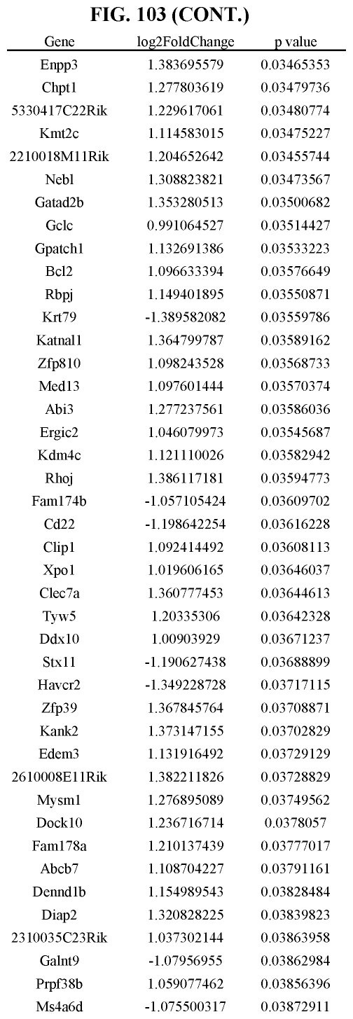

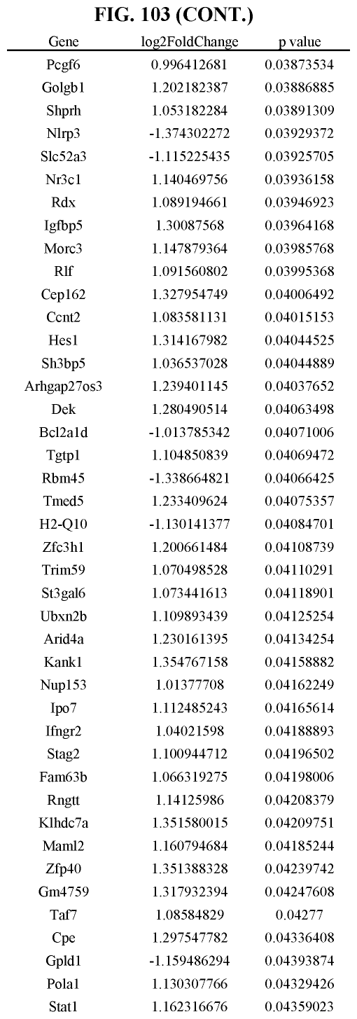

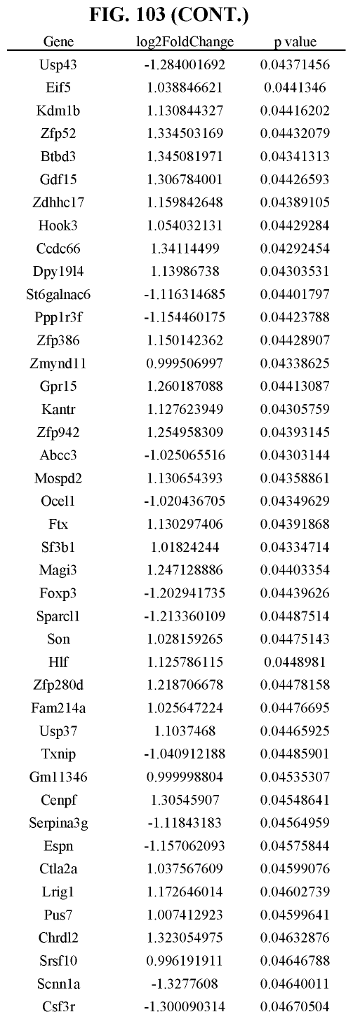

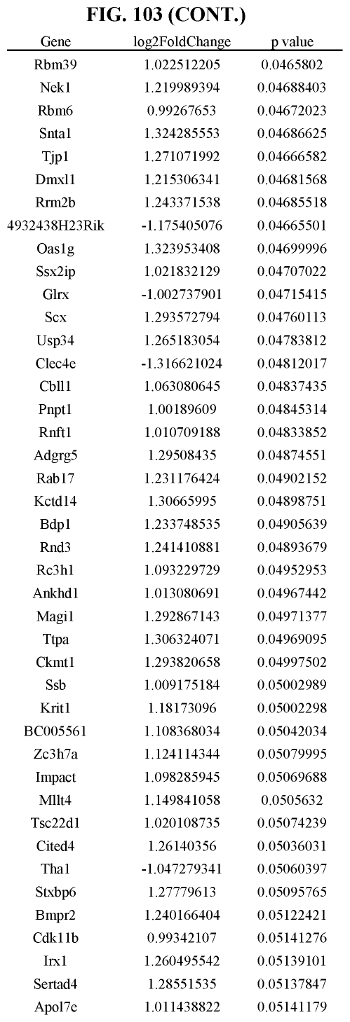

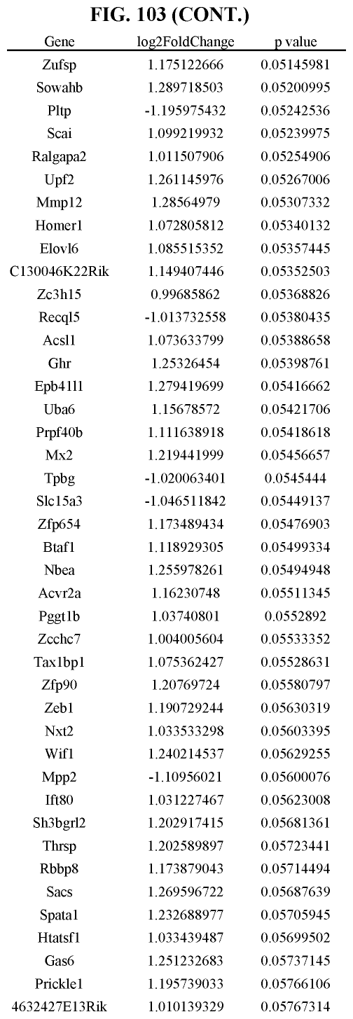

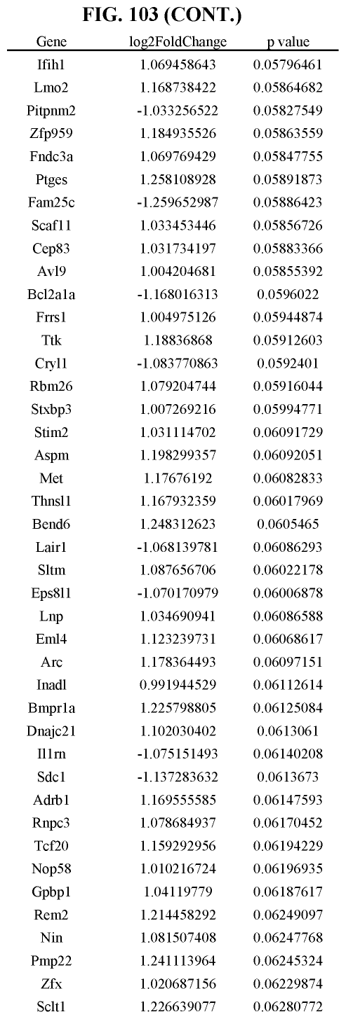

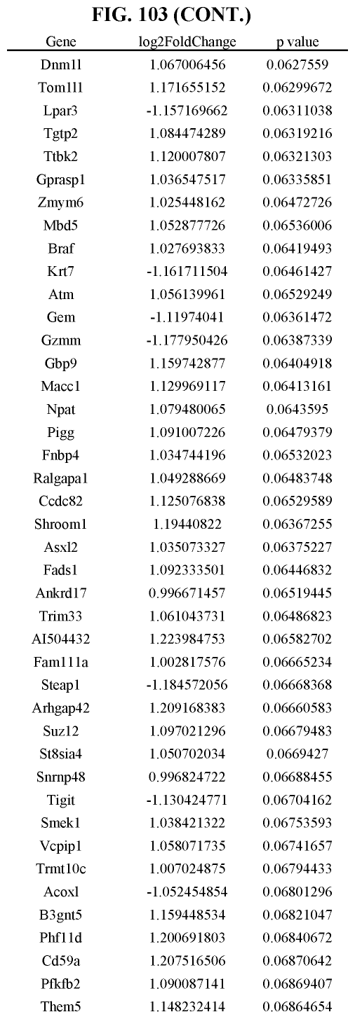

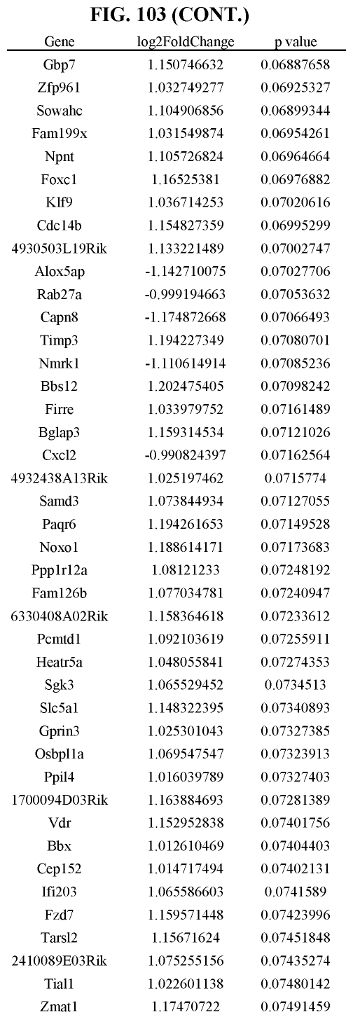

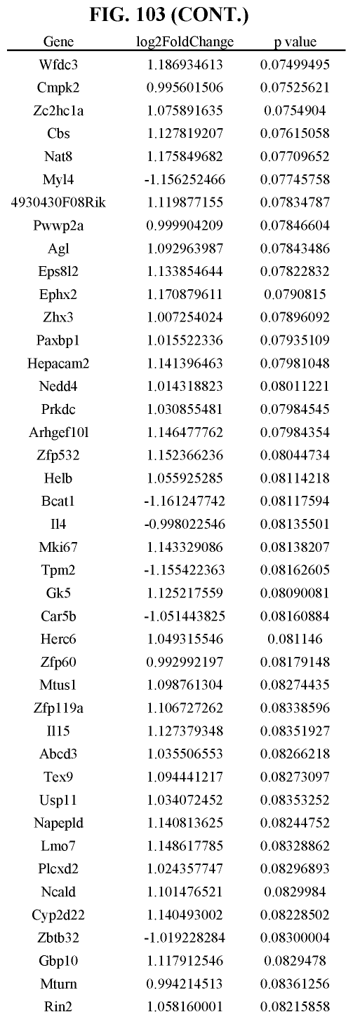

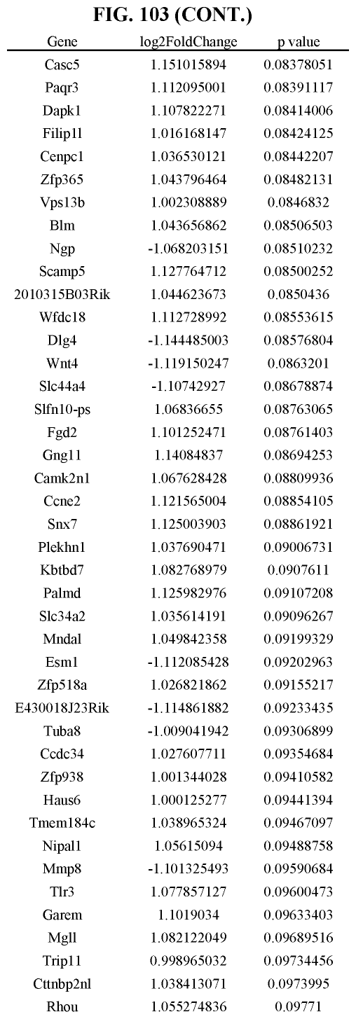

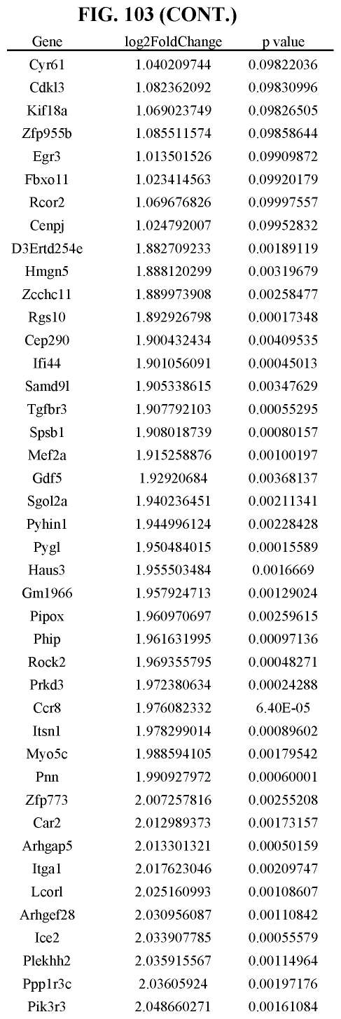

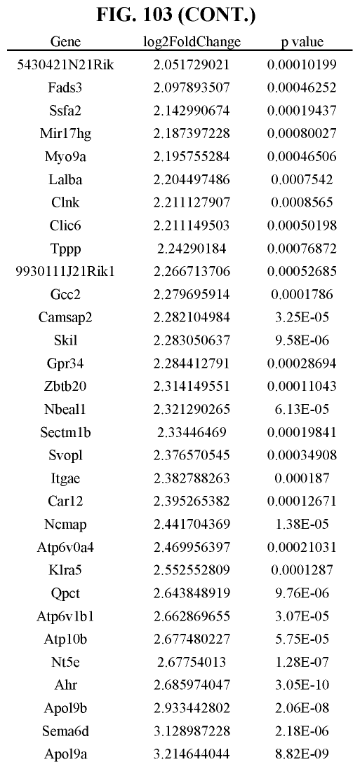

Statistical analysis. Related to FIG. 24, FIG. 25 and FIG. 103, differentially expressed genes were compared between tumor-infiltrating CD4⁺CD25⁻ T cells from Tgfbr2^{fl/fl}PyMT and ThPOK^{Cre}Tgfbr2^{fl/fl}PyMT mice. A gene list was generated if passing the following filters: mean expression >50 and log₂ fold change >1 or <−1. (All statistical measurements are displayed as mean±SEM.) For comparisons, unpaired student t test, two-tailed was conducted using GraphPad Prism software; for paired distance comparisons, paired t-test was conducted using GraphPad Prism software. For tumor growth, 2-way ANOVA was performed using GraphPad Prism software.

### Example 2: TGF-β Acts on CD4⁺ Cells to Foster Tumor Growth

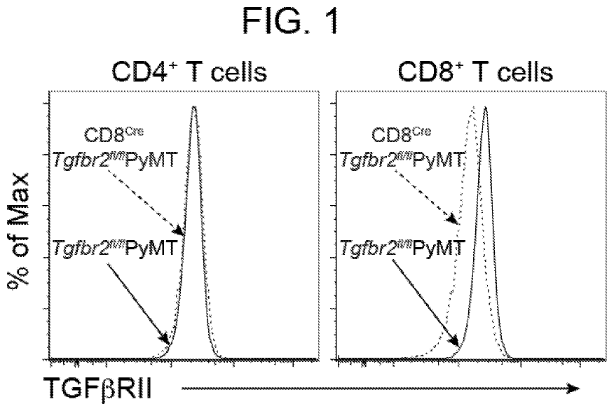

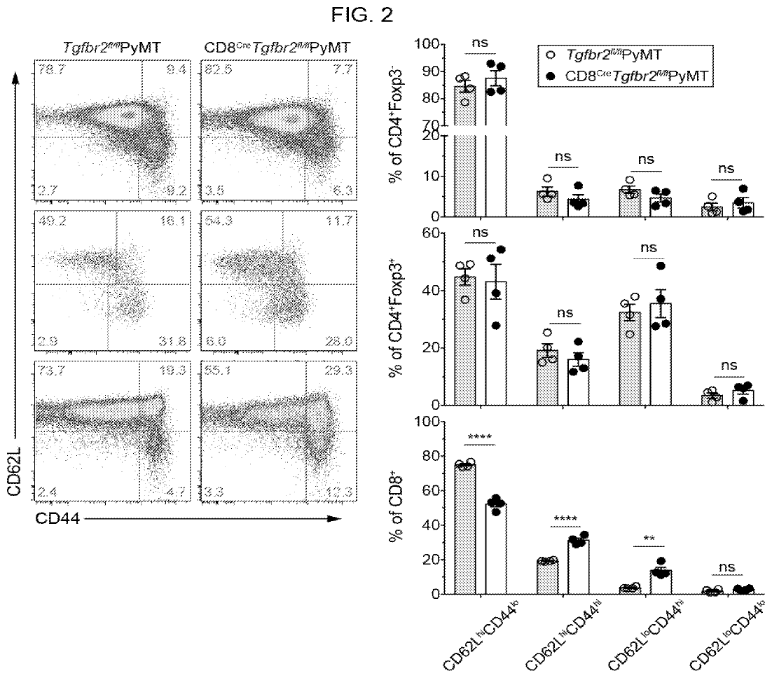

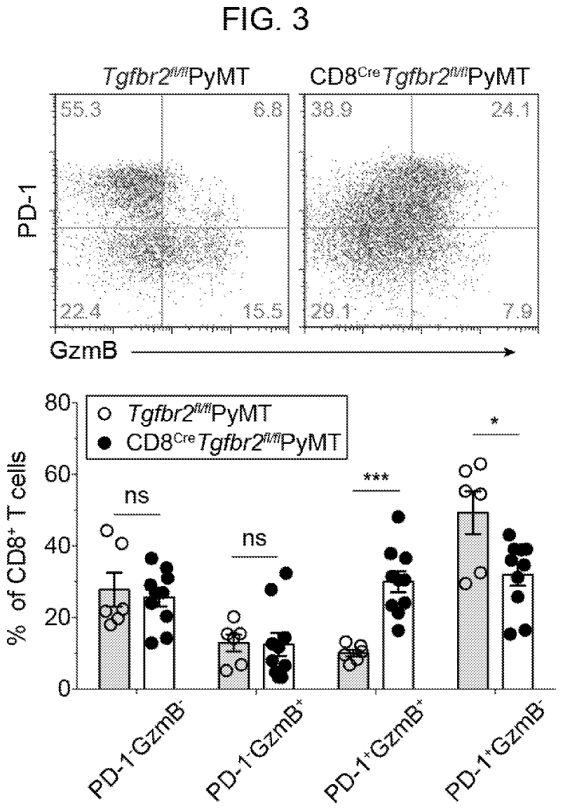

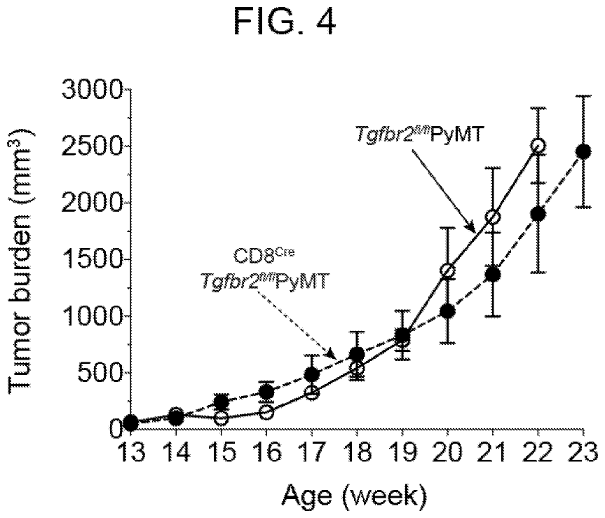

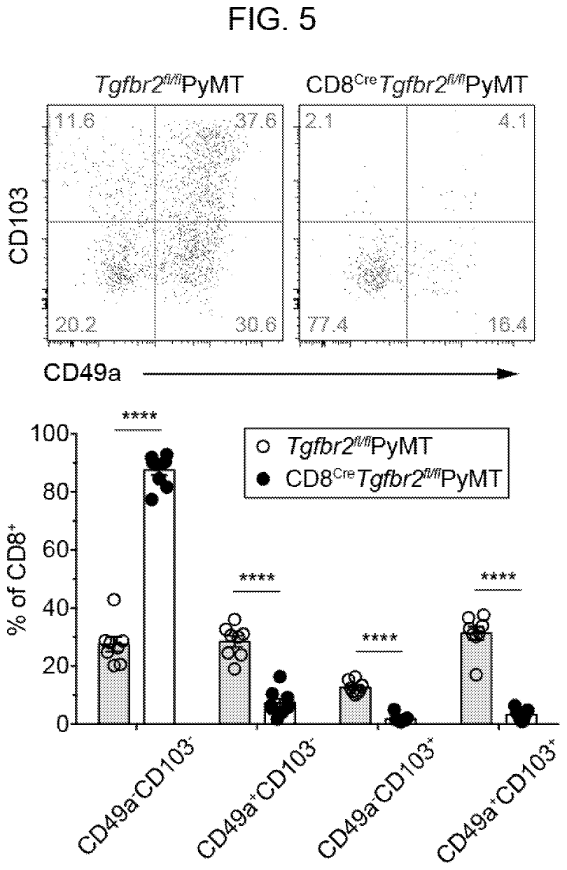

Studies were first conducted to determine whether TGF-β directly suppressed CTL-mediated cancer surveillance in the MMTV-PyMT (PyMT) model of breast cancer. Mice carrying a floxed allele of the Tgfbr2 gene (Tgfbr2^{fl/fl}) encoding the TGF-β receptor II (TGF-βRII) were crossed with CD8^{Cre} transgenic mice, which were further bred to the PyMT background. Loss of TGF-βRII was observed specifically in CD8⁺ T cells from CD8^{Cre}Tgfbr2^{fl/fl}PyMT mice (FIG. 1), which led to enhanced CD8⁺ T cell activation in the tumor-draining lymph nodes (FIG. 2). Increased expression of the cytolytic enzyme Granzyme B among PD-1-expressing CD8⁺ T cells was also detected in the tumors (FIG. 3). Surprisingly, tumor growth was not suppressed in CD8^{Cre}Tgfbr2^{fl/fl}PyMT mice (FIG. 4). Additionally, TGF-βRII-deficient CD8⁺ T cells expressed lower levels of the tissue residency markers CD49a and CD103 (FIG. 5). Therefore, blockade of TGF-β signaling in CD8⁺ T cells was unable to break tumor immune tolerance, likely because TGF-β-induced tissue residency programs are essential for CTL-mediated cancer resistance.

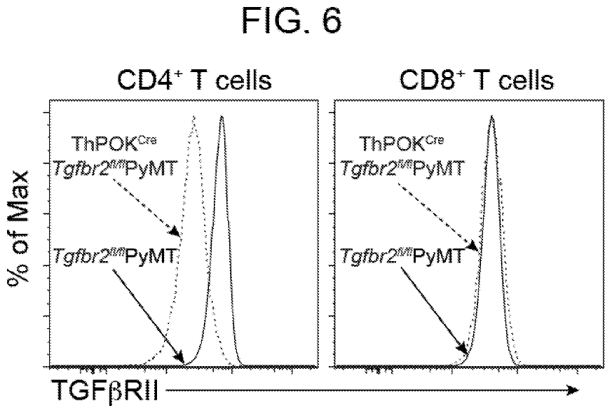

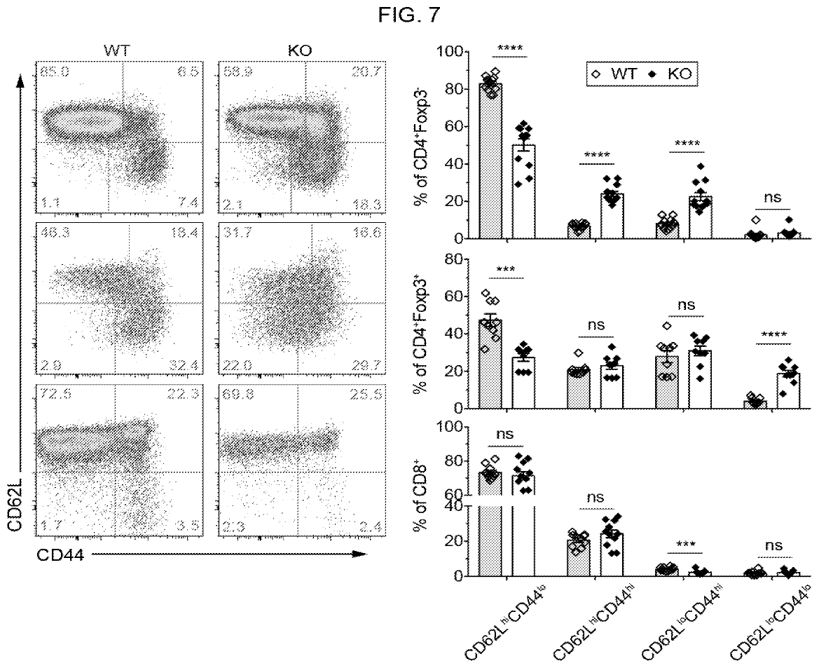

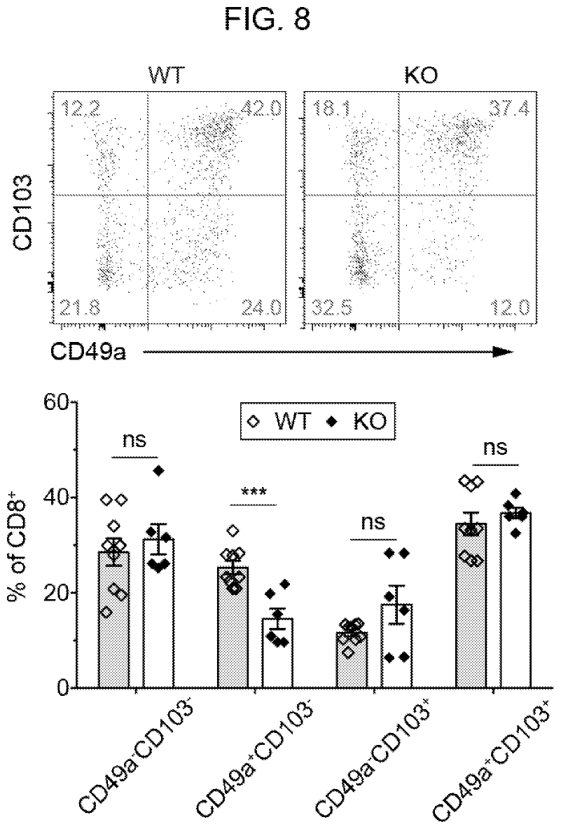

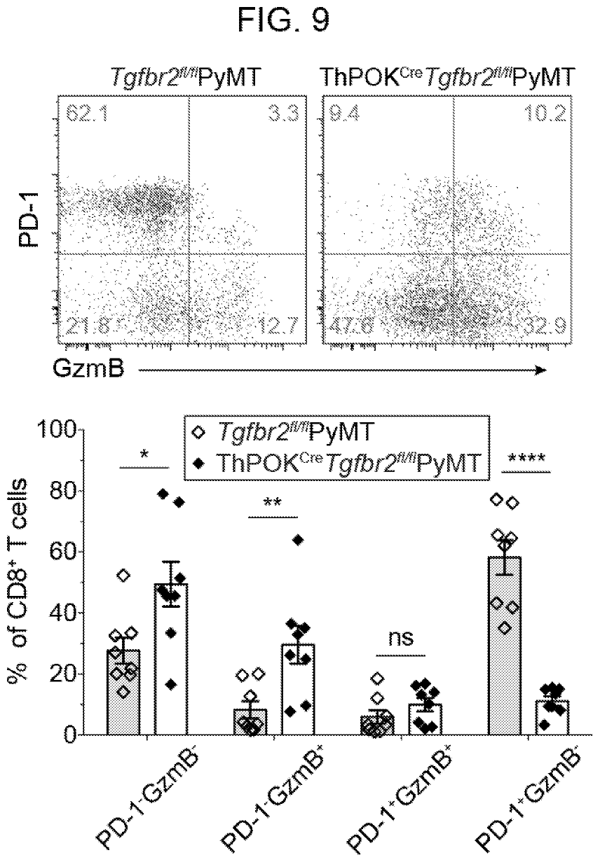

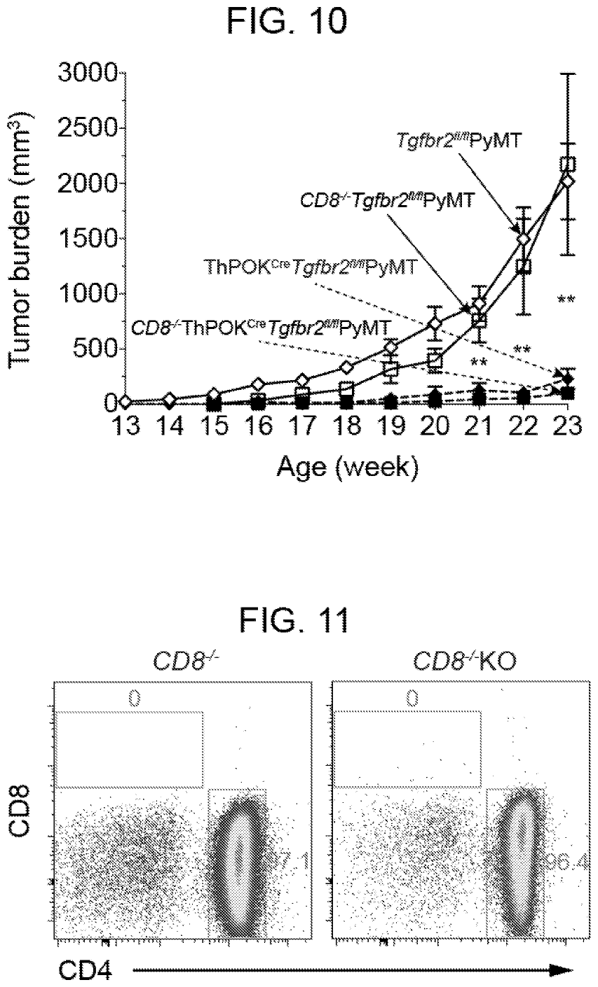

To investigate whether TGF-β targeted helper T cells to indirectly suppress CTL-dependent cancer surveillance, Tgfbr2^{fl/fl}PyMT mice were crossed with ThPOK^{Cre} mice, which ablated TGF-βRII specifically in CD4⁺ T cells (FIG. 6). Compared to control Tgfbr2^{fl/fl}PyMT mice, ThPOK^{Cre}Tgfbr2^{fl/fl}PyMT mice exhibited enhanced activation of conventional CD4⁺ T cells and Treg cells, but not CD8⁺ T cells, in the tumor-draining lymph nodes (FIG. 7). Expression of the tissue residency markers CD49a and CD103 on tumor-infiltrating CD8⁺ T cells was also unaffected (FIG. 8); yet, high levels of Granzyme B and low levels of PD-1 were detected (FIG. 9). Such a phenotypic change was previously observed in T cell-specific TGF-β1-deficient mice that resist tumor growth (see Donkor, M. K. et al., Immunity 35, 123-134 (2011)). Profound inhibition of tumor progression was observed in ThPOK^{Cre}Tgfbr2^{fl/fl}PyMT mice (FIG. 10).

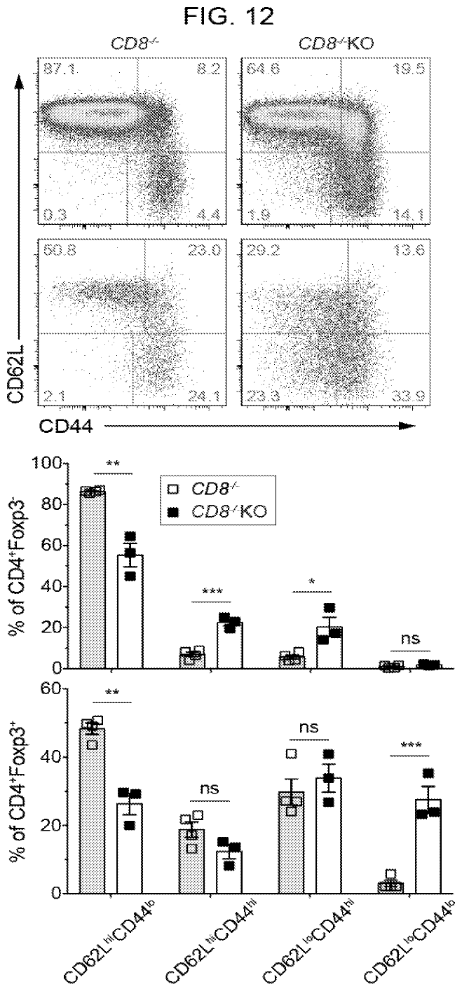

To determine whether CTL responses accounted for the tumor repression, ThPOK^{Cre}Tgfbr2^{fl/fl}PyMT mice were crossed to the CD8-deficient background. Unexpectedly, tumor suppression was unchanged in the absence of CD8⁺ T cells (FIG. 10), and the enhanced activation of CD4⁺ T cells was unperturbed in the tumor-draining lymph nodes (FIGS. 11-12). These findings demonstrate that CD4⁺ T cells are the functional targets of TGF-β in tumor immune tolerance control, and the reduced tumor growth triggered by TGF-βRII-deficient CD4⁺ T cells is not mediated by CTLs.

### Example 3: Tumor Cell Death Occurs with Immune Exclusion

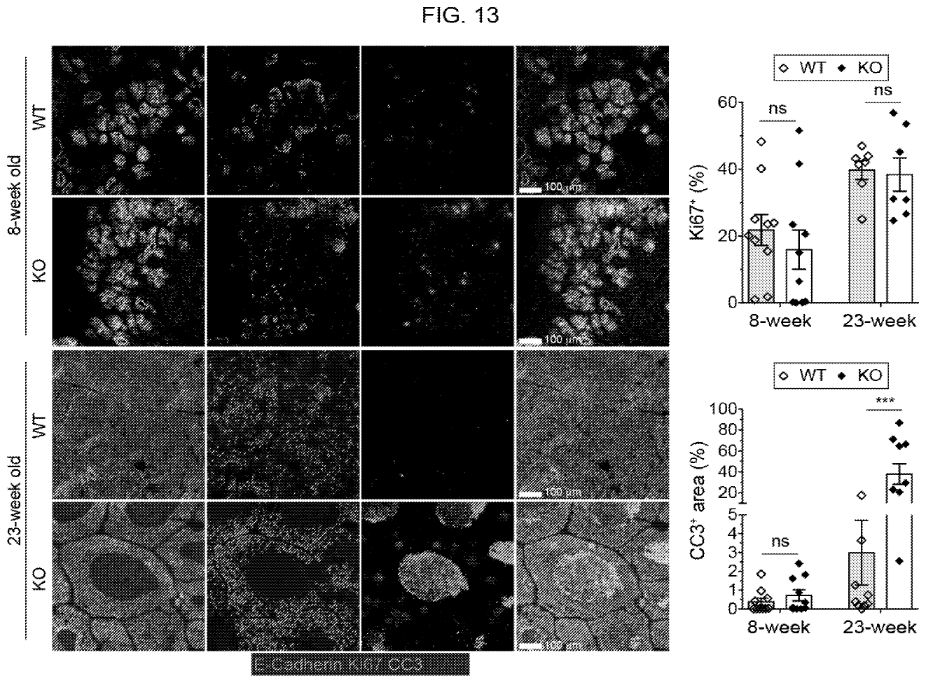

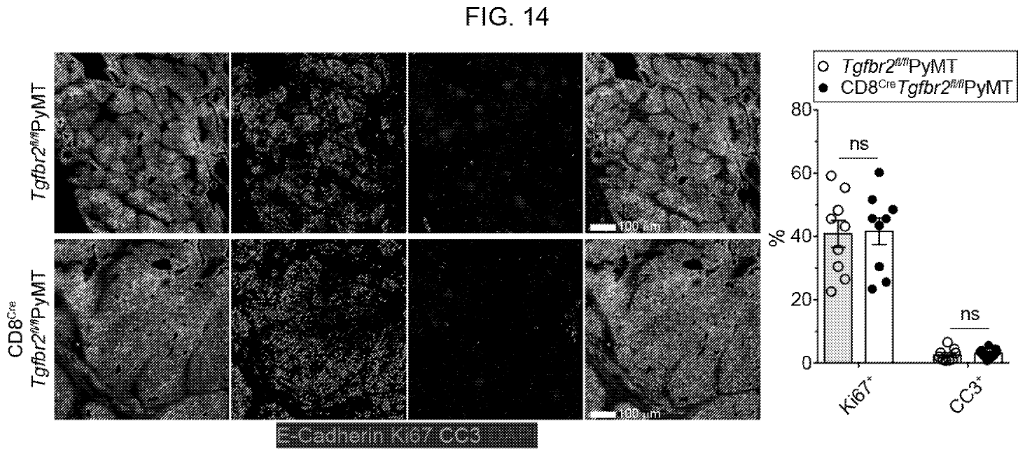

To define the underlying mechanisms of tumor repression in ThPOK^{Cre}Tgfbr2^{fl/fl}PyMT mice, proliferation and death of tumor cells were assessed by the expression of Ki67 and cleaved caspase 3 (CC3), respectively. Ki67 was expressed in about 20% and 40% mammary epithelial cells in tumors from 8-week-old and 23-week-old Tgfbr2_{fl/fl} PyMT mice, which was unaffected in ThPOK^{Cre}Tgfbr2^{fl/fl}PyMT mice (FIG. 13). In contrast, blockade of TGF-β signaling in CD4⁺ T cells resulted in an approximate 13-fold increase of CC3-positive cells at 23 weeks of age (FIG. 13). Notably, dying tumor cells had a clustered distribution pattern (FIG. 13), which was not observed in CD8^{Cre}Tgfbr2^{fl/fl}PyMT mice (FIG. 14) but was preserved in ThPOK^{Cre}Tgfbr2^{fl/fl}PyMT mice on the CD8-deficient background (FIG. 15). These observations demonstrate that TGF-βRII-deficient CD4⁺ T cells inhibit cancer progression via the induction of tumor cell death.

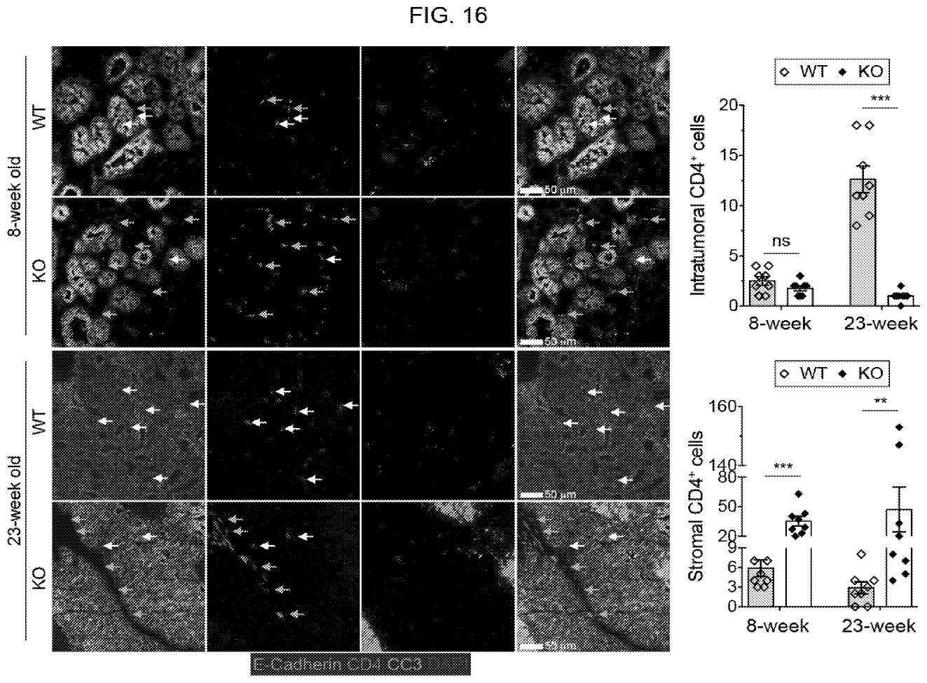

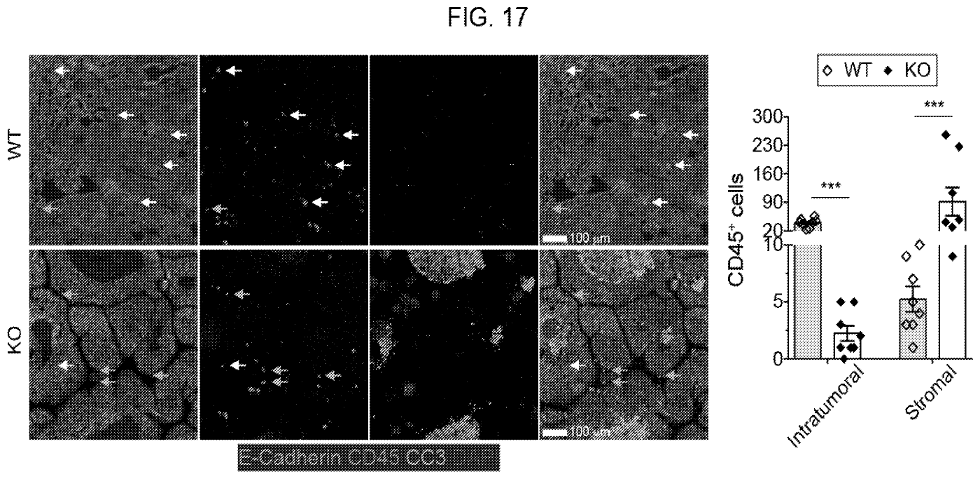

Based on the lack of a role for CTLs in tumor repression in ThPOK^{Cre}Tgfbr2^{fl/fl}PyMT mice, studies were conducted to investigate whether CD4⁺ T cells might directly eradicate tumor cells. To this end, localization of CD4⁺ T cells was examined by immunofluorescence staining. Tumor progression was associated with an approximate 5-fold increase of intratumoral CD4⁺ T cells between 8-week-old and 23-week-old Tgfbr2^{fl/fl}PyMT mice, while stromal CD4⁺ T cells were unaffected (FIG. 16). In contrast, blockade of TGF-β signaling in CD4⁺ T cells led to approximate 6- and 16-fold increases of stromal CD4⁺ T cells in 8-week-old and 23-week-old mice, respectively, while intratumoral CD4⁺ T cells were substantially reduced in 23-week-old ThPOK^{Cre}Tgfbr2^{fl/fl}PyMT mice (FIG. 16). Furthermore, few intratumoral CD4⁺ T cells were localized distant from the tumor cell death region (FIG. 16), suggesting against direct tumor cell killing by TGF-βRII-deficient CD4⁺ T cells. Immunofluorescence staining with the pan-leukocyte marker CD45 was then performed to examine whether the preferential tumor stroma localization of CD4⁺ T cells in ThPOK^{Cre}Tgfbr2^{fl/fl}PyMT mice applied to other hematopoietic lineage cells. In contrast to the dominant tumor parenchyma localization of CD45⁺ cells in Tgfbr2^{fl/fl}PyMT mice, leukocytes were mostly localized in the tumor stroma of ThPOK^{Cre}Tgfbr2^{fl/fl}PyMT mice (FIG. 17). The unexpected immune cell exclusion phenotype implies that TGF-βRII-deficient CD4⁺ T cells unlikely induce tumor cell death directly or indirectly via another effector leukocyte population.

### Example 4: Vessel Organization Triggers Tumor Cell Death

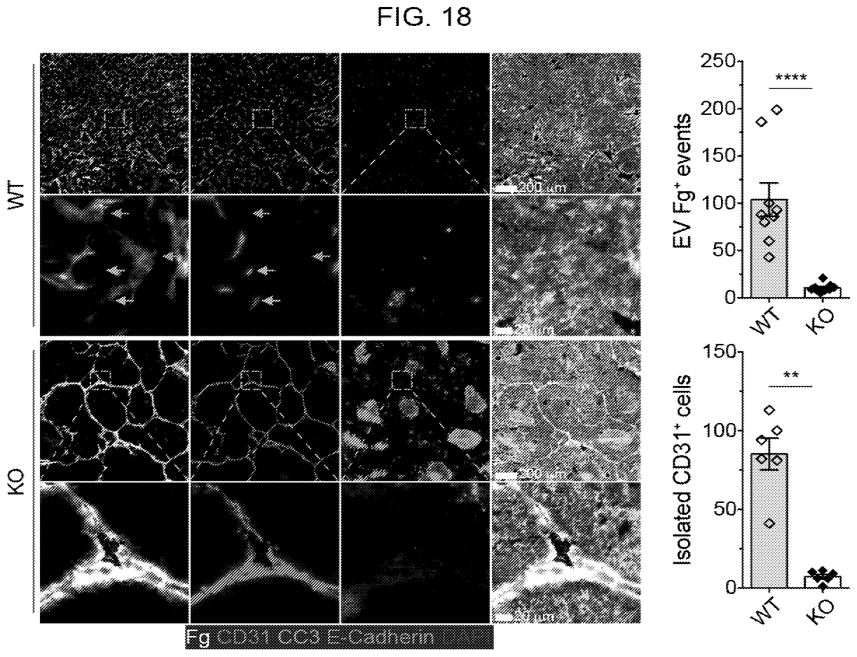

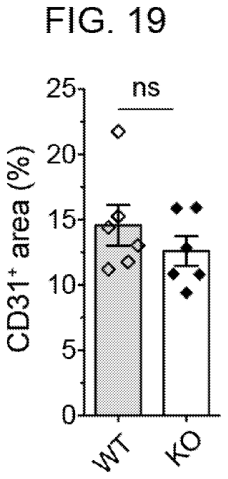

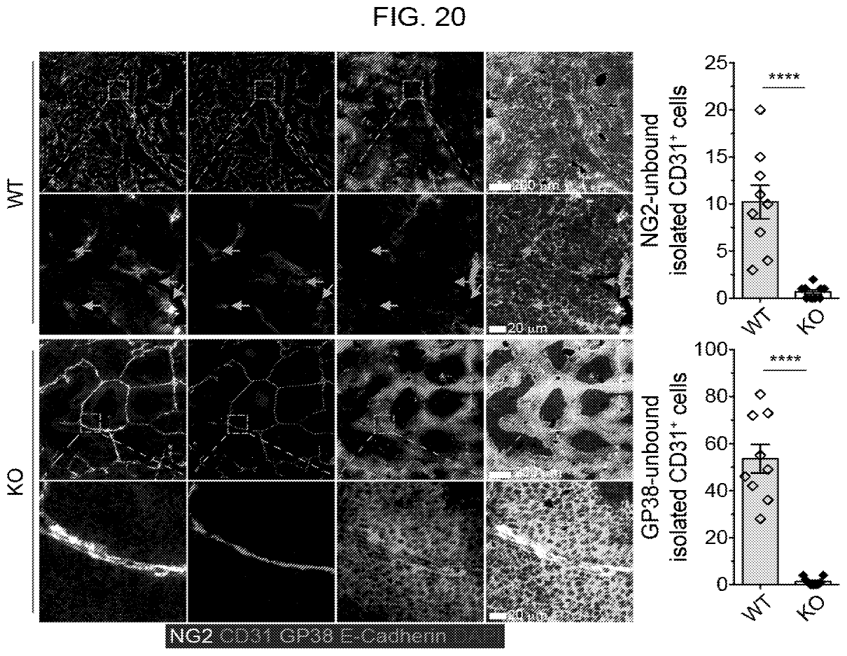

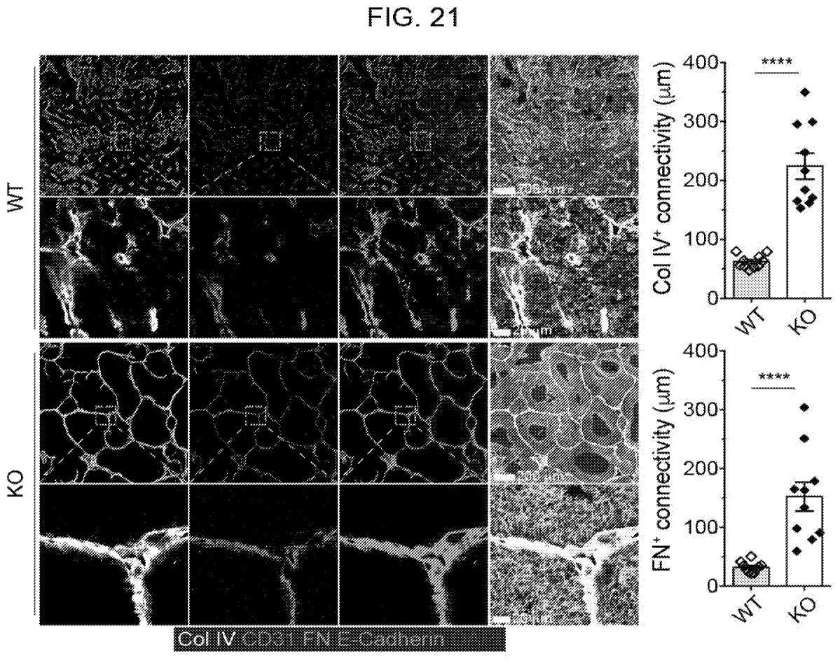

The preferential stroma localization of TGF-βRII-deficient CD4⁺ T cells suggested that they may regulate the host to endure the negative impact of a growing tumor with tumor cell death being a secondary outcome. Fast growing tumors in Tgfbr2^{fl/fl}PyMT mice exhibited extensive extravascular deposition of fibrinogen (FIG. 18), indicative of vasculature damage. In contrast, fibrinogen was predominantly intravascular in ThPOK^{Cre}Tgfbr2^{fl/fl}PyMT mice (FIG. 18). The extravascular fibrinogen distribution in Tgfbr2^{fl/fl}PyMT mice co-occurred with an irregularly shaped and bluntly ended microvasculature manifested by the isolated staining of the endothelium marker CD31 (FIG. 18). Strikingly, although the vessel density was unaffected (FIG. 19), tumor vasculature was much more organized in ThPOK^{Cre}Tgfbr2^{fl/fl}PyMT mice with an approximate 13-fold fewer isolated CD31⁺ endothelial cells (FIG. 18). These observations demonstrate that while tumors from Tgfbr2^{fl/fl}PyMT mice inflict chronic tissue damage resembling “wounds that do not heal” (see Dvorak, H. F. Tumors: wounds that do not heal. Similarities between tumor stroma generation and wound healing, N. Engl. J. Med. 315, 1650-1659, doi:10.1056/NEJM198612253152606 (1986)), tumors from ThPOK^{Cre}Tgfbr2^{fl/fl}PyMT mice are maintained in a healed state associated with an organized vasculature. Pericytes are mesenchyme-derived cells that enwrap and stabilize capillaries and control perfusion. Notably, while approximately 12% of the isolated endothelial cells in tumors from Tgfbr2^{fl/fl}PyMT mice were not bound by NG2⁺ pericytes (FIGS. 18 and 20), the endothelium was tightly ensheathed by pericytes in tumors from ThPOK^{Cre}Tgfbr2^{fl/fl}PyMT mice (FIG. 20). Fibroblasts are heterogenous populations of ‘accessory’ cells that provide structural support for ‘customer’ cell subsets including the endothelium. Remarkably, approximately 63% of the isolated endothelial cells in tumors from Tgfbr2^{fl/fl}PyMT mice were not associated with GP38⁺ fibroblasts (FIGS. 18 and 20), whereas extended layers of GP38⁺ cells enclosed the vasculature in tumors from ThPOK^{Cre}Tgfbr2^{fl/fl}PyMT mice (FIG. 20). In addition to the supportive cellular compartment, acellular components including extracellular matrix proteins regulate vascular integrity. Associated with the aberrant vessel patterning, the vessel basement membrane proteins collagen IV and fibronectin were fragmented and disoriented in tumors from Tgfbr2^{fl/fl}PyMT mice (FIG. 21). In contrast, both collagen IV and fibronectin were highly connected and colocalized with the endothelium in tumors from ThPOK^{Cre}Tgfbr2^{fl/fl}PyMT mice (FIG. 21). Together, these findings reveal that blockade of TGF-β signaling in CD4⁺ T cells promotes the generation of an organized and mature vasculature in the tumor.

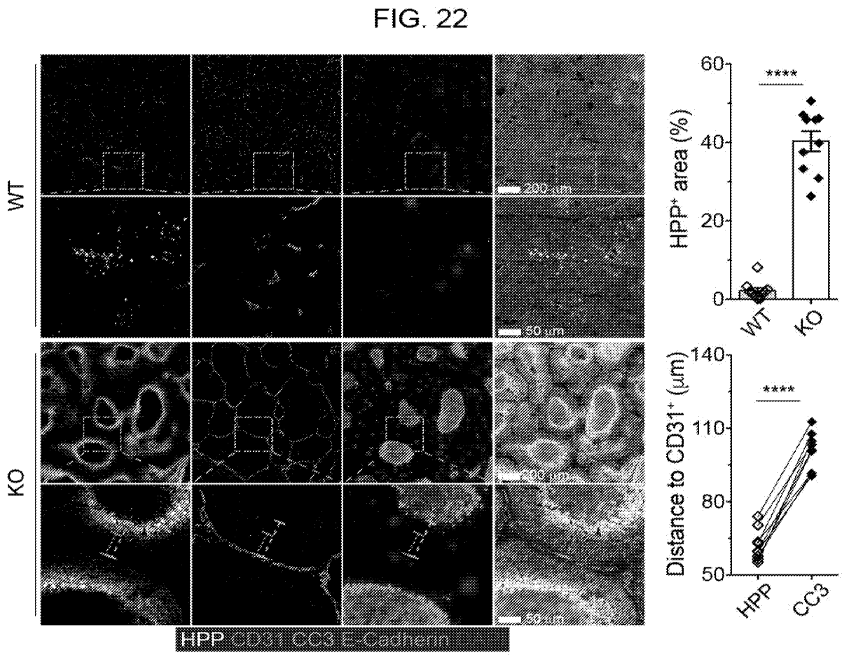

Sprouting angiogenesis is induced in malignant tissues in response to hypoxia and metabolic stresses, which resupplies oxygen and nutrients to a growing tumor (Bergers, G. & Benjamin, Nature reviews. Cancer 3, 401-410 (2003); Carmeliet, P. & Jain, R. K., Nature 473, 298-307 (2011)). Excessive vessel branching in tumors from Tgfbr2^{fl/fl}PyMT mice was associated with few hypoxic spots (FIG. 22). In contrast, approximately 18-fold larger areas were positive for the hypoxic probe in tumors from ThPOK^{Cre}Tgfbr2^{fl/fl}PyMT mice (FIG. 22). Notably, the hypoxic areas exhibited a circular pattern and were localized peripheral to the tumor cell death region with the initiating hypoxia and tumor cell death area positioned about 60 μm and 101 μm inward to the adjacent vasculature, respectively (FIG. 22). Together, these observations demonstrate that tumor cell death in ThPOK^{Cre}Tgfbr2^{fl/fl}PyMT mice is caused by severe hypoxia and/or depletion of nutrients, which is enabled by an organized vasculature refractory to the damaging effect of a growing tumor. Such a tumor microenvironment-targeted host defense strategy is herein classified as a cancer tolerance mechanism.

### Example 5: TGF-β Represses T Helper Cell Responses to Tumors

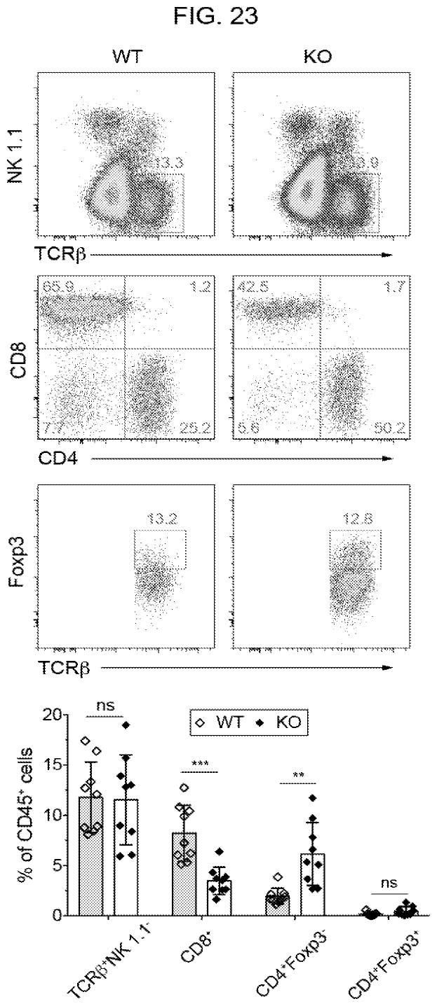

Tumor-infiltrating T cells from Tgfbr2^{fl/fl}PyMT and ThPOK^{Cre}Tgfbr2^{fl/fl}PyMT mice were analyzed to define how TGF-βRII deficiency in CD4⁺ T cells reprograms the tumor microenvironment and induces cancer tolerance. Although the frequencies of tumor-associated CD4⁺Foxp3⁺ Treg cells were not significantly altered, conventional CD4⁺Foxp3⁻ T cells expanded at the expense of CD8⁺ T cells in ThPOK^{Cre}Tgfbr2^{fl/fl}PyMT mice (FIG. 23).

Tumor-infiltrating CD4⁺CD25⁻ T cells from Tgfbr2^{fl/fl}PyMT and ThPOK^{Cre}Tgfbr2^{fl/fl}PyMT mice were purified, and RNAseq experiments were performed, to explore the gene expression program regulated by TGF-β in conventional CD4⁺ T cells. 312 and 976 genes were significantly upregulated and downregulated, respectively, in TGF-βRII-deficient T cells (FIGS. 24 and 25, and 103). Among the upregulated transcripts were those encoding signaling molecules such as Mapk11, Mapkapk2, Pik3ap1, Sh2d1a, Sh3bp2, and Syk, as well as antigen-induced co-stimulatory and co-inhibitory receptors such as Tnfrs4, Tnfrsf9, Tnfrsf18, Ctla4, Havcr2, Lag3 and Pdcd1 (FIG. 24), in agreement with enhanced activation of TGF-βRII-deficient CD4⁺ T cells in the tumor-draining lymph nodes (FIG. 7). Additionally, expression of the blood-homing Sphingosine 1-phosphate receptor Slpr5 was higher in TGF-βRII-deficient T cells (FIG. 24), while genes encoding the tissue retention integrins including Itga1 and Itgae were lower (FIG. 25), in line with their stromal localization in the tumor (FIG. 16). Genes encoding the glucose transporters Slc2a3 and Slc2a6 as well as the glycolytic enzymes Hk2, Gapdh, Pgk1 and Pkm were also induced (FIG. 24), which might promote T helper cell differentiation. Indeed, a larger number of transcripts encoding secreted molecules were upregulated than downregulated in TGF-βRII-deficient T cells (FIG. 24 and FIG. 25), which comprised T helper 1 (Th1) and Th2 cytokines Ifng, Il-4 and Il-5, Ccl and Cxcl chemokines, colony-stimulating factors as well as matrix metalloproteinases, and the Serpin family of serine proteinase inhibitors with important functions in resolving inflammation and healing wounds.

Furthermore, although a smaller number of nuclear factors were induced than repressed in TGF-βRII-deficient T cells (FIG. 24 and FIG. 25), several of them, including Batf, Bhlhe40, Irf4, and Pparg, have recently been shown to reside in major regulatory nodes of T cell activation and Th2 cell differentiation. (See Henriksson, J. et al., Genome-wide CRISPR Screens in T Helper Cells Reveal Pervasive Crosstalk between Activation and Differentiation, Cell 176, 882-896 e818, doi:10.1016/j.cell.2018.11.044 (2019)). Collectively, these findings reveal that blockade of TGF-β signaling reprograms the transcriptome of tumor-infiltrating CD4⁺ T cells with characteristics of enhanced T cell activation, augmented T helper cell differentiation, and attenuated tissue retention.

### Example 6: Type 2 Immunity Promotes Vessel Organization and Inhibits Cancer Progression

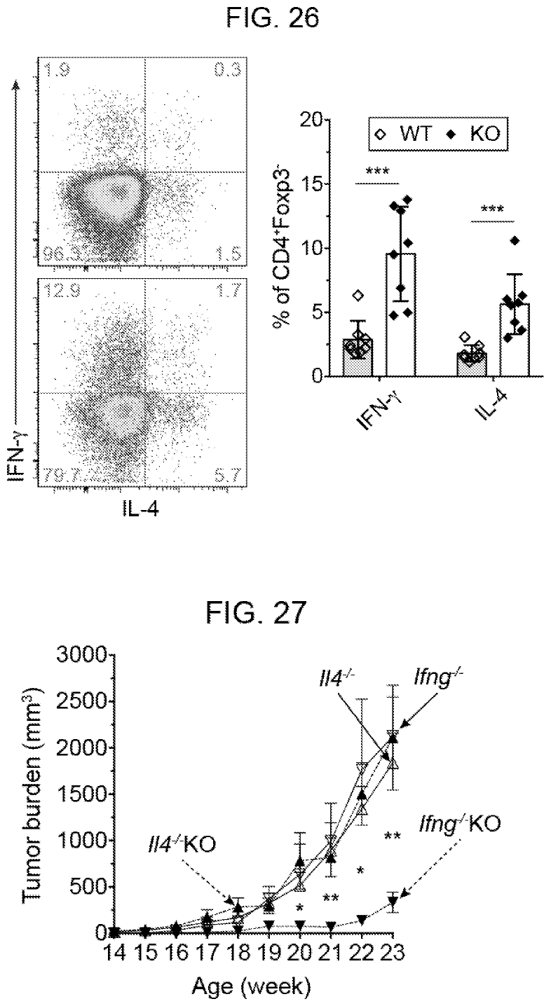

Studies were conducted to help define immune effector programs utilized by TGF-βRII-deficient CD4⁺ T cells to fortify vasculature organization and repress cancer progression. In line with RNAseq experiments, CD4⁺Foxp3⁻ cells from tumor-draining lymph nodes and tumor tissues of ThPOK^{Cre}Tgfbr2^{fl/fl}PyMT mice produced higher levels of Th1 and Th2 signature cytokines IFN-γ and IL-4 (FIG. 26 and data not shown).

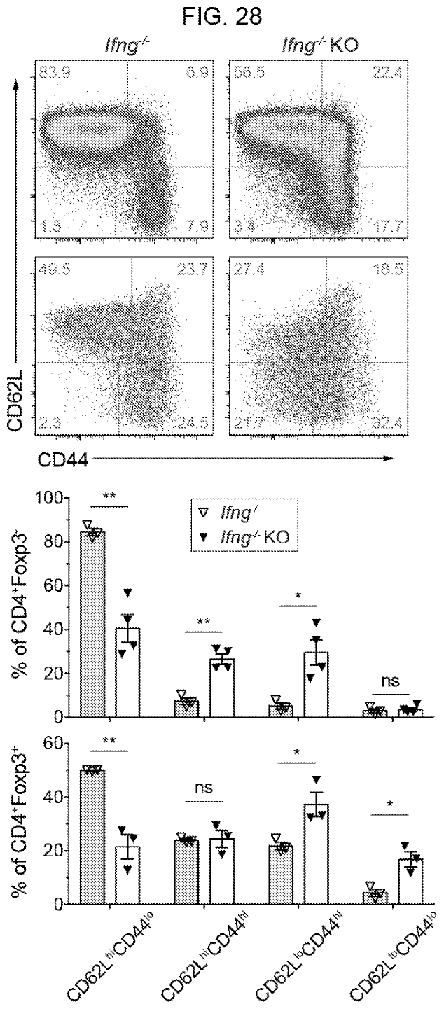

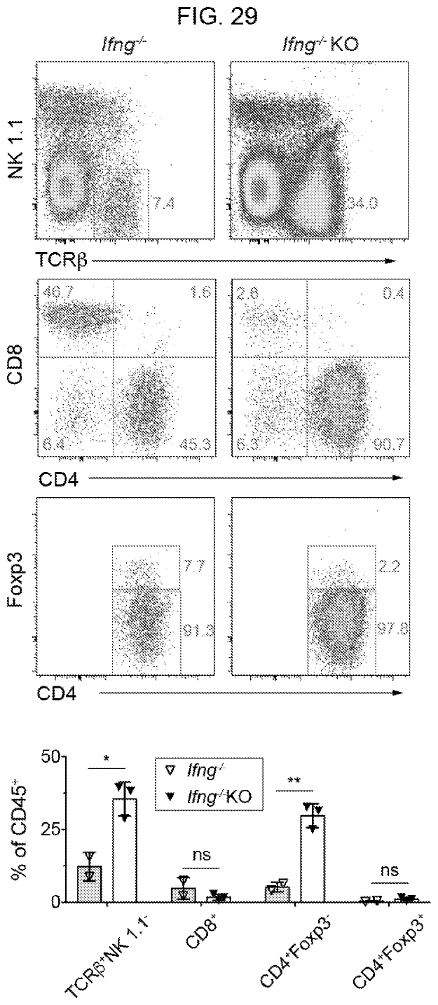

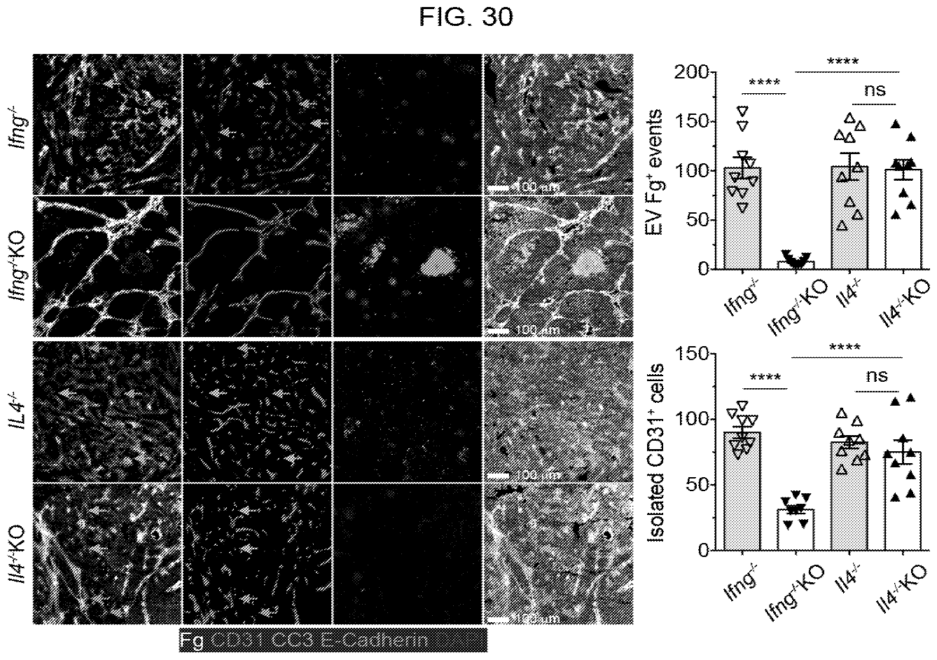

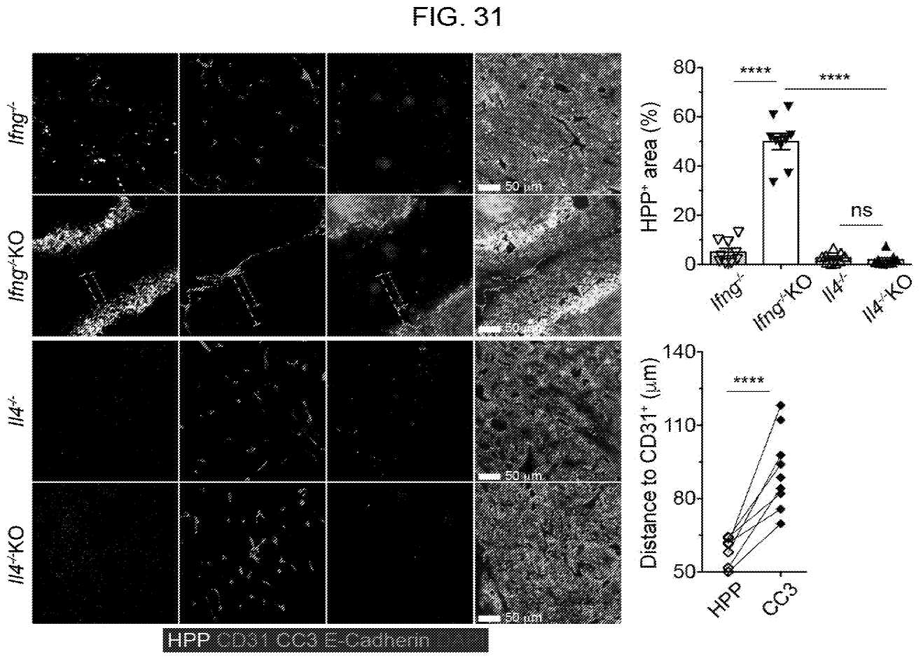

In transplantation models of murine cancer, recent studies have shown that Th1 cells and IFN-γ promote pericyte coverage of the endothelium and vessel regression, respectively (Tian, L. et al. Nature 544, 250-254 (2017); Kammertoens, T. et al., Nature 545, 98-102 (2017)). To interrogate the function of IFN-γ in the transgenic breast cancer model, we crossed ThPOK^{Cre}Tgfbr2^{fl/fl}PyMT mice to the IFN-γ-deficient background. Unexpectedly, the repressed tumor growth was unaffected in the absence of IFN-γ (FIGS. 10 and 27). In addition, IFN-γ deficiency did not perturb the enhanced activation of TGF-βRII-deficient CD4⁺ T cells in the tumor-draining lymph nodes, or their expansion in the tumor (FIGS. 28 and 29). Furthermore, extravascular deposition of fibrinogen and the bluntly ended vasculature were observed in PyMT mice on the IFN-γ-deficient background, but were suppressed in Ifng^{-/-}ThPOK^{Cre}Tgfbr2^{fl/fl}PyMT mice (FIG. 30). Importantly, in the absence of IFN-γ the severe hypoxia response observed in ThPOK^{Cre}Tgfbr2^{fl/fl}PyMT mice was preserved with clustered CC3-positive cells distributed to the inner circle of the hypoxic region (FIG. 31). These observations exclude IFN-γ as a mediator of the cancer tolerance response triggered by TGF-βRII-deficient CD4⁺ T cells.

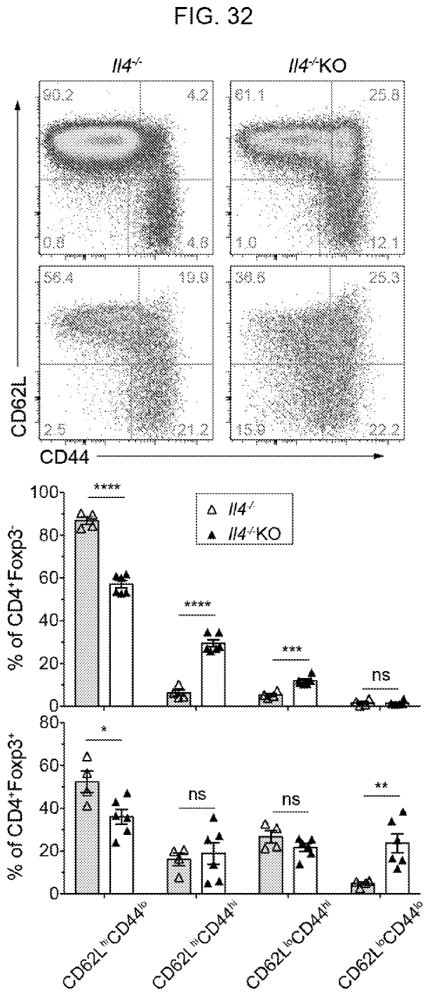

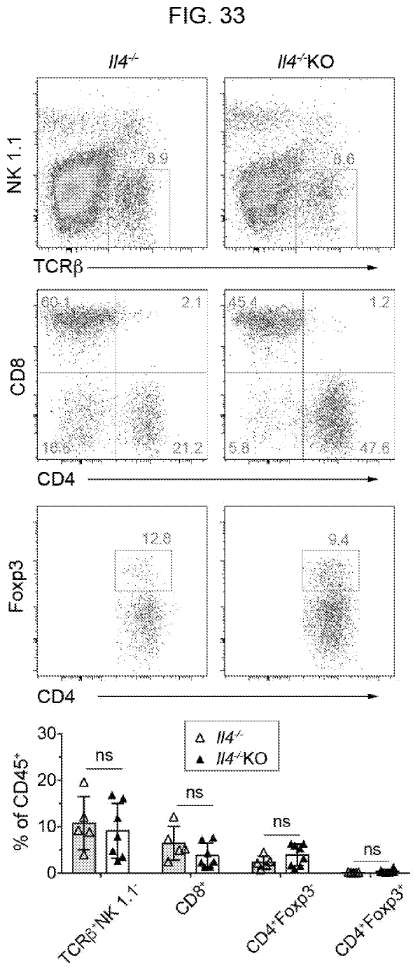

To investigate the function of type 2 immune responses in cancer tolerance regulation, ThPOK^{Cre}Tgfbr2^{fl/fl}PyMT mice were crossed to the IL-4-deficient background. IL-4 deficiency did not affect the enhanced activation of TGF-βRII-deficient CD4⁺ T cells in the tumor-draining lymph nodes, but their expansion in the tumor was attenuated (FIGS. 23, 32 and 33). In contrast to Ifng^{-/-}ThPOK^{Cre}Tgfbr2^{fl/fl}PyMT mice, Il-4^{-/-}ThPOK^{Cre}Tgfbr2^{fl/fl}PyMT mice had widespread extravascular deposition of fibrinogen associated with a torturous and irregularly shaped vasculature (FIG. 30). Furthermore, the enhanced hypoxia response and increased tumor cell death observed in ThPOK^{Cre}Tgfbr2^{fl/fl}PyMT mice were inhibited in the absence of IL-4 (FIG. 31), concomitant with accelerated tumor growth (FIG. 27). These findings demonstrate a pivotal function for the type 2 immune cytokine IL-4 in promoting vasculature organization and tumor suppression elicited by TGF-βRII-deficient CD4⁺ T cells.

### Example 7: Materials and Methods (for Example 8)

Mice. CD4^{CreERT2} mice were purchased from the Jackson Laboratory. Tgfrbr2^{fl/fl} and MMTV-PyMT (PyMT) mice were maintained in the laboratory as previously described. (See Ouyang, W., Beckett, O., Ma, Q. & Li, M. O., Transforming growth factor-beta signaling curbs thymic negative selection promoting regulatory T cell development, Immunity 32, 642-653, doi:10.1016/j.immuni.2010.04.012 (2010); Sarkar, A., Donkor, M. K. & Li, M. O., Oncotarget 2, 1339-1351 (2011)). The human CD4 (hCD4) transgenic mice were generated by pronuclear microinjection of fertilized eggs with a modified bacterial artificial chromosome (BAC) containing the human CD4 gene locus with the proximal enhancer region replaced by its murine equivalent. Briefly, a BAC harboring the human CD4 gene locus was recombineered with a pLD53.SC-AB shuttle plasmid containing the mouse Cd4 proximal enhancer flanked by two homologous arms of the human CD4 gene. (See Killeen, N., Sawada, S. & Littman, D. R., Regulated expression of human CD4 rescues helper T cell development in mice lacking expression of endogenous CD4, The EMBO Journal, 12, 1547-1553 (1993).) Founder hCD4 mouse strains were screened by PCR with human CD4-specific primers. All mice were backcrossed to the C57BL/6 background and maintained under specific pathogen-free conditions. Animal experimentation was conducted in accordance with procedures approved by the Institutional Animal Care and Use Committee of Memorial Sloan Kettering Cancer Center.

Tumor measurement. Mammary tumors in female PyMT mice were measured weekly with a caliper. Tumor burden was calculated using the equation [(L×W²)×(π/6)], in which L and W denote length and width. Total tumor burden was calculated by summing up individual tumor volumes of each mouse with an end-point defined when total burden reached 3,000 mm³ or one tumor reached 2,000 mm³.

Therapeutic treatment. (a) Tamoxifen (Sigma) was dissolved in corn oil at 50 mg/mL. Tgfbr2^{fl/fl}PyMT and CD4^{CreERT2}Tgfbr2^{fl/fl}PyMT mice bearing 5×5-6×6 mm (L×W) tumors were left untreated or were treated with 100 μL tamoxifen by oral gavage twice a week for 6 weeks. (b) CD4⁺CD25⁻ T cells were isolated from either Tgfbr2^{fl/fl} (wild-type, WT) or ThPOK^{Cre}Tgfbr2^{fl/fl} (knockout, KO) mice, and activated in vitro with immobilized CD3 and CD28 antibodies. Activated T cells were transferred to PyMT mice bearing 5×5 mm (L×W) tumors by intravenous injection. 1 million cells were transferred per mice, once a week for 6 weeks. (c) Antibodies were administered to hCD4PyMT mice bearing 5×5 mm tumors by intravenous injection twice a week for 5 weeks. The IL-4 neutralizing antibody (11D11, Bio X Cell) and IFN-γ neutralizing antibody (R4-6A2, Bio X Cell) were co-administered in some experiments.

Immune cell isolation from tissues. Single-cell suspensions were prepared from lymph nodes and spleens by tissue disruption with glass slides. The dissociated cells were passed through 70 μm filters and pelleted. Tumor-infiltrating immune cells were isolated from mammary tumors as previously described. (See Franklin, R. A. et al., The cellular and molecular origin of tumor-associated macrophages, Science 344, 921-925, doi:10.1126/science.1252510 (2014).) Briefly, tumor tissues were minced with a razor blade then digested in 280 U/mL Collagenase Type 3 (Worthington Biochemical) and 4 μg/mL DNase I (Sigma) in HBSS at 37° C. for 1 h and 15 min with periodic vortex every 20 min. Digested tissues were passed through 70 μm filters and pelleted. Cells were resuspended in 40% Percoll (Sigma) and were layered above 60% Percoll. Samples were centrifuged at 1,900 g at 4° C. for 30 min without brake. Cells at the interface were collected, stained, and analyzed by flow cytometry.

Flow cytometry. Flurochrome-conjugated or biotinylated antibodies against mouse CD4 (RM4-5), CD8 (53-6.7), Foxp3 (FJK-165), IFN-γ (XMG1.2), IL-4 (BVD6-24G2), NK1.1 (PK136) and TCRβ (H57-595) were purchased from eBioscience. Antibodies against mouse CD45 (clone 30-F11), CD11b (M1/70), CD11c (N418), Lytic (AL-21), Ly6G (1A8), MHC-II I-A/I-E (M5/114.15.2) were purchased from BD Biosciences. All antibodies were tested with their respective isotype controls. Cell-surface staining was conducted by incubating cells with antibodies for 30 min on ice in the presence of 2.4G2 mAb to block FcγR binding. For Foxp3 staining, a transcription factor-staining kit (Tonbo Biosciences) was used. To assess cytokine production, T cells were stimulated with 50 ng/mL phorbol 12-myristate 13-acetate (Sigma), 1 mM ionomycin (Sigma) in the presence of Golgi-Stop (BD Biosciences) for 4 h at 37° C. as previously described (See Oh, S. A. et al., Proc. Nat'l Acad. Sci. U.S.A., 114, E7536-E7544, doi:10.1073/pnas.1706356114 (2017)). T cells were subsequently stained for cell surface markers before intracellular cytokine staining. All data were acquired using an LSRII flow cytometer (Becton Dickinson) and analyzed with FlowJo software (Tree Star, Inc.).

Immunofluorescence staining. Antibodies against CD31 (MEC13.3) and GP38 (8.1.1) were purchased from Biolegend. Antibodies against Col IV (Cat. #2150-1470), fibrinogen (Cat. #4440-8004) and NG2 (Cat. #AB5320) were obtained from Bio-rad. Antibodies against fibronectin (Cat. #AB2033) and cleaved caspase 3 (Cat. #9661S) were purchased from EMD and Cell Signaling Technology, respectively. Antibodies against E-Cadherin (DECMA-1) and Ki67 (SolA15) were obtained from eBioscience. Antibody against VEGFA (Cat. #AF-493-NA) was purchased from R&D Systems. Tumor tissues were frozen in O.C.T. medium (Sakura Finetek USA) and sectioned at the thickness of 10 μm. Tumor sections were fixed and stained with antibodies. Subsequently, they were mounted with VECTORSHIELD anti-fade mounting media (Vector Laboratories) and scanned by Pannoramic Digital Slide Scanners (3DHISTECH LTD). Immunofluorescence images were analyzed with CaseViewer and Fiji software, and further processed in Adobe Photoshop and Illustrator software. To assess hypoxia response, 60 mg/kg pimonidazole hydrochloride was administered to mice via intraperitoneal injection. 1 h later, mice were sacrificed and tumor tissues were harvested. To detect the formation of pimonidazole adducts, tumor cryosections were immunostained with a Hypoxyprobe kit (Hypoxyprobe, Inc.) following the manufacturer's instructions.

Cell lines. HEK293 cells were purchased from ATCC (CRL-1573). FreeStyle 293-F cells were obtained from ThermoFisher Scientific. Sf9 and Hi5 insect cell lines were obtained from Prof. Morgan Huse (MSKCC). HEK293 cells stably expressing human CD4 were generated by retrovirus-mediated gene transfer. Briefly, HEK293 cells (5×10⁶) plated on 10 cm dishes were transfected with a human CD4-expressing retroviral vector containing an EGFP reporter (10 μg) together with a helper plasmid (5 μg). Two days after transfection, the viruses were harvested and used to infect HEK293 cells in the presence of 4 μg/mL polybrene (Sigma). Infection was repeated twice to enhance the transduction efficiency, and cells were selected by flow cytometry sorting based on EGFP signals.

Antibody cloning. DNA fragments of the ibalizumab VH domain and VL domain were synthesized by Genewiz. Antibody protein fusion constructs were generated by overlapping PCR of DNA fragments encoding the ibalizumab or mGO53 VH domain and mouse IgG1 constant regions. To abrogate IgG1 Fc effector functions, a D265A mutation was introduced by site-directed mutagenesis (Agilent Technologies). A DNA fragment encoding the human TGF-βRII extracellular domain (ECD) was chemically synthesized (Genewiz), and fused to antibody expression constructs via a DNA fragment encoding a (Gly₃Ser)₃ linker (SEQ ID NO: 46). αCD4 ScFv was generated by fusion of the ibalizumab VH domain with VL domain via a (Gly₃Ser)₃ linker (SEQ ID NO: 46). αTGF-β ScFv construct was generated by fusion of the fresolimuma VH domain with VL domain via a (Gly₃Ser)₃ linker (SEQ ID NO: 46). (See Moulin, A. et al., Protein sci: a publication of the Protein Society. 23, 1698-707, doi:10.1002/pro.2548 (2014)). αCD4/αTGF-β bispecific antibody was generated by fusion of αCD4 ScFv or αTGF-13 ScFv with a mouse IgG1 Fc domain containing a D265A mutation to block FcγR binding. The knob-into-hole strategy was utilized to promote heterodimerization between αCD4 ScFv-Fc and αTGF-β ScFv-Fc. The VEGF-Trap expressing construct was created by overlapping PCR of DNA fragments encoding a mouse IgG2a Fc domain, the second Ig domain of human VEGFR1, and the third Ig domain of human VEGFR2, as previously described. (See Holash, J. et al., VEGF-Trap: a VEGF blocker with potent antitumor effects, Proc. Nat'l Acad. Sci., U.S.A. 99, 11393-11398, doi:10.1073/pnas.172398299 (2002)).

Antibody expression and purification. Antibody-encoding plasmids were transiently transfected into FreeStyle 293-F cell lines. Cell culture supernatants were collected 4 days post-transfection, cleared by low-speed centrifugation and 0.45 μm filters, diluted with a 10× binding buffer (0.2 M Na₃PO₄, pH 7.0) and passed through a protein A/G prepackaged gravity flow column (GE Healthcare). Antibodies were eluted with 0.1 M glycine-HCl (pH 2.7) into a neutralizing buffer (1 M Tris-HCl, pH 9.0), concentrated by centrifugation, and buffer-exchanged into PBS (pH 7.4). Antibodies were quantified by spectrophotometry, and their purities were assessed by electrophoresis followed by Coomassie Blue staining. Size exclusion chromatography was used to further assess physicochemical homogeneity of antibodies and to resolve monomers from non-monomeric species. Briefly, antibodies were passed through an AKTA purifier (GE Healthcare) on a Superdex S200 10/300 GL column (GE Healthcare) with a mobile phase of PBS at a flow rate of 0.5 mL/min. Percent monomer was calculated as the area of the monomeric peak divided by the total area of monomeric plus nonmonomeric peaks at 280 nm. Antibody solutions were filtered through 0.22 μm filterers and validated for low endotoxin levels using a LAL chromogenic endotoxin quantification kit (Thermo Scientific) before further experimentation. To biotinylate antibodies, the C-terminus of antibody light chain was fused with a biotin-binding peptide and subjected to in vitro biotinylation with a BirA biotin-protein ligase reaction kit (Avidity).

Recombinant CD4 expression and purification. A DNA fragment encoding the extracellular domain of human CD4 (residues 26-390) with N-terminal fusion of the gp67 secretion signal and C-terminal fusion of a His tag was cloned into the baculovirus expression vector pAcGP67-A (BD Biosciences). Recombinant baculovirus was packaged in Sf9 cells, and used to infect Hi5 cells for CD4 protein expression. In a typical preparation, 500 mL liquid culture of Hi5 cells at a concentration of 2×10⁶ cells/mL were inoculated with 12 mL recombinant baculovirus at a concentration of 2×10⁸ pfu/mL. Supernatants were harvested 2 days after infection and loaded onto a Ni²⁺-NTA column (GE Healthcare) for affinity purification. Recombinant CD4 was further purified by sequential Superdex S-75 and MonoQ columns.

Surface plasmon resonance. Binding affinity analyses of 4T-Trap, αCD4, and an anti-TGF-β (αTGF-β, 1D11 clone purified from 1D11.16.8 hybridoma cell line from ATCC) were performed with a previously described protocol. (See Mouquet, H., Warncke, M., Scheid, J. F., Seaman, M. S. & Nussenzweig, M. C., Enhanced HIV-1 neutralization by antibody heteroligation, Proc. Nat'l Acad. Sci. U.S.A., 109, 875-880 (2012).) Briefly, recombinant human CD4 or TGF-β1 (Cat. #240-B-010, R&D systems) was immobilized to CMS sensor chips. The binding kinetics were monitored by flowing 4T-Trap, αCD4 and αTGF-β over the chip for association, which was further monitored for their dissociation, with the surface being washed for 5 min.

4T-Trap binding to cell surface CD4. Serial dilutions of 4T-Trap or αCD4 were prepared in 96-well U-bottom plates in DMEM medium. 2×10⁵HEK293-hCD4 cells were added to each well and incubated on ice for 1 h with shaking every 10 min. Cells were washed, resuspended, and incubated in PE-conjugated donkey anti-mouse IgG (Cat. #12-4012-82, eBioscience) on ice for 30 min with shaking every 10 min. Cells were rewashed and analyzed by flow cytometry. The mean fluorescence intensity value was quantified.

Enzyme-Linked Immunosorbent Assay (ELISA). Costar 96-well ELISA plates (Corning) were coated with 50 ng recombinant human CD4 or TGF-β1 for 18 h at 4° C. Plates were washed four times with 0.05% Tween-20 in PBS and blocked with 0.5% BSA in PBS for 1 h at room temperature. Serial dilutions of 4T-Trap or control antibodies were plated in triplicate and incubated at room temperature for 2 h. Plates were washed four times and incubated with peroxidase-conjugated goat anti-mouse IgG (Cat. #115-035-003, Jackson Immuno Research) at 37° C. for 1 h. To detect CD4 and TGF-β1 co-binding, CD4-coated plates that had been incubated with 4T-Trap or control antibodies were incubated with 100 ng recombinant TGF-β1 for 2 h. Plates were washed and incubated with a biotinylated TGF-β1 antibody (Cat. #BAF240, R&D systems) at room temperature for 2 h. Plates were further washed and incubated with peroxidase-conjugated streptavidin (Jackson Immuno Research) at 37° C. for 1 h. After final washes, plates were incubated in a TMB solution at room temperature for 5 to 20 min, and the reaction was terminated with 1 M HCl. Plate absorbance at 450 nm with background correction at 570 nm was detected with a SpectraMax 384 Plus Microplate Reader (Molecular Devices).

Pharmacokinetic analysis. Plasma samples were drawn from hCD4 transgenic mice after intravenous injection of biotinylated 4T-Trap or control antibodies for 1, 24, 48, 72 and 96 h. Streptavidin-coated plates (ThermoFisher Scientific) were incubated with plasma samples and standards at 37° C. for 1 h, washed four times and incubated with peroxidase-conjugated goat anti-mouse IgG at 37° C. for 1 h. The plates were rewashed four times, incubated in a TMB solution at room temperature for 5 to 20 min, and the reaction was terminated with 1 M HCl. Plate absorbance at 450 nm with a background correction at 570 nm was detected in a SpectraMax 384 Plus Microplate Reader (Molecular Devices).

Luciferase reporter assays. To assess TGF-β signaling, HEK293 cells or HEK293-hCD4 cells transfected with a TGF-β/SMAD Firefly luciferase reporter plasmid (see Zhou, S., Zawel, L., Lengauer, C., Kinzler, K. W. & Vogelstein, B., Characterization of human FAST-1, a TGF-β and activin signal transducer, Molecular Cell 2, 121-127 (1998)), and a pRL-TK Renilla luciferase reporter plasmid were plated in 24-well plates at 2×10⁵ cells per well in 500 μL of DMEM medium, and cultured for 18 h at 37° C. Plates were subsequently incubated with serial dilutions of 4T-Trap or control antibodies in DMEM medium for 30 min, which were left unwashed or washed and re-cultured with 10 ng/mL recombinant human TGF-β1 in DMEM medium for 12 h. Cells were subsequently lysed, and assayed for luciferase activities with a dual-specific luciferase reporter assay system (Promega). To validate VEGF-Trap inhibition of VEGF signaling, HEK293 cells were co-transfected with a VEGF-responsive NFAT Firefly luciferase reporter plasmid (see Clipstone, N. A. & Crabtree, G. R., Identification of calcineurin as a key signalling enzyme in T-lymphocyte activation, Nature 357, 695-697, doi:10.1038/357695a0 (1992)), a VEGFR2 expression plasmid, and a pRL-TK Renilla luciferase reporter plasmid. Plates were subsequently incubated with serial dilutions of VEGF-Trap and 10 ng/mL recombinant mouse VEGF 165 (Cat. #450-32, Peprotech) for 12 h before luciferase activities were measured.

Immunoblotting. CD4⁺ T cells from hCD4 transgenic mice were purified using a Magnisort Mouse CD4 T Cell Enrichment Kit (Affymetrix) and incubated with 10 ng/mL, 50 ng/mL, 100 ng/mL or 500 ng/mL 4T-Trap for 10 min. Cells were washed, cultured with 10 ng/mL recombinant human TGF-β1 for 1 h, and collected into a cell lysis buffer (50 mM Tris-HCl, pH 7.6, 150 mM NaCl, 0.5% Triton-X-100, 2 mM EGTA, 10 mM NaF, 1 mM Na₃VO₄ and 2 mM DTT) supplemented with protease inhibitors. Protein extracts were made, separated by SDS-PAGE gel and blotted with SMAD2/3 (D7G7) and phospho-SMAD2(Ser465/467)/SMAD3(S423/S425) (D27F4) antibodies from Cell Signaling Technology.

CD4 target occupancy assay. 100 μL blood was collected retro-orbitally in EDTA-coated Eppendorf tubes from mice that had been intravenously administered with biotinylated 4T-Trap. The blood samples were divided into two groups. The first group was spiked with 1 μg biotinylated 4T-Trap for 30 min at 4° C. as a 100% target occupancy (TO) control, while the second group was left untreated. All samples were washed twice with 1% FBS in PBS and stained with PE-conjugated streptavidin and a cocktail of antibodies against T cell surface markers for 30 min at 4° C. Cells were rewashed and analyzed by flow cytometry. The TO percentage was calculated as 100×[Mean Fluorescence Intensity (MFI) of sample PE signal/MFI of spiked sample PE signal].

Statistical analysis. All statistical measurements are displayed as mean±SEM. For comparisons, unpaired student t test, two-tailed was conducted using GraphPad Prism software; for paired distance comparisons, paired t-test was conducted using GraphPad Prism software. For tumor growth, 2-way ANOVA was performed using GraphPad Prism software.

### Example 8: Healing Anti-Tumor Immunity by Blockade of TGF-β Signaling in Helper T Cells

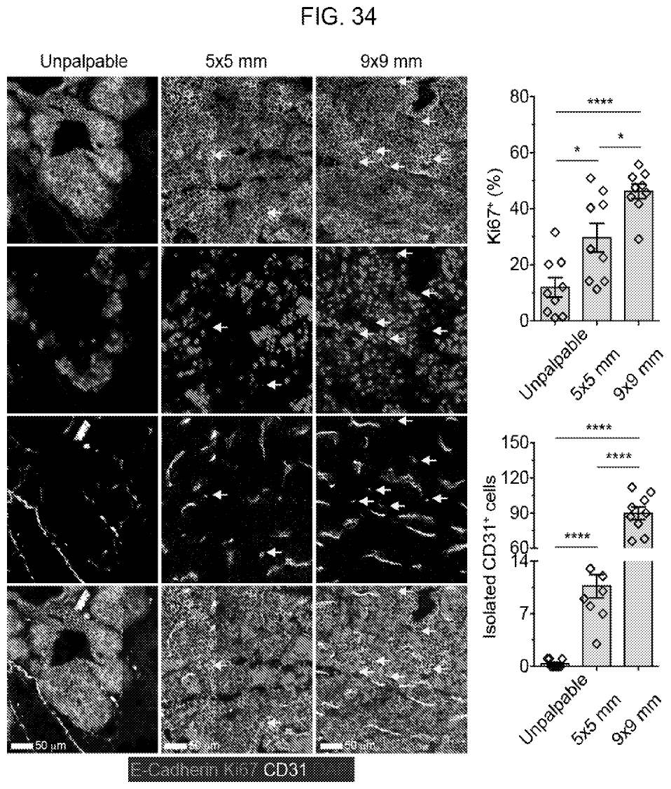

To explore immunological means to reprogram the tumor vasculature, studies were conducted to examine the MMTV-PyMT (PyMT) transgenic model of murine breast cancer. Immunohistological analyses revealed that sprouting angiogenesis in the tumor parenchyma increased substantially early on when mammary tumors reached a size of 5×5 mm in length and width (FIG. 34), which was associated with increased frequencies of tumor cells expressing the proliferation marker Ki67 (FIG. 34). Torturous and bluntly ended vasculature became more abundant in later stage 9×9 mm tumors, accompanied by enhanced tumor cell proliferation (FIG. 34).

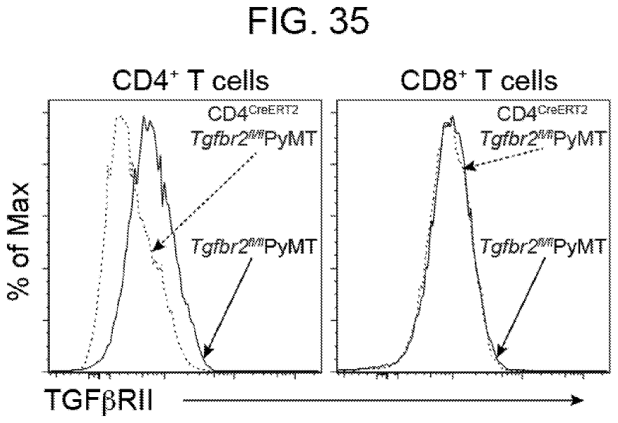

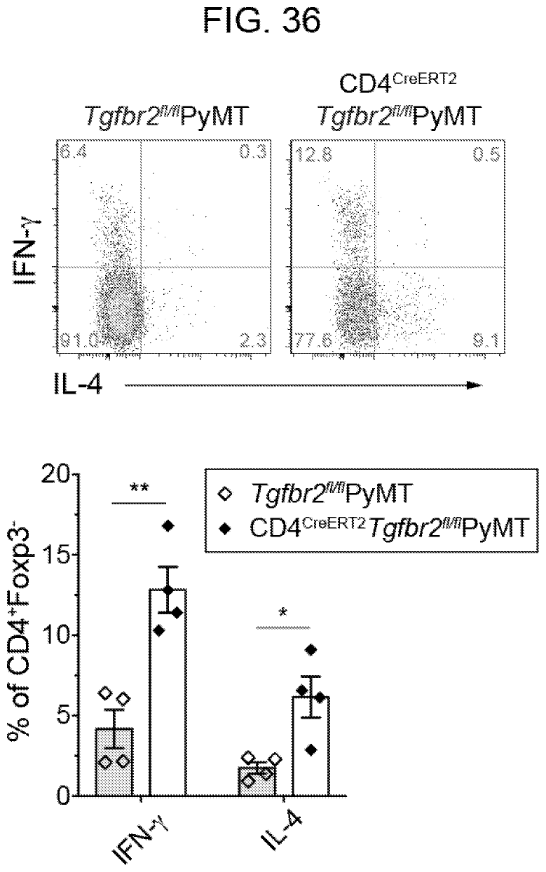

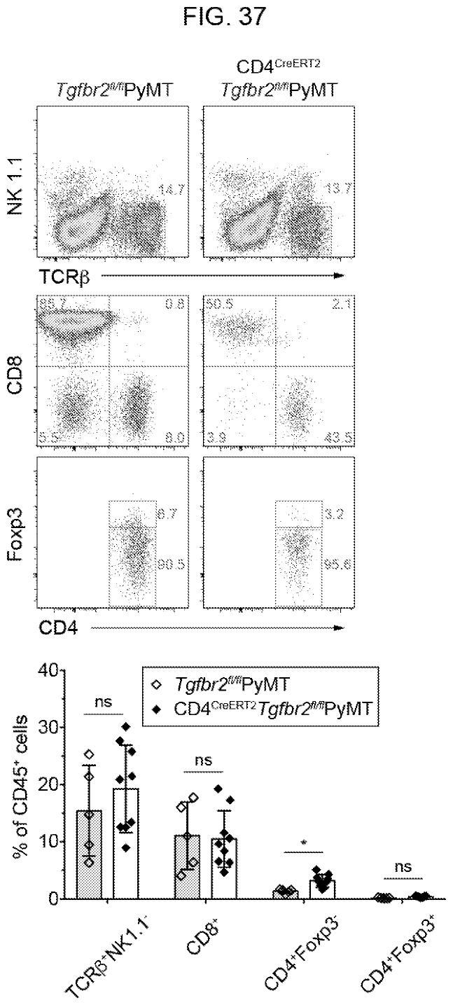

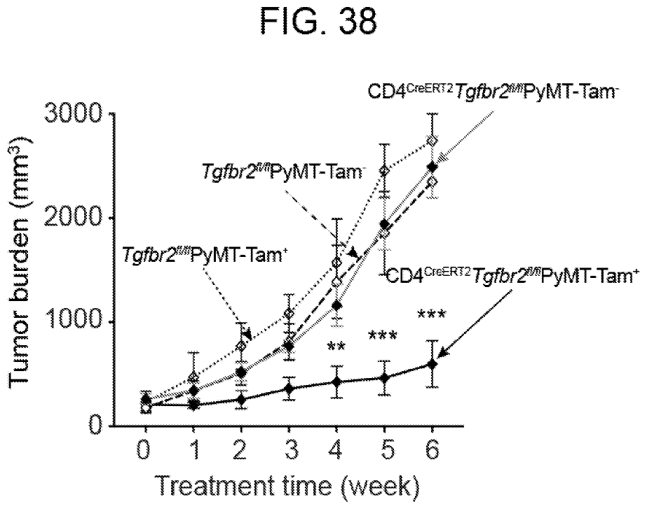

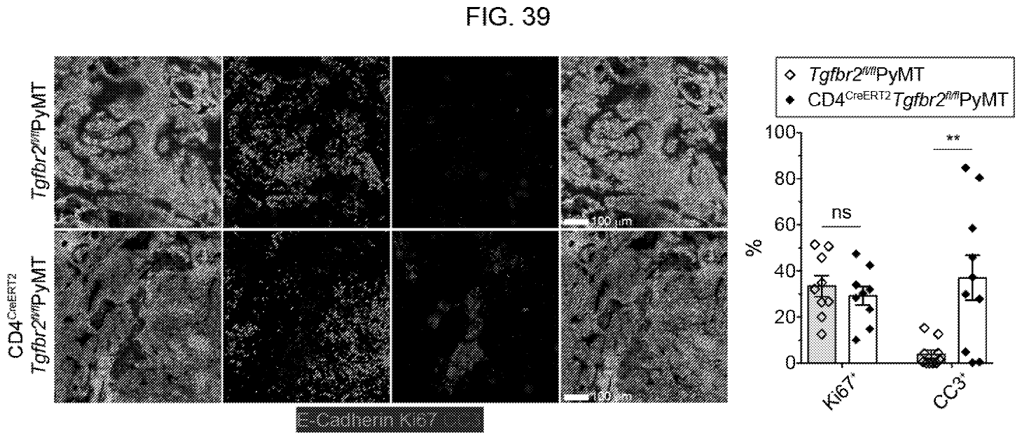

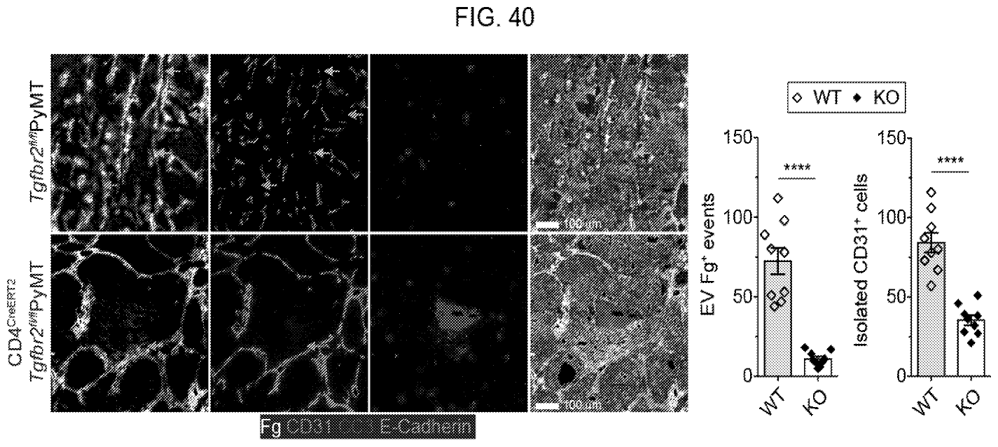

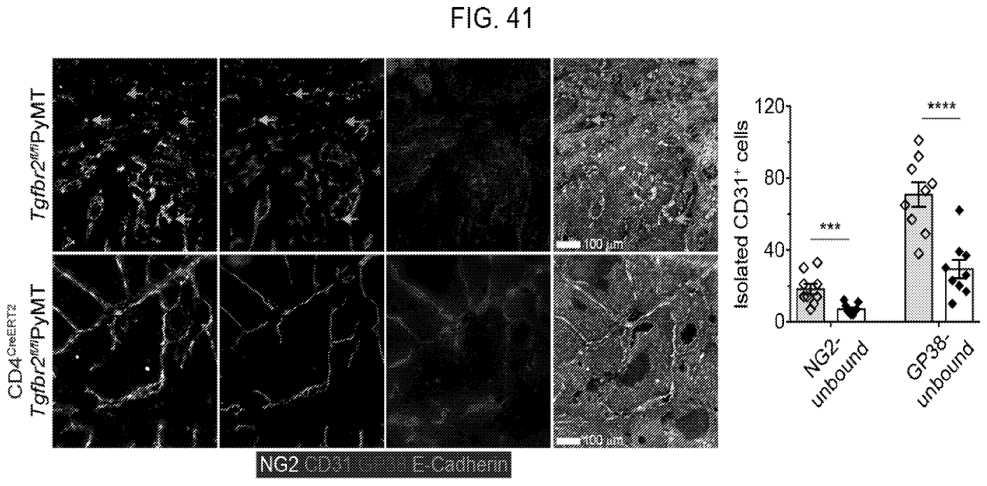

The studies described in Examples 1-6 revealed that constitutive inhibition of TGF-β signaling in CD4⁺ helper T cells reinforces tumor vasculature organization and suppresses cancer progression. To investigate the therapeutic potential of TGF-β blockade in CD4⁺ T cells, PyMT mice carrying a foxed allele of the Tgfbr2 gene (Tgfbr2^{fl/fl}) were crossed with CD4^{CreERT2} transgenic mice in which the Cre recombinase can be activated in CD4⁺ T cells by tamoxifen. (See Sledzinska, A. et al., PLoS Biology 11, e1001674, doi:10.1371/journal.pbio.1001674 (2013).) Cohorts of CD4^{CreERT2}Tgfbr2-01PyMT and control Tgfbr2^{fl/fl}PyMT mice bearing 5×5 mm tumors were left untreated or treated with tamoxifen, and monitored for tumor growth. Tamoxifen treatment of CD4^{CreERT2}Tgfbr2^{fl/fl}PyMT, but not Tgfbr2^{fl/fl}PyMT mice, ablated TGF-βRII expression specifically in CD4⁺ T cells (FIG. 35) which resulted in enhanced differentiation of IFN-γ-producing Th1 and IL-4-producing Th2 cells, as well as increased tumor infiltration of conventional CD4⁺Foxp3⁻ T cells, but not CD4⁺Foxp3⁺ regulatory T (Treg) cells or CD8⁺ T cells (FIGS. 36 and 37). With the augmented T helper cell response, tumor burden was reduced by 5-fold at 6 weeks post-treatment (FIG. 38), associated with increased tumor cell death revealed by cleaved caspase 3 (CC3) staining, while tumor cell proliferation was unaffected (FIG. 39).

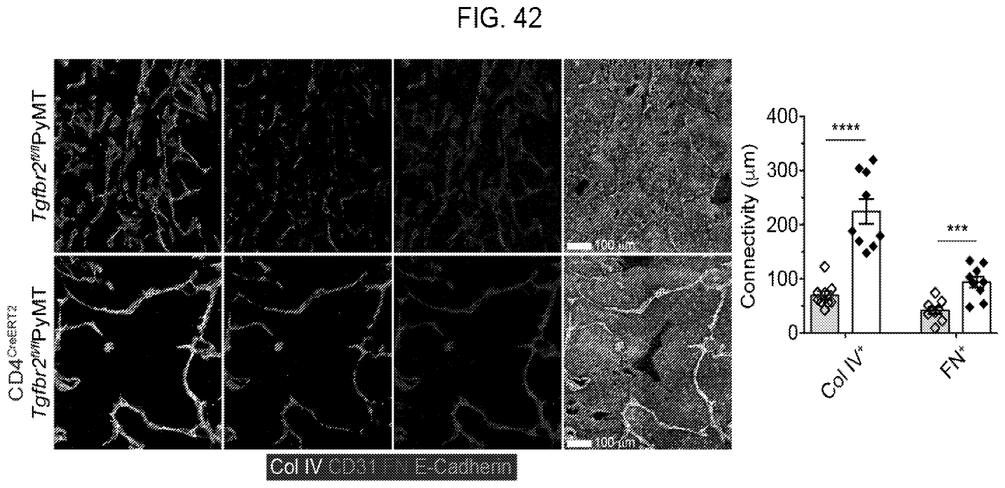

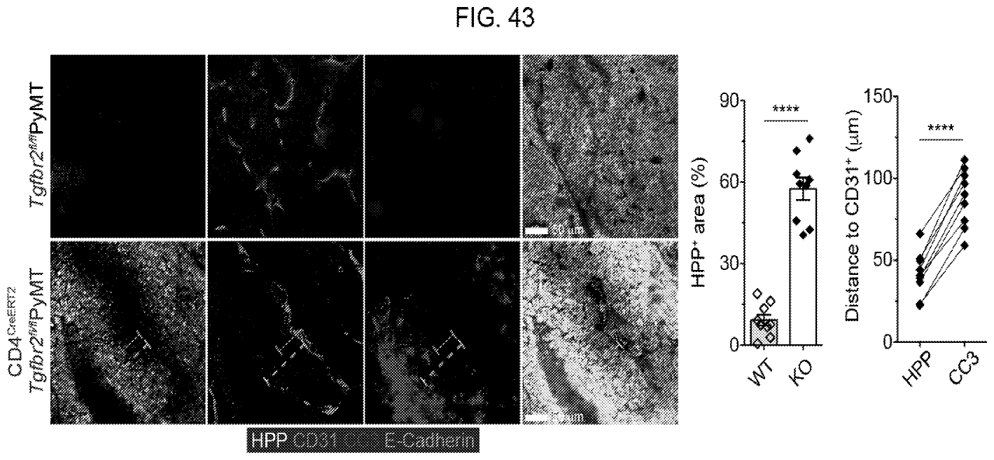

Compared to tumors from tamoxifen-treated Tgfbr2^{fl/fl}PyMT mice showing extravascular fibrinogen, a sign of vessel leakage and tissue wounding, and irregularly shaped vasculature, these structures were reduced in tumors from tamoxifen-treated CD4^{CreERT2}Tgfbr2^{fl/fl}PyMT mice (FIG. 40). In addition, the vessels in CD4^{CreERT2}Tgfbr2^{fl/fl}PyMT tumors were surrounded by abundant NG2⁺ pericytes and GP38⁺ fibroblasts (FIG. 41), and ensheathed by highly connected basement membrane proteins collagen IV and fibronectin (FIG. 42). With this mature vasculature phenotype, there was a 6-fold increase of hypoxic areas adjacent to the dying tumor region (FIG. 43). Thus, genetic blockade of TGF-β signaling in CD4⁺ T cells is sufficient to restore an organized tumor vasculature leading to tumor hypoxia, tumor cell death and suppression of cancer progression.

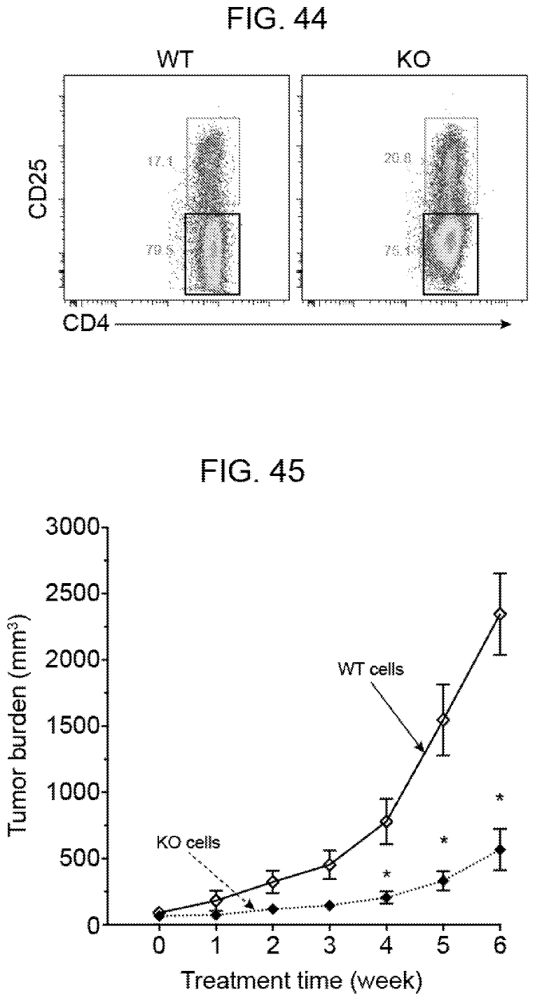

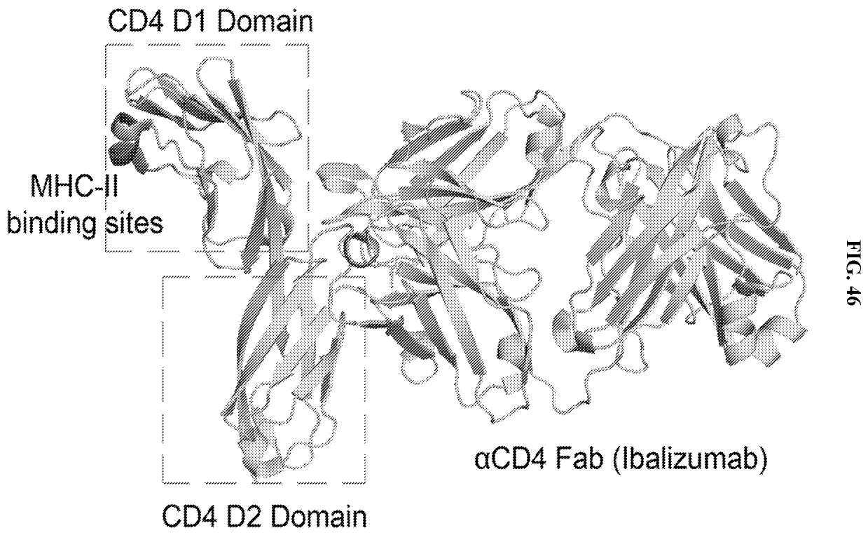

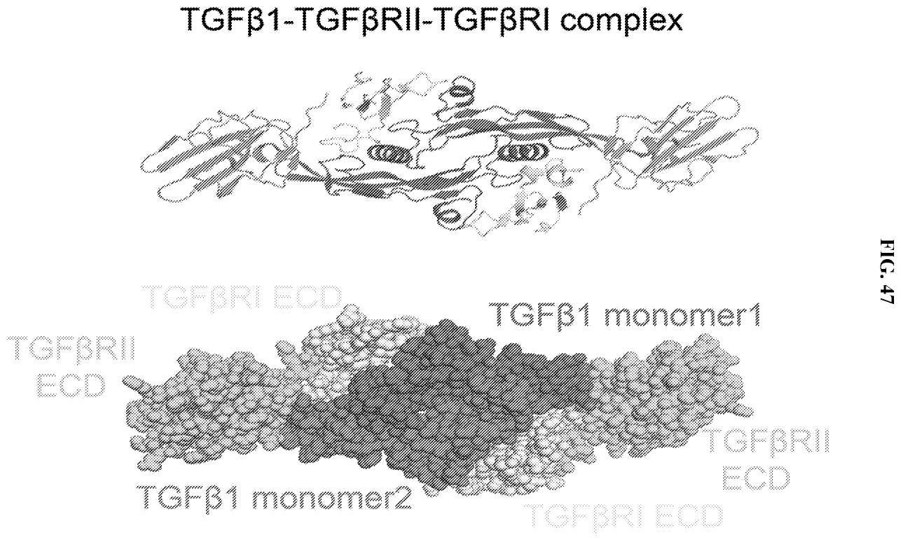

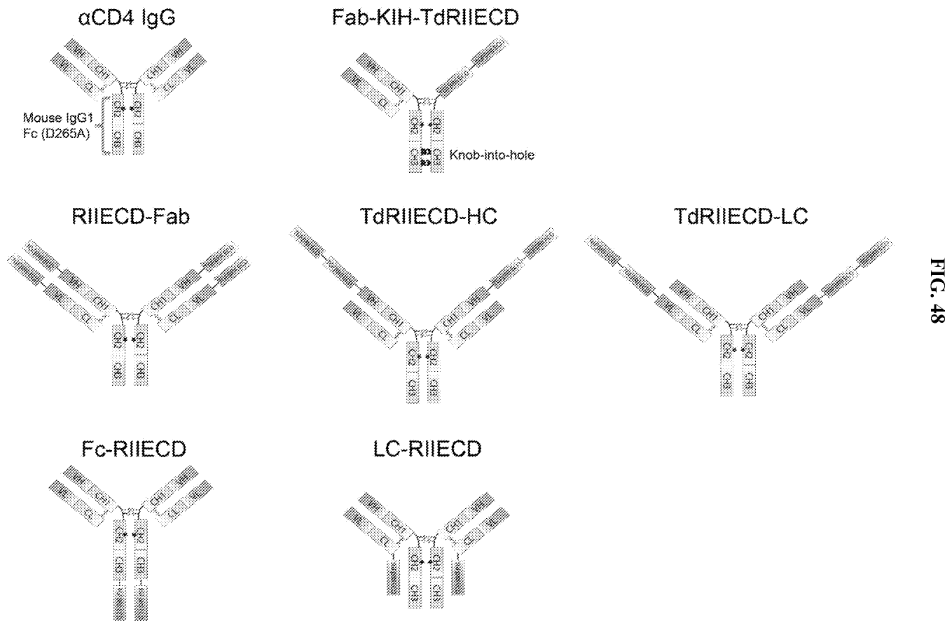

To investigate whether inhibition of TGF-β signaling in helper T cells could be harnessed for the adoptive cell transfer-based cancer immunotherapy, CD4⁺CD25⁻ T cells were purified from Tgfbr2^{fl/fl} (wild-type, WT) and ThPOK^{Cre}Tgfbr2^{fl/fl} (knockout, KO) mice (FIG. 44), and activated in vitro. Activated T cells were transferred into PyMT mice bearing 5×5 mm tumors. Compared to PyMT recipients of WT T cells, recipients of KO T cells exhibited slow tumor growth (FIG. 45), revealing that transfer of helper T cells with blocked TGF-β signaling represents an effective cancer therapy approach.

To investigate whether blockade of TGF-β signaling in helper T cells with biologics could be a viable therapeutic approach, protein-engineering techniques were employed to generate bispecific antibodies, one specific for CD4 and one for TGF-β. Ibalizumab, an anti-human CD4 (αCD4) that recognizes an epitope in the C2 domain of CD4 distinct from its major histocompatibility complex class II binding site, was used to achieve CD4⁺ T cell targeting (FIG. 46). (See Burkly, L. C. et al., Inhibition of HIV infection by a novel CD4 domain 2-specific monoclonal antibody. Dissecting the basis for its inhibitory effect on HIV-induced cell fusion, J. Immunol. 149, 1779-1787 (1992); Song, R. et al., Epitope mapping of ibalizumab, a humanized anti-CD4 monoclonal antibody with anti-HIV-1 activity in infected patients, J. Virol. 84, 6935-6942, doi:10.1128/JVI.00453-10 (2010).) To block TGF-β signaling, the TGF-βRII ECD was utilized, as it would not be immunogenic and its binding to TGF-β could exert a dominant negative function by recruiting endogenous TGF-β receptor I (TGF-βRI) (FIG. 47).

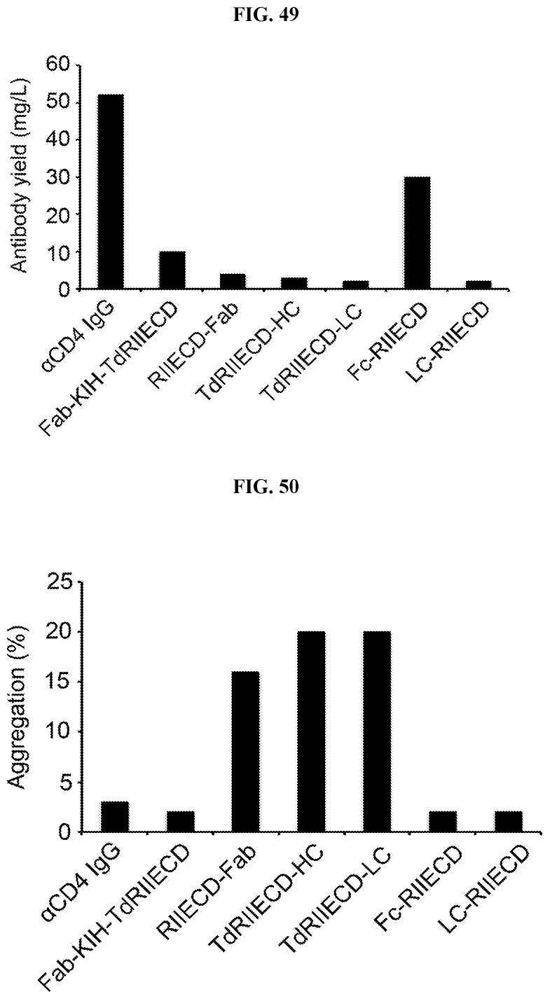

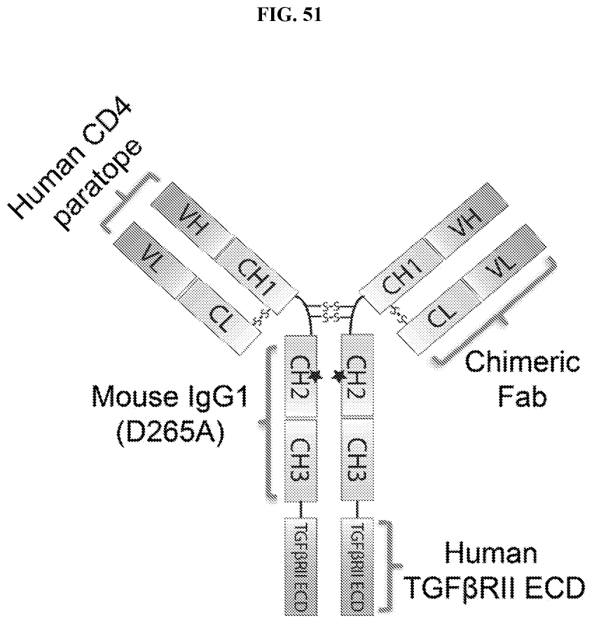

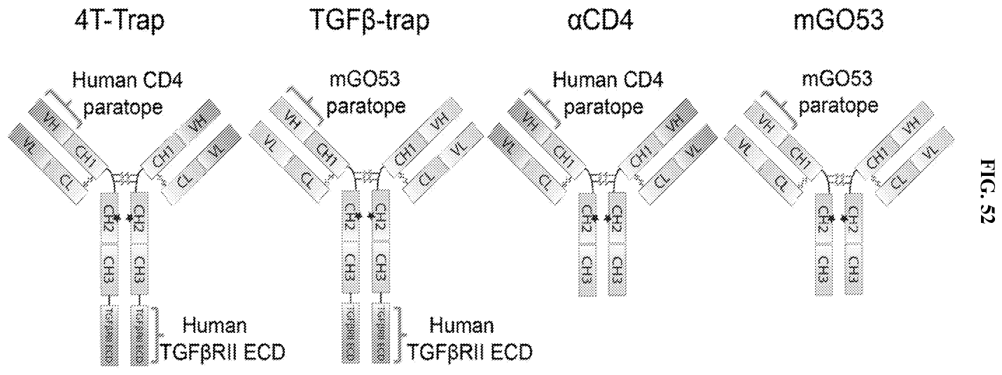

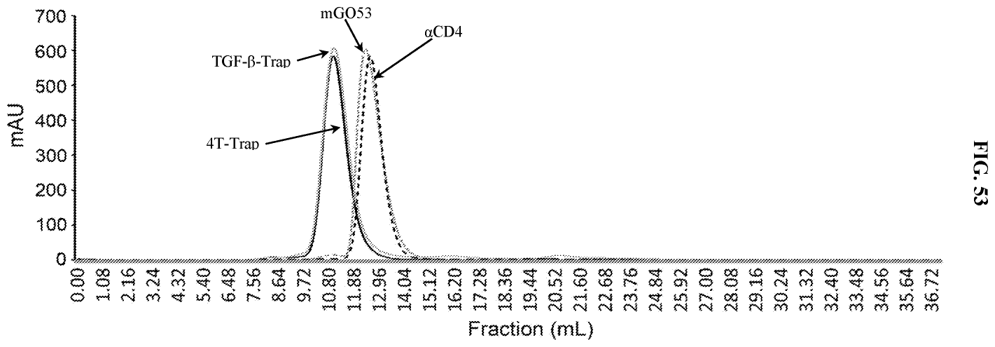

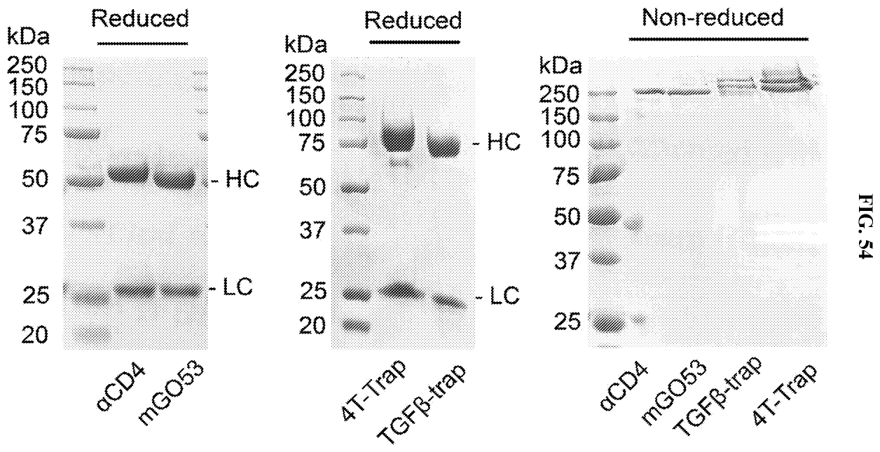

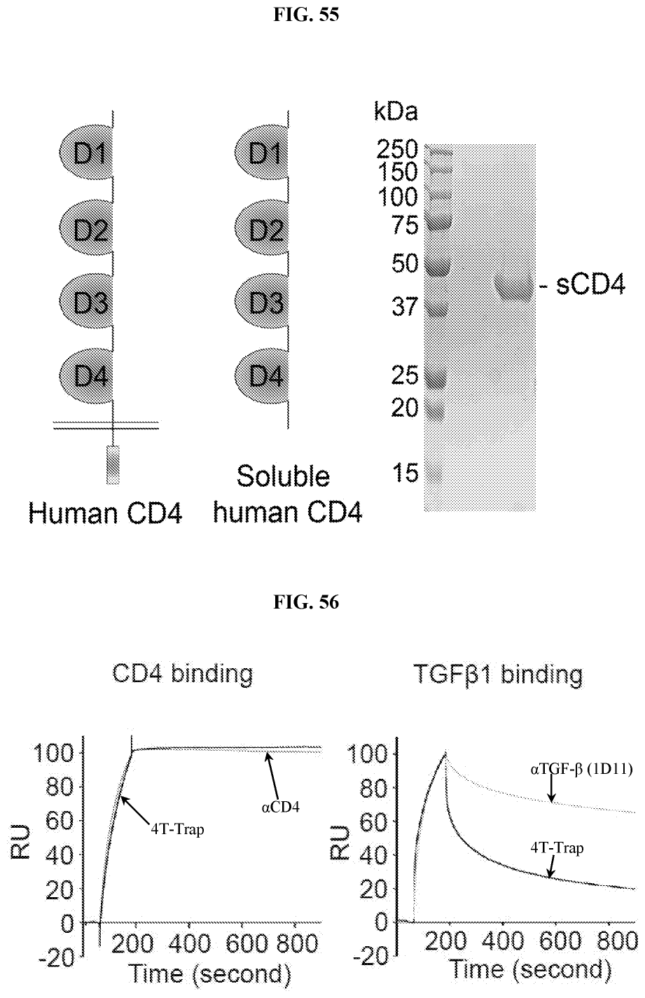

Bispecific formats were engineered with fusion of human TGF-βRII ECD to the antigen-binding (Fab) region of ibalizumab fused to a murine IgG1-Fc (fragment crystallized), where position 265 was mutated to alanine to prevent Fc receptor binding (FIG. 48). One of the formats with fusion of TGF-βRII ECD to the C-terminus of the antibody heavy chain, Fc-RIIECD, had high yield and exhibited low aggregation (FIGS. 49 and 50). This format was chosen for further development as “CD4 TGF-β Trap” (4T-Trap) (FIG. 51). αCD4 and 4T-Trap, as well as a non-CD4-binding control antibody mGO53, and the mGO53 TGF-βRII ECD bispecifics (named as TGF-β-Trap), were expressed and purified to homogeneity (FIGS. 52-54). Binding of 4T-Trap and αCD4 to immobilized CD4 was similar, with dissociation constants (Kd) around 0.1 nM (FIGS. 55-57), which was corroborated by their comparable binding to plasma membrane human CD4 ectopically expressed in HEK293 (293-hCD4) cells (FIG. 58).

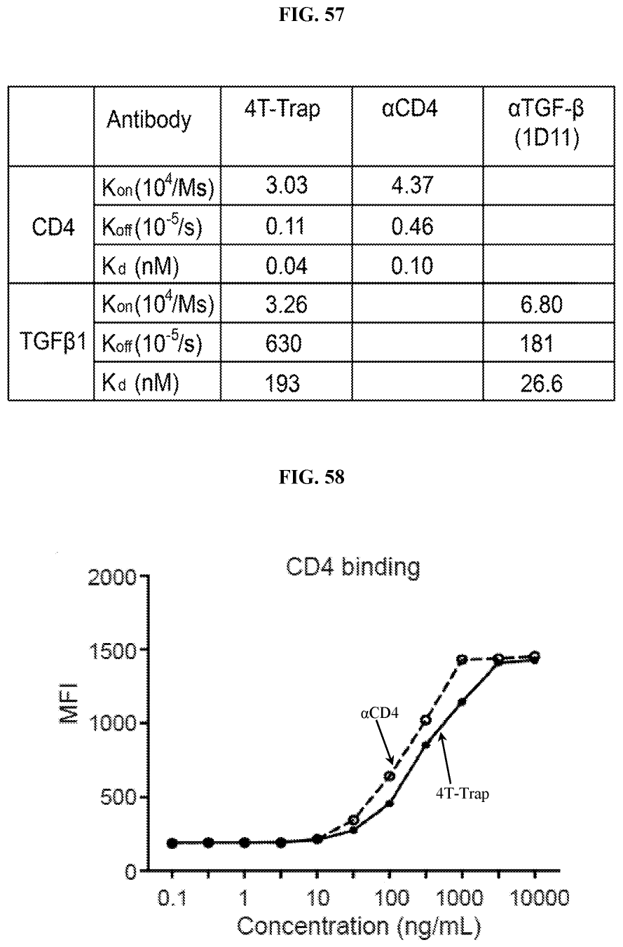

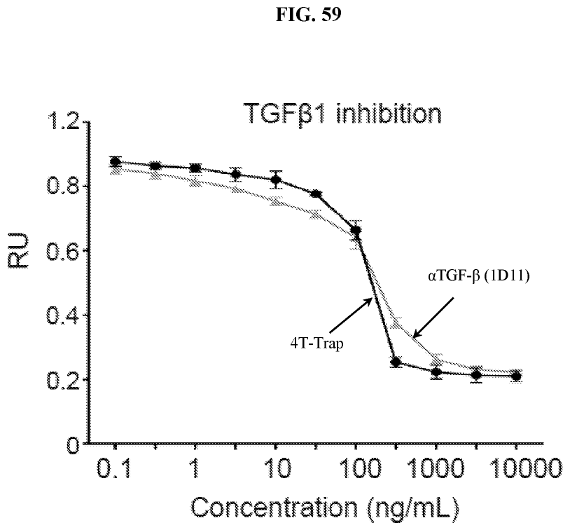

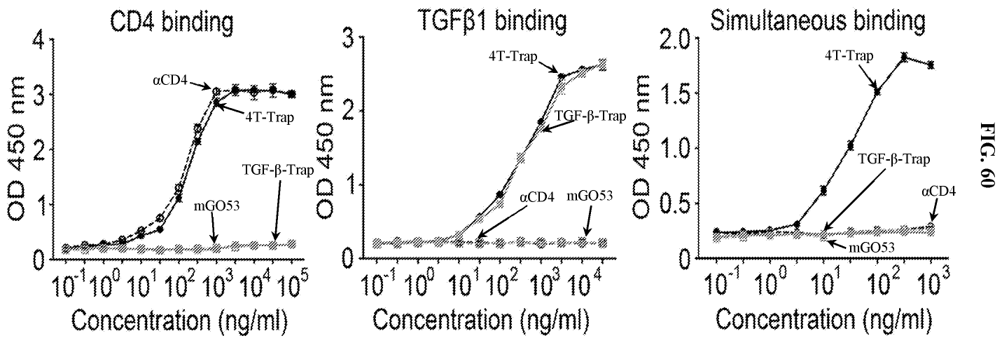

When compared to an anti-TGF-β (αTGF-β, 1D11 clone), 4T-Trap had a comparable association rate (kₒₙ), but a faster dissociation rate (k_{off}) of binding to immobilized TGF-β1 (FIGS. 56 and 57). However, in a TGF-β signaling reporter assay, 4T-Trap was a more effective inhibitor than αTGF-β, showing 80% maximal inhibition (IC₈₀) at 1.3 nM and 25 nM, respectively (FIG. 59), possibly due to its dominant negative effect on TGF-βRI.

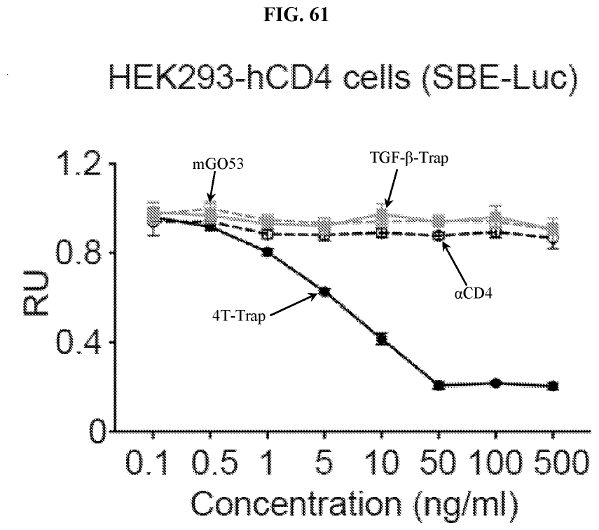

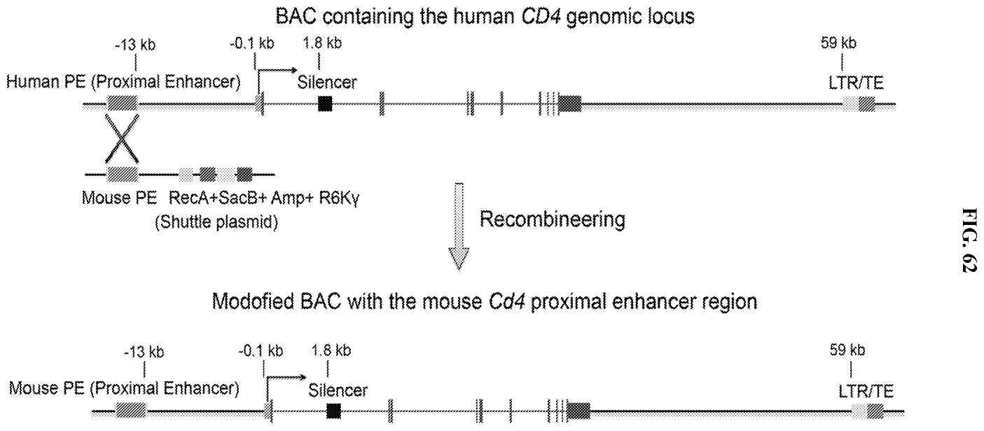

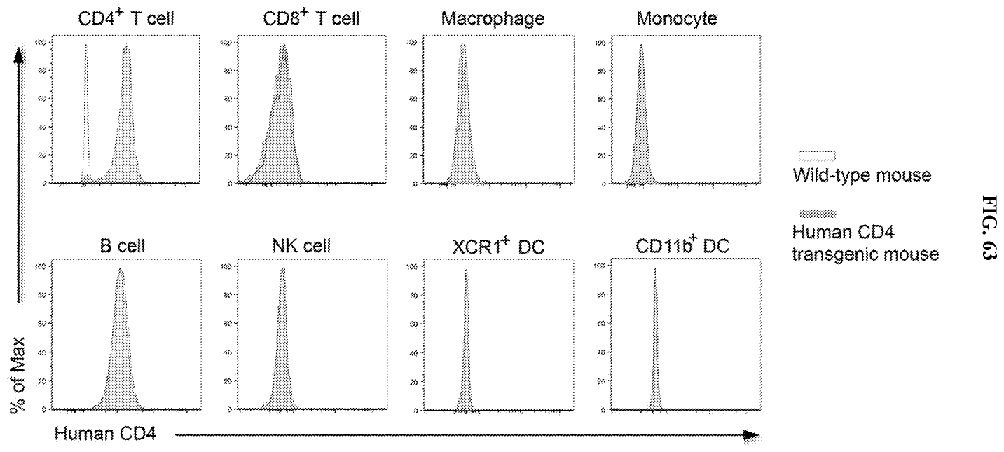

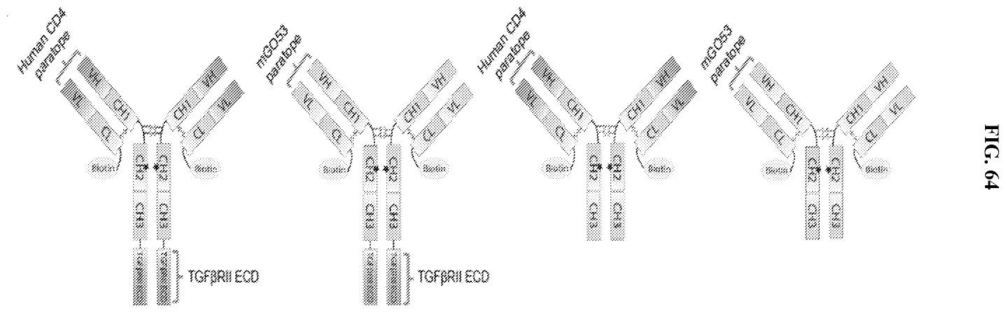

Enzyme-linked immunosorbent assays showed that CD4 binding for 4T-Trap versus αCD4 and TGF-β1 binding for 4T-Trap versus TGF-β-Trap were comparable (FIG. 60). Importantly, using a pretreatment scheme of incubation followed by washing, 4T-Trap, but not TGF-β-Trap, inhibited TGF-β signaling in 293-hCD4 cells (FIG. 61). These findings demonstrate that 4T-Trap preserves efficient CD4 binding and potent TGF-β signaling inhibition properties.

The human CD4 epitope recognized by ibalizumab is not conserved in mice. (See Burkly et al., supra.) To test the therapeutic efficacy of 4T-Trap in vivo, a strain of human CD4 transgenic (hCD4) mice was generated using a bacterial artificial chromosome harboring the human CD4 locus with the proximal enhancer region replaced by the murine equivalent to augment its expression (FIG. 62). Flow cytometry experiments revealed exclusive expression of human CD4 on mouse CD4⁺ T cells at a level comparable to that on human CD4⁺ T cells (FIG. 63 and data not shown).

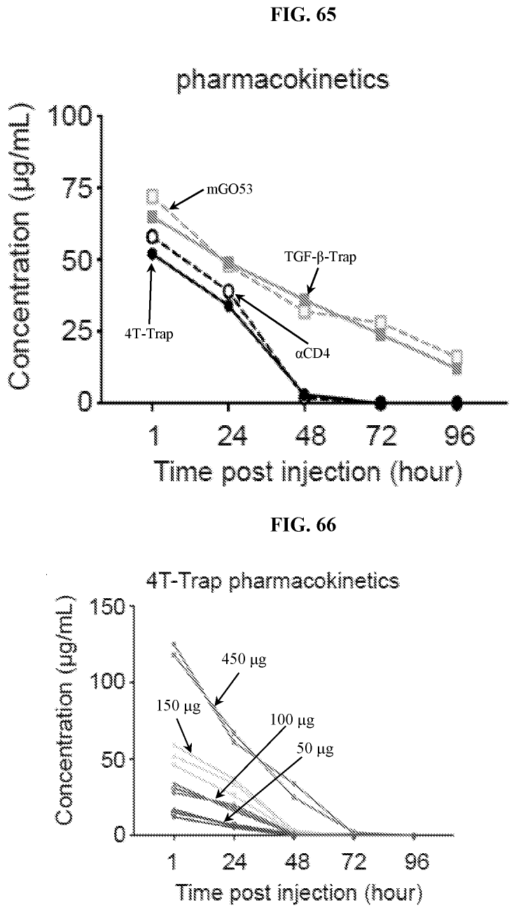

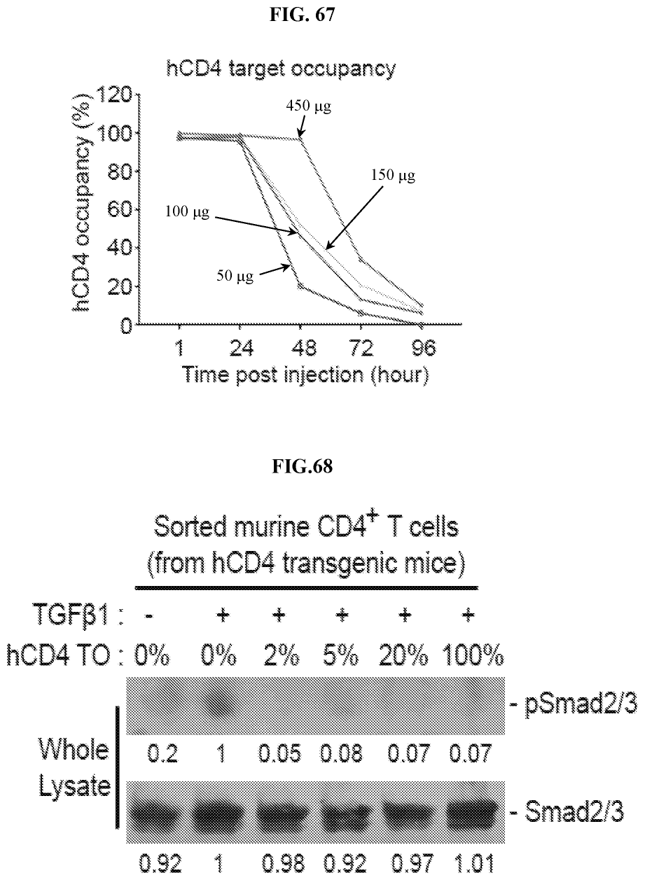

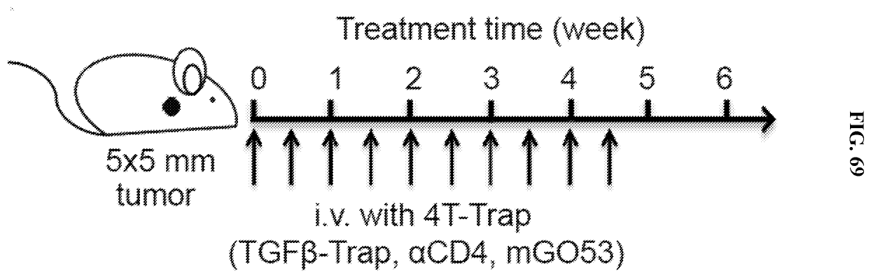

The in vivo pharmacokinetics (PK) of biotinylated 4T-Trap and control antibodies were then assessed in hCD4 mice (FIG. 64). Following administration at a single dose of 150 μg, mGO53, and TGF-β-Trap showed a linear PK and long half-life (t_{1/2}=48 hr) in a 96 hr-testing window (FIG. 65). In contrast, αCD4 and 4T-Trap exhibited a nonlinear PK and short half-life (t_{1/2}=20 hr), irrespective of antibody doses (FIGS. 65 and 66), possibly due to antibody internalization following CD4 binding. Moreover, 4T-Trap target occupancy (TO) in hCD4⁺ T cells approached 100% at 1 hr and 24 hr for all doses tested, which declined substantially at later time points (FIG. 67). In particular, the 100 μg dose had an approximate 5% TO at 72 hr post-administration (FIG. 67), which was sufficient to inhibit TGF-β signaling in CD4⁺ T cells (FIG. 68). These findings revealed that 4T-Trap was efficiently delivered to CD4⁺ T cells in vivo and potently suppressed TGF-β signaling with desirable pharmacodynamics (PD).

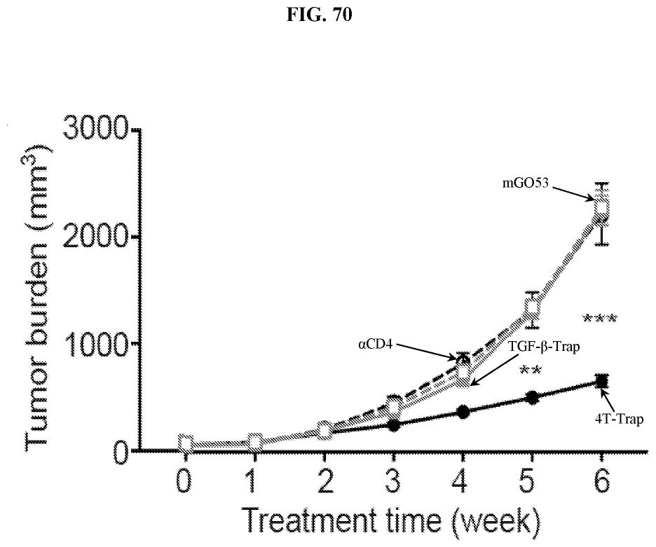

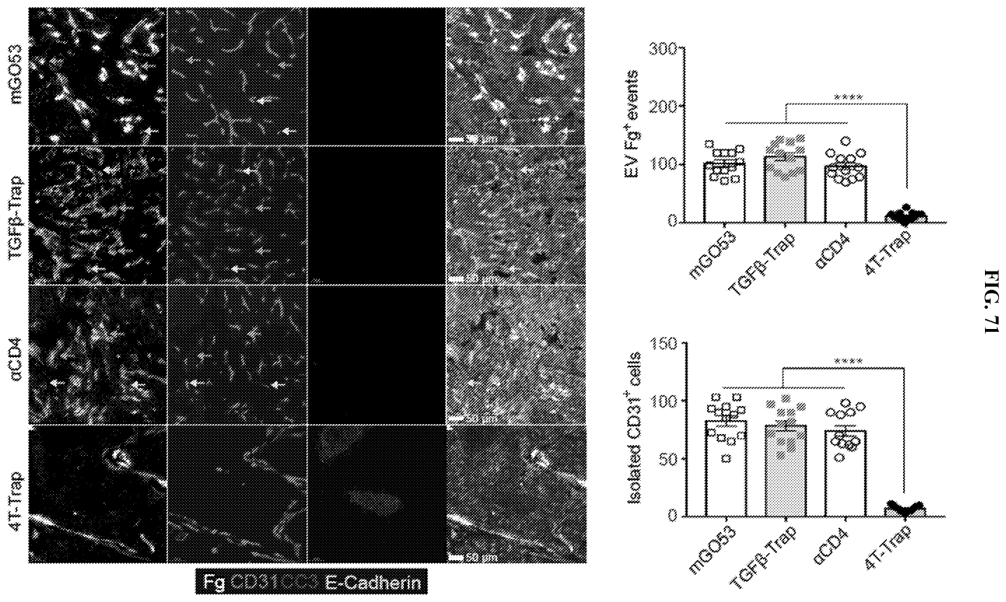

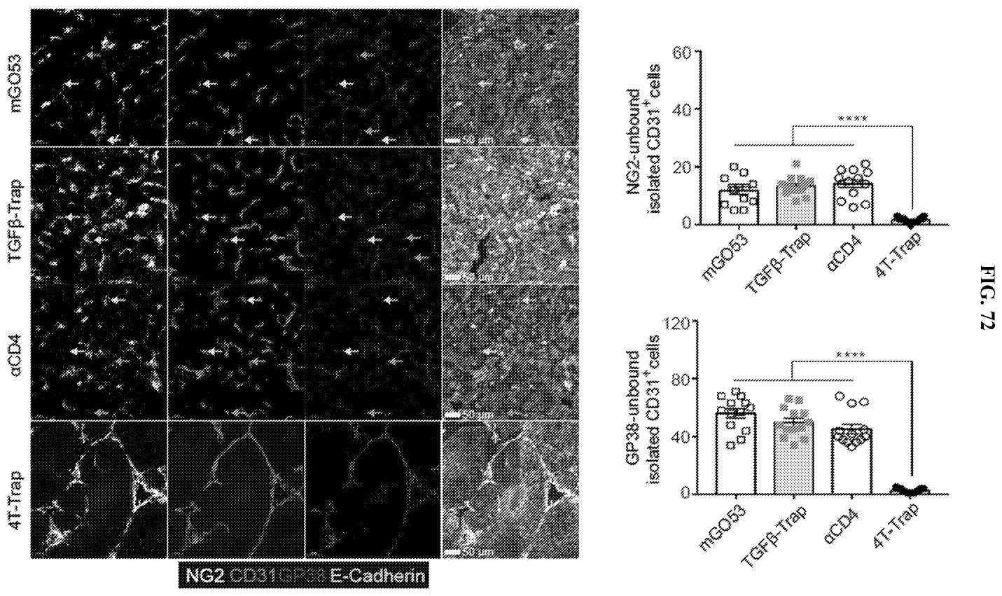

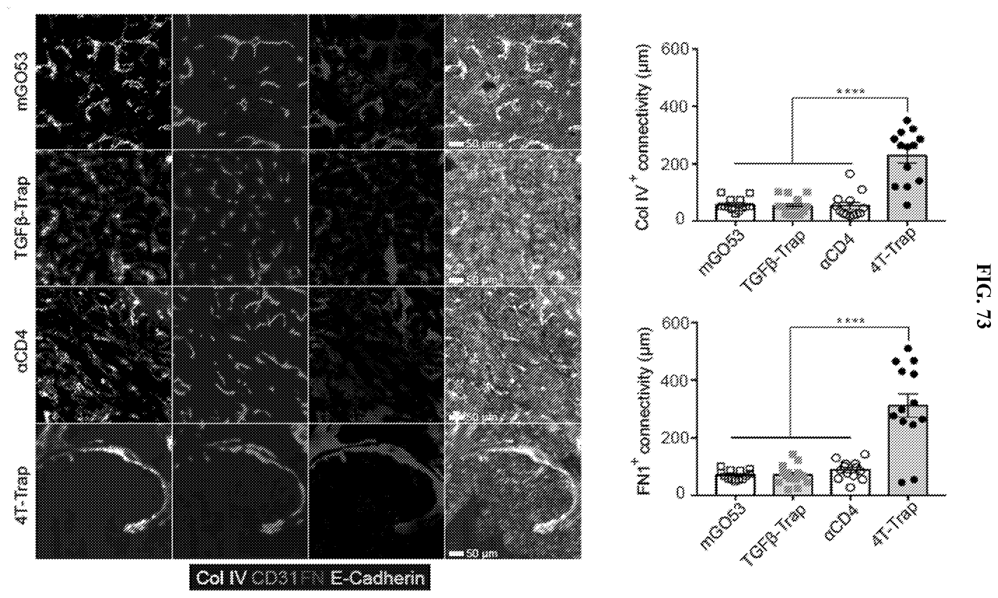

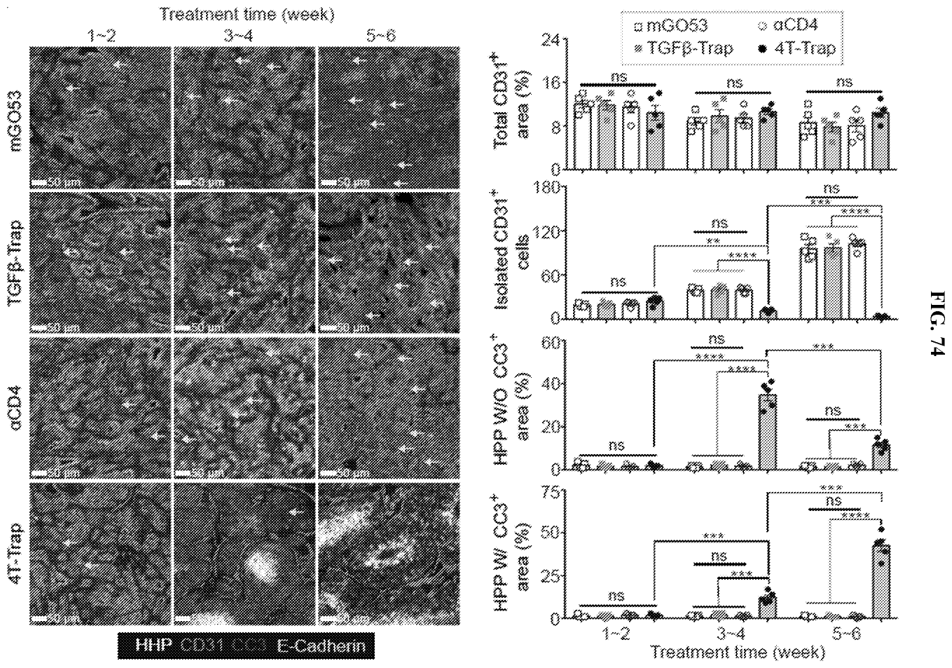

Based on the PK and PD properties of 4T-Trap, a treatment protocol of 100 μg/dose at twice a week was selected to explore its cancer therapeutic potential in hCD4 mice bred onto the PyMT background. hCD4PyMT mice bearing 5×5 mm tumors were treated with intravenous 4T-Trap or control antibodies including TGF-β-Trap, αCD4, and mGO53, for a total of 10 doses, and monitored for tumor growth for 6 weeks (FIG. 69). Compared to control antibodies, 4T-Trap caused profound inhibition of mammary tumor growth (FIG. 70). By immunohistological analyses, tumor tissue healing was only detected in the 4T-Trap group, manifested by diminished extravascular deposition of fibrinogen (FIG. 71). Reduced abundance of the irregularly shaped vasculature was also observed, concomitant with increased tumor cell death (FIG. 71). Furthermore, blood vessels in the 4T-Trap group were tightly enwrapped by NG2⁺ pericytes and GP38⁺ fibroblasts, as well as the highly connected basement membrane proteins collagen IV and fibronectin (FIGS. 72 and 73). These findings demonstrated that 4T-Trap promoted vasculature organization, tumor tissue healing and cancer repression.

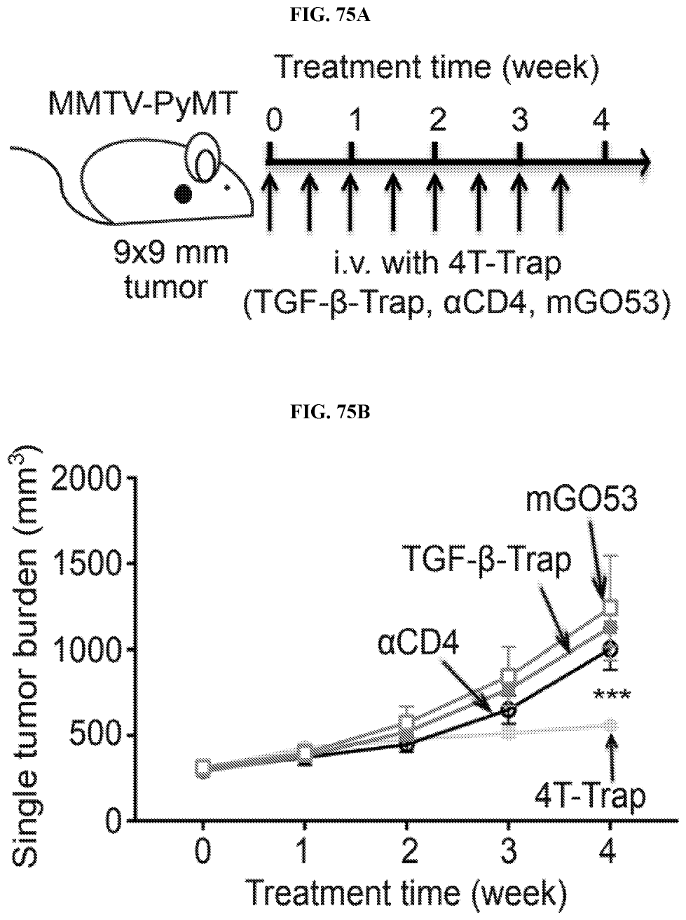

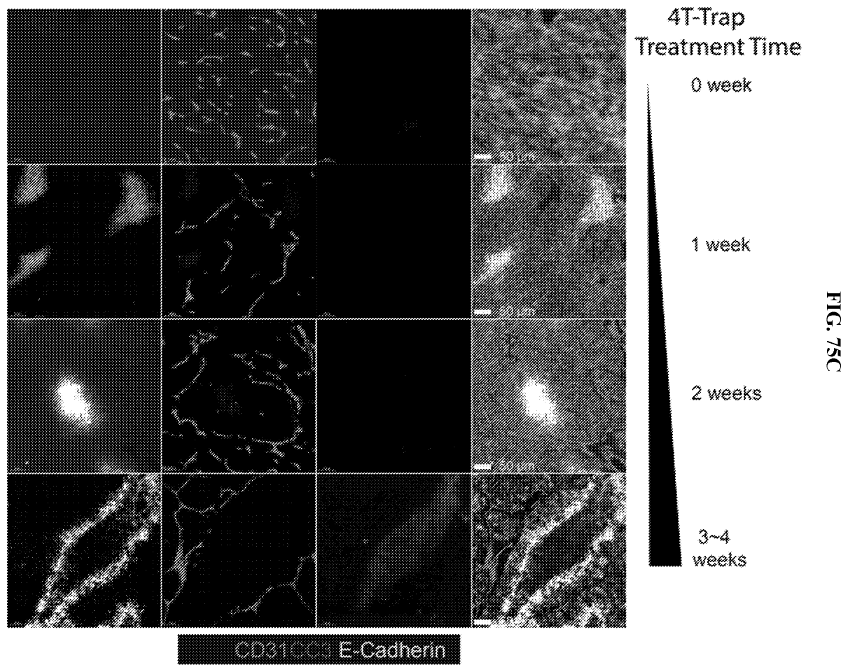

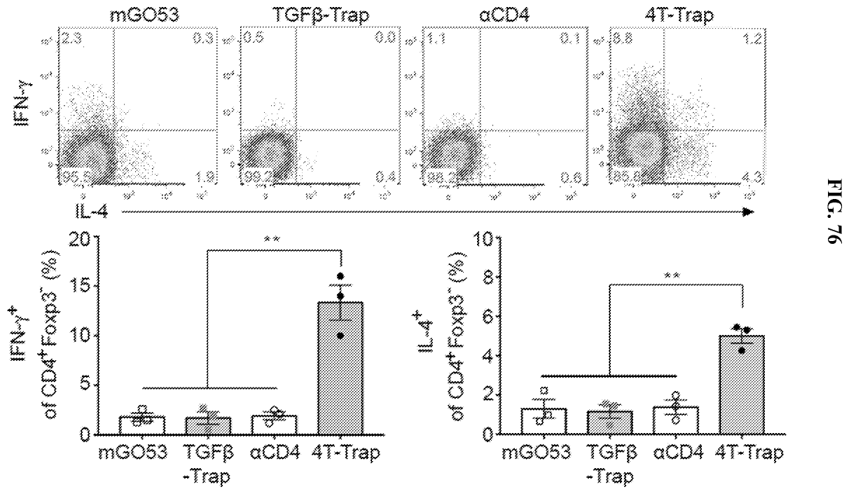

To better characterize the therapeutic effects of 4T-Trap, tumor vasculature dynamics and tumor cell fates were monitored in hCD4PyMT mice treated with 4T-Trap or control antibodies. At 1-2 weeks post-treatment, comparable sprouting angiogenesis in the tumor parenchyma was observed in all groups of mice (FIG. 74). At 3-4 weeks, while vessel density was unchanged, the number of isolated endothelial cells was increased in mice treated with mGO53, TGF-β-Trap, or αCD4 control antibodies (FIG. 74). In contrast, the bluntly ended blood vasculature was substantially repressed in mice treated with 4T-Trap (FIG. 74), associated with an approximate 30-fold increase of hypoxic areas with minimal tumor cell death (FIG. 74). By 5-6 weeks, the irregularly shaped blood vasculature was much exaggerated in all control groups (FIG. 74), but was further suppressed in 4T-Trap-treated mice (FIG. 74), triggering catastrophic tumor cell death in hypoxic areas distant to the vasculature (FIG. 74). These findings suggested that 4T-Trap restrains tumor progression by inducing vasculature pruning and reorganization, which results in hypoxia and starvation-triggered tumor cell death. Similar 4T-Trap-triggered inhibition of tumor growth, vasculature remodeling and cancer cell death were observed in mice bearing advanced 9×9 mm tumors (FIGS. 75A-75C).

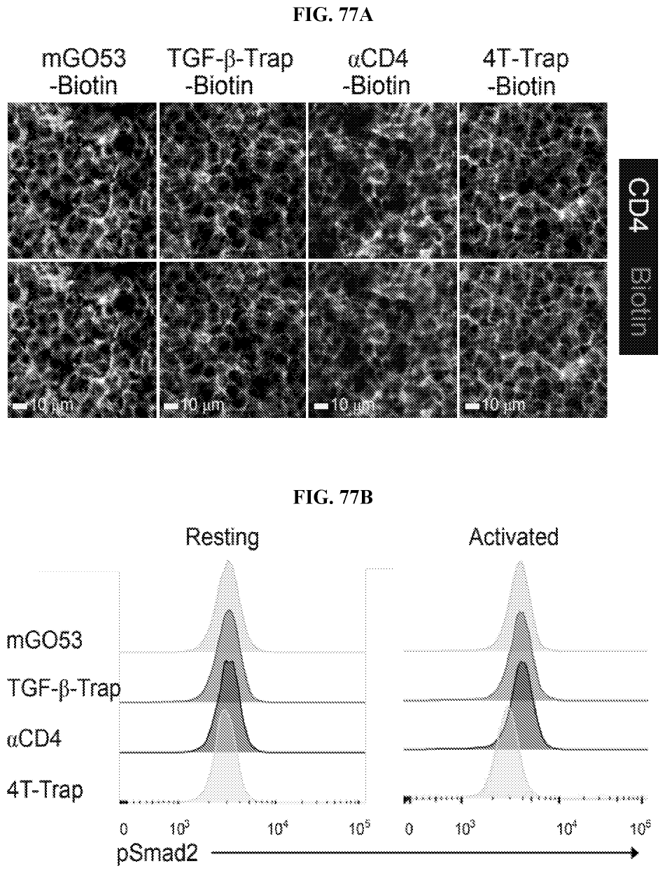

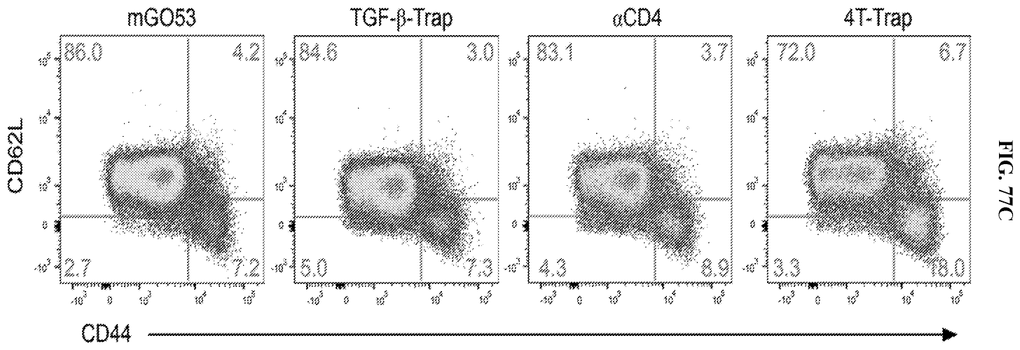

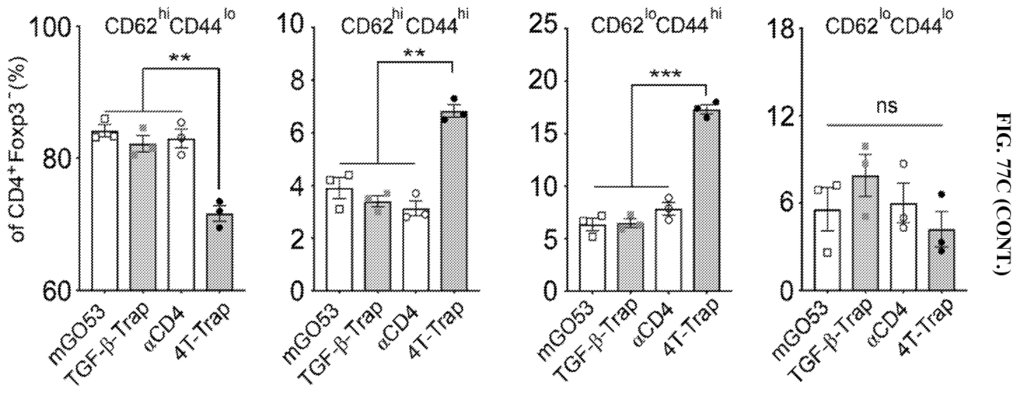

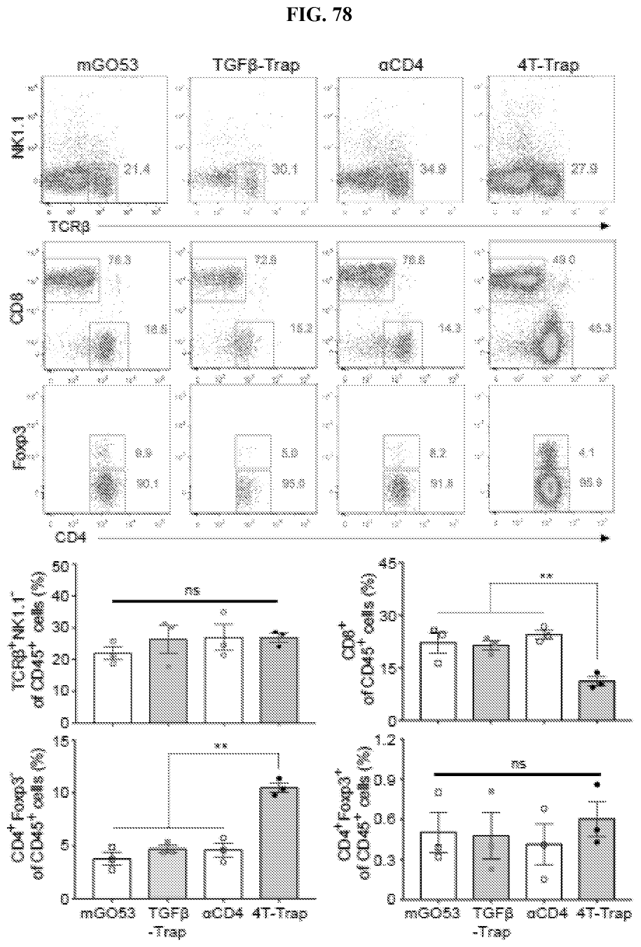

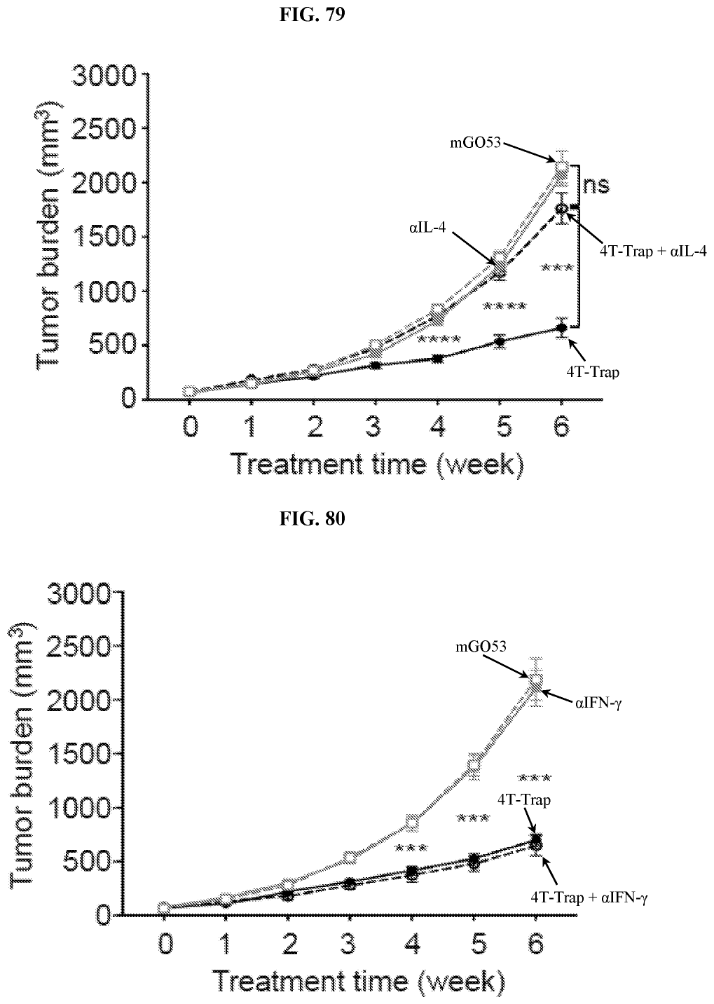

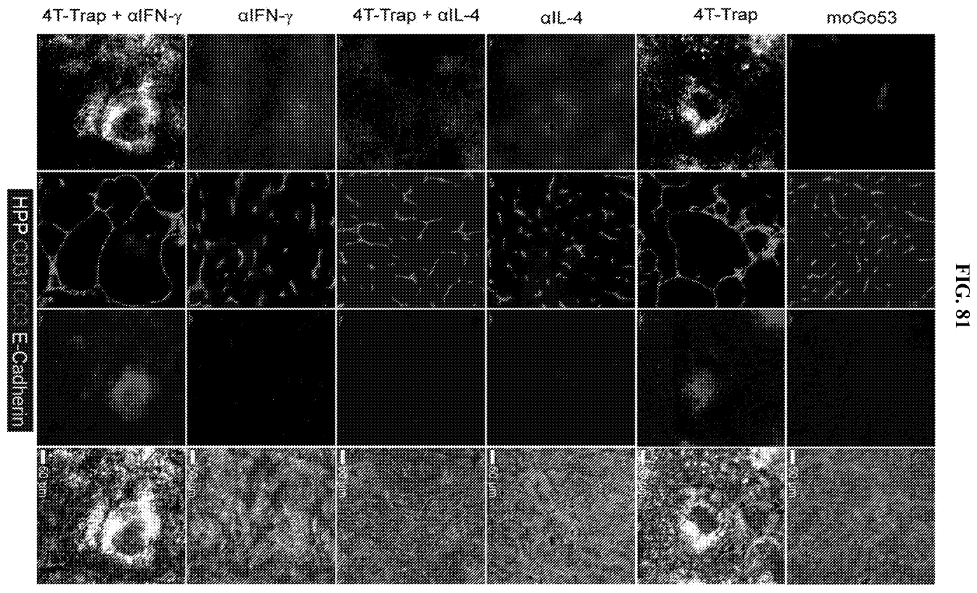

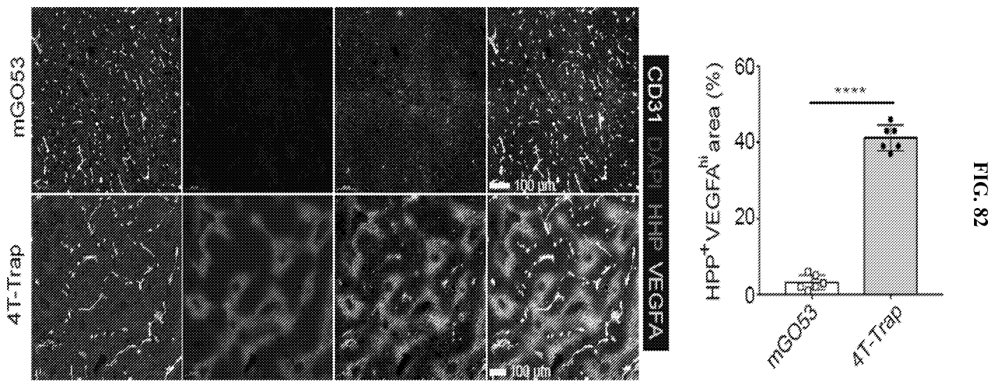

4T-Trap inhibition of tumor progression was associated with enhanced differentiation of IFN-γ-producing Th1 and IL-4-producing Th2 cells, as well as increased tumor infiltration of conventional CD4⁺Foxp3⁻ T cells at the expense of CD8⁺ T cells (FIGS. 76 and 78). 4T-Trap, but not αCD4, TGF-β-Trap or mGO53, blocks TGF-β signaling in tumor-draining lymph node CD4⁺ T cells, and induces enhanced effector/memory CD4⁺ T cell differentiation (FIGS. 77A-77C). In particular, neutralization of IL-4, but not IFN-γ, reversed the tumor suppression phenotype (FIGS. 79 and 80), which was associated with attenuated vessel organization, diminished hypoxia, and reduced tumor cell death (FIG. 81). Thus, as in the genetic model of TGF-βRII ablation (see Examples 1-6), it is type 2 immunity that mediates the 4T-Trap-induced anti-tumor immune response.

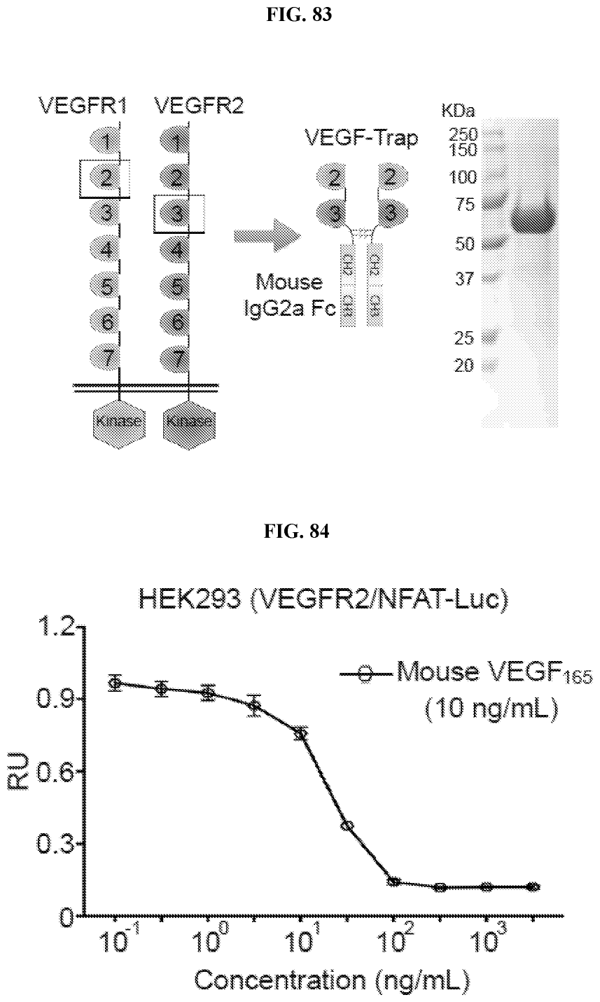

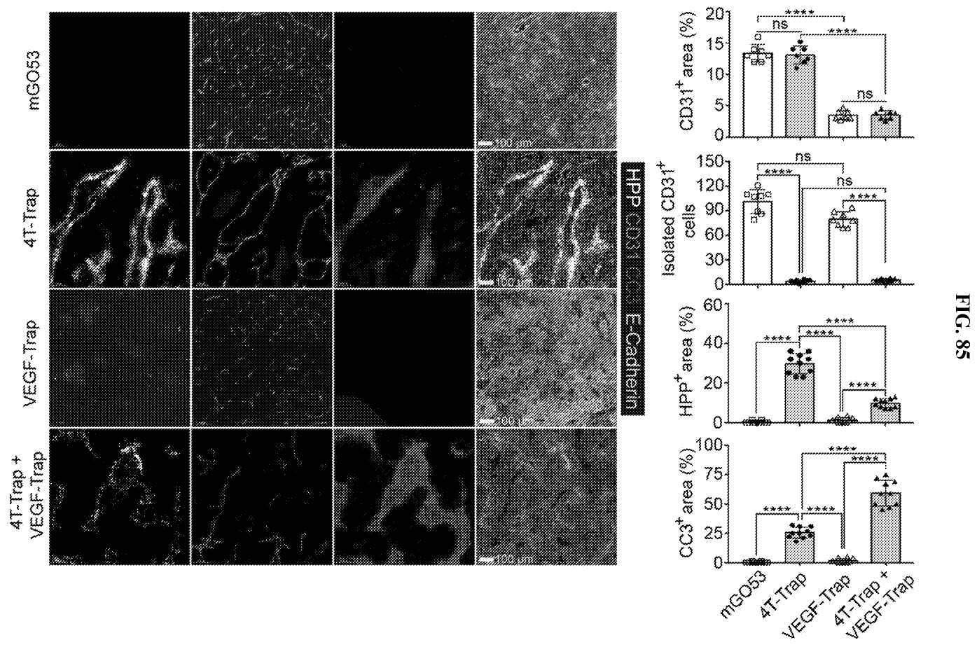

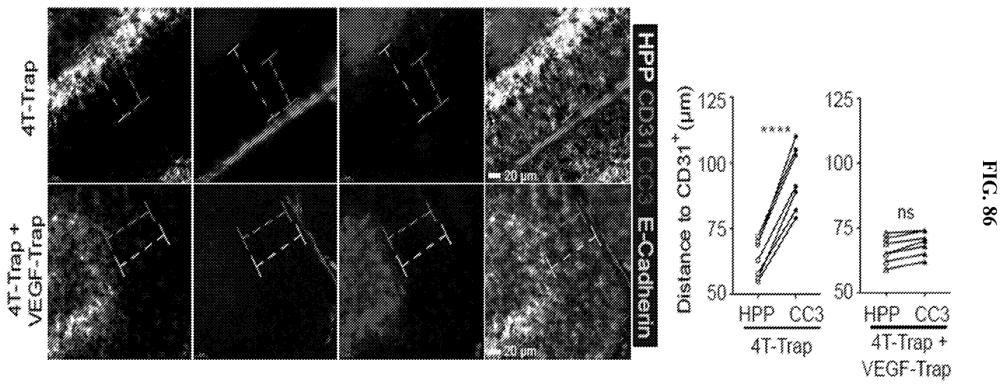

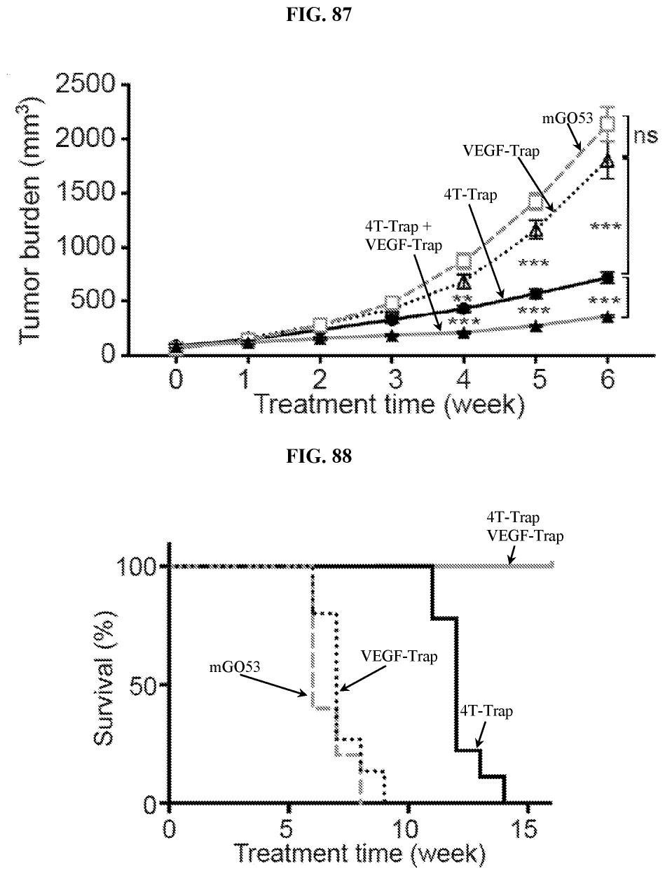

Hypoxia triggers cellular adaptive responses to resolve ischemia in part via the induction of angiogenic factors such as VEGFA. (See Semenza, G. L., Oxygen sensing, hypoxia-inducible factors, and disease pathophysiology, Annual Rev. Pathol. 9, 47-71, doi:10.1146/annurev-pathol-012513-104720 (2014).) Indeed, while low level VEGFA expression with a diffusive pattern was observed in tumors from hCD4PyMT mice treated with control antibodies (FIG. 82), enhanced VEGFA expression in hypoxic areas was detected in mice treated with 4T-Trap (FIG. 82). To investigate functional significance of such an adaptive response, a VEGF receptor decoy called VEGF-Trap was engineered, wherein a VEGF receptor (see Holash, J. et al., VEGF-Trap: a VEGF blocker with potent antitumor effects, Proc. Nat'l Acad. Sci. U.S.A. 99, 11393-11398, doi:10.1073/pnas.172398299 (2002)) was fused to a murine IgG2a-Fc (FIGS. 83 and 84). Compared to mGO53 control, VEGF-Trap treatment diminished tumor vessel density, but did not affect vessel patterning, and negligible effects on tumor tissue oxygenation or tumor cell survival were observed (FIG. 85). Notably, co-administration of VEGF-Trap with 4T-Trap resulted in low vessel density in addition to its reorganization (FIG. 85), which expanded tumor cell death regions at the expense of hypoxic areas (FIG. 85). Moreover, while a hypoxic zone at the periphery of the tumor cell death region was detected in 4T-Trap-treated mice, with 4T-Trap plus VEGF-Trap treatment, the tumor cell death region expanded to the outer boundary of hypoxic areas (FIG. 86). Although VEGF-Trap had no impact on mammary tumor growth or survival of hCD4PyMT mice (FIGS. 87 and 88), it enhanced the tumor suppression and survival benefits of 4T-Trap (FIGS. 87 and 88). These findings demonstrated that 4T-Trap can be combined with VEGF inhibitors to further restrain the tumor vasculature-mediated cancer progression.

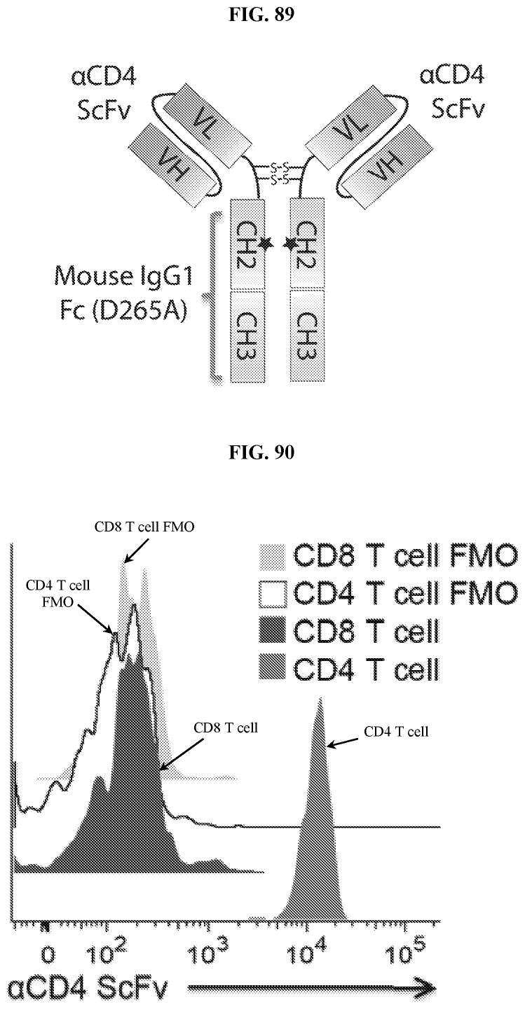

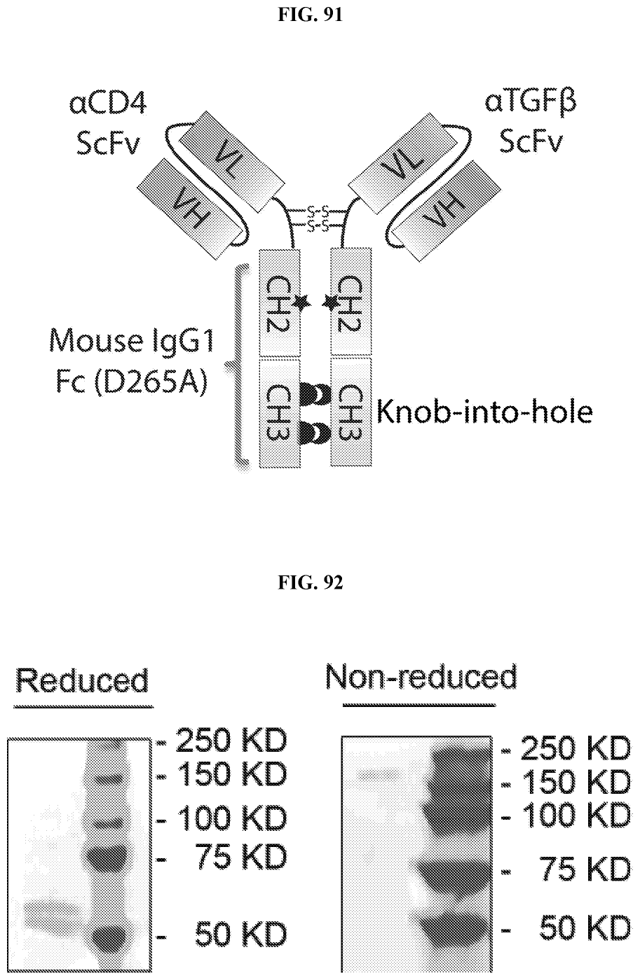

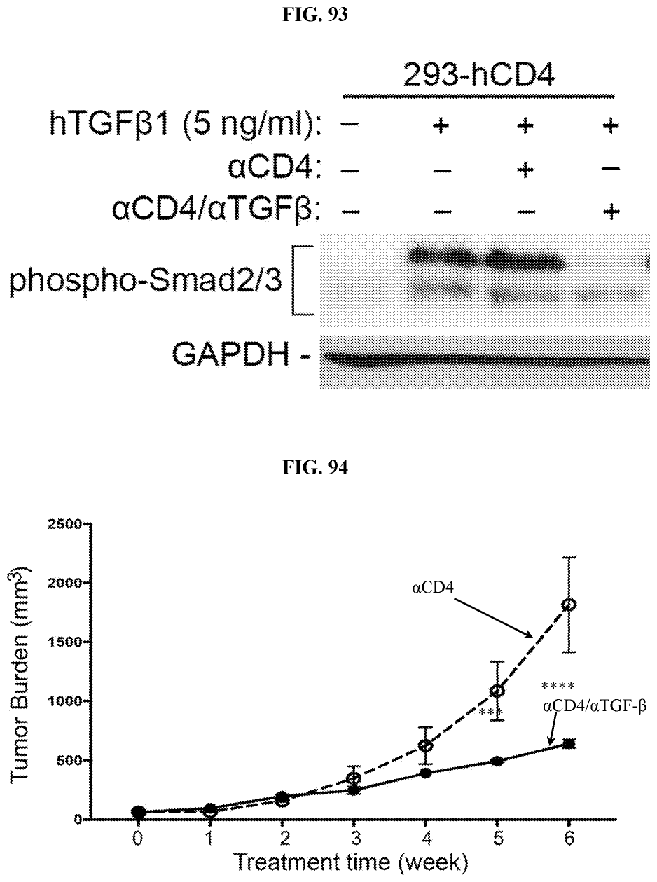

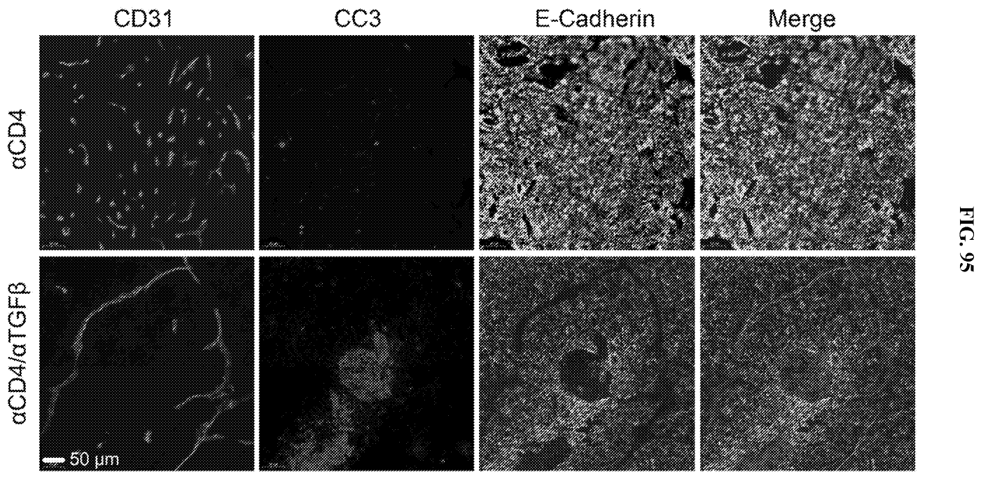

To explore additional modalities of TGF-β inhibition in helper T cells, we engineered a single-chain variable fragment (ScFv) of ibalizumab and constructed an ScFv-Fc fusion protein (FIG. 89). Flow cytometry experiments revealed that anti-CD4 ScFv specifically binds to CD4⁺ T cells from human PBMC (FIG. 90). A bispecific antibody fusion with the anti-CD4 ScFv and an anti-TGF-β ScFv adapted from fresolimumab was subsequently generated in a framework of mouse IgG1 Fc that harbors a D265A mutation and a knob-into-hole configuration (FIG. 91). Production and purity of the αCD4/αTGF-β bispecific antibody were validated by SDS-PAGE under reduced and non-reduced conditions (FIG. 92). Its TGF-β inhibitory function was validated by the phosphorylation levels of Smad2/3 in 293-hCD4 cells (FIG. 93). hCD4PyMT mice bearing 5×5 mm tumors were treated with intravenous αCD4 or αCD4/αTGF-β, and monitored for tumor growth for 6 weeks (FIG. 94). Compared to αCD4, αCD4/αTGF-β caused profound inhibition of mammary tumor growth (FIG. 94). Furthermore, immunofluorescent staining showed that αCD4/αTGF-β treatment induced a tumor tissue healing response revealed by the organized vasculature and tumor cell death (FIG. 95). These findings demonstrated that a formality of αCD4/αTGF-β and its like could as well be developed as an effective cancer therapeutic agent.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A fusion protein comprising a CD4 targeting moiety fused with an immunomodulatory moiety, wherein:
the CD4 targeting moiety comprises a heavy chain immunoglobulin variable domain (V_{H}) and a light chain immunoglobulin variable domain (V_{L}), wherein:
(a) the V_{H} comprises a V_{H}-CDR1 sequence of GYTFTSYVIH (SEQ ID NO: 6), a V_{H}-CDR2 sequence of YINPYNDGTDYDEKFKG (SEQ ID NO: 7), and a V_{H}-CDR3 sequence of EKDNYATGAWFAY (SEQ ID NO: 8), and
(b) the V_{L} comprises a V_{L}-CDR1 sequence of KSSQSLLYSTNQKNYLA (SEQ ID NO: 2), a V_{L}-CDR2 sequence of WASTRES (SEQ ID NO: 3), and a V_{L}-CDR3 sequence of QQYYSYRT (SEQ ID NO: 4), optionally wherein the V_{H} comprises an amino acid sequence that is at least 80%, at least 85%, at least 95%, or 100% identical to SEQ ID NO: 5; and/or the V_{L} comprises an amino acid sequence that is at least 80%, at least 85%, at least 95%, or 100% identical to SEQ ID NO: 1; and
the immunomodulatory moiety comprises an amino acid sequence of TGF-β receptor II (TGF-βRII) selected from the group consisting of SEQ ID NOs: 11-12 and 15-17.

2. The fusion protein of claim 1, wherein the CD4 targeting moiety comprises an antibody or an antigen binding fragment that specifically binds a CD4 epitope optionally wherein
the antibody or antigen binding fragment further comprises a Fc domain of an isotype selected from the group consisting of IgG1, IgG2, IgG3, IgG4, IgA1, IgA2, IgM, IgD, and IgE; or
the antibody or antigen binding fragment comprises an IgG1 constant region comprising one or more amino acid substitutions selected from the group consisting of D265A, N297A, K322A, L234F, L235E and P331S; or
the antibody or antigen binding fragment comprises an IgG4 constant region comprising a S228P mutation; or
the antigen binding fragment is selected from the group consisting of Fab, F(ab′)₂, Fab′, scFᵥ, and Fᵥ, or
the antibody is a monoclonal antibody, a chimeric antibody, or a humanized antibody; or
the antibody comprises a heavy chain (HC) amino acid sequence that is at least 95% identical to the HC sequence present in any one of SEQ ID NOs: 24-26; and/or a LC sequence that is at least 95% identical to the LC sequence present in SEQ ID NO: 27; or
the antibody comprises a HC amino acid sequence and a LC amino acid sequence selected from the group consisting of: SEQ ID NO: 24 and SEQ ID NO: 27; SEQ ID NO: 25 and SEQ ID NO: 27; and SEQ ID NO: 26 and SEQ ID NO: 27, respectively.

3. (canceled)

4. (canceled)

5. (canceled)

6. (canceled)

7. (canceled)

8. (canceled)

9. The fusion protein of claim 1, wherein the immunomodulatory moiety is fused to the C-terminus or the N-terminus of the CD4 targeting moiety; or
wherein the CD4 targeting moiety is fused with the immunomodulatory moiety via a peptide linker, optionally wherein the peptide linker comprises the amino acid sequence GGGGS (SEQ ID NO: 44).

10. (canceled)

11. (canceled)

12. (canceled)

13. (canceled)

14. (canceled)

15. A fusion protein comprising
(a) an immunomodulatory moiety fused to a first heterodimerization domain, wherein
(i) the first heterodimerization domain is incapable of forming a stable homodimer with another first heterodimerization domain, and
(ii) the immunomodulatory moiety comprises an amino acid sequence of TGF-β receptor II (TGF-βRII) selected from the group consisting of SEQ ID NOs: 11-12 and 15-17; and
(b) a CD4 targeting moiety fused to a second heterodimerization domain, wherein
(i) the second heterodimerization domain comprises an amino acid sequence or a nucleic acid sequence that is distinct from the first heterodimerization domain,
(ii) the second heterodimerization domain is incapable of forming a stable homodimer with another second heterodimerization domain,
(iii) the second heterodimerization domain is configured to form a heterodimer with the first heterodimerization domain, and
(iv) the CD4 targeting moiety comprises a heavy chain immunoglobulin variable domain (V_{H}) and a light chain immunoglobulin variable domain (V_{L}), wherein:
the V_{H} comprises a V_{H}-CDR1 sequence of GYTFTSYVIH (SEQ ID NO: 6), a V_{H}-CDR2 sequence of YINPYNDGTDYDEKFKG (SEQ ID NO: 7), and a V_{H}-CDR3 sequence of EKDNYATGAWFAY (SEQ ID NO: 8), and
the V_{L} comprises a V_{L}-CDR1 sequence of KSSQSLLYSTNQKNYLA (SEQ ID NO: 2), a V_{L}-CDR2 sequence of WASTRES (SEQ ID NO: 3), and a V_{L}-CDR3 sequence of QQYYSYRT (SEQ ID NO: 4), optionally wherein the V_{H} comprises an amino acid sequence that is at least 80%, at least 85%, at least 95%, or 100% identical to SEQ ID NO: 5; and/or the V_{L} comprises an amino acid sequence that is at least 80%, at least 85%, at least 95%, or 100% identical to SEQ ID NO: 1.

16. The fusion protein of claim 15, wherein the first heterodimerization domain and/or the second heterodimerization domain is a CH2-CH3 domain and has an isotype selected from the group consisting of IgG1, IgG2, IgG3, IgG4, IgA1, IgA2, IgM, IgD, and IgE; or
wherein the first heterodimerization domain is a CH2-CH3 domain comprising T366W/S354C mutations and the second heterodimerization domain is a CH2-CH3 domain comprising T366S/L368A/Y407V/Y349C mutations; or
wherein the first heterodimerization domain and/or the second heterodimerization domain comprises one or more amino acid substitutions selected from the group consisting of D265A, N297A, K322A, L234F, L235E and P331S; or
wherein the CD4 targeting moiety comprises an antibody that includes a heavy chain (HC) amino acid sequence and a light chain (LC) amino acid sequence, optionally wherein
the heavy chain (HC) amino acid sequence is at least 95% identical to the HC sequence present in any one of SEQ ID NOs: 24-26; and/or the LC sequence is at least 95% identical to the LC sequence present in SEQ ID NO: 27, or
the HC amino acid sequence and the LC amino acid sequence is selected from the group consisting of: SEQ ID NO: 24 and SEQ ID NO: 27; SEQ ID NO: 25 and SEQ ID NO: 27; and SEQ ID NO: 26 and SEQ ID NO: 27, respectively.

17. (canceled)

18. The fusion protein of claim 15, wherein the V_{H} of the CD4 targeting moiety is linked to a CH1 domain and/or the V_{L} of the CD4 targeting moiety is linked to a CL domain.

19. (canceled)

20. (canceled)

21. (canceled)

22. (canceled)

23. (canceled)

24. (canceled)

25. (canceled)

26. (canceled)

27. A recombinant nucleic acid sequence encoding the fusion protein of claim 1.

28. A host cell or expression vector comprising the recombinant nucleic acid sequence of claim 27.

29. A composition comprising the fusion protein of claim 1 and a pharmaceutically-acceptable carrier, wherein the fusion protein is optionally conjugated to an agent selected from the group consisting of isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof.

30. A method for treating cancer in a subject in need thereof, comprising administering to the subject an effective amount of the fusion protein of claim 1, optionally wherein
the cancer is prostate cancer, pancreatic cancer, biliary cancer, colon cancer, rectal cancer, liver cancer, kidney cancer, lung cancer, testicular cancer, breast cancer, ovarian cancer, brain cancer, bladder cancer, head and neck cancers, melanoma, sarcoma, multiple myeloma, leukemia, or lymphoma; or
the fusion protein is administered to the subject separately, sequentially or simultaneously with
one or more of targeted therapies (e.g. apoptosis-inducing proteasome inhibitor, selective estrogen-receptor modulator, BCR-ABL inhibitors, BTK inhibitor, EGFR inhibitors, Janus kinase inhibitors, ALK inhibitors, Bcl-2 inhibitors, PARP inhibitors, PI3K inhibitors, MEK inhibitors, CDK inhibitors, Hsp90 inhibitors, DNA-targeting agent, NTRK inhibitors, mTOR inhibitors, BRAF inhibitors, aromatase inhibitors, cytostatic alkaloids, cytotoxic antibiotics, antimetabolites, endocrine/hormonal agents, topoisomerase inhibitors, bisphosphonate therapy agents), antiangiogenic agents (e.g., VEGF/VEGFR inhibitors), cancer immunotherapies (e.g. anti-PD-1, anti-PD-L1, anti-CTLA-4) or chemotherapeutic agents; or
one or more antiangiogenic agents selected from among antibodies, small molecule inhibitors, decoy receptors and decoy ligands (e.g., Traps).

31. (canceled)

32. (canceled)

33. (canceled)

34. A method for increasing tumor sensitivity to a therapy in a subject suffering from cancer comprising
(a) administering to the subject an effective amount of the fusion protein of claim 1; and
(b) administering to the subject an effective amount of an anti-cancer therapeutic agent optionally wherein
the anti-cancer therapeutic agent is a chemotherapeutic agent selected from the group consisting of cyclophosphamide, fluorouracil (or 5-fluorouracil or 5-FU), Leucovorin, methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, tykerb, anthracyclines (e.g., daunorubicin and doxorubicin), oxaliplatin, melphalan, etoposide, mechlorethamine, bleomycin, microtubule poisons, annonaceous acetogenins, or combinations thereof; or
the cancer is prostate cancer, pancreatic cancer, biliary cancer, colon cancer, rectal cancer, liver cancer, kidney cancer, lung cancer, testicular cancer, breast cancer, ovarian cancer, brain cancer, bladder cancer, head and neck cancers, melanoma, sarcoma, multiple myeloma, leukemia, or lymphoma.

35. (canceled)

36. (canceled)

37. A kit comprising the fusion protein of claim 1 and instructions for use.

38. A method for monitoring cancer progression in a patient in need thereof comprising
(a) administering to the patient an effective amount of the fusion protein of claim 1; and
(b) detecting tumor growth in the patient, wherein a reduction in tumor size relative to that observed in the patient prior to administration of the fusion protein is indicative of cancer arrest or cancer regression.

39. An engineered helper T cell, wherein the cell lacks detectable expression or activity of a TGF-β receptor II that comprises an amino acid sequence of any one of SEQ ID NOs: 11-12, or
wherein the cell expresses an inhibitory nucleic acid that specifically targets and inhibits the expression of a TGF-β receptor II nucleic acid sequence selected from among SEQ ID NOs: 13-14, 18-20, and 21-23, optionally wherein the inhibitory nucleic acid is an antisense oligonucleotide, a siRNA, a sgRNA or a shRNA, or
wherein the cell comprises a transgene that encodes a dominant negative TGF-β receptor II or the inhibitory nucleic acid, optionally wherein the transgene is operably linked to an ubiquitous promoter, a constitutive promoter, a T cell-specific promoter, or an inducible promoter; or
wherein the cell comprises a deletion, insertion, inversion, or frameshift mutation in a TGF-β receptor II gene encoded by the nucleic acid sequence of SEQ ID NO: 13 or SEQ ID NO: 14.

40. (canceled)

41. (canceled)

42. (canceled)

43. (canceled)

44. (canceled)

45. The engineered helper T cell of claim 39, wherein the engineered helper T cell is derived from an autologous donor or an allogeneic donor.

46. A method for inhibiting tumor growth or metastasis in a subject with cancer comprising administering to the subject an effective amount of the engineered helper T cell of claim 39, optionally wherein the engineered helper T cell is administered intravenously, intraperitoneally, subcutaneously, intramuscularly, or intratumorally.

47. (canceled)

48. The method of claim 46, further comprising
administering an additional cancer therapy selected from among chemotherapy, radiation therapy, immunotherapy, monoclonal antibodies, anti-cancer nucleic acids or proteins, anti-cancer viruses or microorganisms, and any combinations thereof to the subject; or
administering a cytokine agonist or antagonist to the subject; or
sequentially, separately, or simultaneously administering to the subject at least one chemotherapeutic agent, optionally wherein the at least one chemotherapeutic agent is selected from the group consisting of cyclophosphamide, fluorouracil (or 5-fluorouracil or 5-FU), Leucovorin, methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, tykerb, anthracyclines (e.g., daunorubicin and doxorubicin), oxaliplatin, melphalan, etoposide, mechlorethamine, bleomycin, microtubule poisons, annonaceous acetogenins, or combinations thereof.

49. (canceled)

50. (canceled)

51. The method of claim 46, wherein the cancer is prostate cancer, pancreatic cancer, biliary cancer, colon cancer, rectal cancer, liver cancer, kidney cancer, lung cancer, testicular cancer, breast cancer, ovarian cancer, brain cancer, bladder cancer, head and neck cancers, melanoma, sarcoma, multiple myeloma, leukemia, or lymphoma.

52. (canceled)

53. (canceled)

54. (canceled)

55. (canceled)

56. (canceled)

57. (canceled)

58. (canceled)

59. (canceled)

60. A kit comprising the engineered helper T cell of claim 39, and instructions for use.
